(19) United States
(12) Patent Application Publication
(10) Pub. No.: US 2021/0122739 A1
(43) Pub. Date: Apr. 29, 2021

(54) NOVEL SULFONAMIDE CARBOXAMIDE COMPOUNDS

(71) Applicant: Inflazome Limited, Dublin 2 (IE)

(72) Inventors: Matthew COOPER, Cambridge (GB); David MILLER, Cambridge (GB); Angus MACLEOD, Cambridge (GB); Stephen THOM, Nottingham (GB); Stephen ST-GALLAY, Nottingham (GB); Jonathan SHANNON, Nottingham (GB)

(73) Assignee: Inflazome Limited, Dublin 2 (IE)

(21) Appl. No.: 16/638,704

(22) PCT Filed: Aug. 15, 2018
(86) PCT No.: PCT/EP2018/072115

§ 371 Date: Feb. 12, 2020

(30) Foreign Application Priority Data
Aug. 15, 2017 (GB) 1713082.4
Nov. 9, 2017 (GB) 1718563.8
Dec. 22, 2017 (GB) 1721726.6
Dec. 22, 2017 (GB) 1721729.0
Dec. 22, 2017 (GB) 1721731.6
Jul. 4, 2018 (GB) 1810983.5

(51) Int. Cl. C07D 403/06; C07D 231/18; C07D 403/04; C07D 487/04; C07D 401/04; C07D 405/12; C07D 413/06; C07D 401/06; C07D 403/12; C07D 417/12

(52) U.S. Cl. CPC C07D 403/06; C07D 231/18; C07D 403/04; C07D 487/04; C07D 417/12; C07D 405/12; C07D 413/06; C07D 401/06; C07D 403/12; C07D 401/04

(57) ABSTRACT

The present invention relates to sulfonylureas and sulfonylthioureas comprising a 5-membered heteroaryl group substituted with an amide-containing group. The present invention further relates to salts, solvates and prodrugs of such compounds, to pharmaceutical compositions comprising such compounds, and to the use of such compounds in the treatment and prevention of medical disorders and diseases, most especially by the inhibition of NLRP₃.

### FIELD OF THE INVENTION

The present invention relates to sulfonylureas and sulfonylthioureas comprising a 5-membered heteroaryl group substituted with an amide-containing group, and to associated salts, solvates, prodrugs and pharmaceutical compositions. The present invention further relates to the use of such compounds in the treatment and prevention of medical disorders and diseases, most especially by NLRP3 inhibition.

### BACKGROUND

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1p and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlr3−/− mice, but there have also been insights into the specific activation of NLRP3 in these diseases. In type 2 diabetes mellitus (T2D), the deposition of islet amyloid polypeptide in the pancreas activates NLRP3 and IL-1β signaling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-p-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Some diarylsulfonylurea-containing compounds have been identified as cytokine release inhibitory drugs (CRIDs) (Perregaux et al.; J. Pharmacol. Exp. Ther. 299, 187-197, 2001). CRIDs are a class of diarylsulfonylurea containing compounds that inhibit the post-translational processing of IL-1β. Post-translational processing of IL-1β is accompanied by activation of caspase-1 and cell death. CRIDs arrest activated monocytes so that caspase-1 remains inactive and plasma membrane latency is preserved.

Certain sulfonylurea-containing compounds are also disclosed as inhibitors of NLRP3 (see for example, Baldwin et al., J. Med. Chem., 59(5), 1691-1710, 2016; and WO 2016/131098 A1, WO 2017/129897 A1, WO 2017/140778 A1, WO 2017/184604 A1, WO 2017/184623 A1, WO 2017/184624 A1, WO 2018/136890 A1 and WO 2018/015445 A1).

There is a need to provide compounds with improved pharmacological and/or physiological and/or physicochemical properties and/or those that provide a useful alternative to known compounds.

### SUMMARY OF THE INVENTION

A first aspect of the invention provides a compound of formula (I):

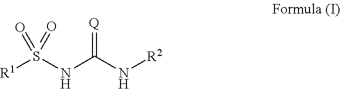

wherein:
Q is selected from O or S;
R¹ is a 5-membered heteroaryl group substituted with at least one group R^{X}, wherein R^{X} is any group comprising an amide group, wherein the 5-membered heteroaryl group may optionally be further substituted; and
R² is a cyclic group substituted at the α-position, wherein R² may optionally be further substituted;
provided that the compound is not:

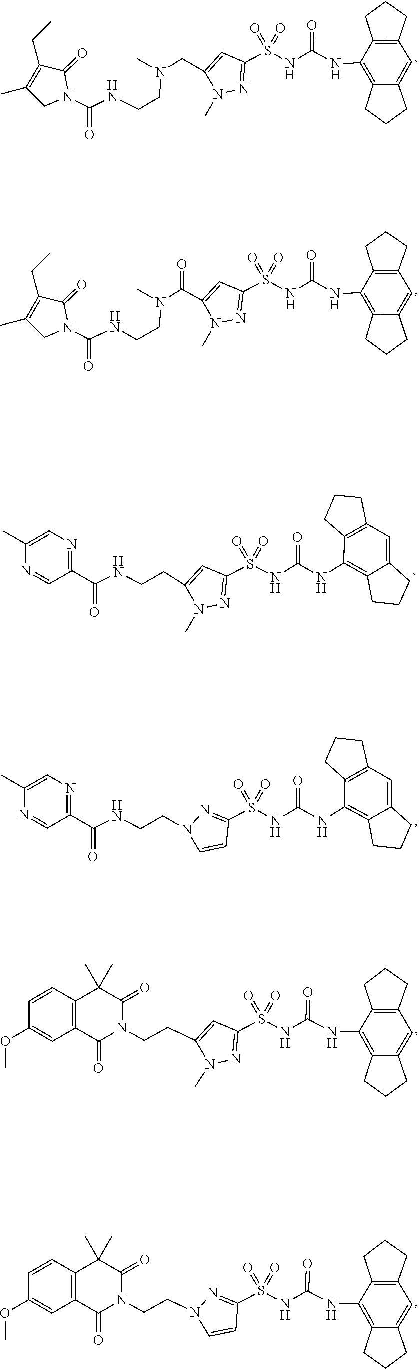 [SMILES: CCC1=C(C)CN(C(=O)NCCN(C)Cc2cc(S(=O)(=O)NC(=O)Nc3c4c(cc5c3CCC5)CCC4)nn2C)C1=O ; CCC1=C(C)CN(C(=O)NCCN(C)C(=O)c2cc(S(=O)(=O)NC(=O)Nc3c4c(cc5c3CCC5)CCC4)nn2C)C1=O ; Cc1cnc(C(=O)NCCc2cc(S(=O)(=O)NC(=O)Nc3c4c(cc5c3CCC5)CCC4)nn2C)cn1 ; Cc1cnc(C(=O)NCCn2ccc(S(=O)(=O)NC(=O)Nc3c4c(cc5c3CCC5)CCC4)n2)cn1 ; COc1ccc2c(c1)C(=O)N(CCc1cc(S(=O)(=O)NC(=O)Nc3c4c(cc5c3CCC5)CCC4)nn1C)C(=O)C2(C)C ; COc1ccc2c(c1)C(=O)N(CCn1ccc(S(=O)(=O)NC(=O)Nc3c4c(cc5c3CCC5)CCC4)n1)C(=O)C2(C)C]

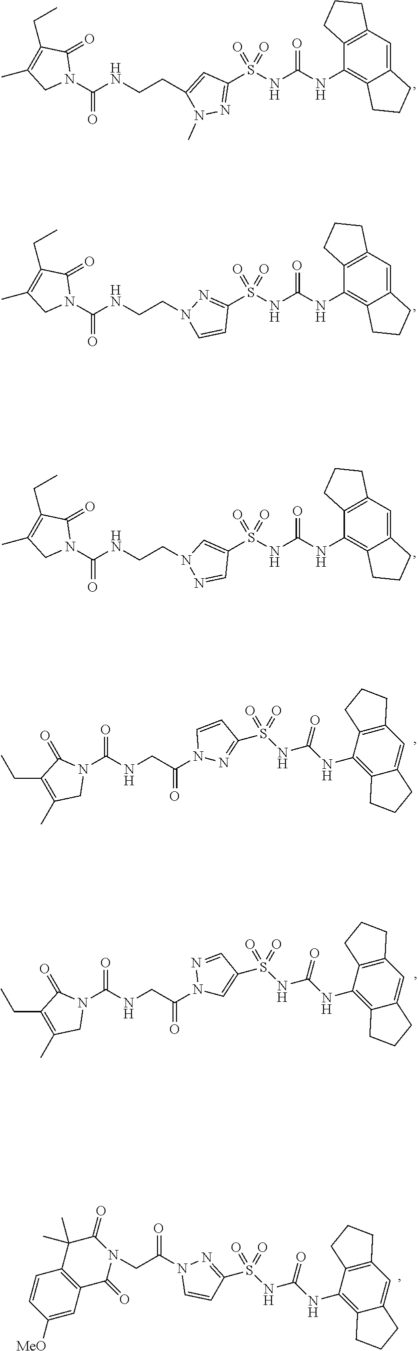 [SMILES: CCC1=C(C)CN(C(=O)NCCc2cc(S(=O)(=O)NC(=O)Nc3c4c(cc5c3CCC5)CCC4)nn2C)C1=O ; CCC1=C(C)CN(C(=O)NCCn2ccc(S(=O)(=O)NC(=O)Nc3c4c(cc5c3CCC5)CCC4)n2)C1=O ; CCC1=C(C)CN(C(=O)NCCn2cc(S(=O)(=O)NC(=O)Nc3c4c(cc5c3CCC5)CCC4)cn2)C1=O ; CCC1=C(C)CN(C(=O)NCC(=O)n2ccc(S(=O)(=O)NC(=O)Nc3c4c(cc5c3CCC5)CCC4)n2)C1=O ; CCC1=C(C)CN(C(=O)NCC(=O)n2cc(S(=O)(=O)NC(=O)Nc3c4c(cc5c3CCC5)CCC4)cn2)C1=O ; COc1ccc2c(c1)C(=O)N(CC(=O)n1ccc(S(=O)(=O)NC(=O)Nc3c4c(cc5c3CCC5)CCC4)n1)C(=O)C2(C)C]

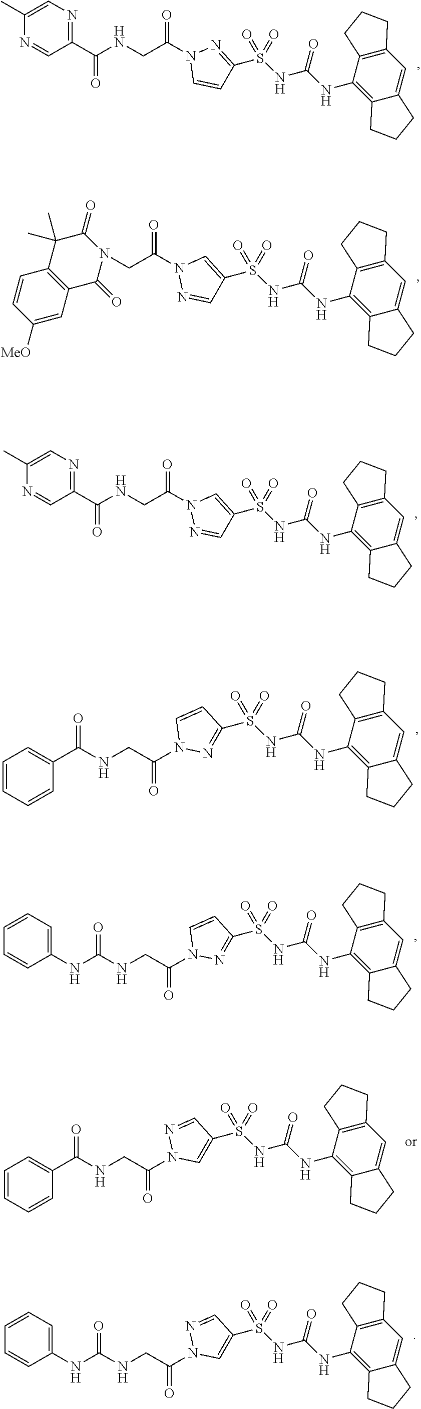

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

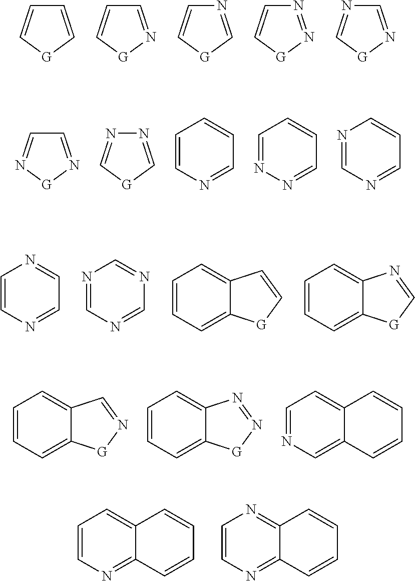

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety:

(i) each hydrogen atom may optionally be replaced by a group independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR³; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(N₂)R^{β}; —R^{α}—C(═NH)R^{β}; —R^{α}—C(═NH)NH₂; —R^{α}—C(═NH)NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(N₂)R^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR—COR^{β}; —R^{α}—CONH₂; —R^{α}—CONHR^{β}; —R^{β}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; and/or
(ii) any two hydrogen atoms attached to the same atom may optionally be replaced by a π-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N═N—, —N(R^{β})—, —N(O)(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —COO(C₁-C₄ alkyl), —COO(C₁-C₄ haloalkyl), halo, —OH, —NH₂, —CN, —C≡CH, oxo (═O), or 4- to 6-membered heterocyclic group.

Typically, the compounds of the present invention comprise at most one quaternary ammonium group such as —N⁺(R^{β})₃ or —N⁺(R^{β})₂—.

Where reference is made to a —R^{α}—C(N₂)R^{β} group, what is intended is:

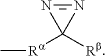

Typically, in an optionally substituted group or moiety:

(i) each hydrogen atom may optionally be replaced by a group independently selected from halo; —CN; —NO₂; —N₃; —R; —OH; —OR; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —NH—R^{α}—OH; —NH—R^{α}—OR^{β}; —NH—R^{α}—NH₂; —NH—R^{α}—NHR^{β}; —NH—R^{α}—N(R^{β})₂; —NR^{β}—R^{α}—OH; —NR^{β}—R^{α}—OR^{β}; —NR^{β}—R^{α}—NH₂; —NR^{β}—R^{α}—NHR^{β}; or —NR^{β}—R^{α}—N(R^{β})₂; and/or
(ii) any two hydrogen atoms attached to the same carbon atom may optionally be replaced by a π-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH, oxo (═O), or 4- to 6-membered heterocyclic group.

Alternately in the optionally substituted groups or moieties defined immediately above, each —R^{β} may be independently selected from a C₁-C₆ alkyl, C₂-C₆ alkenyl, C₂-C₆ alkynyl or C₂-C₆ cyclic group, or any two —R^{β} attached to the same nitrogen atom may, together with the nitrogen atom to which they are attached, form a C₂-C₇ cyclic group, wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), halo, —OH, —NH₂, —CN, —C≡CH, oxo (═O), or 4- to 6-membered heterocyclic group.

More typically, in an optionally substituted group or moiety:

(i) each hydrogen atom may optionally be replaced by a group independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β}; and/or
(ii) any two hydrogen atoms attached to the same carbon atom may optionally be replaced by a π-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH, oxo (═O), or 4- to 6-membered heterocyclic group.

Alternately in the optionally substituted groups or moieties defined immediately above, each —R^{β} may be independently selected from a C₁-C₆ alkyl, C₂-C₆ alkenyl, C₂-C₆ alkynyl or C₂-C₆ cyclic group, or any two —R^{β} attached to the same nitrogen atom may, together with the nitrogen atom to which they are attached, form a C₂-C₇ cyclic group, wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), halo, —OH, —NH₂, —CN, —C≡CH, oxo (═O), or 4- to 6-membered heterocyclic group.

More typically, in an optionally substituted group or moiety:

(i) each hydrogen atom may optionally be replaced by a group independently selected from halo; —CN; —R^{β}; —OH; —OR^{β}; —R^{α}-halo; —R^{α}—CN; —R^{α}—R^{β}; —R^{α}—OH; —R^{α}—OR^{β}; —SR^{β}; —SOR^{β}; —SO₂H; —SO₂R^{β}; —SO₂NH₂; —SO₂NHR^{β}; —SO₂N(R^{β})₂; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}: —R^{α}—COOH; —R^{α}—COOR^{β}; or —R^{α}—OCOR^{β}; and/or
(ii) any two hydrogen atoms attached to the same carbon atom may optionally be replaced by a π-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, halo, —OH, or 4- to 6-membered heterocyclic group.

Alternately in the optionally substituted groups or moieties defined immediately above, each —R^{β} may be independently selected from a C₁-C₆ alkyl, C₂-C₆ alkenyl, C₂-C₆ alkynyl or C₂-C₆ cyclic group, or any two —R^{β} attached to the same nitrogen atom may, together with the nitrogen atom to which they are attached, form a C₂-C₇ cyclic group, wherein any —R^{β} may optionally be substituted with one or more C₁-C₄ alkyl, C₁-C₄ haloalkyl, halo, —OH, or 4- to 6-membered heterocyclic group.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{β})—, —N(O)(R^{β})—, —N⁺(R^{β})₂— or —R^{α}—) of an optionally substituted group or moiety (e.g. R¹) must only be attached to the specified group or moiety and may not be attached to a second group or moiety (e.g. R²), even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

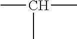

is replaced by

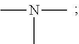

—CH₂— is replaced by —NH—, —O— or —S—;
—CH₃ is replaced by —NH₂, —OH or —SH;
—CH═ is replaced by —N═;
CH₂═ is replaced by NH═, O═ or S═; or
CH≡ is replaced by N≡;
   provided that the resultant group comprises at least one carbon atom. For example, methoxy, dimethylamino and aminoethyl groups are considered to be hydrocarbyl groups including one or more heteroatoms N, O or S in their carbon skeleton.

Where reference is made to a —CH₂— group in the backbone of a hydrocarbyl or other group being replaced by a —N(O)(R^{β})— or —N⁺(R^{β})₂— group, what is intended is that:
—CH₂— is replaced by

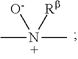

or
—CH₂— is replaced by

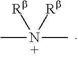

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are not to be counted as carbon atoms when calculating the number of carbon atoms in a Cₓ-C_{y} group. For example, a morpholinyl group is to be considered a C₄ heterocyclic group, not a C₆ heterocyclic group.

For the purposes of the present specification, where it is stated that a first atom or group is “directly attached” to a second atom or group it is to be understood that the first atom or group is covalently bonded to the second atom or group with no intervening atom(s) or groups being present. So, for example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

As stated, R¹ is a 5-membered heteroaryl group substituted with at least one group R^{X}, wherein R^{X} is any group comprising an amide group, wherein the 5-membered heteroaryl group may optionally be further substituted.

For the purposes of the present specification, where it is stated that a substituent, group or moiety “is a” specific group, it is to be understood that the specific group is directly attached to the remainder of the molecule, i.e. via a covalent bond with no intervening atom(s) or groups being present. Thus, in the first aspect of the invention, where it is stated that “R¹ is a 5-membered heteroaryl group” it is to be understood that a ring atom of the 5-membered ring of the 5-membered heteroaryl group is directly attached to the sulfur atom of the sulfonyl group, with no intervening atom(s) or groups being present. Similarly, where it is stated that “R² is a cyclic group”, it is to be understood that a ring atom of the cyclic group is directly attached to the nitrogen atom of the (thio)urea group, with no intervening atom(s) or groups being present. For the avoidance of doubt, R¹ is not attached to the sulfur atom of the sulfonyl group via the group R^{X} or any optional substituent.

Conversely, in the first aspect of the invention, where it is stated that “R^{X} is any group comprising an amide group”, it is to be understood only that the group R^{X} includes an amide group; such a group R^{X} may further comprise additional atoms, groups or moieties, which may connect the amide group to the 5-membered heteroaryl group of R¹.

For the purposes of the present specification, an “amide group” is considered to be any group comprising the structure:

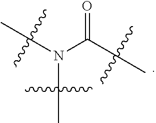 [SMILES: *C(=O)N(*)*]

Accordingly, the term “amide group” includes urea groups. Typically however, R^{X} is any group comprising an amide group wherein the carbon atom of the amide group is directly attached to another carbon atom or a hydrogen atom. More typically, R^{X} is any group comprising an amide group wherein the carbon atom of the amide group is directly attached to another carbon atom.

For the avoidance of doubt, R^{X} must comprise both the nitrogen atom and the carbonyl group of the amide moiety; an acetyl substituent directly attached to a nitrogen ring atom of the 5-membered heteroaryl group of R¹ is not considered to be a substituent R^{X}. The group R^{X} may contain a single amide group or more than one amide group. Typically the group R^{X} contains a single amide group.

Unless stated otherwise, R^{X} may be a monovalent substituent or a divalent or multivalent substituent. Where R^{X} is divalent or multivalent, R^{X} may be attached to the 5-membered heteroaryl group of R¹ via a fused ring structure.

Typically, R^{X} is monovalent.

In one embodiment, where R^{X} is monovalent, —R^{X} is any saturated or unsaturated hydrocarbyl group, wherein the hydrocarbyl group may be straight-chained or branched, or be or include cyclic groups, wherein the hydrocarbyl group may optionally be substituted, wherein the hydrocarbyl group includes at least one amide group in its carbon skeleton, and wherein the hydrocarbyl group may optionally include one or more further heteroatoms N, O or S in its carbon skeleton. Where the hydrocarbyl group of —R^{X} is optionally substituted, typically it is substituted with one or more groups selected from halo, —CN, —OH, —NH₂, oxo (═O) and ═NH.

In one embodiment, where R^{X} is monovalent, —R^{X} is any saturated hydrocarbyl group (such as a C₁-C₁₅ or a C₂-C₇ saturated hydrocarbyl group), wherein the hydrocarbyl group may be straight-chained or branched, or be or include cyclic groups, wherein the hydrocarbyl group may optionally be substituted with one or more (such as one, two or three) groups selected from halo, —CN, —OH, —NH₂, oxo (═O) and ═NH, wherein the hydrocarbyl group includes at least one amide group in its carbon skeleton, and wherein the hydrocarbyl group may optionally include one, two or three further heteroatoms N and/or O in its carbon skeleton.

In another embodiment, where R^{X} is monovalent, —R^{X} is:

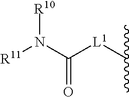

wherein:
L¹ is a bond or an alkylene, alkenylene or alkynylene group, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted;
R¹⁰ and R¹¹ are each independently selected from hydrogen or an alkyl, alkenyl, alkynyl or R¹²-L²- group, wherein the alkyl, alkenyl or alkynyl group may optionally be substituted, or R¹⁰ and R¹¹ together with the nitrogen atom to which they are attached form a heterocyclic group, wherein the heterocyclic group may optionally be substituted;
or R¹⁰ and L¹, together with the carbon and nitrogen atoms to which they are attached, form a divalent heterocyclic, (divalent heterocyclic)alkylene, (divalent heterocyclic)alkenylene or (divalent heterocyclic)alkynylene group, wherein one or more carbon atoms in the backbone of the alkylene, alkenylene or alkynylene group may optionally be replaced by one or more heteroatoms N, O or S, and wherein the divalent heterocyclic, (divalent heterocyclic)alkylene, (divalent heterocyclic)alkenylene or (divalent heterocyclic)alkynylene group may optionally be substituted;
each L² is independently selected from a bond or an alkylene, alkenylene or alkynylene group, wherein the alkylene, alkenylene or alkynylene group may optionally be substituted; and
each R¹² is independently selected from any cyclic group, wherein the cyclic group may optionally be substituted.

In one aspect of the above embodiment:
L¹ is a bond or an alkylene group, wherein one or more carbon atoms in the backbone of the alkylene group may optionally be replaced by one or more heteroatoms N, O or S, and wherein the alkylene group may optionally be substituted;
R¹⁰ and R¹¹ are each independently selected from hydrogen or an alkyl or R¹²-L²- group, wherein the alkyl group may optionally be substituted, or R¹⁰ and R¹¹ together with the nitrogen atom to which they are attached form a saturated heterocyclic group, wherein the saturated heterocyclic group may optionally be substituted;
or R¹⁰ and L¹, together with the carbon and nitrogen atoms to which they are attached, form a divalent saturated heterocyclic or (divalent saturated heterocyclic)alkylene group, wherein one or more carbon atoms in the backbone of the alkylene group may optionally be replaced by one or more heteroatoms N, O or S, and wherein the divalent saturated heterocyclic or (divalent saturated heterocyclic)alkylene group may optionally be substituted;
each L² is independently selected from a bond or an alkylene group, wherein the alkylene group may optionally be substituted; and
each R¹² is independently selected from a cycloalkyl or saturated heterocyclic group, wherein the cycloalkyl or saturated heterocyclic group may optionally be substituted.

Typically:
L¹ is a bond or a C₁-C₆ alkylene group;
R¹⁰ and R¹¹ are each independently selected from hydrogen or a C₁-C₆ alkyl or R¹²-L²- group, or R¹⁰ and R¹¹ together with the nitrogen atom to which they are attached form a 3 to 7 membered heterocyclic group;
each L² is independently selected from a bond or a C₁-C₆ alkylene group;
each R¹² is independently any 3 to 7 membered cyclic group;
any C₁-C₆ alkylene or C₁-C₆ alkyl group may optionally be substituted with one or more groups independently selected from halo, —CN, —OH, —NH₂, oxo (═O), —OR¹³, —NHR¹³, —N(R¹³)₂, —NHCOR¹³ and —N(R¹³)COR¹³;
any 3 to 7 membered heterocyclic or 3 to 7 membered cyclic group may optionally be substituted with one or more groups independently selected from halo, —CN, —OH, —NH₂, oxo (═O), —R¹³, —OR¹³, —NHR¹³, —N(R¹³)₂, —NHCOR¹³ and —N(R¹³)COR¹³; and
each R¹³ is independently selected from a C₁-C₄ alkyl or C₁-C₄ haloalkyl group.

In one aspect of the above embodiment:
L is a bond or a C₁-C₆ alkylene group;
R¹⁰ and R¹¹ are each independently selected from hydrogen or a C₁-C₆ alkyl or R¹²-L²- group, or R¹⁰ and R¹¹ together with the nitrogen atom to which they are attached form a 3 to 7 membered saturated heterocyclic group;
each L² is independently selected from a bond or a C₁-C₆ alkylene group;
each R¹² is independently a 3 to 7 membered cycloalkyl or a 3 to 7 membered saturated heterocyclic group;
any C₁-C₆ alkylene or C₁-C₆ alkyl group may optionally be substituted with one or more groups independently selected from halo, —CN, —OH, —NH₂, oxo (═O), —OR¹³, —NHR¹³, —N(R¹³)₂, —NHCOR¹³ and —N(R¹³)COR¹³;
any 3 to 7 membered cycloalkyl or a 3 to 7 membered saturated heterocyclic group may optionally be substituted with one or more groups independently selected from halo, —CN, —OH, —NH₂, oxo (═O), —R¹³, —OR¹³, —NHR¹³, —N(R¹³)₂, —NHCOR¹³ and —N(R¹³)COR¹³; and
each R¹³ is independently selected from a C₁-C₄ alkyl or C₁-C₄ haloalkyl group.

More typically:
L¹ is a bond or a C₁-C₄ alkylene group;
R¹⁰ is hydrogen or a C₁-C₄ alkyl or R¹²-L²- group, and R¹¹ is a C₁-C₄ alkyl or R¹²-L²- group, or R¹⁰ and R¹¹ together with the nitrogen atom to which they are attached form a 3 to 6 membered saturated heterocyclic group;
each L² is independently selected from a bond or a C₁-C₄ alkylene group;
each R¹² is independently selected from a 3 to 6 membered cycloalkyl, a 3 to 6 membered saturated heterocyclic, a phenyl or a 5 or 6 membered heteroaryl group;
any C₁-C₄ alkylene or C₁-C₄ alkyl group may optionally be substituted with one or more groups independently selected from halo, —CN, —OH, —NH₂, oxo (═O), —OR¹³, —NHR¹³, —N(R¹³)₂, —NHCOR¹³ and —N(R¹³)COR¹³;
any 3 to 6 membered cycloalkyl, 3 to 6 membered saturated heterocyclic, phenyl or 5 or 6 membered heteroaryl group may optionally be substituted with one or more groups independently selected from halo, —CN, —OH, —NH₂, oxo (═O), —R¹³, —OR¹³, —NHR¹³, —N(R¹³)₂, —NHCOR¹³ and —N(R¹³)COR¹³; and
each R¹³ is independently selected from a methyl or halomethyl group.

In one aspect of the above embodiment, each R¹² is independently selected from a 3 to 6 membered cycloalkyl group or a 3 to 6 membered saturated heterocyclic group, wherein any 3 to 6 membered cycloalkyl or 3 to 6 membered saturated heterocyclic group may optionally be substituted with one or more groups independently selected from halo, —CN, —OH, —NH₂, oxo (═O), —R¹³, —OR¹³, —NHR¹³, —N(R¹³)₂, —NHCOR¹³ and —N(R¹³)COR¹³, and wherein each R¹³ is independently selected from a methyl or halomethyl group.

More typically still:
L¹ is a bond or a —CH₂— group;
R¹⁰ is hydrogen or a C₁-C₃ alkyl group, and R¹ is a C₁-C₃ alkyl or R¹²-L²- group, or R¹⁰ and R¹¹ together form a C₂-C₄ alkylene group, wherein the C₂-C₄ alkylene group may optionally include an oxygen atom in its carbon skeleton;
each L² is independently selected from a bond or a —CH₂— group;
each R¹² is independently selected from a 3 to 5 membered cycloalkyl group, a 4 or 5 membered saturated heterocyclic group, or a 5 membered heteroaryl group;
any C₁-C₃ alkyl group may optionally be substituted with one or more groups independently selected from fluoro, chloro, —CN, —OH, oxo (═O), —OR¹³, —NHR¹³, —N(R¹³)₂, —NHCOR¹³ and —N(R¹³)COR¹³;
any 3 to 5 membered cycloalkyl, 4 or 5 membered saturated heterocyclic, or 5 membered heteroaryl group may optionally be substituted with one or more groups independently selected from fluoro, chloro, —CN, —OH, oxo (═O), —R¹³, —OR¹³, —NHR¹³, —N(R¹³)₂, —NHCOR¹³ and —N(R¹³)COR¹³;
any —CH₂— group may optionally be substituted with one or more groups independently selected from fluoro, chloro and —R¹³; and
each R¹³ is a methyl group, wherein any methyl group may optionally be substituted with one or more fluoro and/or chloro groups.

In one aspect of the above embodiment, each R¹² is independently selected from a 3 to 5 membered cycloalkyl group or a 4 or 5 membered saturated heterocyclic group, wherein any 3 to 5 membered cycloalkyl or 4 or 5 membered saturated heterocyclic group may optionally be substituted with one or more groups independently selected from fluoro, chloro, —CN, —OH, oxo (═O), —R¹³, —OR¹³, —NHR¹³, —N(R¹³)₂, —NHCOR¹³ and —N(R¹³)COR¹³, wherein each R¹³ is a methyl group, and wherein any methyl group may optionally be substituted with one or more fluoro and/or chloro groups.

In another embodiment,
L is a bond or a —CH₂—, —CH(R^{13a})— or —C(R^{13a})₂— group;
R¹⁰ is hydrogen or a C₁-C₃ alkyl group, and R¹ is a C₁-C₃ alkyl or R¹²-L²- group, or R¹⁰ and R¹¹ together with the nitrogen atom to which they are attached form a 3 to 5 membered saturated heterocyclic group;
each L² is independently selected from a bond or a —CH₂—, —CH(R^{13a})— or —C(R^{13a})₂— group;
each R¹² is independently selected from a 3 to 5 membered cycloalkyl group, or a 3 to 5 membered saturated heterocyclic group;
any C₁-C₃ alkyl group may optionally be substituted with one or more groups independently selected from fluoro, chloro, —CN, —OH, oxo (═O), —OR¹³, —NHR¹³, —N(R¹³)₂, —NHCOR¹³ and —N(R¹³)COR¹³;
any 3 to 5 membered cycloalkyl or 3 to 5 membered saturated heterocyclic group may optionally be substituted with one or more groups independently selected from fluoro, chloro, —CN, —OH, oxo (═O), —R¹³, —OR¹³, —NHR¹³, —N(R¹³)₂, —NHCOR¹³ and —N(R¹³)COR¹³;
each R^{13a} is independently selected from a fluoro, chloro or R¹³ group; and
each R¹³ is a methyl group, wherein any methyl group may optionally be substituted with one or more fluoro and/or chloro groups.

Most typically, in any of the above embodiments, L is a bond.

In another embodiment, where R^{X} is monovalent, —R^{X} is:

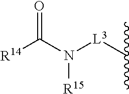

wherein:
L³ is a bond or an alkylene, alkenylene or alkynylene group, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted;
R¹⁴ is hydrogen or an alkyl, alkenyl, alkynyl or R¹⁶-L⁴- group, wherein the alkyl, alkenyl or alkynyl group may optionally be substituted;
R¹⁵ is hydrogen or an alkyl, alkenyl, alkynyl or R¹⁶-L⁴- group, wherein the alkyl, alkenyl or alkynyl group may optionally be substituted;
or R¹⁴ and R¹⁵ together with the carbon and nitrogen atoms to which they are attached form a heterocyclic group, wherein the heterocyclic group may optionally be substituted;
or R¹⁴ and L³, together with the carbon and nitrogen atoms to which they are attached, form a heterocyclic, (heterocyclic)alkylene, (heterocyclic)alkenylene or (heterocyclic)alkynylene group, wherein one or more carbon atoms in the backbone of the alkylene, alkenylene or alkynylene group may optionally be replaced by one or more heteroatoms N, O or S, and wherein the heterocyclic, (heterocyclic)alkylene, (heterocyclic)alkenylene or (heterocyclic)alkynylene group may optionally be substituted;
or R¹⁵ and L³, together with the nitrogen atom to which they are attached, form a divalent heterocyclic, (divalent heterocyclic)alkylene, (divalent heterocyclic)alkenylene or (divalent heterocyclic)alkynylene group, wherein one or more carbon atoms in the backbone of the alkylene, alkenylene or alkynylene group may optionally be replaced by one or more heteroatoms N, O or S, and wherein the divalent heterocyclic, (divalent heterocyclic)alkylene, (divalent heterocyclic)alkenylene or (divalent heterocyclic)alkynylene group may optionally be substituted;
each L⁴ is independently selected from a bond or an alkylene, alkenylene or alkynylene group, wherein the alkylene, alkenylene or alkynylene group may optionally be substituted; and
each R¹⁶ is independently selected from any cyclic group, wherein the cyclic group may optionally be substituted.

In one aspect of the above embodiment:
L³ is a bond or an alkylene group, wherein one or more carbon atoms in the backbone of the alkylene group may optionally be replaced by one or more heteroatoms N, O or S, and wherein the alkylene group may optionally be substituted;
R¹⁴ is hydrogen or an alkyl or R¹⁶-L⁴- group, wherein the alkyl group may optionally be substituted;
R¹⁵ is hydrogen or an alkyl or R¹⁶-L⁴- group, wherein the alkyl group may optionally be substituted;
or R¹⁴ and R¹⁵ together with the carbon and nitrogen atoms to which they are attached form a saturated heterocyclic group, wherein the saturated heterocyclic group may optionally be substituted;
or R¹⁴ and L³, together with the carbon and nitrogen atoms to which they are attached, form a saturated heterocyclic or (saturated heterocyclic)alkylene group, wherein one or more carbon atoms in the backbone of the alkylene group may optionally be replaced by one or more heteroatoms N, O or S, and wherein the saturated heterocyclic or (saturated heterocyclic)alkylene group may optionally be substituted;
or R¹⁵ and L³, together with the nitrogen atom to which they are attached, form a divalent saturated heterocyclic or (divalent saturated heterocyclic)alkylene group, wherein one or more carbon atoms in the backbone of the alkylene group may optionally be replaced by one or more heteroatoms N, O or S, and wherein the divalent saturated heterocyclic or (divalent saturated heterocyclic)alkylene group may optionally be substituted;
each L⁴ is independently selected from a bond or an alkylene group, wherein the alkylene group may optionally be substituted; and
each R¹⁶ is independently selected from a cycloalkyl or saturated heterocyclic group, wherein the cycloalkyl or saturated heterocyclic group may optionally be substituted.

Typically:
L³ is:

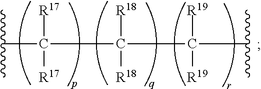

p is 0 or 1;
q is 0 or 1;
r is 0 or 1;
each R¹⁷, R¹⁸ and R¹⁹ is independently selected from hydrogen or a halo, —CN, —OH, —NH₂, —R²⁰, —OR²⁰, —NHR²⁰, —N(R²⁰)₂, —NHCOR²⁰ or —N(R²⁰)COR²⁰ group, and/or any two R¹⁷, two R¹⁸ or two R¹⁹ may together with the carbon atom to which they are attached form a C═O group, and/or any two R¹⁷, R¹⁸ or R¹⁹ may together with the carbon atom or carbon atoms to which they are attached form a 3 to 6 membered cycloalkyl or a 3 to 6 membered saturated heterocyclic group;
R¹⁴ is a C₁-C₆ alkyl or R¹⁶-L⁴- group;
R¹⁵ is hydrogen or a C₁-C₆ alkyl or R¹⁶-L⁴- group;
or R¹⁵ together with any one of R¹⁷, R¹⁸ or R¹⁹ may, together with the carbon and nitrogen atoms to which they are attached, form a 4 to 6 membered saturated heterocyclic group;
each L⁴ is independently selected from a bond or a C₁-C₆ alkylene group;
each R¹⁶ is independently any 3 to 6 membered cyclic group;
any C₁-C₆ alkylene or C₁-C₆ alkyl group may optionally be substituted with one or more groups independently selected from halo, —CN, —OH, —NH₂, oxo (═O), —OR²⁰, —NHR²⁰, —N(R²⁰)₂, —NHCOR²⁰ and —N(R²⁰)COR²⁰;
any 3 to 6 membered cycloalkyl, 3 to 6 membered saturated heterocyclic, 4 to 6 membered saturated heterocyclic or 3 to 6 membered cyclic group may optionally be substituted with one or more groups independently selected from halo, —CN, —OH, —NH₂, oxo (═O), —R², —OR²⁰, —NHR²⁰, —N(R²⁰)₂, —NHCOR²⁰ and —N(R²⁰)COR²⁰; and
each R²⁰ is independently selected from a C₁-C₄ alkyl or C₁-C₄ haloalkyl group.

In one aspect of the above embodiment, each R¹⁶ is independently selected from a 3 to 6 membered cycloalkyl group or a 3 to 6 membered saturated heterocyclic group, wherein any 3 to 6 membered cycloalkyl or 3 to 6 membered saturated heterocyclic group may optionally be substituted with one or more groups independently selected from halo, —CN, —OH, —NH₂, oxo (═O), —R²⁰, —OR²⁰, —NHR², —N(R²⁰)₂, —NHCOR²⁰ and —N(R²⁰)COR², wherein each R²⁰ is independently selected from a C₁-C₄ alkyl or C₁-C₄ haloalkyl group.

More typically:
L³ is:

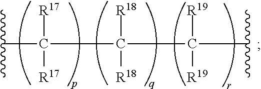

p is 1;
q is 0 or 1;
r is 0 or 1;
each R¹⁷, R¹⁸ and R¹⁹ is independently selected from hydrogen or a halo, —CN, —OH, —NH₂, —R²⁰, —OR²⁰, —NHR²⁰ or —N(R²⁰)₂ group, and/or any two R¹⁷, two R¹⁸ or two R¹⁹ may together with the carbon atom to which they are attached form a C═O group, and/or any two R¹⁷, R¹⁸ or R¹⁹ may together form a C₁-C₄ alkylene group, wherein the C₁-C₄ alkylene group may optionally include an oxygen atom in its carbon skeleton;
R¹⁴ is a C₁-C₄ alkyl or R¹⁶-L⁴- group;
R¹⁵ is hydrogen or a C₁-C₄ alkyl or R¹⁶-L⁴- group;
or R¹⁵ together with any one of R¹⁷, R¹⁸ or R¹⁹ forms a C₁-C₄ alkylene group, wherein the C₁-C₄ alkylene group may optionally include an oxygen atom in its carbon skeleton;
each L⁴ is independently selected from a bond or a C₁-C₄ alkylene group;
each R¹⁶ is independently selected from a 3 to 6 membered cycloalkyl, a 3 to 6 membered saturated heterocyclic, a phenyl or a 5 or 6 membered heteroaryl group;
any C₁-C₄ alkylene or C₁-C₄ alkyl group may optionally be substituted with one or more groups independently selected from halo, —CN, —OH, —NH₂, —OR², —NHR²⁰ and —N(R²⁰)₂;
any 3 to 6 membered cycloalkyl, 3 to 6 membered saturated heterocyclic, phenyl or 5 or 6 membered heteroaryl group may optionally be substituted with one or more groups independently selected from halo, —CN, —OH, —NH₂, —R²⁰, —OR²⁰, —NHR²⁰ and —N(R²⁰)₂; and
each R²⁰ is independently selected from a methyl or halomethyl group.

In one aspect of the above embodiment, each R¹⁶ is independently selected from a 3 to 6 membered cycloalkyl group or a 3 to 6 membered saturated heterocyclic group, wherein any 3 to 6 membered cycloalkyl or 3 to 6 membered saturated heterocyclic group may optionally be substituted with one or more groups independently selected from halo, —CN, —OH, —NH₂, —R², —OR²⁰, —NHR²⁰ and —N(R²⁰)₂, wherein each R²⁰ is independently selected from a methyl or halomethyl group.

More typically still:
L³ is:

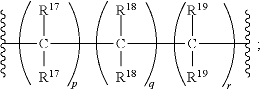

p is 1;
q is 0 or 1;
r is 0 or 1;
each R¹⁷, R¹⁸ and R¹⁹ is independently selected from hydrogen or a fluoro, chloro or —R²⁰ group;
R¹⁴ is a C₁-C₄ alkyl or C₃-C₆ cycloalkyl group;
R¹⁵ is hydrogen or a C₁-C₄ alkyl or C₃-C₆ cycloalkyl group;
or R¹⁵ together with any one of R¹⁸ or R¹⁹ forms a C₁-C₂ alkylene group;
any C₁-C₂ alkylene or C₁-C₄ alkyl group may optionally be substituted with one or more groups independently selected from fluoro, chloro, —CN, —OH, —OR², —NHR² and —N(R²⁰)₂;
any C₃-C₆ cycloalkyl group may optionally be substituted with one or more groups independently selected from fluoro, chloro, —CN, —OH, —R², —OR², —NHR²⁰ and —N(R²⁰)₂; and
each R²⁰ is a methyl group, wherein any methyl group may optionally be substituted with one or more fluoro and/or chloro groups.

Most typically,
L³ is:

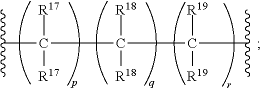

p is 1;
q is 0 or 1;
r is 0 or 1;
each R¹⁷, R¹⁸ and R¹⁹ is independently selected from hydrogen or a fluoro group;
R¹⁴ is a methyl, ethyl, isopropyl or cyclopropyl group;
R¹⁵ is a methyl, ethyl, isopropyl or cyclopropyl group;
or R¹⁵ together with any one of R¹⁸ or R¹⁹ forms a C₁-C₂ alkylene group; and
any methyl, ethyl, isopropyl, cyclopropyl or C₁-C₂ alkylene group may optionally be substituted with one or more fluoro groups.

In one embodiment, where R^{X} is a monovalent group, R^{X} is selected from the group consisting of:

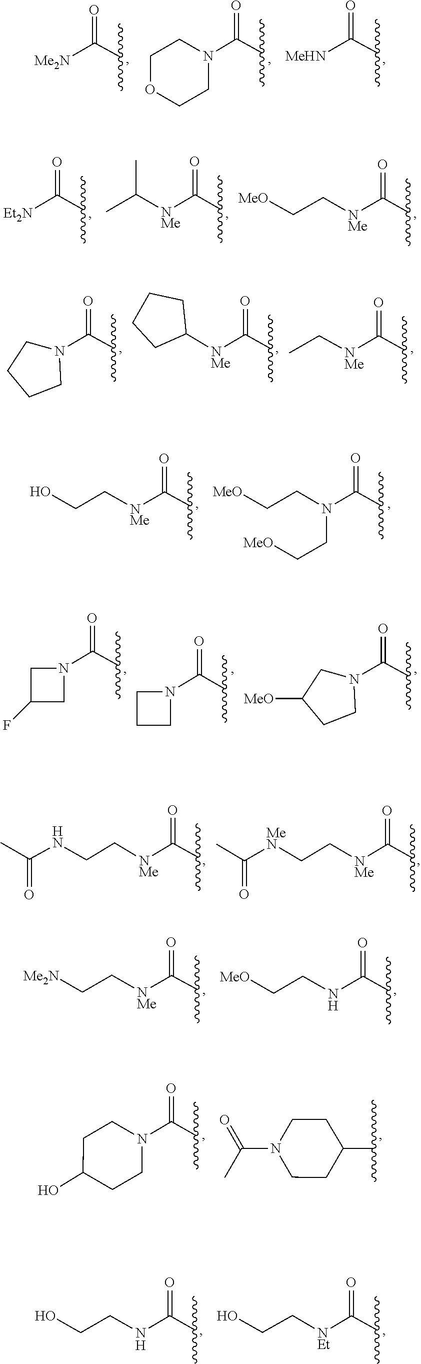

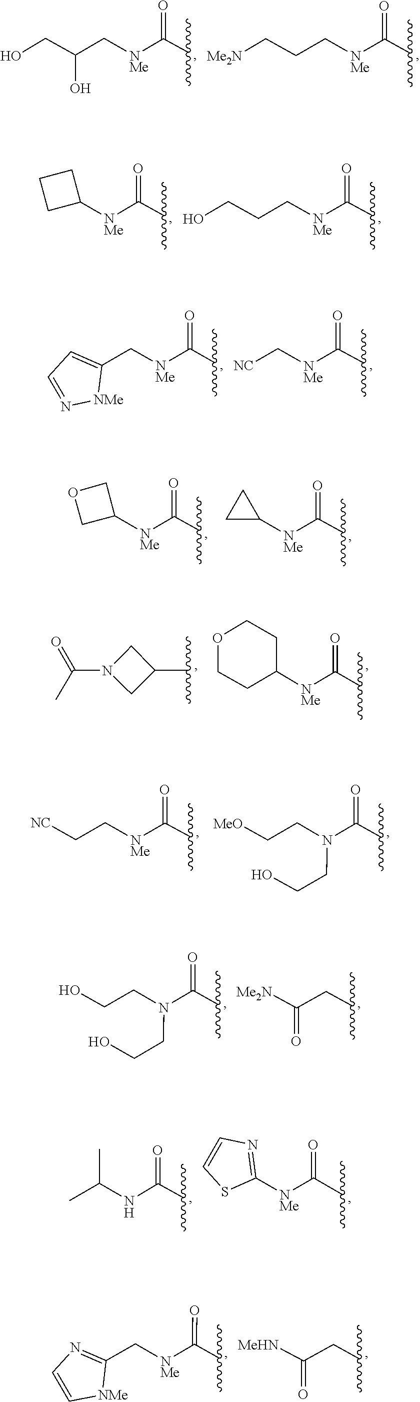

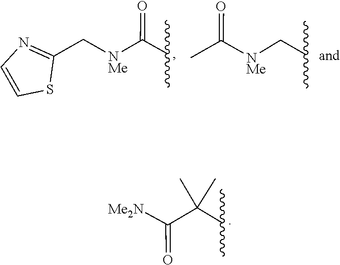

In another embodiment, R^{X} is divalent. For example, where R^{X} is divalent, —R^{X}— may be any saturated or unsaturated hydrocarbylene group, wherein the hydrocarbylene group may be straight-chained or branched, or be or include cyclic groups, wherein the hydrocarbylene group may optionally be substituted, wherein the hydrocarbylene group includes at least one amide group in its carbon skeleton, and wherein the hydrocarbylene group may optionally include one or more further heteroatoms N, O or S in its carbon skeleton. Where the hydrocarbylene group of —R^{X}— is optionally substituted, typically it is substituted with one or more groups selected from halo, —CN, —OH, —NH₂, oxo (═O) and ═NH.

Typically, where R^{X} is divalent, —R^{X}— together with the atoms of the 5-membered heteroaryl group of R¹ to which —R^{X}— is attached forms a 5- or 6-membered fused ring. Typically, —R^{X}— is attached to adjacent ring atoms of the 5-membered heteroaryl group of R¹. Typically, where R^{X} is divalent, R¹ is bicyclic.

In one embodiment, where R^{X} is divalent, —R^{X}— is:

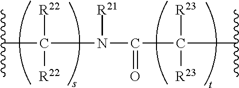

wherein:
S is 0, 1 or 2;
t is 0, 1 or 2;
3≥+t≥1;
R²¹ is hydrogen or an alkyl, cycloalkyl or saturated heterocyclic group;
each R²² and R²³ is independently selected from hydrogen or a halo, —CN, —OH, alkyl, —O-alkyl, cycloalkyl, —O-cycloalkyl, saturated heterocyclic or —O-(saturated heterocyclic) group, and/or any two R²² or two R²³ attached to the same carbon atom may together with the carbon atom to which they are attached form a C═O group, and/or any two R²² or two R²³ may together with the carbon atom or carbon atoms to which they are attached form a cycloalkyl or saturated heterocyclic group;
wherein optionally R²¹ together with any one of R²² or R²³ may together with the carbon and nitrogen atoms to which they are attached form a saturated heterocyclic group;
wherein any alkyl, cycloalkyl or saturated heterocyclic group may optionally be substituted with one or more halo, —CN, —OH, oxo (═O), alkyl, haloalkyl, —O-alkyl and/or —O-haloalkyl groups.

Typically, 2≥s+t≥1.

Typically, s is 1 or 2 and t is 0.

In one embodiment:
R²¹ is a C₁-C₄ alkyl or C₃-C₄ cycloalkyl group, or R²¹ together with any one of R²² or R²³ forms a C₁-C₄ alkylene group;
each R²² and R²³ is independently selected from hydrogen or a halo, —OH, C₁-C₃ alkyl, or —O—(C₁-C₃ alkyl) group, and/or any two R²² or two R²³ attached to the same carbon atom may together with the carbon atom to which they are attached form a C═O group, and/or any two R²² or two R²³ may together form a C₁-C₃ alkylene group; and
wherein any alkyl or alkylene group may optionally be substituted with one or more halo, —CN, —OH, oxo (═O), —OMe and/or —O-halomethyl groups, and wherein any cycloalkyl group may optionally be substituted with one or more halo, —CN, —OH, oxo (═O), methyl, halomethyl, —OMe and/or —O-halomethyl groups.

More typically:
R²¹ is a C₁-C₃ alkyl or cyclopropyl group;
each R²² and R²³ is independently selected from hydrogen or a fluoro, chloro, —OH, methyl, ethyl, —OMe or —OEt group, and/or any two R²² or two R²³ attached to the same carbon atom may together with the carbon atom to which they are attached form a C═O group or a cyclopropyl group;
wherein the C₁-C₃ alkyl group may optionally be substituted with one or more fluoro, chloro, —OH, oxo (═O), —OMe and/or —OEt groups;
wherein any cyclopropyl group may optionally be substituted with one or more fluoro, chloro, —OH or methyl groups; and
wherein any methyl or ethyl group may optionally be substituted with one or more fluoro and/or chloro groups.

More typically still:
R²¹ is a methyl, ethyl, isopropyl or cyclopropyl group;
each R²² and R²³ is independently selected from hydrogen or a fluoro, methyl or ethyl group, and/or any two R²² or two R²³ attached to the same carbon atom may together with the carbon atom to which they are attached form a C═O or cyclopropyl group; and
any methyl, ethyl, isopropyl or cyclopropyl group may optionally be substituted with one or more fluoro groups.

In one embodiment, —R^{X}— is:

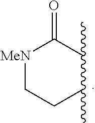

In one embodiment, R^{X} contains only atoms selected from the group consisting of carbon, hydrogen, nitrogen, oxygen and halogen atoms. For example, R^{X} may be a monovalent group that contains only atoms selected from the group consisting of carbon, hydrogen, nitrogen, oxygen and halogen atoms. Alternatively R^{X} may be a divalent group that contains only atoms selected from the group consisting of carbon, hydrogen, nitrogen, oxygen and halogen atoms.

In one embodiment, R^{X} contains from 1 to 15 carbon atoms. More typically, R^{X} contains from 2 to 7 carbon atoms.

In another embodiment, R^{X} contains from 3 to 20 atoms other than hydrogen or halogen. Most typically, in any embodiment, R^{X} contains from 4 to 11 atoms other than hydrogen or halogen.

As stated, R¹ is a 5-membered heteroaryl group substituted with at least one group R^{X}. Thus, the 5-membered heteroaryl group of R¹ may be substituted with one, two, three or four groups R^{X}. Where the 5-membered heteroaryl group of R¹ is substituted with more than one group R^{X}, each R^{X} may be the same or different, and each R^{X} may be independently selected from any R^{X} as defined above. Typically, the 5-membered heteroaryl group of R¹ is substituted with one, two or three groups R^{X}. More typically, the 5-membered heteroaryl group of R¹ is substituted with one or two groups R^{X}. Most typically, the 5-membered heteroaryl group of R¹ is substituted with a single group R^{X}. Where the 5-membered heteroaryl group of R¹ is substituted with less than four groups R^{X}, the 5-membered heteroaryl group may optionally be further substituted.

In one embodiment, the 5-membered heteroaryl group of R¹ is monocyclic. In such an embodiment, the groups R^{X} and, if present, any optional further substituents are monovalent, but may be or include cyclic groups. Examples of monocyclic 5-membered heteroaryl groups include furanyl, thiophenyl, pyrrolyl, imidazolyl, pyrazolyl, triazolyl, tetrazolyl, oxazolyl, isoxazolyl, oxadiazolyl, thiazolyl, isothiazolyl and thiadiazolyl groups.

In another embodiment, R¹ is:

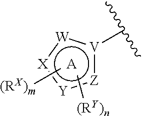

wherein:
V is independently selected from C and N, and W, X, Y and Z are each independently selected from N, O, S, NH and CH, provided that at least one of V, W, X, Y and Z is N, O, S or NH;
m is 1, 2, 3 or 4;
n is 0, 1, 2 or 3;
each R^{X} is independently selected from any monovalent R^{X} as defined herein; and
each R^{Y} is independently selected from any monovalent optional substituent as defined herein.

As will be understood, ring A is a 5-membered heteroaryl group.

For the purposes of the present specification, where it is stated that W, X, Y or Z may be NH or CH, it is to be understood that this refers to W, X, Y and Z before possible substitution with R^{X} or R^{Y} is considered. Thus, where it is stated that W, X, Y or Z may be NH, it is to be understood that W, X, Y or Z may be NH, or N—R^{X} or N—R^{Y} after substitution is considered. Similarly, where it is stated that W, X, Y or Z may be CH, it is to be understood that W, X, Y or Z may be CH, or C—R^{X} or C—R^{Y} after substitution is considered.

Typically, V is C.

Typically, m is 1 or 2 and n is 0, 1 or 2. More typically, m is 1 and n is 0 or 1. Typically, at least two of V, W, X, Y and Z are C or CH. More typically, three of V, W, X, Y and Z are C or CH. For example, where V is C, two of W, X, Y and Z may be CH.

In one embodiment, the 5-membered heteroaryl group of R¹ contains at least one nitrogen or sulfur atom in the 5-membered ring structure. For example, in the above embodiment at least one of V, W, X, Y and Z may be N, S or NH. Typically, V is C and at least one of W, X, Y and Z is N, S or NH.

In another embodiment, the 5-membered heteroaryl group of R¹ contains at least one nitrogen atom in the 5-membered ring structure. For instance, in the above embodiment at least one of V, W, X, Y and Z may be N or NH. Typically, V is C and at least one of W, X, Y and Z is N or NH. Examples of 5-membered heteroaryl groups that contain at least one nitrogen atom in the 5-membered ring structure include pyrrolyl, imidazolyl, pyrazolyl, triazolyl, tetrazolyl, oxazolyl, isoxazolyl, oxadiazolyl, thiazolyl, isothiazolyl and thiadiazolyl groups.

More typically, the 5-membered heteroaryl group of R¹ contains at least two nitrogen atoms in the 5-membered ring structure. For example, in the above embodiment at least two of V, W, X, Y and Z may be N or NH. More typically still, V is C and at least two of W, X, Y and Z are N or NH.

In one embodiment, the 5-membered heteroaryl group of R¹ contains only carbon and nitrogen atoms in the 5-membered ring structure. For example, in the above embodiment, V may be independently selected from C and N, and W, X, Y and Z may each be independently selected from N, NH and CH, provided that at least one of V, W, X, Y and Z is N or NH. Typically, in such an embodiment, V is C. Typically, at least two of V, W, X, Y and Z are N or NH. More typically, two of V, W, X, Y and Z are N or NH. Typically, one of X and Y is NH and one of X and Y is CH. Most typically, ring A is an imidazolyl or pyrazolyl group. For example, in one embodiment V is C, X is CH, Y is NH, one of W and Z is CH and one of W and Z is N. In another embodiment, V is C, X is NH, Y is CH, one of W and Z is CH and one of W and Z is N.

Typically, in any embodiment where W, X, Y or Z is NH, the NH is substituted, either by R^{X} or R^{Y}.

Where m is 1, in a typical embodiment R¹ is:

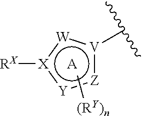

wherein V, W, X, Y, Z, R^{X}, R^{Y} and n are as defined above. Typically in such an embodiment, n is 0 or 1. More typically, R¹ is:

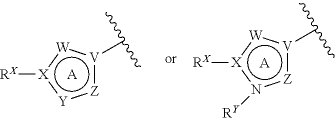

wherein V, W, X, Y, Z, R^{X} and R^{Y} are as defined above. More typically still, R¹ is:

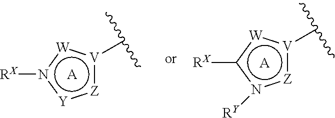

wherein V, W, Y, Z, R^{X} and R^{Y} are as defined above.

In the above embodiments, the 5-membered heteroaryl group of R¹ may be further substituted with one or more substituents, optionally referred to as R^{Y}, independently selected from halo; —CN; —NO₂; —N₃; —R; —OH; —OR; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —CORP; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(N₂)R^{β}; —R^{α}—C(═NH)R^{β}; —R^{α}—C(═NH)NH₂; —R^{α}—C(═NH)NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(N₂)R^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{a}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{a}—N(R^{β})₂; or —N⁺(R^{β})₂—R^{α}—N(O)(R^{β})₂;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —COO(C₁-C₄ alkyl), —COO(C₁-C₄ haloalkyl), halo, —OH, —NH₂, —CN, —C≡CH, oxo (═O), or 4- to 6-membered heterocyclic group.

In one embodiment, the 5-membered heteroaryl group of R¹ may be further substituted with one or more substituents, optionally referred to as R^{Y}, independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —CORP; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{a}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —NH—R^{α}—OH; —NH—R^{α}—OR^{β}; —NH—R^{α}—NH₂; —NH—R^{α}—NHR^{β}; —NH—R^{α}—N(R^{β})₂; —NR^{β}—R^{α}—OH; —NR^{β}—R^{α}—OR^{β}; —NR^{β}—R^{α}—NH₂; —NR^{β}—R^{α}—NHR^{β}; or —NR^{β}—R^{α}—N(R^{β})₂;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH, oxo (═O), or 4- to 6-membered heterocyclic group.

In another embodiment, the 5-membered heteroaryl group of R¹ is further substituted with one, two or three substituents independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —CORP; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β};
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH, oxo (═O), or 4- to 6-membered heterocyclic group.

Alternatively, the 5-membered heteroaryl group of R¹ may be further substituted with one, two or three substituents independently selected from halo; —CN; —R^{β}; —OH; —OR^{β}; —R^{α}-halo; —R^{α}—CN; —R^{α}—R^{β}; —R^{α}—OH; —R^{α}—OR; —SOR^{β}; —SO₂H; —SO₂R^{β}; —SO₂NH₂; —SO₂NHR^{β}; —SO₂N(R^{β})₂; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —CORP; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β};
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, halo, —OH, or 4- to 6-membered heterocyclic group.

Typically, where the 5-membered heteroaryl group of R¹ is further substituted with one or more optional substituents R^{Y}, R^{Y} is a monovalent group. Typically, where R^{Y} is a monovalent group, R^{Y} contains from 1 to 11 atoms other than hydrogen or halogen. More typically, R^{Y} contains from 1 to 8 atoms other than hydrogen or halogen. Most typically, R^{Y} contains from 1 to 6 atoms other than hydrogen or halogen.

In one embodiment, each R^{Y} is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton. Where the hydrocarbyl group of R^{Y} is optionally substituted, typically it is substituted with one or more groups independently selected from halo, —CN, —OH, —NH₂, oxo (═O) and ═NH.

Typically, each R^{Y} is a saturated hydrocarbyl group, wherein the saturated hydrocarbyl group may be straight-chained or branched, or be or include cyclic groups, wherein the saturated hydrocarbyl group may optionally be substituted with one or more groups independently selected from halo, —CN, —OH, —NH₂ and oxo (═O), and wherein the saturated hydrocarbyl group may optionally include one or two heteroatoms N or O in its carbon skeleton.

More typically, each R^{Y} is independently selected from a C₁-C₆ alkyl or C₃-C₆ cycloalkyl group, wherein any C₁-C₆ alkyl or C₃-C₆ cycloalkyl group may optionally be substituted with one or more fluoro, chloro, —CN, —OH, —NH₂, —OMe, —NHMe and/or —NMe₂ groups, wherein any methyl group may optionally be substituted with one or more fluoro and/or chloro groups.

More typically still, each R^{Y} is independently selected from a C₁-C₄ alkyl or C₃-C₄ cycloalkyl group, wherein any C₁-C₄ alkyl or C₃-C₄ cycloalkyl group may optionally be substituted with one or more fluoro and/or chloro groups.

Most typically each R^{Y} is independently selected from a methyl, ethyl, isopropyl or cyclopropyl group.

In one aspect of any of the above embodiments, R¹ contains from 8 to 30 atoms other than hydrogen. More typically, R¹ contains from 8 to 25 atoms other than hydrogen. More typically, R¹, contains from 9 to 20 atoms other than hydrogen. More typically, R¹ contains from 9 to 17 atoms other than hydrogen.

In one aspect of any of the above embodiments, R¹ contains from 8 to 25 atoms other than hydrogen or halogen. More typically, R¹ contains from 9 to 20 atoms other than hydrogen or halogen. More typically still, R¹ contains from 9 to 16 atoms other than hydrogen or halogen.

R² is a cyclic group substituted at the α-position, wherein R² may optionally be further substituted. For the avoidance of doubt, it is noted that it is a ring atom of the cyclic group of R² that is directly attached to the nitrogen atom of the urea or thiourea group, not any substituent.

In one embodiment of the first aspect of the invention, R² is an aryl or a heteroaryl group, wherein the aryl or the heteroaryl group is substituted at the α-position, and wherein R² may optionally be further substituted. Typically, R² is a phenyl or a 5- or 6-membered heteroaryl group, wherein the phenyl or the heteroaryl group is substituted at the α-position, and wherein R² may optionally be further substituted. Typically, R² is an aryl or a heteroaryl group, wherein the aryl or the heteroaryl group is substituted at the α and a′ positions, and wherein R² may optionally be further substituted. Typically, R² is a phenyl or a 5- or 6-membered heteroaryl group, wherein the phenyl or the heteroaryl group is substituted at the α and a′ positions, and wherein R² may optionally be further substituted. For example, R² may be a phenyl group substituted at the 2- and 6-positions or a phenyl group substituted at the 2-, 4- and 6-positions.

In one embodiment, the parent phenyl or 5- or 6-membered heteroaryl group of R² may be selected from phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl. Typically, the parent phenyl or 5- or 6-membered heteroaryl group of R² may be selected from phenyl, pyridinyl, pyridazinyl, pyrimidinyl, pyrrolyl, pyrazolyl, imidazolyl or triazolyl. Typically, the parent phenyl or 5- or 6-membered heteroaryl group of R² may be selected from phenyl, pyridinyl, pyridazinyl, pyrimidinyl or pyrazolyl.

As used herein, the nomenclature α, β, α′, β′ refers to the position of the atoms of a cyclic group, such as —R², relative to the point of attachment of the cyclic group to the remainder of the molecule. For example, where —R² is a 1,2,3,5,6,7-hexahydro-s-indacen-4-yl moiety, the α, β, α′ and β′ positions are as follows:

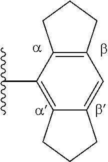 [SMILES: *c1c2c(cc3c1CCC3)CCC2]

For the avoidance of doubt, where it is stated that a cyclic group, such as an aryl or a heteroaryl group, is substituted at the α and/or α′ positions, it is to be understood that one or more hydrogen atoms at the α and/or α′ positions respectively are replaced by one or more substituents, such as any optional substituent as defined above. Unless stated otherwise the term “substituted” does not include the replacement of one or more ring carbon atoms by one or more ring heteroatoms.

In another embodiment, R² is a cyclic group substituted at the α and α′ positions, wherein R² may optionally be further substituted. For example, R² may be a cycloalkyl, cycloalkenyl or non-aromatic heterocyclic group substituted at the α and α′ positions.

In any of the above embodiments, typical substituents at the α and/or α′ positions of the parent cyclic group of R² comprise a carbon atom. For example, typical substituents at the α and/or α′ positions may be independently selected from —R⁴, —OR⁴ and —COR⁴ groups, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. More typically, the substituents at the α and/or α′ positions are independently selected from alkyl and cycloalkyl groups, such as C₃-C₆ branched alkyl and C₃-C₆ cycloalkyl groups, e.g. isopropyl, cyclopropyl, cyclohexyl or t-butyl groups, wherein the alkyl and cycloalkyl groups are optionally further substituted with one or more fluoro and/or chloro groups.

In one aspect of any of the above embodiments, each substituent at the α and α′ positions comprises a carbon atom.

Other typical substituents at the α and/or α′ positions of the parent cyclic group of R² may include cycloalkyl, cycloalkenyl, non-aromatic heterocyclic, aryl or heteroaryl rings which are fused to the parent cyclic group across the α,β and/or α′,β′ positions respectively. Such fused cyclic groups are described in greater detail below.

In one embodiment, R² is a fused aryl or a fused heteroaryl group, wherein the aryl or heteroaryl group is fused to one or more cycloalkyl, cycloalkenyl, non-aromatic heterocyclic, aryl or heteroaryl rings, wherein R² may optionally be further substituted. Typically, a cycloalkyl, cycloalkenyl, non-aromatic heterocyclic, aryl or heteroaryl ring is fused to the aryl or heteroaryl group across the α,β positions. Typically, the aryl or heteroaryl group is also substituted at the α′ position, for example with a substituent selected from —R⁴, —OR⁴ and —COR⁴, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. Typically in such an embodiment, R² is bicyclic or tricyclic.

More typically, R² is a fused phenyl or a fused 5- or 6-membered heteroaryl group, wherein the phenyl or the 5- or 6-membered heteroaryl group is fused to one or more cycloalkyl, cycloalkenyl, non-aromatic heterocyclic, aryl or heteroaryl rings, wherein R² may optionally be further substituted. Typically, a cycloalkyl, cycloalkenyl, non-aromatic heterocyclic, aryl or heteroaryl ring is fused to the phenyl or the 5- or 6-membered heteroaryl group across the α,β positions so as to form a 4- to 6-membered fused ring structure. Typically, the phenyl or the 5- or 6-membered heteroaryl group is also substituted at the α′ position, for example with a substituent selected from —R⁴, —OR⁴ and —COR⁴, wherein each R⁴ is independently selected from a C₁-C₆ alkyl, C₂-C₆ alkenyl, C₂-C₆ alkynyl or C₂-C₆ cyclic group and wherein each R⁴ is optionally further substituted with one or more halo groups. Typically in such an embodiment, R² is bicyclic or tricyclic.

In another embodiment, R² is a fused aryl or a fused heteroaryl group, wherein the aryl or heteroaryl group is fused to two or more independently selected cycloalkyl, cycloalkenyl, non-aromatic heterocyclic, aryl or heteroaryl rings, wherein R² may optionally be further substituted. Typically, the two or more cycloalkyl, cycloalkenyl, non-aromatic heterocyclic, aryl or heteroaryl rings are each ortho-fused to the aryl or heteroaryl group, i.e. each fused cycloalkyl, cycloalkenyl, non-aromatic heterocyclic, aryl or heteroaryl ring has only two atoms and one bond in common with the aryl or heteroaryl group. Typically, R² is tricyclic.

In yet another embodiment, R² is a fused aryl or a fused heteroaryl group, wherein a first cycloalkyl, cycloalkenyl, non-aromatic heterocyclic, aryl or heteroaryl ring is fused to the aryl or heteroaryl group across the α,β positions and a second cycloalkyl, cycloalkenyl, non-aromatic heterocyclic, aryl or heteroaryl ring is fused to the aryl or heteroaryl group across the α′,β′ positions, wherein R² may optionally be further substituted. Typically in such an embodiment, R² is tricyclic.

More typically, R² is a fused phenyl or a fused 5- or 6-membered heteroaryl group, wherein a first cycloalkyl, cycloalkenyl, non-aromatic heterocyclic, aryl or heteroaryl ring is fused to the phenyl or the 5- or 6-membered heteroaryl group across the α,β positions so as to form a first 4- to 6-membered fused ring structure, and a second cycloalkyl, cycloalkenyl, non-aromatic heterocyclic, aryl or heteroaryl ring is fused to the phenyl or the 5- or 6-membered heteroaryl group across the α′,β′ positions so as to form a second 4- to 6-membered fused ring structure, wherein R² may optionally be further substituted. Typically in such an embodiment, R² is tricyclic.

In one embodiment, —R² has a formula selected from:

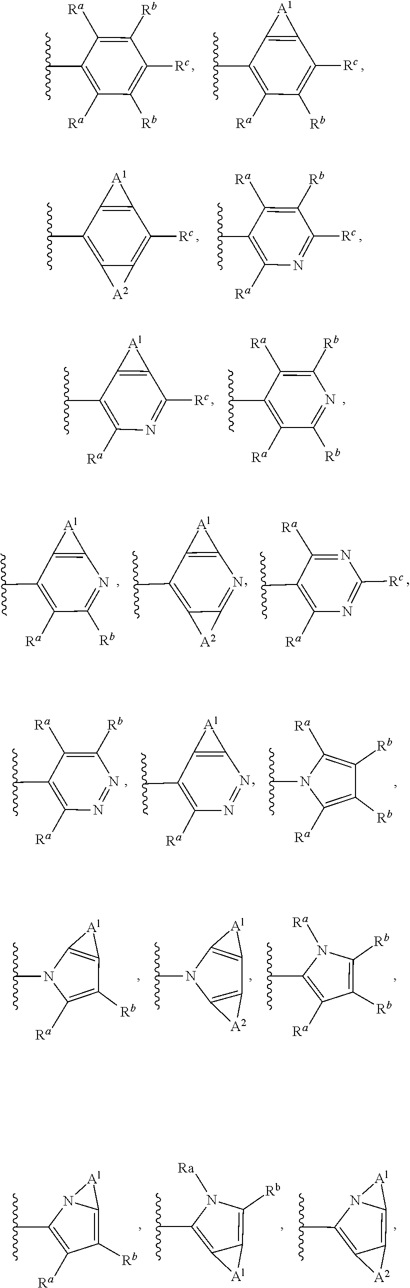

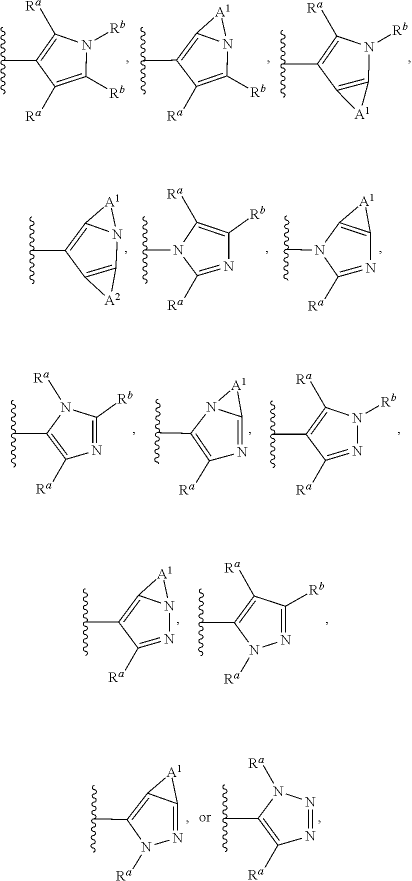

wherein:
A¹ and A² are each independently selected from an optionally substituted alkylene or alkenylene group, wherein one or more carbon atoms in the backbone of the alkylene or alkenylene group may optionally be replaced by one or more heteroatoms N, O or S;
each R^{a} is independently selected from —R^{aa}, —OR^{aa} or —COR^{aa};
each R^{b} is independently selected from hydrogen, halo, —NO₂, —CN, —R^{aa}, —OR^{aa} or —COR^{aa};
provided that any R^{a} or R^{b} that is directly attached to a ring nitrogen atom is not halo, —NO₂, —CN, or —OR^{aa};
each R^{c} is independently selected from hydrogen, halo, —OH, —NO₂, —CN, —R^{cc}, —OR^{cc}, —COR^{cc}, —COOR^{cc}, —CONH₂, —CONHR^{cc} or —CON(R^{cc})₂;
each R^{aa} is independently selected from a C₁-C₆ alkyl, C₂-C₆ alkenyl, C₂-C₆ alkynyl or a 3- to 7-membered cyclic group, wherein each R^{aa} is optionally substituted; and
each R^{cc} is independently selected from a C₁-C₆ alkyl, C₂-C₆ alkenyl, C₂-C₆ alkynyl or a 3- to 7-membered cyclic group, or any two R^{cc} attached to the same nitrogen atom may, together with the nitrogen atom to which they are attached, form a 3- to 7-membered heterocyclic group, wherein each R^{cc} is optionally substituted.

Typically, any ring containing A¹ or A² is a 5- or 6-membered ring. Typically, A¹ and A² are each independently selected from an optionally substituted straight-chained alkylene group or an optionally substituted straight-chained alkenylene group, wherein one or two carbon atoms in the backbone of the alkylene or alkenylene group may optionally be replaced by one or two heteroatoms independently selected from nitrogen and oxygen. More typically, A¹ and A² are each independently selected from an optionally substituted straight-chained alkylene group, wherein one carbon atom in the backbone of the alkylene group may optionally be replaced by an oxygen atom. Typically, no heteroatom in A¹ or A² is directly attached to another ring heteroatom. Typically, A¹ and A² are unsubstituted or substituted with one or more substituents independently selected from halo, —OH, —CN, —NO₂, C₁-C₄ alkyl, C₁-C₄ haloalkyl, —O(C₁-C₄ alkyl) or —O(C₁-C₄ haloalkyl). More typically, A¹ and A² are unsubstituted or substituted with one or more fluoro and/or chloro groups. Where R² contains both A¹ and A² groups, A¹ and A² may be the same or different. Typically, A¹ and A² are the same.

Where R^{aa} is a substituted C₁-C₆ alkyl, C₂-C₆ alkenyl or C₂-C₆ alkynyl group, typically the C₁-C₆ alkyl, C₂-C₆ alkenyl or C₂-C₆ alkynyl group is substituted with one or more (e.g. one or two) substituents independently selected from halo, —OH, —CN, —NO₂, —O(C₁-C₄ alkyl) or —O(C₁-C₄ haloalkyl).

Where R^{aa} is a substituted 3- to 7-membered cyclic group, typically the 3- to 7-membered cyclic group is substituted with one or more (e.g. one or two) substituents independently selected from halo, —OH, —NH₂, —CN, —NO₂, —B¹, —OB¹, —NHB¹, —N(B¹)₂, —CONH₂, —CONHB¹, —CON(B¹)₂, —NHCOB¹, —NB¹COB¹, or —B¹¹—;
wherein each B¹ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B¹ together with the nitrogen atom to which they are attached may form a 4- to 6-membered heterocyclic group containing one or two ring heteroatoms N and/or O, wherein any B¹ may optionally be halo-substituted and/or substituted with one or two substituents independently selected from —OH, —NH₂, —OB¹², —NHB¹² or —N(B¹²)₂;
wherein each B¹¹ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB¹², —NHB¹² or —N(B¹²)₂; and
wherein each B¹² is independently selected from a C₁-C₃ alkyl or C₁-C₃ haloalkyl group. Typically, any divalent group —B¹¹— forms a 4- to 6-membered fused ring.

Typically, each R^{a} is —R^{aa}. More typically, each R^{a} is independently selected from a C₁-C₆ alkyl (in particular C₃-C₆ branched alkyl) or C₃-C₆ cycloalkyl group, wherein each R^{a} is optionally further substituted with one or more halo groups. More typically, each R^{a} is independently selected from a C₁-C₄ alkyl, C₁-C₄ haloalkyl, C₃-C₄ cycloalkyl or C₃-C₄ halocycloalkyl group. Where a group R^{a} is present at both the α- and α′-positions, each R^{a} may be the same or different. Typically, each R^{a} is the same.

Typically, each R^{b} is independently selected from hydrogen or halo. More typically, each R^{b} is hydrogen.

Typically, each R^{c} is independently selected from hydrogen, halo, —OH, —NO₂, —CN, —R^{cc} or —OR^{cc}. More typically, each R^{c} is independently selected from hydrogen, halo, —CN, C₁-C₃ alkyl, C₁-C₃ haloalkyl, cyclopropyl or halocyclopropyl. Most typically, each R^{c} is independently selected from hydrogen or halo.

Typically, each R^{cc} is independently selected from a C₁-C₄ alkyl or C₃-C₆ cycloalkyl group, or any two R^{cc} attached to the same nitrogen atom may, together with the nitrogen atom to which they are attached, form a 3- to 6-membered saturated heterocyclic group, wherein each R^{cc} is optionally substituted. Where R is substituted, typically R^{cc} is substituted with one or more halo, —OH, —CN, —NO₂, —O(C₁-C₄ alkyl) or —O(C₁-C₄ haloalkyl) groups. More typically, each R^{cc} is independently selected from a C₁-C₄ alkyl, C₁-C₄ haloalkyl, C₃-C₄ cycloalkyl or C₃-C₄ halocycloalkyl group.

In one embodiment, —R² has a formula selected from:

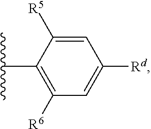

wherein R⁵ and R⁶ are independently selected from C₁-C₄ alkyl, C₁-C₄ haloalkyl, C₃-C₄ cycloalkyl and C₃-C₄ halocycloalkyl, and R^{d} is hydrogen, halo, —OH, —NO₂, —CN, —R^{dd}, —OR^{dd}, —COR^{dd}, —COOR^{dd}, —CONH₂, —CONHR^{d}d or —CON(R^{dd})₂, wherein each —R^{dd} is independently selected from C₁-C₄ alkyl, C₁-C₄ haloalkyl, C₃-C₄ cycloalkyl and C₃-C₄ halocycloalkyl. Typically, R⁵ and R⁶ are independently selected from C₁-C₄ alkyl, and R^{d} is hydrogen, halo, —CN, C₁-C₃ alkyl, C₁-C₃ haloalkyl, cyclopropyl or halocyclopropyl. More typically, R⁵ and R⁶ are independently selected from C₁-C₄ alkyl, and R^{d} is hydrogen or halo. In one aspect of such an embodiment, R⁵ and R⁶ are independently selected from C₁-C₄ alkyl, and R^{d} is halo.

Typically, —R² has a formula selected from:

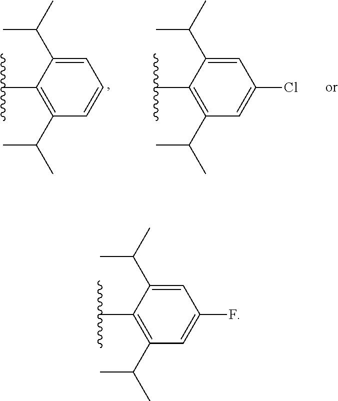 [SMILES: *c1c(C(C)C)cccc1C(C)C ; *c1c(C(C)C)cc(Cl)cc1C(C)C ; *c1c(C(C)C)cc(F)cc1C(C)C]

In one embodiment, —R² has a formula selected from:

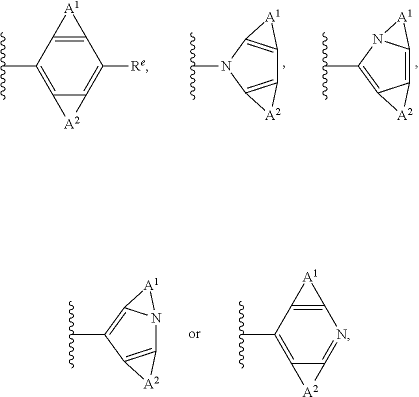

wherein A¹ and A² are each independently selected from an optionally substituted alkylene or alkenylene group, wherein one or more carbon atoms in the backbone of the alkylene or alkenylene group may optionally be replaced by one or more heteroatoms N, O or S, and wherein R^{e} is hydrogen or any optional substituent. R^{e} and any optional substituent attached to A¹ or A² may together with the atoms to which they are attached form a further fused cycloalkyl, cycloalkenyl, non-aromatic heterocyclic, aryl or heteroaryl ring which may itself be optionally substituted. Similarly, any optional substituent attached to A¹ and any optional substituent attached to A² may also together with the atoms to which they are attached form a further fused cycloalkyl, cycloalkenyl, non-aromatic heterocyclic, aryl or heteroaryl ring which may itself be optionally substituted.

In one embodiment, R^{e} is hydrogen, halo, —OH, —NO₂, —CN, —R^{ee}, —OR^{ee}, —COR^{ee}, —COOR^{ee}, —CONH₂, —CONHR^{ee} or —CON(R^{ee})₂, wherein each —R^{ee} is independently selected from C₁-C₄ alkyl, C₁-C₄ haloalkyl, C₃-C₄ cycloalkyl and C₃-C₄ halocycloalkyl.

Typically, R^{e} is hydrogen or a halo, hydroxyl, —CN, —NO₂, —R^{ee} or —OR^{ee} group, wherein R^{ee} is a C₁-C₄ alkyl group which may optionally be halo-substituted. More typically, R^{e} is hydrogen or a halo, hydroxyl, —CN, —R^{ee} or —OR^{ee} group, wherein R^{ee} is a C₁-C₄ alkyl group which may optionally be halo-substituted. More typically, R^{e} is hydrogen or halo.

Typically, any ring containing A¹ or A² is a 5- or 6-membered ring.

Typically, A¹ and A² are each independently selected from an optionally substituted straight-chained alkylene group or an optionally substituted straight-chained alkenylene group, wherein one or two carbon atoms in the backbone of the alkylene or alkenylene group may optionally be replaced by one or two heteroatoms independently selected from nitrogen and oxygen. More typically, A¹ and A² are each independently selected from an optionally substituted straight-chained alkylene group, wherein one carbon atom in the backbone of the alkylene group may optionally be replaced by an oxygen atom. Typically, no heteroatom in A¹ or A² is directly attached to another ring heteroatom. Typically, A¹ and A² are unsubstituted or substituted with one or more halo, hydroxyl, —CN, —NO₂, —B³ or —OB³ groups, wherein B³ is a C₁-C₄ alkyl group which may optionally be halo-substituted. More typically, A¹ and A² are unsubstituted or substituted with one or more halo, hydroxyl, —CN, —B³ or —OB³ groups, wherein B³ is a C₁-C₄ alkyl group which may optionally be halo-substituted. More typically, A¹ and A² are unsubstituted or substituted with one or more fluoro and/or chloro groups. Where R² contains both A¹ and A² groups, A¹ and A² may be the same or different. Typically, A¹ and A² are the same.

In a further embodiment, —R² has a formula selected from:

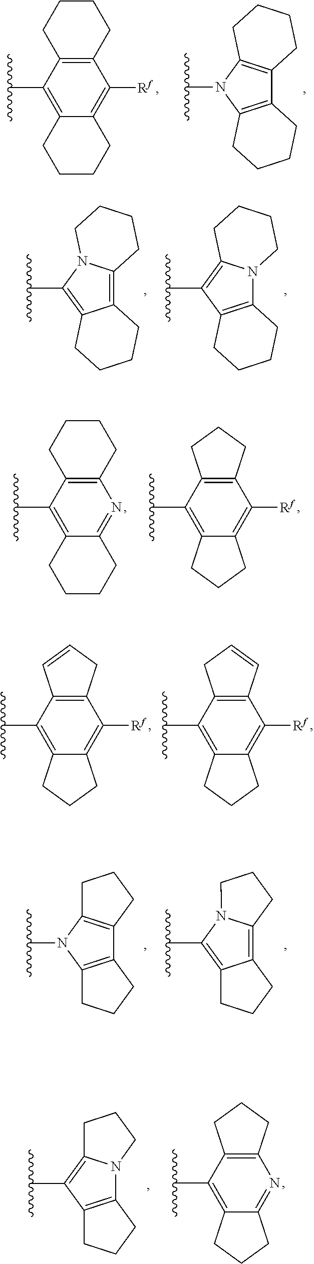

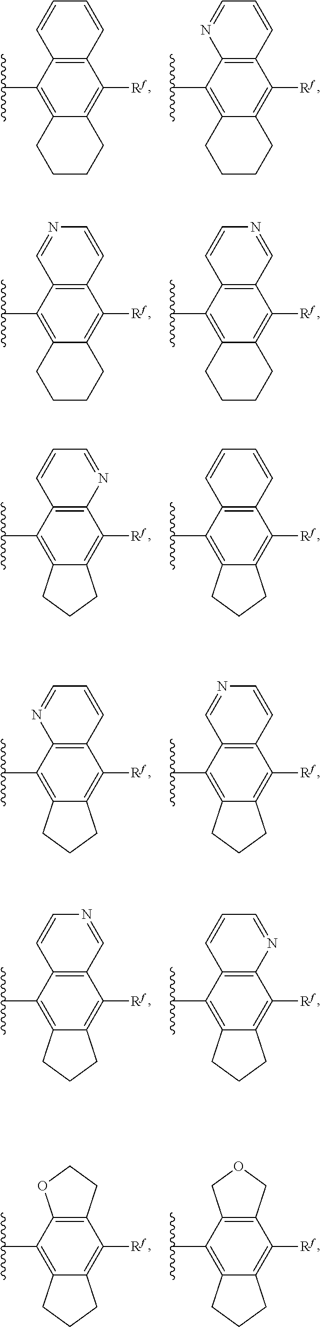

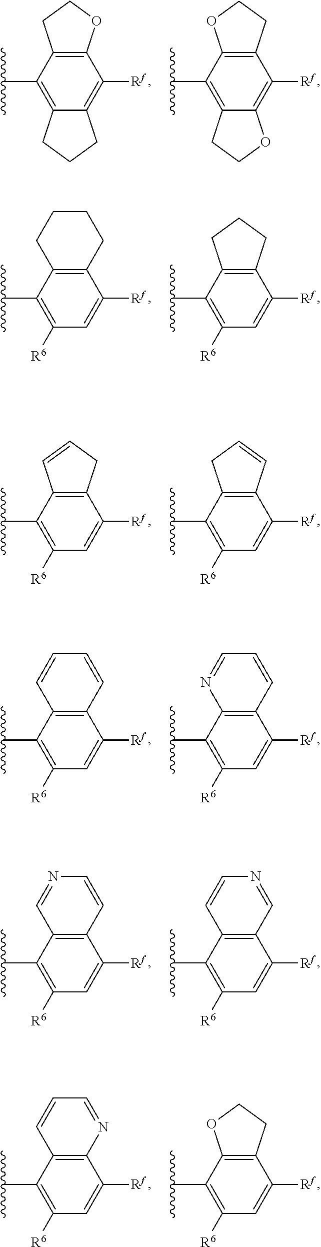

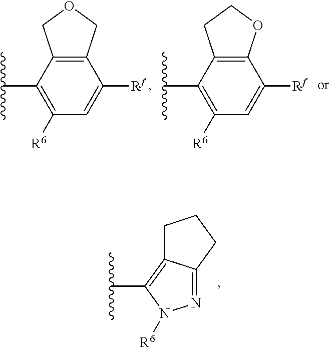

wherein R⁶ is C₁-C₄ alkyl, C₁-C₄ haloalkyl, C₃-C₄ cycloalkyl or C₃-C₄ halocycloalkyl, and R^{f} is hydrogen, halo, —OH, —NO₂, —CN, —R^{ff}, —OR^{ff}, —COR^{ff}, —COOR^{ff}, —CONH₂, —CONHR^{ff} or —CON(R^{ff})₂, wherein each —R^{ff} is independently selected from C₁-C₄ alkyl, C₁-C₄ haloalkyl, C₃-C₄ cycloalkyl and C₃-C₄ halocycloalkyl. Typically, R⁶ is C₁-C₄ alkyl, and R^{f} is hydrogen, halo, —CN, C₁-C₃ alkyl, C₁-C₃ haloalkyl, cyclopropyl or halocyclopropyl. Typically, R⁶ is C₁-C₄ alkyl, and R^{f} is hydrogen or halo.

Typically, —R² has the formula:

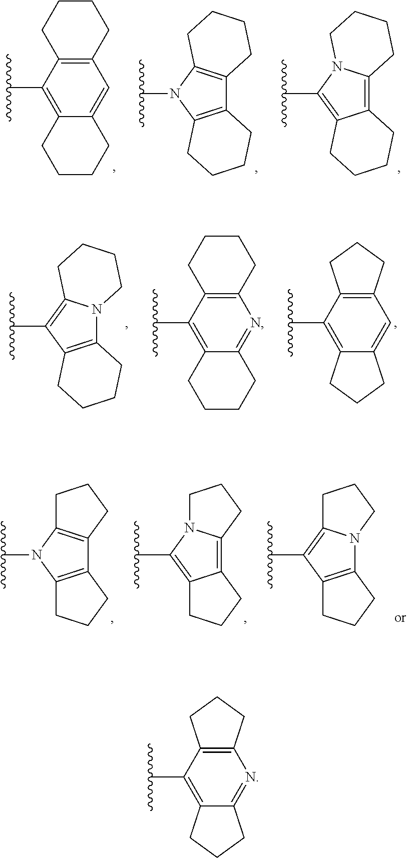

More typically, —R² has the formula:

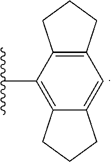 [SMILES: *c1c2c(cc3c1CCC3)CCC2]

Yet other typical substituents at the α-position of the parent cyclic group of R² may include monovalent heterocyclic groups and monovalent aromatic groups, wherein a ring atom of the heterocyclic or aromatic group is directly attached via a single bond to the α-ring atom of the parent cyclic group, wherein the heterocyclic or aromatic group may optionally be substituted, and wherein the parent cyclic group may optionally be further substituted. Such R² groups are described in greater detail below.

In one embodiment, the α-substituted parent cyclic group of R² is a 5- or 6-membered cyclic group, wherein the cyclic group may optionally be further substituted. In one embodiment, the α-substituted parent cyclic group of R² is an aryl or a heteroaryl group, all of which may optionally be further substituted. In one embodiment, the α-substituted parent cyclic group of R² is a phenyl or a 5- or 6-membered heteroaryl group, all of which may optionally be further substituted. In one embodiment, the α-substituted parent cyclic group of R² is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl or oxadiazolyl group, all of which may optionally be further substituted. In one embodiment, the α-substituted parent cyclic group of R² is a phenyl or pyrazolyl group, both of which may optionally be further substituted. In a further embodiment, the α-substituted parent cyclic group of R² is a phenyl group, which may optionally be further substituted.

In one embodiment, the α-substituted parent cyclic group of R² is substituted at the α and α′ positions, and may optionally be further substituted. For example, the α-substituted parent cyclic group of R² may be a phenyl group substituted at the 2- and 6-positions or a phenyl group substituted at the 2-, 4- and 6-positions.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. In one embodiment, the monovalent heterocyclic or aromatic group at the α-position is a phenyl or a 5- or 6-membered heterocyclic group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, thianyl, morpholinyl, thiomorpholinyl or 1-methyl-2-oxo-1,2-dihydropyridinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, thianyl, piperazinyl, 1,4-dioxanyl, morpholinyl or thiomorpholinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, piperidinyl or tetrahydropyranyl group, all of which may optionally be substituted. In one embodiment, the monovalent heterocyclic or aromatic group at the α-position is a phenyl, pyridinyl, pyridazinyl, pyrimidinyl, pyrazolyl, imidazolyl, isoxazolyl, thiazolyl, tetrahydropyranyl or 1-methyl-2-oxo-1,2-dihydropyridinyl group, all of which may optionally be substituted. In one embodiment, the monovalent heterocyclic or aromatic group at the α-position is a phenyl, pyridinyl, pyrimidinyl, pyrazolyl, imidazolyl, isoxazolyl, thiazolyl or tetrahydropyranyl group, all of which may optionally be substituted. In one embodiment, the monovalent heterocyclic or aromatic group at the α-position is a phenyl, pyridinyl, pyrimidinyl, pyrazolyl, imidazolyl, isoxazolyl, thiazolyl or tetrahydropyranyl group, all of which may optionally be substituted. In one embodiment, the monovalent heterocyclic or aromatic group at the α-position is a phenyl, pyridinyl, pyrimidinyl or pyrazolyl group, all of which may optionally be substituted. In one embodiment, the monovalent heterocyclic or aromatic group at the α-position is an unsubstituted phenyl, pyridinyl, pyrimidinyl or pyrazolyl group. In one embodiment, the monovalent heterocyclic group at the α-position is a pyridin-2-yl, pyridin-3-yl or pyridin-4-yl group, all of which may optionally be substituted. In one embodiment, the monovalent heterocyclic group at the α-position is an unsubstituted pyridin-3-yl group or an optionally substituted pyridin-4-yl group.

For any of these monovalent heterocyclic or aromatic groups at the α-position mentioned in the immediately preceding paragraph, the monovalent heterocyclic or aromatic group may optionally be substituted with one or two substituents independently selected from halo, —OH, —NH₂, —CN, —NO₂, —B⁴, —OB⁴, —NHB⁴, —N(B⁴)₂, —CONH₂, —CONHB⁴, —CON(B⁴)₂, —NHCOB⁴, —NB⁴COB⁴, or —B⁴⁴—;
wherein each B⁴ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁴ together with the nitrogen atom to which they are attached may form a 4- to 6-membered heterocyclic group containing one or two ring heteroatoms N and/or O, wherein any B⁴ may optionally be halo-substituted and/or substituted with one or two substituents independently selected from —OH, —NH₂, —OB⁴⁵, —NHB⁴⁵ or —N(B⁴⁵)₂;
wherein each B⁴⁴ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁴⁵, —NHB⁴⁵ or —N(B⁴⁵)₂; and wherein each B⁴⁵ is independently selected from a C₁-C₃ alkyl or C₁-C₃ haloalkyl group.

Typically, any divalent group —B⁴⁴— forms a 4- to 6-membered fused ring.

In one embodiment, the monovalent heterocyclic or aromatic group at the α-position is a phenyl, pyridinyl, pyrimidinyl or pyrazolyl group, all of which may optionally be substituted with one or two substituents independently selected from halo, —OH, —NH₂, —CN, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted. In one embodiment, the monovalent heterocyclic group at the α-position is a pyridin-2-yl, pyridin-3-yl or pyridin-4-yl group, all of which may optionally be substituted with one or two substituents independently selected from halo, —OH, —NH₂, —CN, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted. In one embodiment, the monovalent heterocyclic group at the α-position is an unsubstituted pyridin-3-yl group or a pyridin-4-yl group optionally substituted with one or two substituents independently selected from halo, —OH, —NH₂, —CN, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. In one embodiment, such further substituents are in the α′ position of the α-substituted parent cyclic group of R². Such further substituents may be independently selected from halo, —R, —OR⁶ or —COR⁶ groups, wherein each R⁶ is independently selected from a C₁-C₆ alkyl, C₂-C₆ alkenyl, C₂-C₆ alkynyl or C₂-C₆ cyclic group and wherein each R⁶ is optionally further substituted with one or more halo groups. Typically, such further substituents on the α-substituted parent cyclic group of R² are independently selected from halo, C₁-C₆ alkyl (in particular C₃-C₆ branched alkyl) or C₃-C₆ cycloalkyl groups, e.g. fluoro, chloro, isopropyl, cyclopropyl, cyclohexyl or t-butyl groups, wherein the alkyl and cycloalkyl groups are optionally further substituted with one or more fluoro and/or chloro groups.

In one embodiment, —R² has a formula selected from:

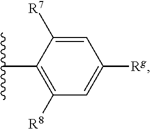

wherein R⁷ is C₁-C₄ alkyl, C₁-C₄ haloalkyl, C₃-C₆ cycloalkyl or C₃-C₆ halocycloalkyl, R⁸ is a 5- or 6-membered, optionally substituted heterocyclic or aromatic group, and R^{g} is hydrogen, halo, —OH, —NO₂, —CN, —R^{gg}, —OR^{gg}, —COR^{gg}, —COOR^{gg}, —CONH₂, —CONHR^{gg} or —CON(R^{gg})₂, wherein each —R^{gg} is independently selected from C₁-C₄ alkyl, C₁-C₄ haloalkyl, C₃-C₄ cycloalkyl and C₃-C₄ halocycloalkyl. In one embodiment, the optional substituents on the heterocyclic or aromatic group are independently selected from halo, —OH, —NH₂, —CN, —NO₂, —B⁵, —OB⁵, —NHB⁵, —N(B⁵)₂, —CONH₂, —CONHB⁵, —CON(B⁵)₂, —NHCOB⁵, —NB⁵COB⁵, or —B⁵⁵—;
wherein each B⁵ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁵ together with the nitrogen atom to which they are attached may form a 4- to 6-membered heterocyclic group containing one or two ring heteroatoms N and/or O, wherein any B⁵ may optionally be halo-substituted and/or substituted with one or two substituents independently selected from —OH, —NH₂, —OB⁵⁶, —NHB⁵⁶ or —N(B⁵⁶)
wherein each B⁵⁵ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁵⁶, —NHB⁵⁶ or —N(B⁵⁶)₂; and
wherein each B⁵⁶ is independently selected from a C₁-C₃ alkyl or C₁-C₃ haloalkyl group.

Typically, any divalent group —B⁵⁵— forms a 4- to 6-membered fused ring. Typically, R⁷ is C₁-C₄ alkyl, R⁸ is a 5- or 6-membered, optionally substituted heterocyclic or aromatic group, and R^{g} is hydrogen, halo, —CN, C₁-C₃ alkyl, C₁-C₃ haloalkyl, cyclopropyl or halocyclopropyl. More typically, R⁷ is C₁-C₄ alkyl, R⁸ is a 5- or 6-membered, optionally substituted heterocyclic or aromatic group, and R^{g} is hydrogen or halo. In one embodiment, the optional substituents on the heterocyclic or aromatic group are independently selected from halo, —OH, —NH₂, —CN, —B⁵, —OB⁵, —NHB⁵ or —N(B⁵)₂, wherein each B⁵ is independently selected from a C₁-C₄ alkyl, C₂-C₄ alkenyl or C₂-C₄ alkynyl group all of which may optionally be halo-substituted.

Typically, —R² has a formula selected from:

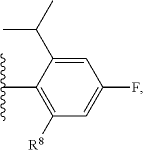

wherein R⁸ is a 5- or 6-membered, optionally substituted heterocyclic or aromatic group. In one embodiment, the optional substituents on the heterocyclic or aromatic group are independently selected from halo, —OH, —NH₂, —CN, —NO₂, —B⁶, —OB⁶, —NHB⁶, —N(B⁶)₂, —CONH₂, —CONHB⁶, —CON(B⁶)₂, —NHCOB⁶, —NB⁶COB⁶, or —B⁶⁶—;
wherein each B⁶ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁶ together with the nitrogen atom to which they are attached may form a 4- to 6-membered heterocyclic group containing one or two ring heteroatoms N and/or O, wherein any B⁶ may optionally be halo-substituted and/or substituted with one or two substituents independently selected from —OH, —NH₂, —OB⁶⁷, —NHB⁶⁷ or —N(B⁶⁷)₂;
wherein each B⁶⁶ is independently selected from a C₁-C₈ alkylene or C₂-C₈ alkenylene group, wherein one or two carbon atoms in the backbone of the alkylene or alkenylene group may optionally be replaced by one or two heteroatoms N and/or 0, and wherein the alkylene or alkenylene group may optionally be halo-substituted and/or substituted with one or two substituents independently selected from —OH, —NH₂, —OB⁶⁷, —NHB⁶⁷ or —N(B⁶⁷)₂; and
wherein each B⁶⁷ is independently selected from a C₁-C₃ alkyl or C₁-C₃ haloalkyl group.

Typically, any divalent group —B⁶⁶— forms a 4- to 6-membered fused ring. Typically, the optional substituents on the heterocyclic or aromatic group are independently selected from halo, —OH, —NH₂, —CN, —B⁶, —OB⁶, —NHB⁶ or —N(B⁶)₂, wherein each B⁶ is independently selected from a C₁-C₄ alkyl, C₂-C₄ alkenyl or C₂-C₄ alkynyl group which may optionally be halo-substituted.

In one embodiment, R² is a parent cyclic group substituted at the α-position with a monovalent heterocyclic group or a monovalent aromatic group, wherein the heterocyclic or aromatic group may optionally be substituted, and wherein the parent cyclic group may optionally be further substituted. The further substituents on the α-substituted parent cyclic group of R² also include cycloalkyl, cycloalkenyl, non-aromatic heterocyclic, aryl or heteroaryl rings which are fused to the α-substituted parent cyclic group of R². Typically, the cycloalkyl, cycloalkenyl, non-aromatic heterocyclic, aryl or heteroaryl rings are ortho-fused to the α-substituted parent cyclic group of R², i.e. each fused cycloalkyl, cycloalkenyl, non-aromatic heterocyclic, aryl or heteroaryl ring has only two atoms and one bond in common with the α-substituted parent cyclic group of R². Typically, the cycloalkyl, cycloalkenyl, non-aromatic heterocyclic, aryl or heteroaryl rings are ortho-fused to the α-substituted parent cyclic group of R² across the α′,β′ positions.

In one embodiment, —R² has a formula selected from:

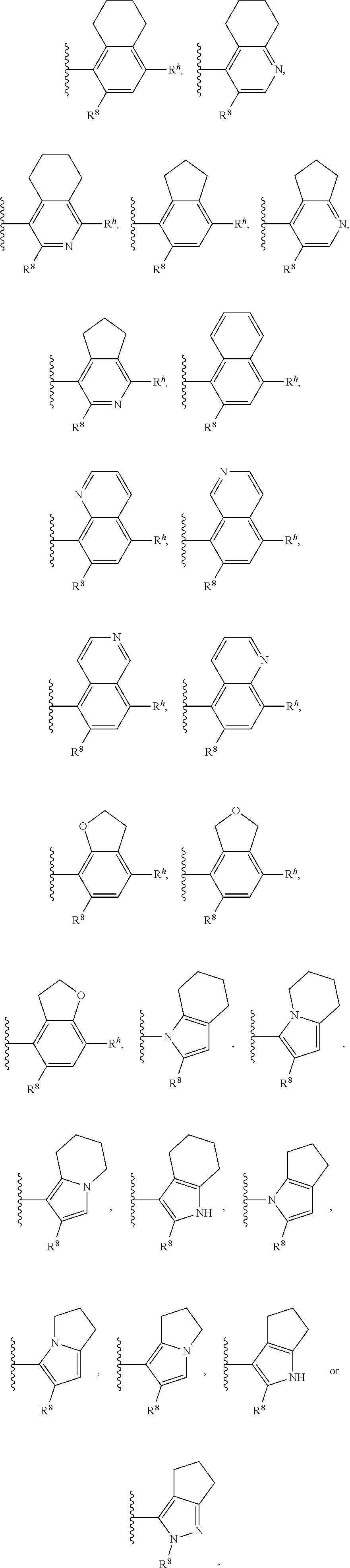

wherein R⁸ is a 5- or 6-membered, optionally substituted heterocyclic or aromatic group, and R^{h} is hydrogen, halo, —OH, —NO₂, —CN, —R^{hh}, —OR^{hh}, —COR^{hh}, —COOR^{hh}, —CONH₂, —CONHR^{hh} or —CON(R^{hh})₂, wherein each —R^{hh} is independently selected from C₁-C₄ alkyl, C₁-C₄ haloalkyl, C₃-C₄ cycloalkyl and C₃-C₄ halocycloalkyl. In one embodiment, the optional substituents on the heterocyclic or aromatic group are independently selected from halo, —OH, —NH₂, —CN, —NO₂, —B⁷, —OB⁷, —NHB⁷, —N(B⁷)₂, —CONH₂, —CONHB⁷, —CON(B⁷)₂, —NHCOB⁷, —NB⁷COB⁷, or —B⁷⁷—;
wherein each B⁷ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁷ together with the nitrogen atom to which they are attached may form a 4- to 6-membered heterocyclic group containing one or two ring heteroatoms N and/or O, wherein any B⁷ may optionally be halo-substituted and/or substituted with one or two substituents independently selected from —OH, —NH₂, —OB⁷⁸, —NHB⁷⁸ or —N(B⁷⁸)
wherein each B⁷⁷ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁷⁸, —NHB⁷⁸ or —N(B⁷⁸)₂; and
wherein each B⁷⁸ is independently selected from a C₁-C₃ alkyl or C₁-C₃ haloalkyl group.

Typically, any divalent group —B⁷⁷— forms a 4- to 6-membered fused ring. Typically, R^{h} is hydrogen, halo, —CN, C₁-C₃ alkyl, C₁-C₃ haloalkyl, cyclopropyl or halocyclopropyl. More typically, R^{h} is hydrogen or halo. Typically, the optional substituents on the heterocyclic or aromatic group are independently selected from halo, —OH, —NH₂, —CN, —B⁷, —OB⁷, —NHB⁷ or —N(B⁷)₂, wherein each B⁷ is independently selected from a C₁-C₄ alkyl, C₂-C₄ alkenyl or C₂-C₄ alkynyl group all of which may optionally be halo-substituted.

In one embodiment, —R² has a formula selected from:

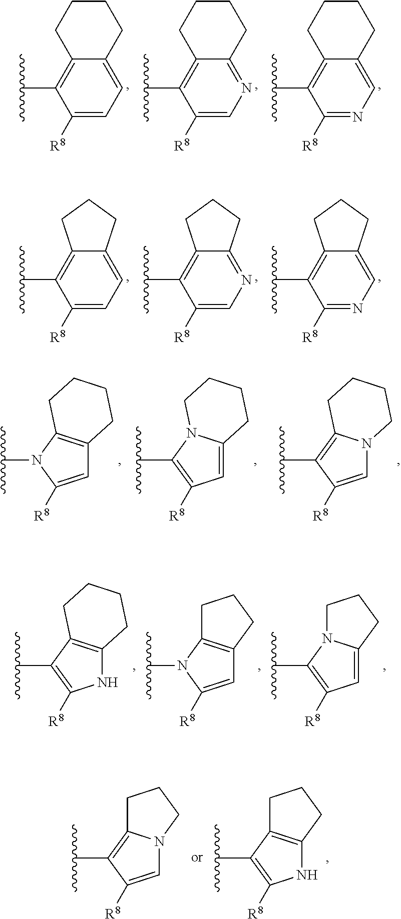

wherein R⁸ is a 5- or 6-membered, optionally substituted heterocyclic or aromatic group. In one embodiment, the optional substituents on the heterocyclic or aromatic group are independently selected from halo, —OH, —NH₂, —CN, —NO₂, —B⁸, —OB⁸, —NHB⁸, —N(B⁸)₂, —CONH₂, —CONHB⁸, —CON(B⁸)₂, —NHCOB⁸, —NB⁸COB⁸, or —B⁸⁸—;
wherein each B⁸ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁸ together with the nitrogen atom to which they are attached may form a 4- to 6-membered heterocyclic group containing one or two ring heteroatoms N and/or O, wherein any B⁸ may optionally be halo-substituted and/or substituted with one or two substituents independently selected from —OH, —NH₂, —OB⁸⁹, —NHB⁸⁹ or —N(B⁸⁹)₂;
wherein each B⁸⁸ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁸⁹, —NHB⁸⁹ or —N(B⁸⁹)₂; and
wherein each B⁸⁹ is independently selected from a C₁-C₃ alkyl or C₁-C₃ haloalkyl group.

Typically, any divalent group —B⁸⁸— forms a 4- to 6-membered fused ring. Typically, the optional substituents on the heterocyclic or aromatic group are independently selected from halo, —OH, —NH₂, —CN, —B⁸, —OB⁸, —NHB⁸ or —N(B⁸)₂, wherein each B⁸ is independently selected from a C₁-C₄ alkyl, C₂-C₄ alkenyl or C₂-C₄ alkynyl group all of which may optionally be halo-substituted.

Typically, —R² has a formula selected from:

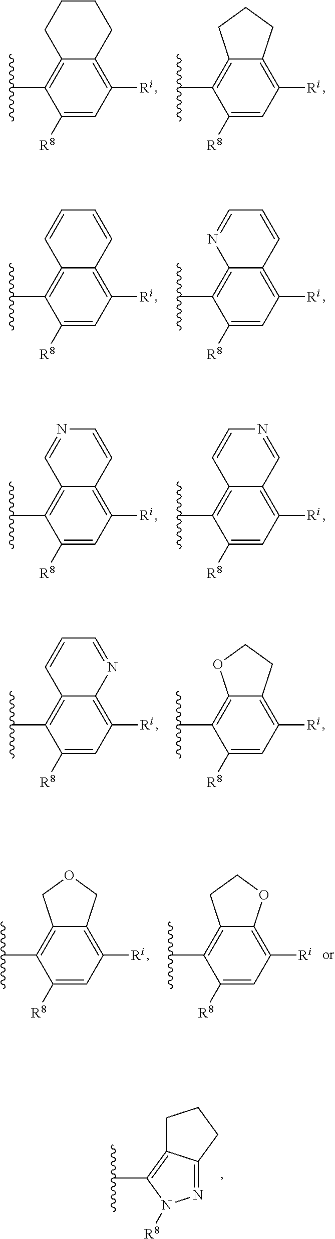

wherein R⁸ is a 5- or 6-membered, optionally substituted heterocyclic or aromatic group, and R¹ is hydrogen, halo, —OH, —NO₂, —CN, —Rⁱⁱ, —ORⁱⁱ, —CORⁱⁱ, —COORⁱⁱ, —CONH₂, —CONHRⁱⁱ or —CON(Rⁱⁱ)₂, wherein each —Rⁱⁱ is independently selected from C₁-C₄ alkyl, C₁-C₄ haloalkyl, C₃-C₄ cycloalkyl and C₃-C₄ halocycloalkyl. In one embodiment, the optional substituents on the heterocyclic or aromatic group are independently selected from halo, —OH, —NH₂, —CN, —NO₂, —B⁹, —OB⁹, —NHB⁹, —N(B⁹)₂, —CONH₂, —CONHB⁹, —CON(B⁹)₂, —NHCOB⁹, —NB⁹COB⁹, or —B^{gg}—;
wherein each B⁹ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁹ together with the nitrogen atom to which they are attached may form a 4- to 6-membered heterocyclic group containing one or two ring heteroatoms N and/or O, wherein any B⁹ may optionally be halo-substituted and/or substituted with one or two substituents independently selected from —OH, —NH₂, —OB⁹⁸, —NHB⁹⁸ or —N(B⁹⁸)
wherein each B^{gg} is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁹⁸, —NHB⁹⁸ or —N(B⁹⁸)₂; and
wherein each B⁹⁸ is independently selected from a C₁-C₃ alkyl or C₁-C₃ haloalkyl group.

Typically, any divalent group —B^{gg}— forms a 4- to 6-membered fused ring. Typically, R¹ is hydrogen, halo, —CN, C₁-C₃ alkyl, C₁-C₃ haloalkyl, cyclopropyl or halocyclopropyl. More typically, R¹ is hydrogen or halo. Typically, the optional substituents on the heterocyclic or aromatic group are independently selected from halo, —OH, —NH₂, —CN, —B⁹, —OB⁹, —NHB⁹ or —N(B⁹)₂, wherein each B⁹ is independently selected from a C₁-C₄ alkyl, C₂-C₄ alkenyl or C₂-C₄ alkynyl group all of which may optionally be halo-substituted.

In one embodiment, R² is phenyl or a 5- or 6-membered heteroaryl group (such as phenyl, pyridinyl, pyridazinyl, pyrimidinyl or pyrazinyl); wherein
(i) the phenyl or 5- or 6-membered heteroaryl group is substituted at the α position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted at the α′ position with a substituent selected from —R³⁴, —OR³⁴ and —COR³⁴, wherein R³⁴ is selected from a C₁-C₆ alkyl, C₂-C₆ alkenyl, C₂-C₆ alkynyl or C₂-C₆ cyclic group and wherein R³⁴ is optionally substituted with one or more halo groups; and
optionally the phenyl or 5- or 6-membered heteroaryl group is further substituted (typically with one, two or three substituents independently selected from halo, —NO₂, —CN, —COOR³⁵, —CONH₂, —CONHR³⁵ or —CON(R³⁵)₂, wherein each —R³⁵ is independently selected from a C₁-C₄ alkyl or C₁-C₄ haloalkyl group); or
(ii) the phenyl or 5- or 6-membered heteroaryl group is substituted with a cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and
optionally the phenyl or 5- or 6-membered heteroaryl group is further substituted at the α′ position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted (typically with one or two substituents independently selected from halo, —NO₂, —CN, —COOR³⁵, —CONH₂, —CONHR³⁵ or —CON(R³⁵)₂, wherein each —R³⁵ is independently selected from a C₁-C₄ alkyl or C₁-C₄ haloalkyl group); or
(iii) the phenyl or 5- or 6-membered heteroaryl group is substituted with a first cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and
the phenyl or 5- or 6-membered heteroaryl group is substituted with a second cycloalkyl, cycloalkenyl, non-aromatic heterocyclic, aryl or heteroaryl ring which is fused to the parent phenyl or 5- or 6-membered heteroaryl group across the α′,β′ positions and which is optionally substituted with one or more halo groups; and
optionally the phenyl group is further substituted (typically with a substituent selected from halo, —NO₂, —CN, —COOR³⁵, —CONH₂, —CONHR³⁵ or —CON(R³⁵)₂, wherein each —R³⁵ is independently selected from a C₁-C₄ alkyl or C₁-C₄ haloalkyl group); or
(iv) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, C₁-C₃ alkyl, C₁-C₃ haloalkyl, —R³²—OR³³, —R³²—N(R³³)₂, —R³²—CN or —R³²—C≡CR³³, and wherein a ring atom of the monovalent heterocyclic or aromatic group is directly attached to the α-ring atom of the parent phenyl or 5- or 6-membered heteroaryl group; wherein R³² is independently selected from a bond or a C₁-C₃ alkylene group; and R³³ is independently selected from hydrogen or a C₁-C₃ alkyl or C₁-C₃ haloalkyl group; and
optionally the phenyl or 5- or 6-membered heteroaryl group is further substituted at the α′ position with a substituent selected from —R⁴, —OR⁴ and —COR⁴, wherein R⁴ is selected from a C₁-C₆ alkyl, C₂-C₆ alkenyl, C₂-C₆ alkynyl or C₂-C₆ cyclic group and wherein R⁴ is optionally substituted with one or more halo groups; and
optionally the phenyl or 5- or 6-membered heteroaryl group is further substituted (typically with one, two or three substituents independently selected from halo, —NO₂, —CN, —COOR³⁵, —CONH₂, —CONHR³⁵ or —CON(R³⁵)₂, wherein each —R³⁵ is independently selected from a C₁-C₄ alkyl or C₁-C₄ haloalkyl group); or
(v) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, C₁-C₃ alkyl, C₁-C₃ haloalkyl, —R³²—OR³³, —R³²—N(R³³)₂, —R³²—CN or —R³²—C≡CR³³, and wherein a ring atom of the monovalent heterocyclic or aromatic group is directly attached to the α-ring atom of the parent phenyl or 5- or 6-membered heteroaryl group; wherein R³² is independently selected from a bond or a C₁-C₃ alkylene group; and R³³ is independently selected from hydrogen or a C₁-C₃ alkyl or C₁-C₃ haloalkyl group; and
optionally the phenyl or 5- or 6-membered heteroaryl group is further substituted with a cycloalkyl, cycloalkenyl, non-aromatic heterocyclic, aryl or heteroaryl ring which is fused to the parent phenyl or 5- or 6-membered heteroaryl group across the α′,β′ positions and which is optionally substituted with one or more halo groups; and
optionally the phenyl or 5- or 6-membered heteroaryl group is further substituted (typically with one or two substituents independently selected from halo, —NO₂, —CN, —COOR³⁵, —CONH₂, —CONHR³⁵ or —CON(R³⁵)₂, wherein each —R³⁵ is independently selected from a C₁-C₄ alkyl or C₁-C₄ haloalkyl group).

In the embodiment directly above, where a group or moiety is optionally substituted with one or more halo groups, it may be substituted for example with one, two, three, four, five or six halo groups.

In one aspect of any of the above embodiments, R² contains from 10 to 50 atoms other than hydrogen. More typically, R² contains from 10 to 40 atoms other than hydrogen.

More typically, R² contains from 10 to 35 atoms other than hydrogen. Most typically, R² contains from 12 to 30 atoms other than hydrogen.

In one aspect of any of the above embodiments, R² contains from 5 to 30 atoms other than hydrogen or halogen. More typically, R² contains from 7 to 25 atoms other than hydrogen or halogen. More typically, R² contains from 9 to 20 atoms other than hydrogen or halogen. More typically still, R² contains from 10 to 20 atoms other than hydrogen or halogen. Most typically, R² contains from 12 to 18 atoms other than hydrogen or halogen.

Q is selected from O or S. In one embodiment of the first aspect of the invention, Q is O.

In one aspect of any of the above embodiments, the compound of formula (I) has a molecular weight of from 250 to 2000 Da. Typically, the compound of formula (I) has a molecular weight of from 300 to 900 Da. More typically, the compound of formula (I) has a molecular weight of from 325 to 650 Da. More typically still, the compound of formula (I) has a molecular weight of from 350 to 600 Da.

A second aspect of the invention provides a compound selected from the group consisting of:

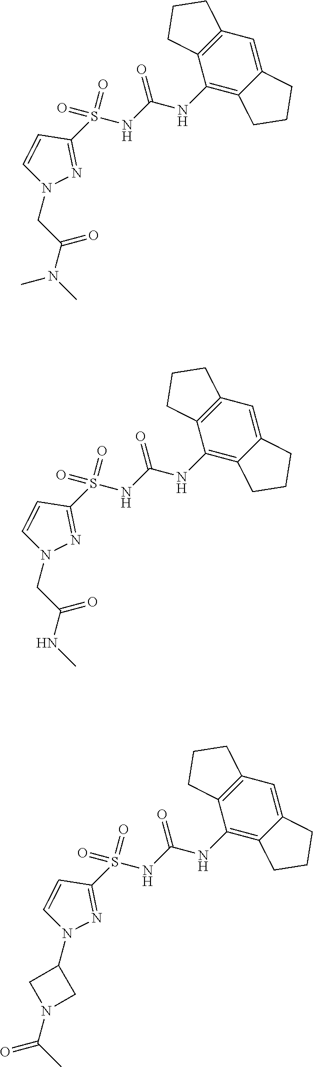 [SMILES: CN(C)C(=O)Cn1ccc(S(=O)(=O)NC(=O)Nc2c3c(cc4c2CCC4)CCC3)n1 ; CNC(=O)Cn1ccc(S(=O)(=O)NC(=O)Nc2c3c(cc4c2CCC4)CCC3)n1 ; CC(=O)N1CC(n2ccc(S(=O)(=O)NC(=O)Nc3c4c(cc5c3CCC5)CCC4)n2)C1]

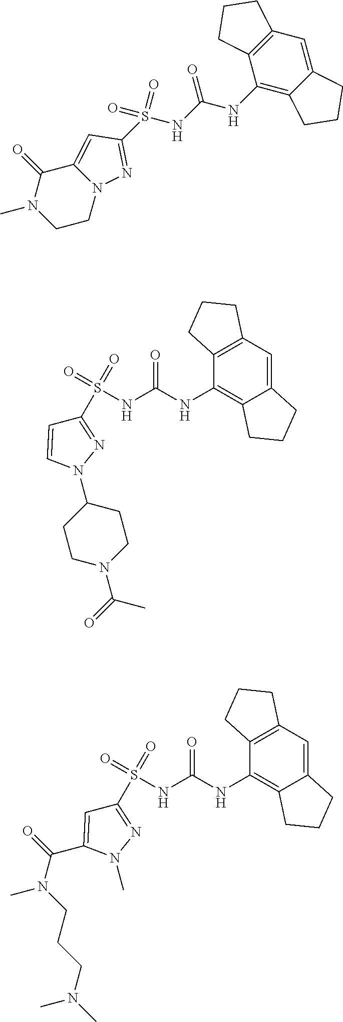 [SMILES: CN1CCn2nc(S(=O)(=O)NC(=O)Nc3c4c(cc5c3CCC5)CCC4)cc2C1=O ; CC(=O)N1CCC(n2ccc(S(=O)(=O)NC(=O)Nc3c4c(cc5c3CCC5)CCC4)n2)CC1 ; CN(C)CCCN(C)C(=O)c1cc(S(=O)(=O)NC(=O)Nc2c3c(cc4c2CCC4)CCC3)nn1C]

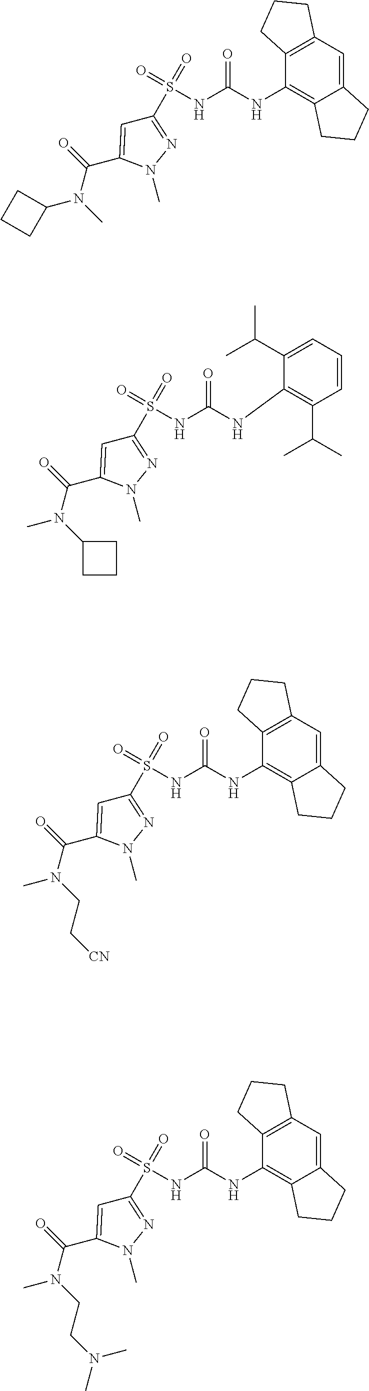

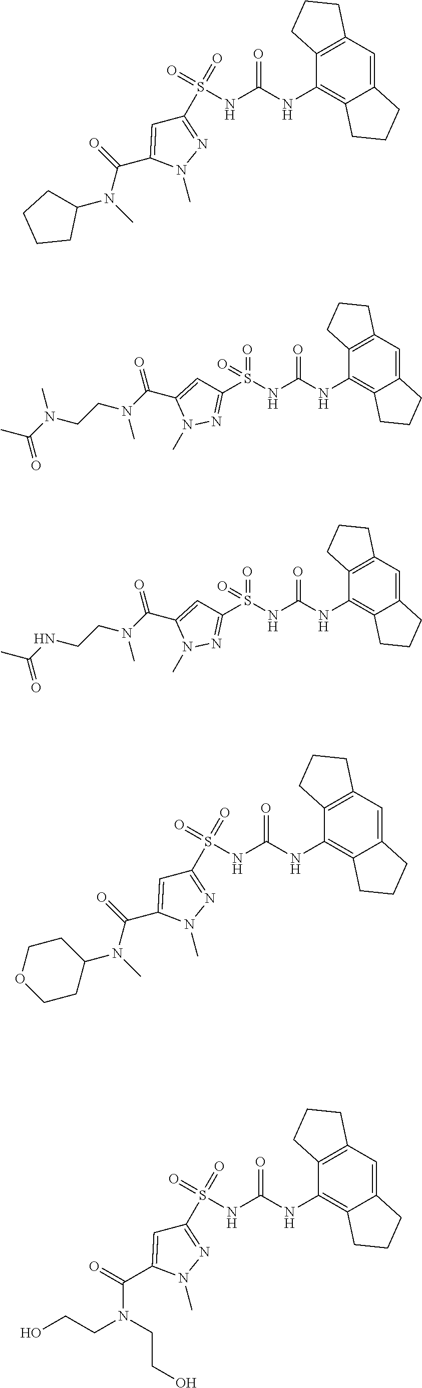 [SMILES: CN(C(=O)c1cc(S(=O)(=O)NC(=O)Nc2c3c(cc4c2CCC4)CCC3)nn1C)C1CCCC1 ; CC(=O)N(C)CCN(C)C(=O)c1cc(S(=O)(=O)NC(=O)Nc2c3c(cc4c2CCC4)CCC3)nn1C ; CC(=O)NCCN(C)C(=O)c1cc(S(=O)(=O)NC(=O)Nc2c3c(cc4c2CCC4)CCC3)nn1C ; CN(C(=O)c1cc(S(=O)(=O)NC(=O)Nc2c3c(cc4c2CCC4)CCC3)nn1C)C1CCOCC1 ; Cn1nc(S(=O)(=O)NC(=O)Nc2c3c(cc4c2CCC4)CCC3)cc1C(=O)N(CCO)CCO]

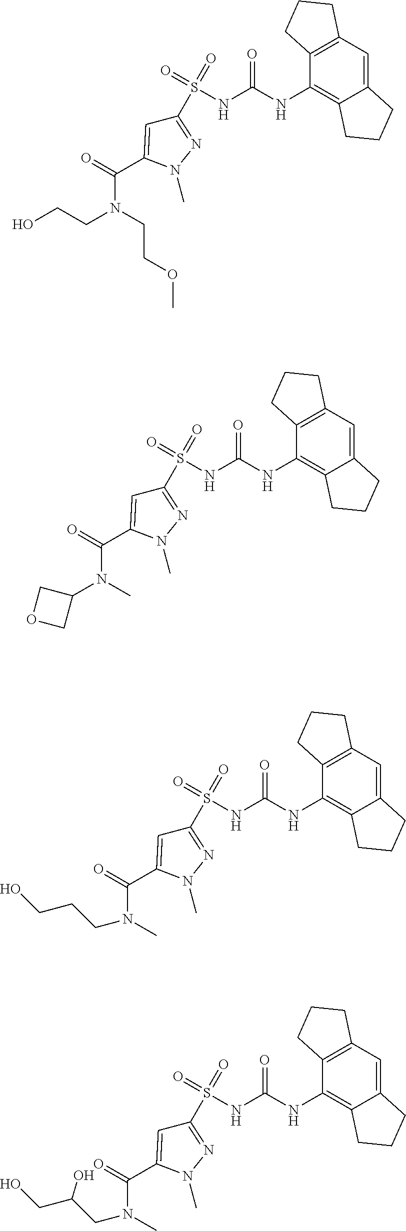 [SMILES: COCCN(CCO)C(=O)c1cc(S(=O)(=O)NC(=O)Nc2c3c(cc4c2CCC4)CCC3)nn1C ; CN(C(=O)c1cc(S(=O)(=O)NC(=O)Nc2c3c(cc4c2CCC4)CCC3)nn1C)C1COC1 ; CN(CCCO)C(=O)c1cc(S(=O)(=O)NC(=O)Nc2c3c(cc4c2CCC4)CCC3)nn1C ; CN(CC(O)CO)C(=O)c1cc(S(=O)(=O)NC(=O)Nc2c3c(cc4c2CCC4)CCC3)nn1C]

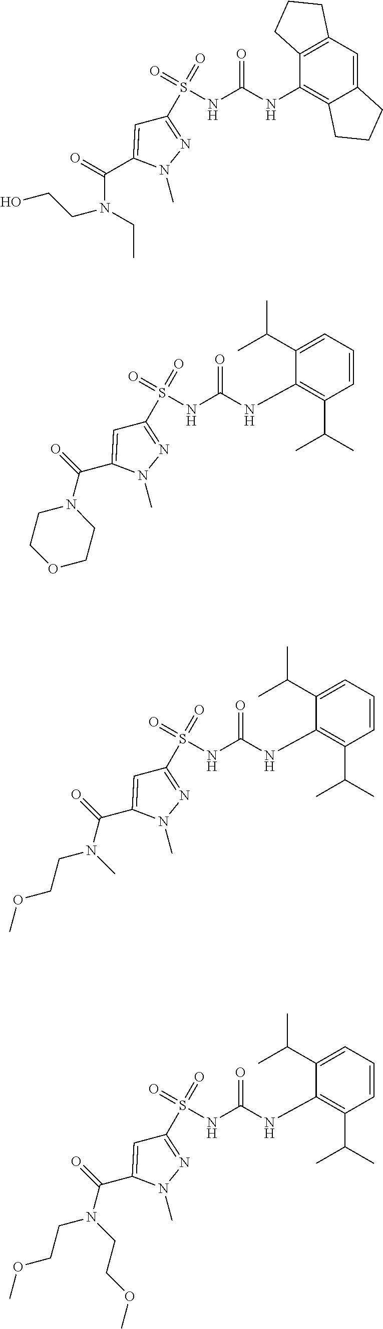

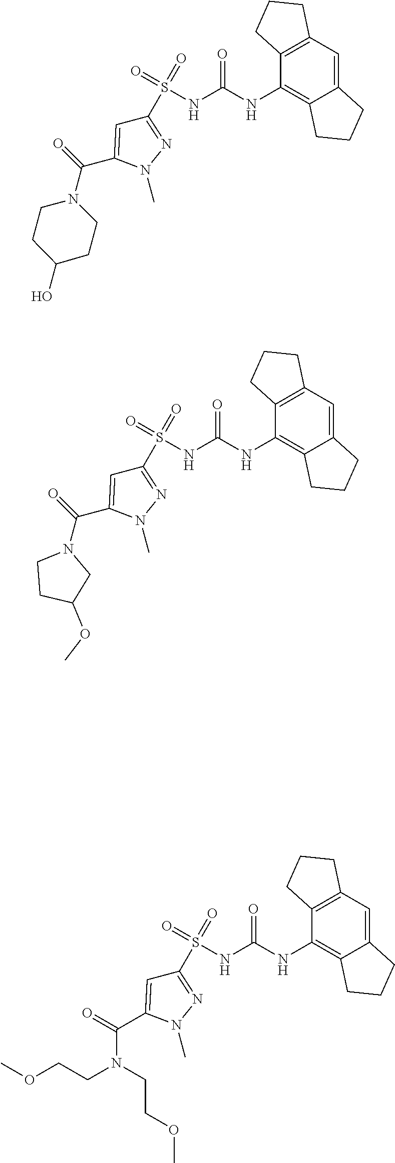 [SMILES: Cn1nc(S(=O)(=O)NC(=O)Nc2c3c(cc4c2CCC4)CCC3)cc1C(=O)N1CCC(O)CC1 ; COC1CCN(C(=O)c2cc(S(=O)(=O)NC(=O)Nc3c4c(cc5c3CCC5)CCC4)nn2C)C1 ; COCCN(CCOC)C(=O)c1cc(S(=O)(=O)NC(=O)Nc2c3c(cc4c2CCC4)CCC3)nn1C]

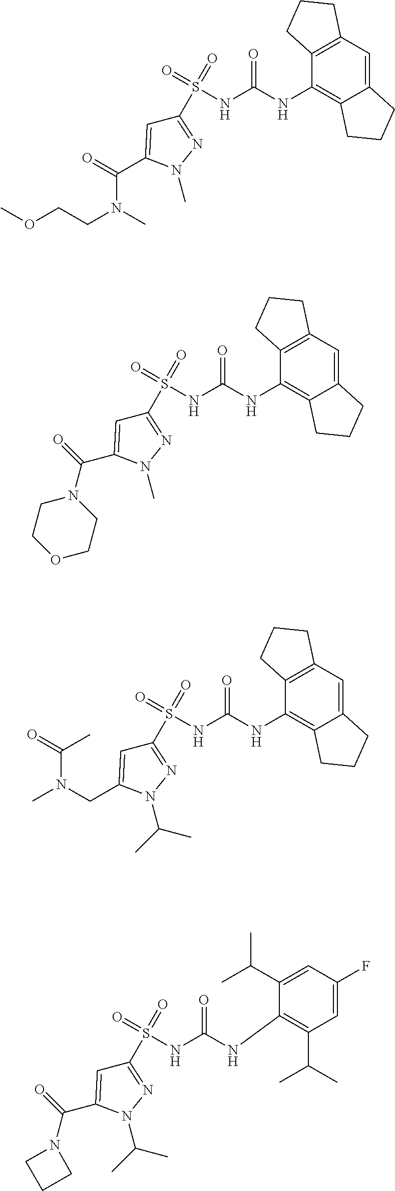 [SMILES: COCCN(C)C(=O)c1cc(S(=O)(=O)NC(=O)Nc2c3c(cc4c2CCC4)CCC3)nn1C ; Cn1nc(S(=O)(=O)NC(=O)Nc2c3c(cc4c2CCC4)CCC3)cc1C(=O)N1CCOCC1 ; CC(=O)N(C)Cc1cc(S(=O)(=O)NC(=O)Nc2c3c(cc4c2CCC4)CCC3)nn1C(C)C ; CC(C)c1cc(F)cc(C(C)C)c1NC(=O)NS(=O)(=O)c1cc(C(=O)N2CCC2)n(C(C)C)n1]

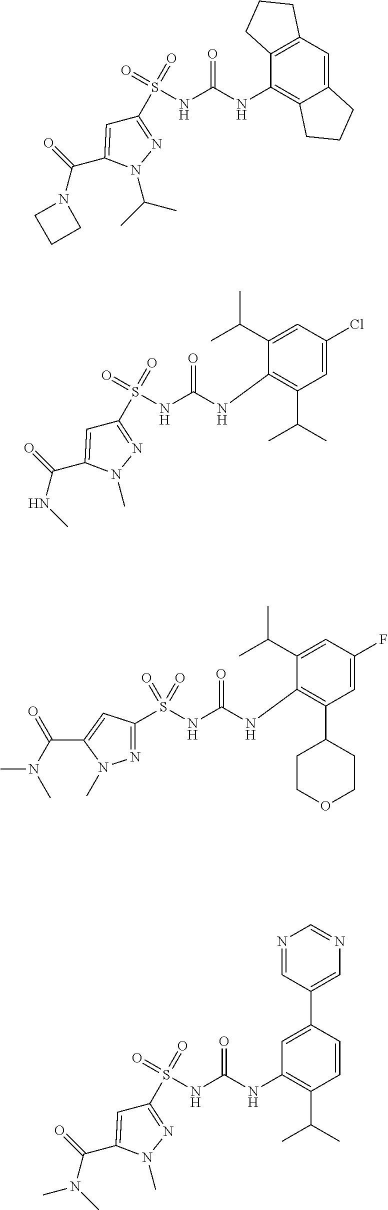 [SMILES: CC(C)n1nc(S(=O)(=O)NC(=O)Nc2c3c(cc4c2CCC4)CCC3)cc1C(=O)N1CCC1 ; CNC(=O)c1cc(S(=O)(=O)NC(=O)Nc2c(C(C)C)cc(Cl)cc2C(C)C)nn1C ; CC(C)c1cc(F)cc(C2CCOCC2)c1NC(=O)NS(=O)(=O)c1cc(C(=O)N(C)C)n(C)n1 ; CC(C)c1ccc(-c2cncnc2)cc1NC(=O)NS(=O)(=O)c1cc(C(=O)N(C)C)n(C)n1]

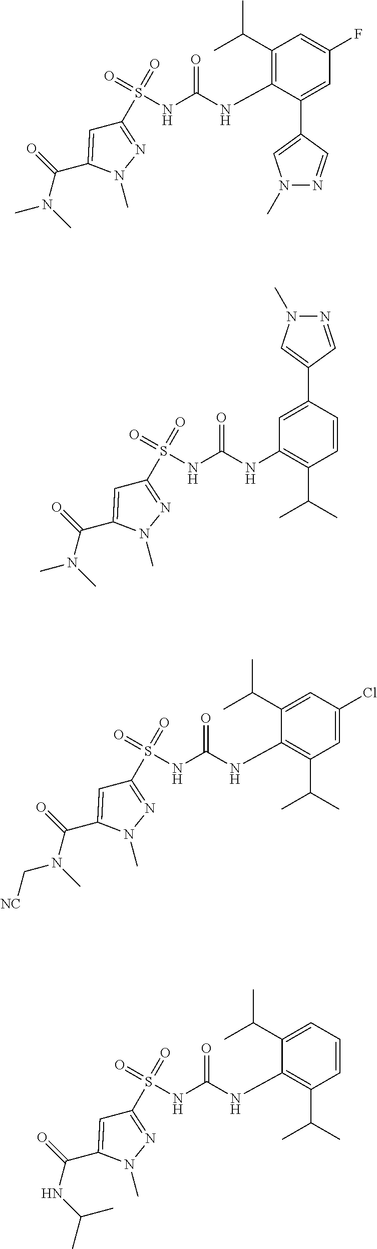 [SMILES: CC(C)c1cc(F)cc(-c2cnn(C)c2)c1NC(=O)NS(=O)(=O)c1cc(C(=O)N(C)C)n(C)n1 ; CC(C)c1ccc(-c2cnn(C)c2)cc1NC(=O)NS(=O)(=O)c1cc(C(=O)N(C)C)n(C)n1 ; CC(C)c1cc(Cl)cc(C(C)C)c1NC(=O)NS(=O)(=O)c1cc(C(=O)N(C)CC#N)n(C)n1 ; CC(C)NC(=O)c1cc(S(=O)(=O)NC(=O)Nc2c(C(C)C)cccc2C(C)C)nn1C]

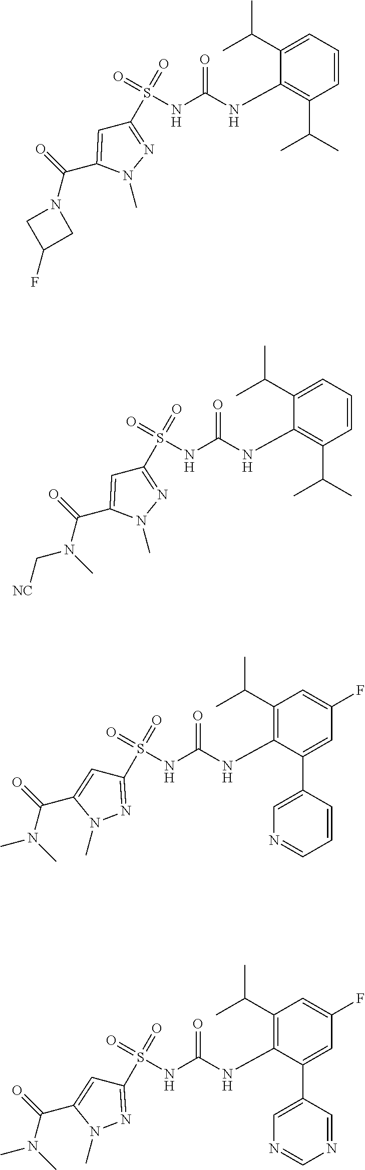

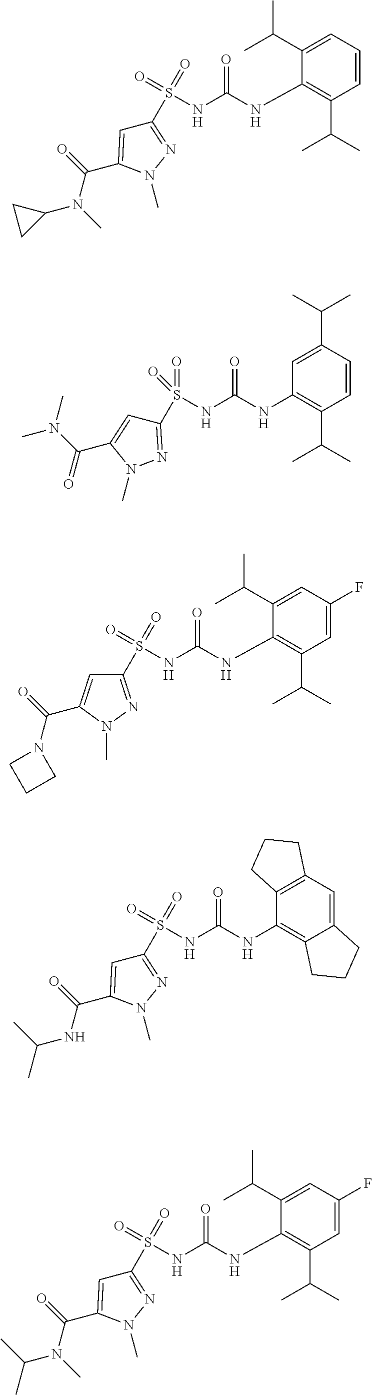

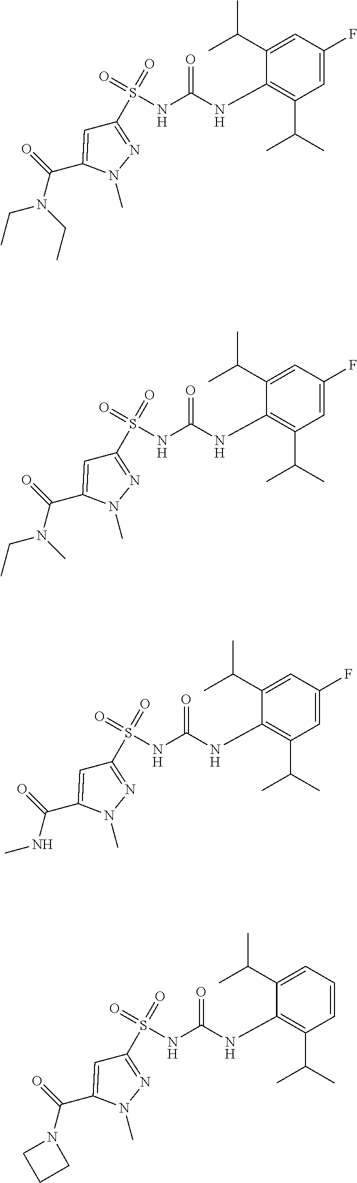 [SMILES: CCN(CC)C(=O)c1cc(S(=O)(=O)NC(=O)Nc2c(C(C)C)cc(F)cc2C(C)C)nn1C ; CCN(C)C(=O)c1cc(S(=O)(=O)NC(=O)Nc2c(C(C)C)cc(F)cc2C(C)C)nn1C ; CNC(=O)c1cc(S(=O)(=O)NC(=O)Nc2c(C(C)C)cc(F)cc2C(C)C)nn1C ; CC(C)c1cccc(C(C)C)c1NC(=O)NS(=O)(=O)c1cc(C(=O)N2CCC2)n(C)n1]

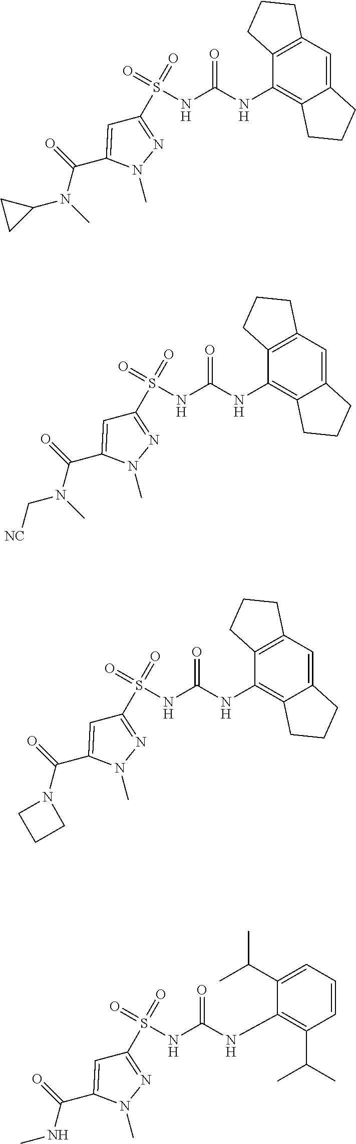 [SMILES: CN(C(=O)c1cc(S(=O)(=O)NC(=O)Nc2c3c(cc4c2CCC4)CCC3)nn1C)C1CC1 ; CN(CC#N)C(=O)c1cc(S(=O)(=O)NC(=O)Nc2c3c(cc4c2CCC4)CCC3)nn1C ; Cn1nc(S(=O)(=O)NC(=O)Nc2c3c(cc4c2CCC4)CCC3)cc1C(=O)N1CCC1 ; CNC(=O)c1cc(S(=O)(=O)NC(=O)Nc2c(C(C)C)cccc2C(C)C)nn1C]

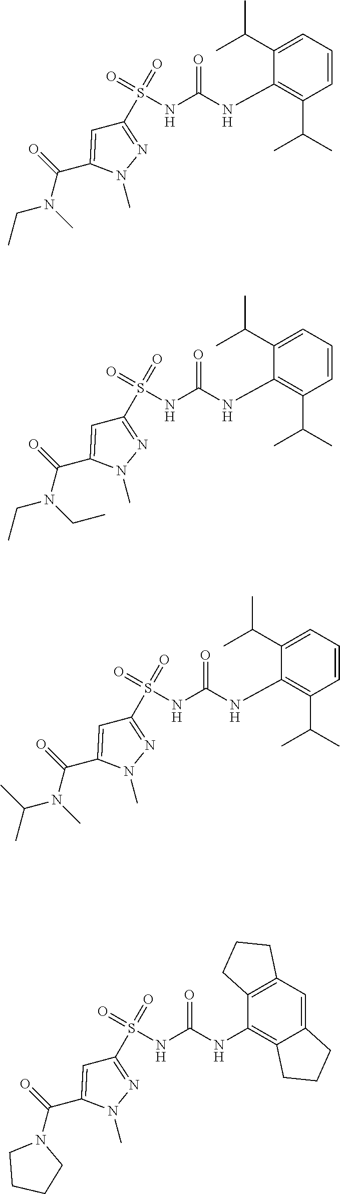

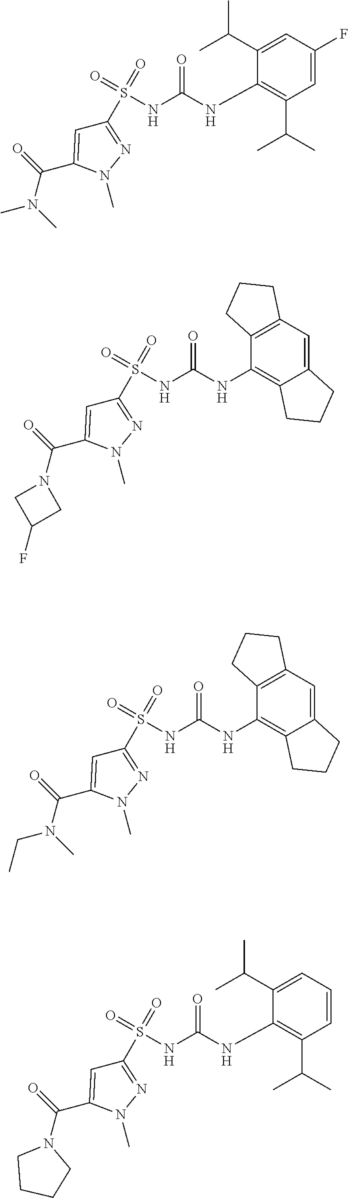 [SMILES: CC(C)c1cc(F)cc(C(C)C)c1NC(=O)NS(=O)(=O)c1cc(C(=O)N(C)C)n(C)n1 ; Cn1nc(S(=O)(=O)NC(=O)Nc2c3c(cc4c2CCC4)CCC3)cc1C(=O)N1CC(F)C1 ; CCN(C)C(=O)c1cc(S(=O)(=O)NC(=O)Nc2c3c(cc4c2CCC4)CCC3)nn1C ; CC(C)c1cccc(C(C)C)c1NC(=O)NS(=O)(=O)c1cc(C(=O)N2CCCC2)n(C)n1]

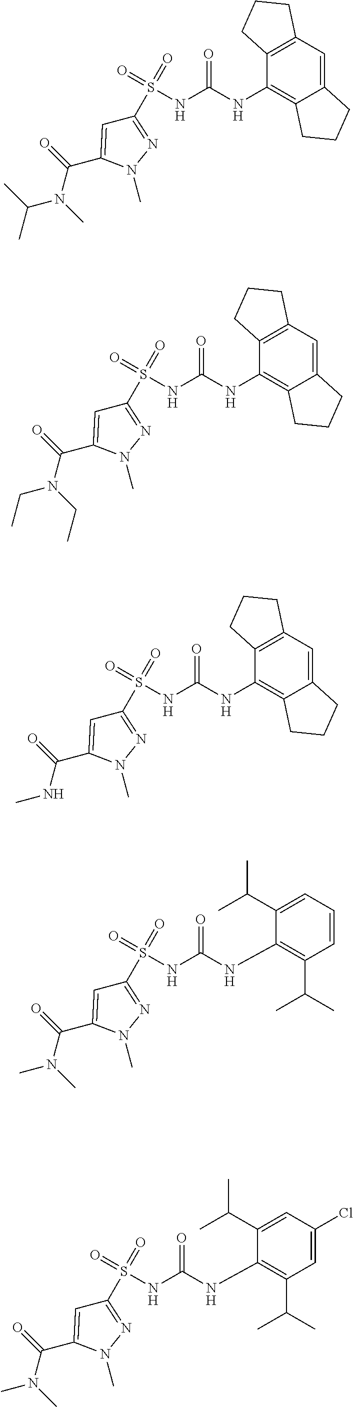

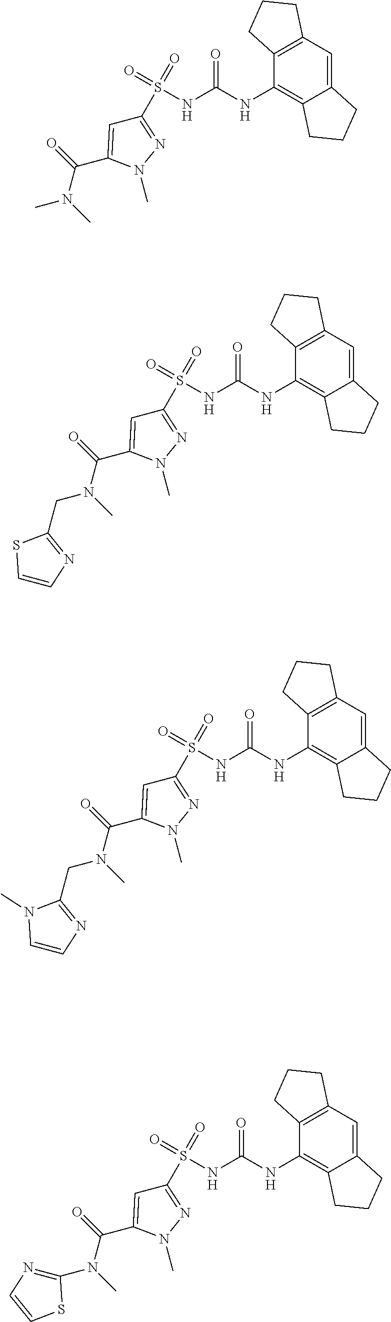 [SMILES: CN(C)C(=O)c1cc(S(=O)(=O)NC(=O)Nc2c3c(cc4c2CCC4)CCC3)nn1C ; CN(Cc1nccs1)C(=O)c1cc(S(=O)(=O)NC(=O)Nc2c3c(cc4c2CCC4)CCC3)nn1C ; CN(Cc1nccn1C)C(=O)c1cc(S(=O)(=O)NC(=O)Nc2c3c(cc4c2CCC4)CCC3)nn1C ; CN(C(=O)c1cc(S(=O)(=O)NC(=O)Nc2c3c(cc4c2CCC4)CCC3)nn1C)c1nccs1]

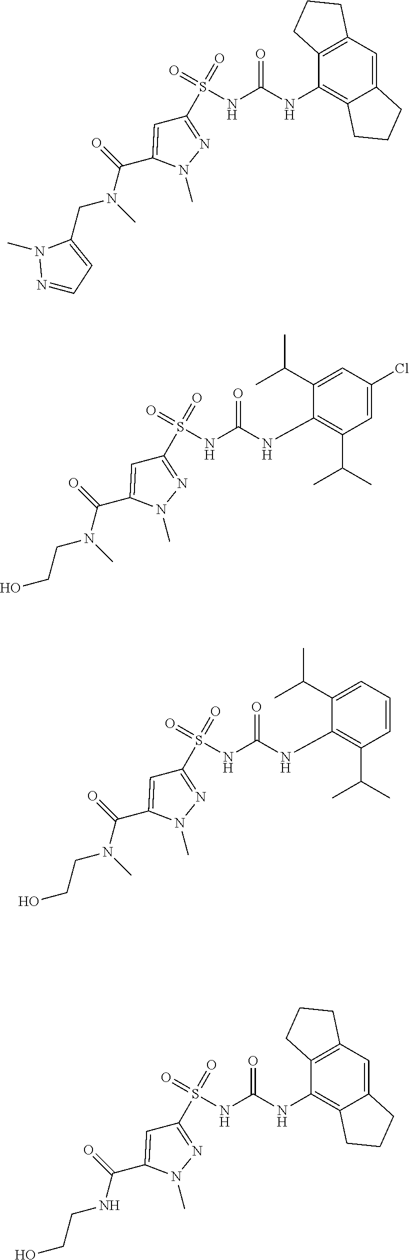 [SMILES: CN(Cc1ccnn1C)C(=O)c1cc(S(=O)(=O)NC(=O)Nc2c3c(cc4c2CCC4)CCC3)nn1C ; CC(C)c1cc(Cl)cc(C(C)C)c1NC(=O)NS(=O)(=O)c1cc(C(=O)N(C)CCO)n(C)n1 ; CC(C)c1cccc(C(C)C)c1NC(=O)NS(=O)(=O)c1cc(C(=O)N(C)CCO)n(C)n1 ; Cn1nc(S(=O)(=O)NC(=O)Nc2c3c(cc4c2CCC4)CCC3)cc1C(=O)NCCO]

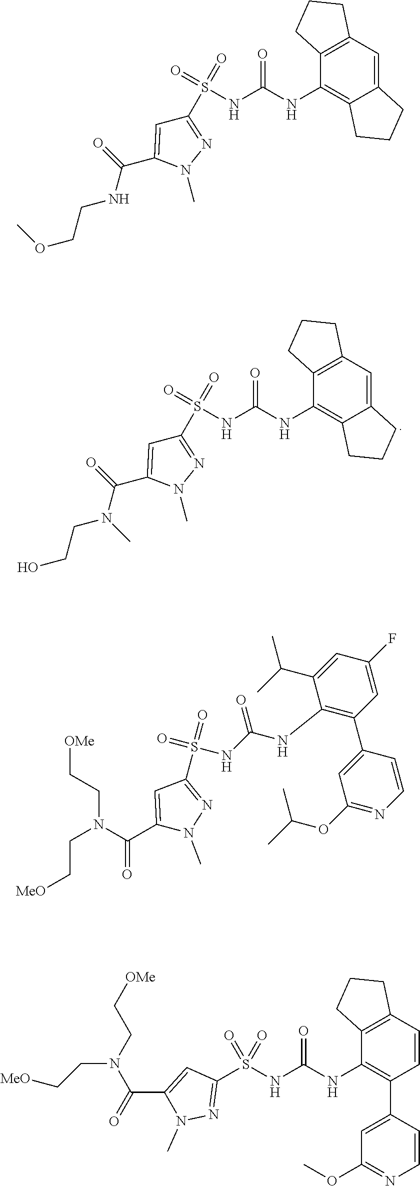 [SMILES: COCCNC(=O)c1cc(S(=O)(=O)NC(=O)Nc2c3c(cc4c2CCC4)CCC3)nn1C ; CN(CCO)C(=O)c1cc(S(=O)(=O)NC(=O)Nc2c3c(cc4c2CCC4)CCC3)nn1C ; COCCN(CCOC)C(=O)c1cc(S(=O)(=O)NC(=O)Nc2c(-c3ccnc(OC(C)C)c3)cc(F)cc2C(C)C)nn1C ; COCCN(CCOC)C(=O)c1cc(S(=O)(=O)NC(=O)Nc2c(-c3ccnc(OC)c3)ccc3c2CCC3)nn1C]

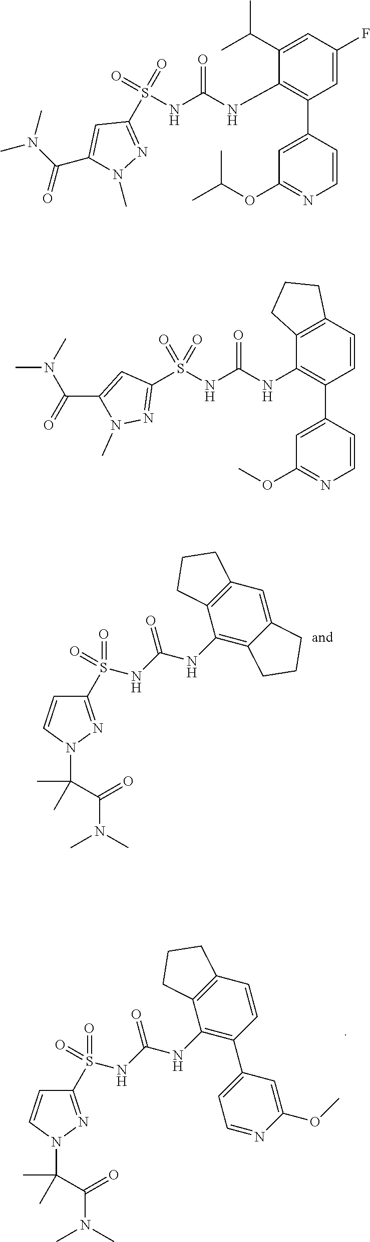 [SMILES: CC(C)Oc1cc(-c2cc(F)cc(C(C)C)c2NC(=O)NS(=O)(=O)c2cc(C(=O)N(C)C)n(C)n2)ccn1 ; COc1cc(-c2ccc3c(c2NC(=O)NS(=O)(=O)c2cc(C(=O)N(C)C)n(C)n2)CCC3)ccn1 ; CN(C)C(=O)C(C)(C)n1ccc(S(=O)(=O)NC(=O)Nc2c3c(cc4c2CCC4)CCC3)n1 ; COc1cc(-c2ccc3c(c2NC(=O)NS(=O)(=O)c2ccn(C(C)(C)C(=O)N(C)C)n2)CCC3)ccn1]

A third aspect of the invention provides a pharmaceutically acceptable salt, solvate or prodrug of any compound of the first or second aspect of the invention.

The compounds of the present invention can be used both in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid-addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation.

Suitable cations include, but are not limited to lithium, sodium, potassium, magnesium, calcium and ammonium. The salt may be a mono-, di-, tri- or multi-salt.

Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound.

Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³J, ¹²⁴J, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention is a topical pharmaceutical composition. For example, the topical pharmaceutical composition may be a dermal pharmaceutical composition or an ocular pharmaceutical composition.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers.

Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL1β and IL18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation.

Typically, identification of the mutation in NLRP3 in the individual may be by any suitable genetic or biochemical means.

A tenth aspect of the invention provides a method of treatment or prevention of a disease, disorder or condition, the method comprising the steps of diagnosing of an individual having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481:278-286, 2012).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur. J. Immunol., 40: 595-653, 2010). In particular, NLRP3 mutations have been found to be responsible for a set of rare autoinflammatory diseases known as CAPS (Ozaki et al., J. Inflammation Research, 8:15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type-1 diabetes (T1D), psoriasis, rheumatoid arthritis (RA), Behcet's disease, Schnitzler syndrome, macrophage activation syndrome (Masters Clin. Immunol. 2013; Braddock et al. Nat. Rev. Drug Disc. 2004 3: 1-10; Inoue et al., Immunology 139: 11-18, Coll et al. Nat. Med. 2015 21(3):248-55; and Scott et al. Clin. Exp. Rheumatol 2016 34(1): 88-93), systemic lupus erythematosus (Lu et al. J Immunol. 2017198(3): 1119-29), and systemic sclerosis (Artlett et al. Arthritis Rheum. 2011; 63(11): 3563-74). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017196(3): 283-97). NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 2017 61: 306-316), intracranial aneurysms (Zhang et al. J. Stroke & Cerebrovascular Dis. 2015 24; 5: 972-979), and traumatic brain injury (Ismael et al. J Neurotrauma. 2018 Jan. 2). NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (Mridha et al. J Hepatol. 2017 66(5): 1037-46). A role for NLRP3 via IL-1β has also been suggested in atherosclerosis, myocardial infarction (van Hout et al. Eur. Heart J. 2017 38(11): 828-36), heart failure (Sano et al. J A M. Coll. Cardiol. 2018 71(8): 875-66), aortic aneurysm and dissection (Wu et al. Arterioscler. Thromb. Vasc. Biol. 2017 37(4): 694-706), and other cardiovascular events (Ridker et al., N Engl J Med., doi: 10.1056/NEJMoa1707914, 2017). Other diseases in which NLRP3 has been shown to be involved include: ocular diseases such as both wet and dry age-related macular degeneration (Doyle et al., Nature Medicine, 18: 791-798, 2012 and Tarallo et al. Cell 2012149(4): 847-59), diabetic retinopathy (Loukovaara et al. Acta Ophthalmol. 2017; 95(8): 803-808) and optic nerve damage (Puyang et al. Sci Rep. 2016 Feb. 19; 6:20998); liver diseases including non-alcoholic steatohepatitis (NASH) (Henao-Meija et al., Nature, 482: 179-185, 2012); inflammatory reactions in the lung and skin (Primiano et al. J Immunol. 2016 197(6): 2421-33) including contact hypersensitivity (such as bullous pemphigoid (Fang et al. J Dermatol Sci. 2016; 83(2): 116-23)), atopic dermatitis (Niebuhr et al. Allergy 2014 69(8): 1058-67), Hidradenitis suppurativa (Alikhan et al. 2009 J Am Acad Dermatol 60(4): 539-61), acne vulgaris (Qin et al. J Invest. Dermatol. 2014 134(2): 381-88), and sarcoidosis (Jager et al. Am J Respir Crit Care Med 2015 191: A5816); inflammatory reactions in the joints (Braddock et al., Nat. Rev. Drug Disc., 3: 1-10, 2004); amyotrophic lateral sclerosis (Gugliandolo et al. Inflammation 2018 41(1): 93-103); cystic fibrosis (Iannitti et al. Nat. Commun. 2016 7: 10791); stroke (Walsh et al., Nature Reviews, 15: 84-97, 2014); chronic kidney disease (Granata et al. PLoS One 2015 10(3): e0122272); and inflammatory bowel diseases including ulcerative colitis and Crohn's disease (Braddock et al., Nat. Rev. Drug Disc., 3: 1-10, 2004, Neudecker et al. J Exp. Med. 2017 214(6): 1737-52, and Lazaridis et al. Dig. Dis. Sci. 2017 62(9): 2348-56). The NLRP3 inflammasome has been found to be activated in response to oxidative stress, and UVB irradiation (Schroder et al., Science, 327: 296-300, 2010). NLRP3 has also been shown to be involved in inflammatory hyperalgesia (Dolunay et al., Inflammation, 40: 366-386, 2017).

The inflammasome, and NLRP3 specifically, has also been proposed as a target for modulation by various pathogens including viruses such as DNA viruses (Amsler et al., Future Virol. (2013) 8(4), 357-370).

NLRP3 has also been implicated in the pathogenesis of many cancers (Menu et al., Clinical and Experimental Immunology 166: 1-15, 2011; and Masters Clin. Immunol. 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al. Lancet, S0140-6736(17)32247-X, 2017). Inhibition of the NLRP3 inflammasome or IL-1β has also been shown to inhibit the proliferation and migration of lung cancer cells in vitro (Wang et al. Oncol Rep. 2016; 35(4): 2053-64). A role for the NLRP3 inflammasome has been suggested in myelodysplastic syndromes (Basiorka et al. Blood. 2016 Dec. 22; 128(25):2960-2975) and also in the carcinogenesis of various other cancers including glioma (Li et al. Am J Cancer Res. 2015; 5(1): 442-449), inflammation-induced tumours (Allen et al. J Exp Med. 2010; 207(5): 1045-56 and Hu et al. PNAS. 2010; 107(50): 21635-40), multiple myeloma (Li et al. Hematology 2016 21(3): 144-51), and squamous cell carcinoma of the head and neck (Huang et al. J Exp Clin Cancer Res. 2017 2; 36(1): 116). Activation of the NLRP3 inflammasome has also been shown to mediate chemoresistance of tumour cells to 5-Fluorouracil (Feng et al. J Exp Clin Cancer Res. 2017 21; 36(1): 81), and activation of NLRP3 inflammasome in peripheral nerve contributes to chemotherapy-induced neuropathic pain (Jia et al. Mol Pain. 2017; 13:1-11).

NLRP3 has also been shown to be required for the efficient control of viral, bacterial, fungal, and helminth pathogen infections (Strowig et al., Nature, 481:278-286, 2012).

Accordingly, examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;
(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barré syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behçet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, vitiligo or vulvodynia;
(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;
(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections;
(v) central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, and amyotrophic lateral sclerosis;
(vi) metabolic diseases such as type 2 diabetes (T2D), atherosclerosis, obesity, gout, and pseudo-gout;
(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, and pericarditis including Dressler's syndrome;
(viii) respiratory diseases including chronic obstructive pulmonary disorder (COPD), asthma such as allergic asthma and steroid-resistant asthma, asbestosis, silicosis, nanoparticle induced inflammation, cystic fibrosis and idiopathic pulmonary fibrosis;
(ix) liver diseases including non-alcoholic fatty liver disease (NAFLD), and non-alcoholic steatohepatitis (NASH) including advanced fibrosis stages F3 and F4, alcoholic fatty liver disease (AFLD), and alcoholic steatohepatitis (ASH);
(x) renal diseases including chronic kidney disease, oxalate nephropathy, nephrocalcinosis, glomerulonephritis, and diabetic nephropathy;
(xi) ocular diseases including those of the ocular epithelium, age-related macular degeneration (AMD) (dry and wet), uveitis, corneal infection, diabetic retinopathy, optic nerve damage, dry eye, and glaucoma;
(xii) skin diseases including dermatitis such as contact dermatitis and atopic dermatitis, contact hypersensitivity, sunburn, skin lesions, hidradenitis suppurativa (HS), other cyst-causing skin diseases, and acne conglobata;
(xiii) lymphatic conditions such as lymphangitis and Castleman's disease;
(xiv) psychological disorders such as depression and psychological stress;
(xv) graft versus host disease;
(xvi) allodynia including mechanical allodynia; and
(xvii) any disease where an individual has been determined to carry a germline or somatic non-silent mutation in NLRP3.

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease; and
(xvi) any disease where an individual has been determined to carry a germline or somatic non-silent mutation in NLRP3.

In another embodiment, the disease, disorder or condition is selected from:
(i) inflammation;
(ii) an auto-immune disease;
(iii) an infection;
(iv) a central nervous system disease;
(v) a metabolic disease;
(vi) a cardiovascular disease;
(vii) a liver disease;
(viii) an ocular disease;
(ix) a skin disease;
(x) a lymphatic condition;
(xi) a psychological disorder;
(xii) graft versus host disease; and
(xiii) any disease where an individual has been determined to carry a germline or somatic non-silent mutation in NLRP3.

In yet another embodiment, the disease, disorder or condition is selected from:
(i) a central nervous system disease;
(ii) a metabolic disease;
(iii) a cardiovascular disease;
(iv) a liver disease;
(v) a renal disease;
(vi) a lymphatic condition;
(vii) a psychological disorder; and
(viii) any disease where an individual has been determined to carry a germline or somatic non-silent mutation in NLRP3.

In one embodiment, the disease, disorder or condition is selected from:
(i) a cardiovascular disease;
(ii) a liver disease;
(iii) a renal disease;
(iv) a psychological disorder;
(v) a lymphatic condition; and/or
(vi) any disease, disorder or condition in which an individual has been determined to carry a germline or somatic non-silent mutation in NLRP3.

More typically in such an embodiment, the disease, disorder or condition is selected from:
(i) a cardiovascular disease;
(ii) a liver disease;
(iii) a psychological disorder;
(iv) a lymphatic condition; and/or
(v) any disease, disorder or condition in which an individual has been determined to carry a germline or somatic non-silent mutation in NLRP3.

In a further embodiment, the disease, disorder or condition is selected from:
(i) cancer;
(ii) an infection;
(iii) a central nervous system disease;
(iv) a cardiovascular disease;
(v) a liver disease;
(vi) an ocular diseases; or
(vii) a skin disease.

More typically, the disease, disorder or condition is selected from:
(i) cancer;
(ii) an infection;
(iii) a central nervous system disease; or
(iv) a cardiovascular disease.

In one embodiment, the disease, disorder or condition is selected from:
(i) acne conglobata;
(ii) atopic dermatitis;
(iii) Alzheimer's disease;
(iv) amyotrophic lateral sclerosis;
(v) age-related macular degeneration (AMD);
(vi) anaplastic thyroid cancer;
(vii) cryopyrin-associated periodic syndromes (CAPS);
(viii) contact dermatitis;
(ix) cystic fibrosis;
(x) congestive heart failure;
(xi) chronic kidney disease;
(xii) Crohn's disease;
(xiii) familial cold autoinflammatory syndrome (FCAS);
(xiv) Huntington's disease;
(xv) heart failure;
(xvi) heart failure with preserved ejection fraction;
(xvii) ischemic reperfusion injury;
(xviii) juvenile idiopathic arthritis;
(xix) myocardial infarction;
(xx) macrophage activation syndrome;
(xxi) myelodysplastic syndrome;
(xxii) multiple myeloma;
(xxiii) motor neuron disease;
(xxiv) multiple sclerosis;
(xxv) Muckle-Wells syndrome;
(xxvi) non-alcoholic steatohepatitis (NASH);
(xxvii) neonatal-onset multisystem inflammatory disease (NOMID);
(xxviii) Parkinson's disease;
(xxix) systemic juvenile idiopathic arthritis;
(xxx) systemic lupus erythematosus;
(xxxi) traumatic brain injury;
(xxxii) transient ischemic attack; and
(xxxiii) ulcerative colitis.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
(vi) a vascular condition such as atherosclerosis, Behcet's disease, vasculitides, or wegener's granulomatosis;
(vii) an autoimmune condition such as systemic lupus erythematosus, Sjogren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, epstein-barr virus, viral encephalitis/aseptic meningitis, or pelvic inflammatory disease;
(xi) a renal condition such as mesangial proliferative glomerulonephritis, nephrotic syndrome, nephritis, glomerular nephritis, acute renal failure, uremia, or nephritic syndrome;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;
(xv) a cancer, including those cancers listed above;
(xvi) a burn, wound, trauma, haemorrhage or stroke;
(xvii) radiation exposure; and/or
(xviii) obesity; and/or
(xix) pain such as inflammatory hyperalgesia.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

In one embodiment of the fifth, sixth, seventh, eighth ninth or tenth aspect of the present invention, the treatment or prevention comprises topically administering a compound of the first or second aspect, or a pharmaceutically acceptable salt, solvate or prodrug of the third aspect, or a pharmaceutical composition of the fourth aspect. For example, the disease, disorder or condition may be a skin disease or condition, wherein the treatment or prevention comprises topically administering a compound of the first or second aspect, or a pharmaceutically acceptable salt, solvate or prodrug of the third aspect, or a pharmaceutical composition of the fourth aspect to the skin. Alternatively, the disease, disorder or condition may be an ocular disease or condition, wherein the treatment or prevention comprises topically administering a compound of the first or second aspect, or a pharmaceutically acceptable salt, solvate or prodrug of the third aspect, or a pharmaceutical composition of the fourth aspect to the eye.

In one embodiment, where the treatment or prevention comprises topically administering a compound of the first or second aspect, or a pharmaceutically acceptable salt, solvate or prodrug of the third aspect, or a pharmaceutical composition of the fourth aspect of the invention, one or more further active agents may be co-administered. The one or more further active agents may also be topically administered, or may be administered via a non-topical route. Typically, the one or more further active agents are also topically administered. For example, where the pharmaceutical composition of the fourth aspect of the invention is a topical pharmaceutical composition, the pharmaceutical composition may further comprise one or more further active agents.

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or pharmaceutical composition to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;
(ii) antibodies;
(iii) alkylating agents;
(iv) anti-metabolites;
(v) anti-angiogenic agents;
(vi) plant alkaloids and/or terpenoids;
(vii) topoisomerase inhibitors;
(viii) mTOR inhibitors;
(ix) stilbenoids;
(x) STING agonists;
(xi) cancer vaccines;
(xii) immunomodulatory agents;
(xiii) antibiotics;
(xiv) anti-fungal agents;
(xv) anti-helminthic agents; and/or
(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

In some embodiments, the one or more chemotherapeutic agents are selected from abiraterone acetate, altretamine, amsacrine, anhydrovinblastine, auristatin, azathioprine, adriamycin, bexarotene, bicalutamide, BMS 184476, bleomycin, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulphonamide, prednimustine, procarbazine, rhizoxin, sertenef, streptozocin, stramustine phosphate, tretinoin, tasonermin, taxol, topotecan, tamoxifen, teniposide, taxane, tegafur/uracil, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), interferon alpha, interferon beta, interferon gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), and/or cytokines (including interleukins, such as interleukin-2 (IL-2), or IL-10).

In some embodiments, the one or more antibodies may comprise one or more monoclonal antibodies. In some embodiments, the one or more antibodies are selected from abciximab, adalimumab, alemtuzumab, atlizumab, basiliximab, belimumab, bevacizumab, bretuximab vedotin, canakinumab, cetuximab, ceertolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumuab, ranibizumab, rituximab, tocilizumab, tositumomab, and/or trastuzumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from endostatin, angiogenin inhibitors, angiostatin, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides, such as cAMP, cGMP, and cGAMP, and/or modified cyclic di-nucleotides that may include one or more of the following modification features: 2′-O/3′-O linkage, phosphorothioate linkage, adenine and/or guanine analogue, and/or 2′-OH modification (e.g. protection of the 2′-OH with a methyl group or replacement of the 2′-OH by —F or —N₃).

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX40L, GITR, GITRL, CD27, CD70, TNFRSF25, TL1A, CD40, CD40L, HVEM, LIGHT, BTLA, CD160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDR001 (PD1), BMS-986016, MGA271, lirilumab, IPH2201, emactuzumab, INCB024360, galunisertib, ulocuplumab, BKT140, bavituximab, CC-90002, bevacizumab, and/or MNRP1685A.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents, anti-inflammatory agents (including nonsteroidal anti-inflammatory agents), anti-psoriatic agents (including anthralin and its derivatives), vitamins and vitamin-derivatives (including retinoinds, and VDR receptor ligands), corticosteroids, ion channel blockers (including potassium channel blockers), immune system regulators (including cyclosporin, FK 506, and glucocorticoids), lutenizing hormone releasing hormone agonists (such as leuprolidine, goserelin, triptorelin, histrelin, bicalutamide, flutamide and/or nilutamide), and/or hormones (including estrogen).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse, etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal and sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or talc. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disorder, disease or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a 30 day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

By way of example, combinations of aspects and embodiments that are typical of the present invention include the following.

In a first combination, a compound of the first aspect of the invention is provided wherein the 5-membered heteroaryl group of R¹ contains at least one nitrogen or sulfur atom in the 5-membered ring structure, and wherein R² is a cyclic group substituted at the α and α′ positions, wherein each substituent at the α and α′ positions comprises a carbon atom and wherein R² may optionally be further substituted.

In a second combination, a compound of the first aspect of the invention is provided wherein the 5-membered heteroaryl group of R¹ contains only carbon and nitrogen atoms in the 5-membered ring structure, and wherein R² is a cyclic group substituted at the α and α′ positions, wherein R² may optionally be further substituted.

In a third combination, a compound of the first aspect of the invention is provided wherein the 5-membered heteroaryl group of R¹ contains only carbon and nitrogen atoms in the 5-membered ring structure, and wherein the substituent at the α position of the cyclic group of R² comprises a carbon atom. Typically in such a combination, R^{X} is monovalent. More typically, the 5-membered heteroaryl group of R¹ is monocyclic.

In a fourth combination, a compound of the first aspect of the invention is provided wherein R¹ contains from 9 to 16 atoms other than hydrogen or halogen, and wherein R² is a cyclic group substituted at the α and α′ positions, wherein each substituent at the α and α′ positions comprises a carbon atom and wherein R² may optionally be further substituted.

In a fifth combination, a compound of the first aspect of the invention is provided wherein R^{X} contains from 4 to 11 atoms other than hydrogen or halogen, and wherein R² is a cyclic group substituted at the α and α′ positions, wherein each substituent at the α and α′ positions comprises a carbon atom and wherein R² may optionally be further substituted. Typically in such a combination, R^{X} is monovalent. More typically, the 5-membered heteroaryl group of R¹ is monocyclic.

In a sixth combination, a compound of the first aspect of the invention is provided wherein R^{X} contains only atoms selected from the group consisting of carbon, hydrogen, nitrogen, oxygen and halogen atoms, and wherein R² is a cyclic group substituted at the α and α′ positions, wherein each substituent at the α and α′ positions comprises a carbon atom and wherein R² may optionally be further substituted.

In a seventh combination, a compound of the first aspect of the invention is provided wherein:
R^{X} is any saturated hydrocarbyl group, wherein the hydrocarbyl group may be straight-chained or branched, or be or include cyclic groups, wherein the hydrocarbyl group may optionally be substituted with one or more groups selected from halo, —CN, —OH, —NH₂, oxo (═O) and ═NH, wherein the hydrocarbyl group includes at least one amide group in its carbon skeleton, and wherein the hydrocarbyl group may optionally include one, two or three further heteroatoms N and/or O in its carbon skeleton; and
R² is a cyclic group substituted at the α and α′ positions, wherein each substituent at the α and α′ positions comprises a carbon atom and wherein R² may optionally be further substituted.

As will be understood, in such a combination R^{X} is monovalent. Typically in such a combination, the 5-membered heteroaryl group of R¹ is monocyclic.

An eighth combination provides a compound of the first or second aspect of the invention, or a pharmaceutically acceptable salt, solvate or prodrug of the third aspect of the invention, or a pharmaceutical composition of the fourth aspect of the invention, wherein R² is a cyclic group substituted at the α and α′ positions, wherein each substituent at the α and α′ positions comprises a carbon atom and wherein R² may optionally be further substituted, for use in medicine.

A ninth combination provides a compound of the first or second aspect of the invention, or a pharmaceutically acceptable salt, solvate or prodrug of the third aspect of the invention, or a pharmaceutical composition of the fourth aspect of the invention, wherein the 5-membered heteroaryl group of R¹ contains only carbon and nitrogen atoms in the 5-membered ring structure, and wherein R^{X} is monovalent, for use in medicine. Typically in such a combination, the 5-membered heteroaryl group of R¹ is monocyclic.

A tenth combination provides a compound of the first or second aspect of the invention, or a pharmaceutically acceptable salt, solvate or prodrug of the third aspect of the invention, or a pharmaceutical composition of the fourth aspect of the invention, wherein the 5-membered heteroaryl group of R¹ contains only carbon and nitrogen atoms in the 5-membered ring structure, and wherein R¹ contains from 9 to 16 atoms other than hydrogen or halogen, for use in medicine. Typically in such a combination, R^{X} is monovalent. More typically, the 5-membered heteroaryl group of R¹ is monocyclic.

An eleventh combination provides a compound of the first or second aspect of the invention, or a pharmaceutically acceptable salt, solvate or prodrug of the third aspect of the invention, or a pharmaceutical composition of the fourth aspect of the invention, wherein the 5-membered heteroaryl group of R¹ contains only carbon and nitrogen atoms in the 5-membered ring structure, and wherein R^{X} contains from 4 to 11 atoms other than hydrogen or halogen, for use in medicine. Typically in such a combination, R^{X} is monovalent. More typically, the 5-membered heteroaryl group of R¹ is monocyclic.

A twelfth combination provides a compound of the first or second aspect of the invention, or a pharmaceutically acceptable salt, solvate or prodrug of the third aspect of the invention, or a pharmaceutical composition of the fourth aspect of the invention, wherein R² is a cyclic group substituted at the α and α′ positions, wherein R² may optionally be further substituted, for use in the treatment or prevention of a disease, disorder or condition, wherein the disease, disorder or condition is selected from:
(i) inflammation;
(ii) an auto-immune disease;
(iii) an infection;
(iv) a central nervous system disease;
(v) a metabolic disease;
(vi) a cardiovascular disease;
(vii) a liver disease;
(viii) an ocular disease;
(ix) a skin disease;
(x) a lymphatic condition;
(xi) a psychological disorder;
(xii) graft versus host disease; and
(xiii) any disease where an individual has been determined to carry a germline or somatic non-silent mutation in NLRP3.

Typically in such a combination, R^{X} is monovalent. More typically, the 5-membered heteroaryl group of R¹ is monocyclic.

A thirteenth combination provides a compound of the first or second aspect of the invention, or a pharmaceutically acceptable salt, solvate or prodrug of the third aspect of the invention, or a pharmaceutical composition of the fourth aspect of the invention, wherein the 5-membered heteroaryl group of R¹ contains only carbon and nitrogen atoms in the 5-membered ring structure, for use in the treatment or prevention of a disease, disorder or condition, wherein the disease, disorder or condition is selected from:
(i) a central nervous system disease;
(ii) a metabolic disease;
(iii) a cardiovascular disease;
(iv) a liver disease;
(v) a renal disease;
(vi) a lymphatic condition;
(vii) a psychological disorder; and
(viii) any disease where an individual has been determined to carry a germline or somatic non-silent mutation in NLRP3.

Typically in such a combination, R^{X} is monovalent. More typically, the 5-membered heteroaryl group of R¹ is monocyclic.

Typically, in any of the above exemplary combinations, Q is O.

Typically, in any of the above exemplary combinations, R² is an aryl or a heteroaryl group, wherein the aryl or the heteroaryl group is substituted at the α and α′ positions, and wherein R² may optionally be further substituted. Typically, each substituent at the α and α′ positions comprises a carbon atom. Typically in any of the above exemplary combinations, R² contains from 9 to 20 atoms other than hydrogen or halogen.

Typically, in any of the above exemplary combinations, the 5-membered heteroaryl group of R¹ contains at least one nitrogen atom in the 5-membered ring structure. More typically, the 5-membered heteroaryl group of R¹ contains only carbon and nitrogen atoms in the 5-membered ring structure.

Typically, in any of the above exemplary combinations, R^{X} contains from 4 to 11 atoms other than hydrogen or halogen. Typically, R¹ contains from 9 to 16 atoms other than hydrogen or halogen.

As will be appreciated the above combinations are exemplary only and other combinations of aspects and embodiments, including combinations of the above combinations, may readily be envisaged.

### EXAMPLES—COMPOUND SYNTHESIS

All solvents, reagents and compounds were purchased and used without further purification unless stated otherwise.

### Abbreviations

2-MeTHF 2-methyltetrahydrofuran
Ac₂O acetic anhydride
AcOH acetic acid
aq aqueous
Boc tert-butyloxycarbonyl
br broad
Cbz carboxybenzyl
CDI 1,1-carbonyl-diimidazole
conc concentrated
d doublet
DABCO 1,4-diazabicyclo[2.2.2]octane
DCE 1,2-dichloroethane, also called ethylene dichloride
DCM dichloromethane
DIPEA N,N-diisopropylethylamine, also called Hünig's base
DMA dimethylacetamide
DMAP 4-dimethylaminopyridine, also called N,N-dimethylpyridin-4-amine
DME dimethoxyethane
DMF N,N-dimethylformamide
DMSO dimethyl sulfoxide
eq or equiv equivalent
(ES+) electrospray ionization, positive mode
Et ethyl
EtOAc ethyl acetate
EtOH ethanol
h hour(s)
HATU 1-[bis(dimethylamino)methylene]-1H-1,2,3-triazolo[4,5-b]pyridinium 3-oxid hexafluorophosphate
HPLC high performance liquid chromatography
LC liquid chromatography
m multiplet
m-CPBA 3-chloroperoxybenzoic acid
Me methyl
MeCN acetonitrile
MeOH methanol
(M+H)+protonated molecular ion
MHz megahertz
min minute(s)
MS mass spectrometry
Ms mesyl, also called methanesulfonyl
MsCl mesyl chloride, also called methanesulfonyl chloride
MTBE methyl tert-butyl ether, also called tert-butyl methyl ether
m/z mass-to-charge ratio
NaO^{t}Bu sodium tert-butoxide
NBS 1-bromopyrrolidine-2,5-dione, also called N-bromosuccinimide
NCS 1-chloropyrrolidine-2,5-dione, also called N-chlorosuccinimide
NMP N-methylpyrrolidine
NMR nuclear magnetic resonance (spectroscopy)
Pd(dba)₃ tris(dibenzylideneacetone) dipalladium(o)
Pd(dppf)Cl₂ [1,1′-bis(diphenylphosphino)ferrocene] dichloropalladium(II)
PE petroleum ether
Ph phenyl
PMB p-methoxybenzyl, also called 4-methoxybenzyl
prep-HPLC preparative high performance liquid chromatography
prep-TLC preparative thin layer chromatography
PTSA p-toluenesulfonic acid
q quartet
RP reversed phase
RT room temperature
s singlet
Sept septuplet
sat saturated
SCX solid supported cation exchange (resin)
t triplet
T3P propylphosphonic anhydride
TBME tert-butyl methyl ether, also called methyl tert-butyl ether
TEA triethylamine
TFA 2,2,2-trifluoroacetic acid
THF tetrahydrofuran
TLC thin layer chromatography
wt % weight percent or percent by weight

### Experimental Methods

Nuclear Magnetic Resonance

NMR spectra were recorded at 300, 400 or 500 MHz. Spectra were measured at 298 K, unless indicated otherwise, and were referenced relative to the solvent resonance. The chemical shifts are reported in parts per million. Spectra were recorded using one of the following machines:
a Bruker Avance III spectrometer at 400 MHz fitted with a BBO 5 mm liquid probe,
a Bruker 400 MHz spectrometers using ICON-NMR, under TopSpin program control,
a Bruker Avance III HD spectrometer at 500 MHz, equipped with a Bruker 5 mm SmartProbe™,
an Agilent VNMRS 300 instrument fitted with a 7.05 Tesla magnet from Oxford instruments, indirect detection probe and direct drive console including PFG module, or
an Agilent MercuryPlus 300 instrument fitted with a 7.05 Tesla magnet from Oxford instruments, 4 nuclei auto-switchable probe and Mercury plus console.

LC-MS

LC-MS Methods: Using SHIMADZU LCMS-2020, Agilent 1200 LC/G1956A MSD and Agilent 1200\G6110A, Agilent 1200 LC & Agilent 6110 MSD. Mobile Phase: A: 0.025% NH₃HO in water (v/v); B: acetonitrile. Column: Kinetex EVO C18 2.1×30 mm, 5 μm.

Reversed Phase HPLC Conditions for the LCMS Analytical Methods

Methods 1a and 1b: Waters Xselect CSH C18 XP column (4.6×30 mm, 2.5 μm) at 40° C.; flow rate 2.5-4.5 mL min⁻¹ eluted with a H₂O-MeCN gradient containing either 0.1% v/v formic acid (Method 1a) or 10 mM NH₄HCO₃ in water (Method 1b) over 4 min employing UV detection at 254 nm. Gradient information: 0-3.00 min, ramped from 95% water-5% acetonitrile to 5% water-95% acetonitrile; 3.00-3.01 min, held at 5% water-95% acetonitrile, flow rate increased to 4.5 mL min⁻¹; 3.01-3.50 min, held at 5% water-95% acetonitrile; 3.50-3.60 min, returned to 95% water-5% acetonitrile, flow rate reduced to 3.50 mL min⁻¹; 3.60-3.90 min, held at 95% water-5% acetonitrile; 3.90-4.00 min, held at 95% water-5% acetonitrile, flow rate reduced to 2.5 mL min⁻¹.

Method 1c: Agilent 1290 series with UV detector and HP 6130 MSD mass detector using Waters XBridge BEH C18 XP column (2.1×50 mm, 2.5 μm) at 35° C.; flow rate 0.6 mL/min; mobile phase A: ammonium acetate (10 mM); water/MeOH/acetonitrile (900:60:40); mobile phase B: ammonium acetate (10 mM); water/MeOH/acetonitrile (100:540:360); over 4 min employing UV detection at 215 and 238 nm. Gradient information: 0-0.5 min, held at 80% A-20% B; 0.5-2.0 min, ramped from 80% A-20% B to 100% B.

Reversed Phase HPLC Conditions for the UPLC Analytical Methods

Methods 2a and 2b: Waters BEH C18 (2.1×30 mm, 1.7 μm) at 40° C.; flow rate 0.77 mL min⁻¹ eluted with a H₂O-MeCN gradient containing either 0.1% v/v formic acid (Method 2a) or 10 mM NH₄HCO₃ in water (Method 2b) over 3 min employing UV detection at 254 nm. Gradient information: 0.11 min, held at 95% water-5% acetonitrile, flow rate 0.77 mL min⁻¹; 0.11-2.15 min, ramped from 95% water-5% acetonitrile to 5% water-95% acetonitrile; 2.15-2.49 min, held at 5% water-95% acetonitrile, flow rate 0.77 mL min⁻¹; 2.49-2.56 min, returned to 95% water-5% acetonitrile; 2.56-3.00 min, held at 95% water-5% acetonitrile, flow rate reduced to 0.77 mL min⁻¹.

Preparative Reversed Phase HPLC General Methods

Method 1 (acidic preparation): Waters X-Select CSH column C18, 5 μm (19×50 mm), flow rate 28 mL min⁻¹ eluting with a H₂O-MeCN gradient containing 0.1% v/v formic acid over 6.5 min using UV detection at 254 nm. Gradient information: 0.0-0.2 min, 20% MeCN; 0.2-5.5 min, ramped from 20% MeCN to 40% MeCN; 5.5-5.6 min, ramped from 40% MeCN to 95% MeCN; 5.6-6.5 min, held at 95% MeCN.

Method 2 (basic preparation): Waters X-Bridge Prep column C18.5 μm (19×50 mm), flow rate 28 mL min⁻¹ eluting with a 10 mM NH₄HCO₃-MeCN gradient over 6.5 min using UV detection at 254 nm. Gradient information: 0.0-0.2 min, 10% MeCN; 0.2-5.5 min, ramped from 10% MeCN to 40% MeCN; 5.5-5.6 min, ramped from 40% MeCN to 95% MeCN; 5.6-6.5 min, held at 95% MeCN.

Method 3: Phenomenex Gemini column, 10 m (150×25 mm), flow rate=25 mL/min eluting with a water-acetonitrile gradient containing 0.04% NH₃ at pH 10 over 9 minutes using UV detection at 220 and 254 nm. Gradient information: 0-9 minutes, ramped from 8% to 35% acetonitrile; 9-9.2 minutes, ramped from 35% to 100% acetonitrile; 9.2-15.2 minutes, held at 100% acetonitrile.

Method 4: Revelis C18 reversed-phase 12 g cartridge [carbon loading 18%; surface area 568 m²/g; pore diameter 65 Angstrom; pH (5% slurry) 5.1; average particle size 40 μm], flow rate=30 mL/min eluting with a water-methanol gradient over 35 minutes using UV detection at 215, 235, 254 and 280 nm. Gradient information: 0-5 minutes, held at 0% methanol; 5-30 minutes, ramped from 0% to 70% methanol; 30-30.1 minutes, ramped from 70% to 100% methanol; 30.1-35 minutes, held at 100% methanol.

### Synthesis of Intermediates

### Intermediate P1: N,N-Dimethyl-2-(3-sulfamoyl-1H-pyrazol-1-yl)acetamide

### Step A: Lithium 1-(tetrahydro-2H-pyran-2-yl)-1H-pyrazole-5-sulfinate

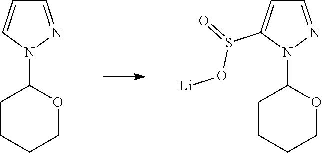

A solution of BuLi (100 mL, 250 mmol, 2.5M in hexanes) was added slowly to a solution of 1-(tetrahydro-2H-pyran-2-yl)-1H-pyrazole (36.2 g, 238 mmol) in THF (500 mL) keeping the temperature below −65° C. The mixture was stirred for 1.5 hours, then sulfur dioxide was bubbled through for 10 minutes. The mixture was allowed to warm to room temperature, the solvent evaporated and the residue triturated with TBME (300 mL) and filtered. The solid was washed with TBME and isohexane and dried to afford the crude title compound (54.89 g, 99%).

¹H NMR (DMSO-d₆) δ 7.26 (d, J=1.6 Hz, 1H), 6.10 (d, J=1.7 Hz, 1H), 5.99 (dd, J=10.0, 2.5 Hz, 1H), 3.92-3.87 (m, 1H), 3.56-3.49 (m, 1H), 2.25-2.15 (m, 1H), 2.00-1.91 (m, 1H), 1.75-1.69 (m, 1H), 1.66-1.46 (m, 3H).

LCMS; m/z 215 (M−H)⁻ (ES⁻).

### Step B: N,N-Bis(4-methoxybenzyl)-1-(tetrahydro-2H-pyran-2-yl)-1H-pyrazole-5-sulfonamide

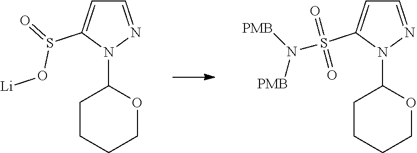

NCS (12.0 g, 90 mmol) was added to a suspension of lithium 1-(tetrahydro-2H-pyran-2-yl)-1H-pyrazole-5-sulfinate (20 g, 90 mmol) in DCM (250 mL) cooled in an ice bath. The mixture was stirred for 4 hours, quenched with water (100 mL), and then partitioned between DCM (300 mL) and water (200 mL). The organic phase was washed with water (200 mL), dried (MgSO₄), filtered and evaporated to ˜50 mL. The solution was added to a mixture of bis(4-methoxybenzyl)amine (24 g, 93 mmol) and triethylamine (40 mL, 287 mmol) in DCM (300 mL) cooled in an ice bath. After stirring for 1 hour, the mixture was warmed to room temperature, and then partitioned between DCM (300 mL) and water (250 mL). The organic layer was washed with water (250 mL), aq 1M HCl (2×250 mL), water (250 mL), dried (MgSO₄), filtered, and evaporated to afford the crude title compound (41.02 g, 97%) as a brown oil.

LCMS; m/z 494.2 (M+Na)⁺ (ES⁺).

### Step C: N,N-Bis(4-methoxybenzyl)-1H-pyrazole-3-sulfonamide

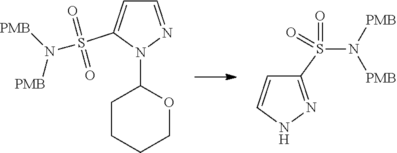

A mixture of N,N-bis(4-methoxybenzyl)-1-(tetrahydro-2H-pyran-2-yl)-1H-pyrazole-5-sulfonamide (41 g, 87 mmol) and aq 1M HCl (30 mL) in THF (300 mL) and MeOH (50 mL) was stirred at room temperature for 18 hours. The solvent was evaporated and the residue partitioned between EtOAc (400 mL) and aq 1M HCl (200 mL). The organic layer was washed with 10% brine (200 mL), dried (MgSO₄), filtered and evaporated. The residue was triturated with TBME, filtered and dried to afford the title compound (24.87 g, 69%) as an off white solid.

¹H NMR (CDCl₃) δ 7.88 (d, J=2.4 Hz, 1H), 7.06-7.02 (m, 4H), 6.79-6.75 (m, 4H), 6.63 (d, J=2.4 Hz, 1H), 4.31 (s, 4H), 3.78 (s, 6H). Exchangeable proton not visible.

LCMS; m/z 388 (M+H)⁺ (ES⁺); 386 (M−H)⁻ (ES⁻).

### Step D: 2-(3-(N,N-Bis(4-methoxybenzyl)sulfamoyl)-H-pyrazol-1-yl)-N,N-dimethylacetamide

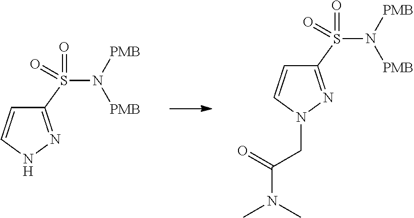

Under nitrogen, a mixture of N,N-bis(4-methoxybenzyl)-1H-pyrazole-3-sulfonamide (500 mg, 1.290 mmol) and K₂CO₃ (350 mg, 2.53 mmol) was suspended in dry acetonitrile (1 mL). 2-Chloro-N,N-dimethylacetamide (0.133 mL, 1.290 mmol) was added in a single portion and the cloudy mixture was heated to 65° C. (bath temperature) for 3 hours. The mixture was diluted with water (5 mL) and extracted with DCM (3×25 mL). The organic phase was dried by passing through a hydrophobic frit and concentrated in vacuo. The crude product was purified by chromatography on silica gel (40 g column, 0-100% EtOAc/isohexane) to afford the title compound (420 mg, 65%) as a pale yellow oil.

¹H NMR (CDCl₃) δ 7.65 (d, J=2.4 Hz, 1H), 7.09-6.99 (m, 4H), 6.85-6.76 (m, 4H), 6.72 (d, J=2.4 Hz, 1H), 5.08 (s, 2H), 4.32 (s, 4H), 3.80 (s, 6H), 3.10 (s, 3H), 3.04 (s, 3H). LCMS; m/z 473 (M+H)⁺ (ES⁺).

### Step E: N,N-Dimethyl-2-(3-sulfamoyl-1H-pyrazol-1-yl)acetamide

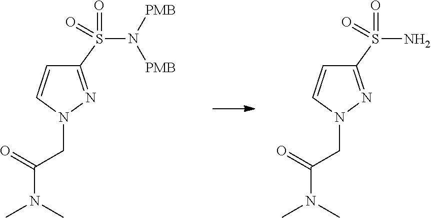

2-(3-(N,N-Bis(4-methoxybenzyl)sulfamoyl)-1H-pyrazol-1-yl)-N,N-dimethylacetamide (440 mg, 0.931 mmol) was dissolved in DCM (1 mL) and water (0.5 mL) and TFA (2 mL, 26.0 mmol) added. The reaction mixture was stirred at room temperature for 15 hours. The mixture was concentrated in vacuo and the crude product purified by chromatography (Companion apparatus, RP Flash C18, 12 g column, 0-10% acetonitrile/10 mM ammonium bicarbonate) to afford the title compound (195 mg, 88%) as a white solid.

¹H NMR (DMSO-d₆) δ 7.76 (d, J=2.4 Hz, 1H), 7.35 (s, 2H), 6.59 (d, J=2.4 Hz, 1H), 5.20 (s, 2H), 3.04 (s, 3H), 2.86 (s, 3H).

### Intermediate P2: N-Methyl-2-(3-sulfamoyl-1H-pyrazol-1-yl)acetamide

### Step A: 2-(3-(N,N-Bis(4-methoxybenzyl)sulfamoyl)-H-pyrazol-1-yl)-N-methylacetamide

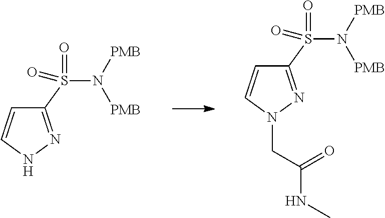

Prepared according to the general procedure of 2-(3-(N,N-bis(4-methoxybenzyl)sulfamoyl)-1H-pyrazol-1-yl)-N,N-dimethylacetamide (Intermediate P1, Step D) from N,N-bis(4-methoxybenzyl)-1H-pyrazole-3-sulfonamide (Intermediate P1, Step C) and 2-chloro-N-methylacetamide to afford the title compound (449 mg, 72%) as a colourless solid.

¹H NMR (CDCl₃) δ 7.54 (d, J=2.4 Hz, 1H), 7.09-7.02 (m, 4H), 6.81-6.76 (m, 4H), 6.71 (d, J=2.4 Hz, 1H), 5.91 (s, 1H), 4.83 (s, 2H), 4.32 (s, 4H), 3.79 (s, 6H), 2.75 (d, J=4.6 Hz, 3H).

LCMS; m/z 480 (M+Na)⁺ (ES⁺), 457 (M−H)⁺ (ES⁻).

### Step B: N-Methyl-2-(3-sulfamoyl-1H-pyrazol-1-yl)acetamide

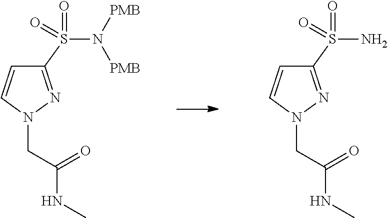

Prepared according to the general procedure of N,N-dimethyl-2-(3-sulfamoyl-1H-pyrazol-1-yl)acetamide (Intermediate P1, Step E) from 2-(3-(N,N-bis(4-methoxybenzyl)sulfamoyl)-1H-pyrazol-1-yl)-N-methylacetamide to afford the title compound (146 mg, 70%) as a colourless crystalline solid.

¹H NMR (DMSO-d₆) δ 8.22-8.11 (br s, 1H), 7.84 (d, J=2.4 Hz, 1H), 7.41 (s, 2H), 6.59 (d, J=2.4 Hz, 1H), 4.85 (s, 2H), 2.64 (d, J=4.6 Hz, 3H).

### Intermediate P3: 1-(1-Acetylazetidin-3-yl)-1H-pyrazole-3-sulfonamide

### Step A: tert-Butyl 3-(3-nitro-H-pyrazol-1-yl)azetidine-1-carboxylate

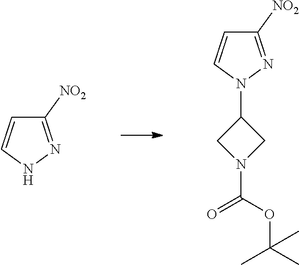 [SMILES: O=[N+]([O-])c1cc[nH]n1 ; CC(C)(C)OC(=O)N1CC(n2ccc([N+](=O)[O-])n2)C1]

Under nitrogen, a mixture of 3-nitro-1H-pyrazole (3 g, 26.5 mmol) and K₂CO₃ (11.00 g, 80 mmol) was suspended in dry DMF (75 mL). tert-Butyl 3-iodoazetidine-1-carboxylate (5.52 mL, 31.8 mmol) was added in a single portion and the cloudy mixture was heated to 100° C. for 4 hours. The mixture was diluted with water (5 mL) and extracted with DCM (3×50 mL). The organic phase was dried by passing through a hydrophobic frit and concentrated in vacuo. The crude product was purified by chromatography on silica gel (40 g column, 0-100% EtOAc/isohexane) to afford the title compound (5.3 g, 74%) as a colourless solid.

¹H NMR (DMSO-d₆) δ 8.20 (d, J=2.6 Hz, 1H), 7.11 (d, J=2.6 Hz, 1H), 5.43-5.28 (m, 1H), 4.35 (t, J=8.6 Hz, 2H), 4.22-4.03 (m, 2H), 1.42 (s, 9H).

LCMS; m/z 269 (M+H)⁺ (ES⁺).

### Step B: 1-(Azetidin-3-yl)-3-nitro-1H-pyrazole, HCl

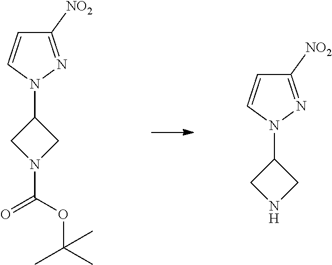 [SMILES: CC(C)(C)OC(=O)N1CC(n2ccc([N+](=O)[O-])n2)C1 ; O=[N+]([O-])c1ccn(C2CNC2)n1]

4M Hydrogen chloride in dioxane (24.70 mL, 99 mmol) was added to a solution of tert-butyl 3-(3-nitro-1H-pyrazol-1-yl)azetidine-1-carboxylate (5-3 g, 19.76 mmol) in 1,4-dioxane (20 mL) and stirred at room temperature for 16 hours. The reaction mixture was concentrated to afford the title compound (4.1 g, 96%) as an off-white solid. LCMS; m/z 169 (M+H)⁺ (ES⁺).

### Step C: 1-(3-(3-Nitro-1H-pyrazol-1-yl)azetidin-1-yl)ethanone

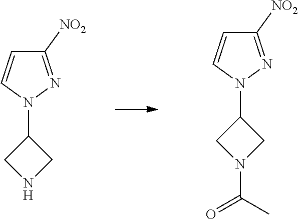

A suspension of 1-(azetidin-3-yl)-3-nitro-1H-pyrazole hydrochloride (2.59 g, 12.66 mmol) in DCM (36 mL) was treated with triethylamine (5.26 mL, 38.0 mmol) and stirred at room temperature for 10 minutes. The mixture was then cooled on ice to 0° C. and acetyl chloride (1.084 mL, 15.19 mmol) was added dropwise at 0° C. The reaction mixture was stirred for 10 minutes at 0° C., then the reaction mixture was left to warm to room temperature with stirring over 18 hours. The solvent was removed under reduced pressure and the residue was suspended in acetonitrile and then filtered and concentrated in vacuo. The crude product was purified by chromatography on silica gel (120 g column, 0-20% MeOH/DCM) to afford the title compound (1.02 g, 35%) as a yellow solid.

¹H NMR (DMSO-d₆) δ 8.22 (d, J=2.6 Hz, 1H), 7.12 (d, J=2.6 Hz, 1H), 5.46-5.34 (m, 1H), 4.66-4.56 (m, 1H), 4.46-4.37 (m, 1H), 4.36-4.27 (m, 1H), 4.11 (dd, J=10.3, 5.2 Hz, 1H), 1.83 (s, 3H).

LCMS; m/z 211 (M+H)⁺ (ES⁺).

### Step D: 1-(3-(3-Amino-1H-pyrazol-1-yl)azetidin-1-yl)ethanone

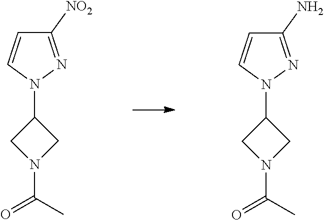

1-(3-(3-Nitro-1H-pyrazol-1-yl)azetidin-1-yl)ethanone (1.02 g, 4.46 mmol) and 10% palladium on carbon (wet Type 87 L) (0.024 g) were suspended in MeOH (10 mL) and EtOAc (10 mL). The reaction mixture was stirred at room temperature under 2 bar of H₂ for 17 hours. The reaction mixture was filtered through a pad of Celite® and the filter cake was washed with EtOAc (2×10 mL). The filtrate was concentrated to dryness to give the title compound (0.95 g, 92%) as a viscous yellow oil.

¹H NMR (DMSO-d₆) δ 7.42 (d, J=2.3 Hz, 1H), 5.41 (d, J=2.3 Hz, 1H), 4.94 (ddd, J=8.0, 5.3, 2.7 Hz, 1H), 4.80 (s, 2H), 4.43 (ddd, J=9.0, 8.0, 1.1 Hz, 1H), 4.29 (dd, J=8.6, 5.4 Hz, 1H), 4.15 (ddd, J=9.4, 8.1, 1.1 Hz, 1H), 4.07-3.93 (m, 1H), 1.78 (s, 3H).

LCMS; m/z 181 (M+H)⁺ (ES⁺).

### Step E: 1-(1-Acetylazetidin-3-yl)-1H-pyrazole-3-sulfonyl Chloride

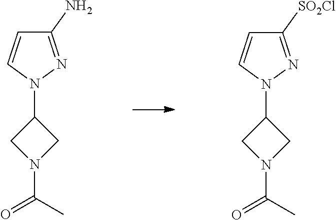 [SMILES: CC(=O)N1CC(n2ccc(N)n2)C1 ; CC(=O)N1CC(n2ccc(S(=O)(=O)Cl)n2)C1]

A mixture of concentrated HCl (1.5 mL) in water (1 mL) and acetonitrile (5.0 mL) was cooled to −10° C. and treated with a solution of sodium nitrite (0.338 g, 4.90 mmol) in water (0.6 mL) dropwise maintaining the internal temperature below 0° C. The solution was stirred for 10 minutes and then treated with a solution of 1-(3-(3-amino-1H-pyrazol-1-yl)azetidin-1-yl)ethanone (0.95 g, 4.09 mmol) in acetonitrile (5.1 mL) (which was pre-cooled to 0° C.) at 0° C. The resulting reaction mixture was stirred at 0° C. for 50 minutes. Cold AcOH (2 mL), CuCl₂.2H₂O (0.275 g, 2.043 mmol) and CuCl (0.02 g, 0.204 mmol) were sequentially added to the reaction mixture and the reaction mixture was purged with SO₂ gas for 20 minutes at 0° C. The reaction was stirred for a further 45 minutes, diluted with water (20 mL) and extracted with EtOAc (2×20 mL). The organic phase was washed with water (25 mL) and saturated brine (25 mL), dried over Na₂SO₄ filtered and concentrated in vacuo to afford a brown oil. The crude product was purified by chromatography on silica gel (24 g column, 0-10% MeOH/DCM) to afford the title compound (528 mg, 32%) as a yellow oil.

¹H NMR (CDCl₃ δ 7.69 (d, J=2.5 Hz, 1H), 6.96 (d, J=2.5 Hz, 1H), 5.25 (p, J=6.7 Hz, 1H), 4.74-4.27 (m, 4H), 1.96 (s, 3H).

### Step F: 1-(1-Acetylazetidin-3-yl)-1H-pyrazole-3-sulfonamide

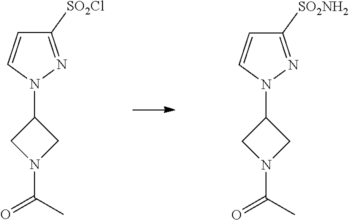 [SMILES: CC(=O)N1CC(n2ccc(S(=O)(=O)Cl)n2)C1 ; CC(=O)N1CC(n2ccc(S(N)(=O)=O)n2)C1]

1-(1-Acetylazetidin-3-yl)-1H-pyrazole-3-sulfonyl chloride (0.52 g, 1.301 mmol) in THF (8 mL) was treated with 0.5 M ammonia in dioxane (7.8 mL, 3.90 mmol) and the reaction mixture was stirred at room temperature for 22 hours. The reaction mixture was concentrated and the crude product was purified by chromatography on silica gel (24 g column, 0-10% MeOH/DCM) to afford the title compound (136 mg, 42%) as a white powder.

¹H NMR (DMSO-d₆) δ 8.06 (d, J=2.4 Hz, 1H), 7.50 (s, 2H), 6.64 (d, J=2.4 Hz, 1H), 5.34 (ddd, J=8.1, 5.3, 2.9 Hz, 1H), 4.75-4.43 (m, 1H), 4.50-4.12 (m, 2H), 4.09 (dd, J=10.0, 5.3 Hz, 1H), 1.82 (s, 3H).

LCMS; m/z 245 (M+H)⁺ (ES⁺).

### Intermediate P4: 5-Methyl-4-oxo-4,5,6,7-tetrahydropyrazolo[1,5-a]pyrazine-2-sulfonamide

### Step A: 2-Amino-5-methyl-6,7-dihydropyrazolo[1,5-a]pyrazin-4(5H)-one

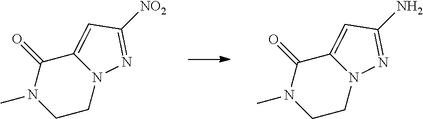

Zinc (167 mg, 2.55 mmol) was added portionwise to 5-methyl-2-nitro-6,7-dihydropyrazolo [1,5-a]pyrazin-4(5H)-one (1.4 g, 7.14 mmol) in AcOH (1.0 mL) and THF (1.5 mL). The reaction mixture was left to stir at room temperature for 2 days. The reaction mixture was filtered through a pad of Celite®, washed with DCM (2×15 mL) and the filtrate concentrated under reduced pressure to give a yellow solid. The solid was suspended in DCM (5 mL), filtered and the filtrate was evaporated to dryness to give the title compound (2.2 g, 74%) as a yellow solid.

¹H NMR (DMSO-d₆) δ 5.80 (s, 1H), 4.83 (s, 2H), 4.10-3.93 (m, 2H), 3.72-3.55 (m, 2H), 2.97 (s, 3H).

LCMS; m/z 167 (M+H)⁺ (ES⁺).

### Step B: 5-Methyl-4-oxo-4,5,6,7-tetrahydropyrazolo[1,5-a]pyrazine-2-sulfonyl Chloride

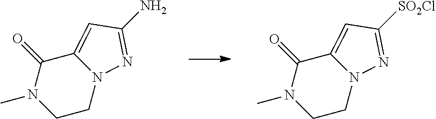

A mixture of aqueous HCl (2.2 mL) in water (8 mL) and acetonitrile (8 mL) was cooled to −10° C. and treated with a solution of NaNO₂ (0.50 g, 7.25 mmol) in water (0.9 mL) dropwise maintaining the internal temperature below 0° C. The solution was stirred for 10 minutes and then treated with a solution of 2-amino-5-methyl-6,7-dihydropyrazolo [1,5-a]pyrazin-4(5H)-one (0.997 g, 6 mmol) in acetonitrile (8 mL) (which was pre-cooled to 0° C.) at 0° C. The resulting reaction mixture was stirred at 0° C. for 50 minutes. Cold AcOH (4.8 mL), CuCl₂ dihydrate (0.30 g, 2.23 mmol) and CuCl (0.03 g, 0.30 mmol) were sequentially added to the reaction mixture and the reaction mixture was purged with SO₂ gas for 20 minutes at 0° C. The reaction was stirred for a further 45 minutes, diluted with water (20 mL) and extracted with EtOAc (2×20 mL). The organic phase was washed with water (25 mL) and saturated brine (25 mL), dried (MgSO₄), filtered and concentrated in vacuo to afford a brown oil. The brown oil was purified by chromatography on silica gel (40 g column, 0-100% EtOAc/isohexane) to afford the title compound (577 mg, 30%) as a yellow crystalline solid.

¹H NMR (CDCl₃) δ 7.39 (s, 1H), 4.63-4.45 (m, 2H), 3.95-3.83 (m, 2H), 3.19 (s, 3H).

### Step C: 5-Methyl-4-oxo-4,5,6,7-tetrahydropyrazolo[1,5-a]pyrazine-2-sulfonamide

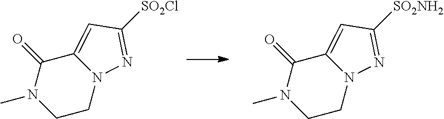

5-Methyl-4-oxo-4,5,6,7-tetrahydropyrazolo[1,5-a]pyrazine-2-sulfonylchloride (577 mg, 1.826 mmol) in THF (4 mL) was treated with 0.5 M ammonia in 1,4-dioxane (11.00 mL, 5.50 mmol). The reaction mixture was stirred at room temperature for 2 hours and concentrated to dryness. The residue was suspended in water (10 mL) and filtered. The yellow powder obtained was then washed with DCM (2×5 mL) and dried under vacuum to afford the title compound (332 mg, 77%) as a white powder.

¹H NMR (DMSO-d₆) δ 7.57 (s, 2H), 6.93 (s, 1H), 4.59-4.34 (m, 2H), 3.90-3.71 (m, 2H), 3.01 (s, 3H).

LCMS; m/z 231 (M+H)⁺ (ES⁺).

### Intermediate P5: N,N,1-Trimethyl-3-sulfamoyl-1H-pyrazole-5-carboxamide

### Step A: 1-Methyl-3-sulfamoyl-1H-pyrazole-5-carboxylic Acid, Sodium Salt

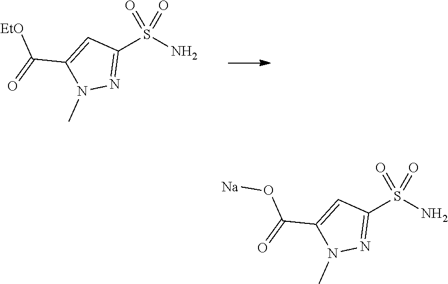

To a suspension of ethyl 1-methyl-3-sulfamoyl-1H-pyrazole-5-carboxylate (3 g, 12.86 mmol) in ethanol (60 mL) was added a solution of sodium hydroxide (2.0 M, 13.5 mL) and the mixture was stirred at room temperature for 2 hours. The resulting precipitate was filtered off, washed with ethanol and dried to afford the title compound (2.92 g, 99%) as a white solid.

¹H NMR (D₂O) δ 6.79 (s, 1H) and 4.01 (s, 3H).

### Step B: N,N,1-Trimethyl-3-sulfamoyl-H-pyrazole-5-carboxamide

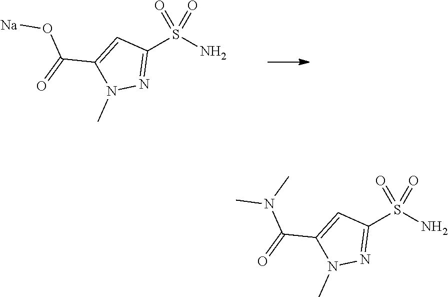

To a mixture of 1-methyl-3-sulfamoyl-1H-pyrazole-5-carboxylic acid, sodium salt (2.38 g, 10.48 mmol) was added T3P (50% in ethyl acetate, 12.47 ml, 20.95 mmol) and N,N-diisopropylethylamine (Hunig's Base, 3.66 ml, 20.95 mmol) in tetrahydrofuran (50 mL). A solution of 2.0 M dimethylamine in THF (15.71 ml, 31.4 mmol) was added and the reaction stirred for 20 hours before being quenched with saturated aqueous ammonium chloride (10 mL) and extracted with ethyl acetate (3×20 ml). The combined extracts were dried (magnesium sulfate), filtered and evaporated in vacuo to afford a yellow gum. The crude product was triturated in dichloromethane (20 mL) and filtered to obtain the title compound (900 mg) as a white solid. The mother layers were evaporated, dissolved in dichloromethane/methanol and purified by chromatography (Companion apparatus, 40 g column, 0-10% methanol/dichloromethane with product eluting at ˜5% methanol) to afford a further batch of the title compound (457 mg) as a white solid. The solids were combined to afford the title compound (1.36 g, 55%).

¹H NMR (DMSO-d₆) δ 7.50 (s, 2H), 6.82 (s, 1H), 3.90 (s, 3H), 3.03 (s, 3H) and 3.01 (s, 3H).

LCMS m/z 233.0 (M+H)⁺ (ES⁺).

### Intermediate P6: 3-(N-((1,2,3,5,6,7-Hexahydro-s-indacen-4-yl)carbamoyl) sulfamoyl)-1-methyl-1H-pyrazole-5-carboxylic Acid

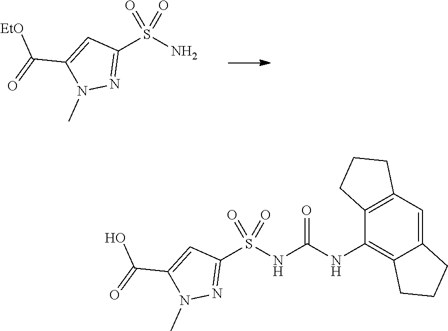 [SMILES: CCOC(=O)c1cc(S(N)(=O)=O)nn1C ; Cn1nc(S(=O)(=O)NC(=O)Nc2c3c(cc4c2CCC4)CCC3)cc1C(=O)O]

Triphosgene (170 mg, 0.573 mmol) was added to a mixture of 1,2,3,5,6,7-hexahydro-s-indacen-4-amine (165 mg, 0.952 mmol) and triethylamine (0.36 mL, 2.58 mmol) in THF (8 mL) and stirred for 15 hours. The reaction mixture was evaporated in vacuo and azeotroped with toluene (3×1 mL). THF (8 mL) was added and the reaction mixture was filtered. The filtrate was added to a mixture of ethyl 1-methyl-3-sulfamoyl-1H-pyrazole-5-carboxylate (200 mg, 0.857 mmol) and sodium hydride (86 mg, 2.150 mmol) in THF (8 mL) and stirred for 20 hours. The reaction was quenched with aqueous Na₂CO₃ (3.5 mL, 1.295 mmol), and evaporated in vacuo to remove the THF. The residual aqueous was washed with MTBE (2×5 mL). The solid that precipitated from the aqueous was filtered off and dried to afford ethyl 3-(N-((1,2,3,5,6,7-hexahydro-s-indacen-4-yl)carbamoyl)sulfamoyl)-1-methyl-1H-pyrazole-5-carboxylate, sodium (100 mg) as a solid. The filtrate was purified by chromatography on RP Flash C18 (12 g column, 5-50% MeCN/10 mM ammonium bicarbonate) to afford the title compound (180 mg) as a white solid. The solids were combined and dissolved in MeOH (3 mL). Aq. NaOH (0.25 mL, 0.500 mmol) was added and the reaction mixture was stirred for 20 hours. The MeOH was evaporated in vacuo. The remaining aqueous was adjusted to pH 8 with NaH₂PO₄ and purified by chromatography on RP Flash C18 (12 g column, 5-50% MeCN/10 mM ammonium bicarbonate) to afford the title compound (140 mg, 39%) as a white solid.

¹H NMR (DMSO-d₆) δ 7.65 (s, 2H), 6.31 (s, 1H), 6.15 (s, 1H), 3.41 (s, 3H), 2.05 (t, J=7.4 Hz, 4H), 1.90 (t, J=7.3 Hz, 4H), 1.24 (quin, J=7.4 Hz, 4H). One exchangeable proton not observed.

LCMS; m/z 405.0 (M+H)⁺ (ES⁺).

### Intermediate P7: 1-Methyl-5-(pyrrolidine-1-carbonyl)-1H-pyrazole-3-sulfonamide

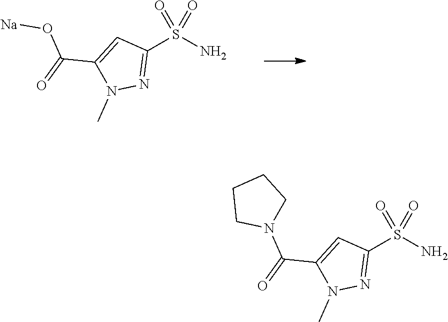

Prepared according to the general procedure for N,N,1-trimethyl-3-sulfamoyl-H-pyrazole-5-carboxamide (Intermediate P5, Step B) from 1-methyl-3-sulfamoyl-1H-pyrazole-5-carboxylic acid, sodium salt (Intermediate P5, Step A) and pyrrolidine to afford the title compound (204 mg, 54%) as a cream solid.

LCMS; m/z 259.3 (M+H)⁺ (ES⁺).

### Intermediate P8: 3-(N-((1,2,3,5,6,7-Hexahydro-s-indacen-4-yl)carbamoyl) sulfamoyl)-1-methyl-1H-pyrazole-5-carboxylic Acid, Disodium Salt

### Step A: Ethyl 3-(N-((1,2,3,5,6,7-hexahydro-s-indacen-4-yl)carbamoyl) sulfamoyl)-1-methyl-1H-pyrazole-5-carboxylate, Sodium Salt

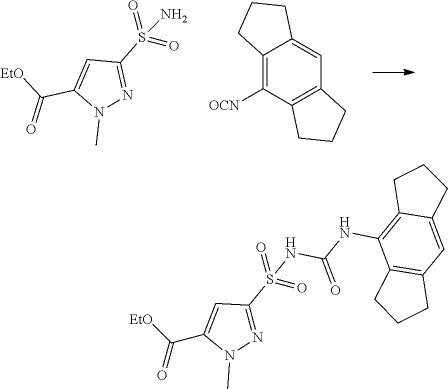

2 M Sodium tert-butoxide in THF (1.005 mL, 2.009 mmol) was added to a solution of ethyl 1-methyl-3-sulfamoyl-1H-pyrazole-5-carboxylate (0.5 g, 1.914 mmol) in THF (15 mL) and stirred at room temperature for 1 hour to give a white suspension. Then 4-isocyanato-1,2,3,5,6,7-hexahydro-s-indacene (Intermediate A1) (0.419 g, 2.105 mmol) in THF (5 mL) was added and stirred at room temperature overnight. The resultant colourless precipitate was collected by filtration, washing with THF (4 mL), and dried in vacuo to afford the title compound (930 mg, 91%) as a colourless solid. ¹H NMR (DMSO-d₆) δ 7.51 (s, 1H), 6.96 (s, 1H), 6.77 (s, 1H), 4.28 (q, J=7.1 Hz, 2H), 4.05 (s, 3H), 2.74 (t, J=7.4 Hz, 4H), 2.66 (t, J=7.3 Hz, 4H), 1.90 (p, J=7.4 Hz, 4H), 1.30 (t, J=7.1 Hz, 3H).

LCMS; m/z 433.4 (M+H)⁺ (ES⁺).

### Step B: 3-(N-((1,2,3,5,6,7-Hexahydro-s-indacen-4-yl)carbamoyl) sulfamoyl)-1-methyl-1H-pyrazole-5-carboxylic Acid, Disodium Salt

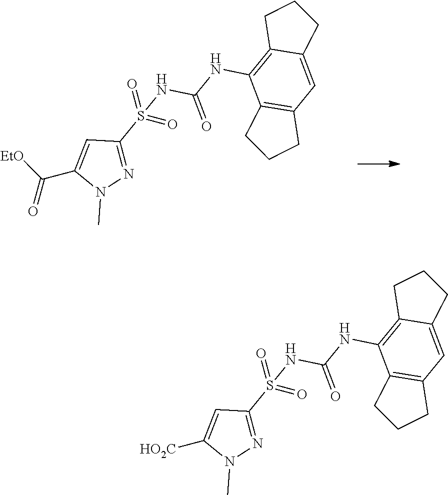 [SMILES: CCOC(=O)c1cc(S(=O)(=O)NC(=O)Nc2c3c(cc4c2CCC4)CCC3)nn1C ; Cn1nc(S(=O)(=O)NC(=O)Nc2c3c(cc4c2CCC4)CCC3)cc1C(=O)O]

Ethyl 3-(N-((1,2,3,5,6,7-hexahydro-s-indacen-4-yl)carbamoyl)sulfamoyl)-1-methyl-1H-pyrazole-5-carboxylate, sodium salt (3.15 g, 6.24 mmol) was dissolved in MeOH (20 mL), 2 M aqueous NaOH (3.12 mL, 6.24 mmol) was added and stirred for 6 hours. The reaction mixture was concentrated under reduced pressure to afford the title compound (2.80 g, 99%) as a colourless solid.

¹H NMR (DMSO-d₆) δ 7.57 (s, 1H), 6.76 (s, 1H), 6.44 (s, 1H), 4.02 (s, 3H), 2.74 (t, J=7.4 Hz, 4H), 2.65 (t, J=7.4 Hz, 4H), 1.89 (p, J=7.4 Hz, 4H).

LCMS; m/z 405.4 (M+H)⁺ (ES⁺).

### Intermediate P9: 3-(N-((2,6-Diisopropylphenyl)carbamoyl)sulfamoyl)-1-methyl-1H-pyrazole-5-carboxylic Acid, Disodium Salt

### Step A: Ethyl 3-(N-((2,6-diisopropylphenyl)carbamoyl)sulfamoyl)-1-methyl-1H-pyrazole-5-carboxylate, Sodium Salt

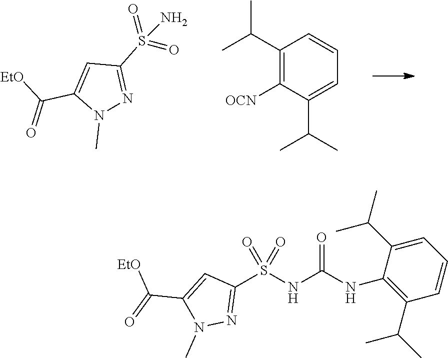

Ethyl 1-methyl-3-sulfamoyl-1H-pyrazole-5-carboxylate (2 g, 7.65 mmol) was dissolved in THF (80 mL, 986 mmol). Sodium hydride (0.367 g, 9.18 mmol) was added and stirred at room temperature for 30 minutes to give a white suspension. Then 2-isocyanato-1,3-diisopropylbenzene (Intermediate A9) (1.712 g, 8.42 mmol) in THF (20 mL) was added and stirred at room temperature overnight. The resultant colourless precipitate was collected by filtration, washing with THF (2×20 mL), and dried in vacuo to afford the title compound (2.16 g, 60%) as a colourless solid.

¹H NMR (DMSO-d₆) δ 7.35 (s, 1H), 7.15-7.05 (m, 1H), 7.05-6.95 (m, 2H), 6.93 (s, 1H), 4.28 (q, J=7.1 Hz, 2H), 4.05 (s, 3H), 3.20-3.02 (m, 2H), 1.28 (t, J=7.1 Hz, 3H), 1.03 (d, J=6.9 Hz, 12H).

LCMS; m/z 437.4 (M+H)⁺ (ES⁺).

### Step B: 3-(N-((2,6-Diisopropylphenyl)carbamoyl)sulfamoyl)-1-methyl-1H-pyrazole-5-carboxylic Acid, Disodium Salt

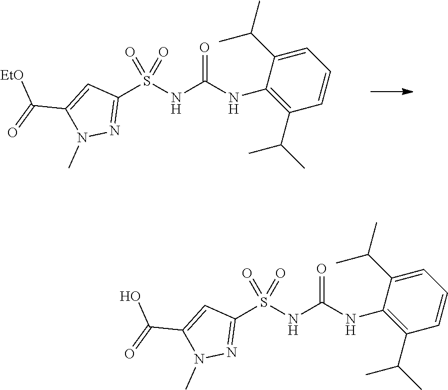 [SMILES: CCOC(=O)c1cc(S(=O)(=O)NC(=O)Nc2c(C(C)C)cccc2C(C)C)nn1C ; CC(C)c1cccc(C(C)C)c1NC(=O)NS(=O)(=O)c1cc(C(=O)O)n(C)n1]

Prepared according to the general procedure for 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) from ethyl 3-(N-((2,6-diisopropylphenyl)carbamoyl) sulfamoyl)-1-methyl-1H-pyrazole-5-carboxylate, sodium salt to afford the title compound (2.0 g, 99%) as a colourless solid.

¹H NMR (DMSO-d₆) δ 7.44 (s, 1H), 7.13-7.05 (m, 1H), 7.05-6.94 (m, 2H), 6.42 (s, 1H), 4.00 (s, 3H), 3.16-3.03 (m, 2H), 1.01 (d, J=6.8 Hz, 12H).

LCMS; m/z 409.4 (M+H)⁺ (ES⁺).

### Intermediate P10: 3-(N-((4-Fluoro-2,6-diisopropylphenyl)carbamoyl) sulfamoyl)-1-methyl-1H-pyrazole-5-carboxylic Acid, Disodium Salt

### Step A: Ethyl 3-(N-((4-fluoro-2,6-diisopropylphenyl)carbamoyl)sulfamoyl)-1-methyl-1H-pyrazole-5-carboxylate, Sodium Salt

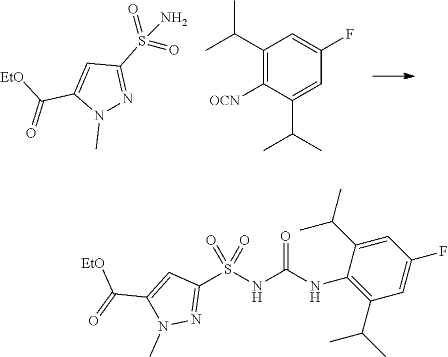

Prepared according to the general procedure for ethyl 3-(N-((1,2,3,5,6,7-hexahydro-s-indacen-4-yl)carbamoyl)sulfamoyl)-1-methyl-1H-pyrazole-5-carboxylate, sodium salt (Intermediate P8, Step A) from ethyl 1-methyl-3-sulfamoyl-H-pyrazole-5-carboxylate and 5-fluoro-2-isocyanato-1,3-diisopropylbenzene (Intermediate A2) to afford the title compound (1.7 g, 92%) as a colourless solid.

¹H NMR (DMSO-d₆) δ 7.32 (s, 1H), 6.93 (s, 1H), 6.79 (d, J=10.1 Hz, 2H), 4.29 (q, J=7.1 Hz, 2H), 4.05 (s, 3H), 3.21-2.94 (m, 2H), 1.29 (t, J=7.1 Hz, 3H), 1.03 (d, J=6.8 Hz, 12H).

LCMS; m/z 455.4 (M+H)⁺ (ES⁺).

### Step B: 3-(N-((4-Fluoro-2,6-diisopropylphenyl)carbamoyl)sulfamoyl)-1-methyl-1H-pyrazole-5-carboxylic Acid, Disodium Salt

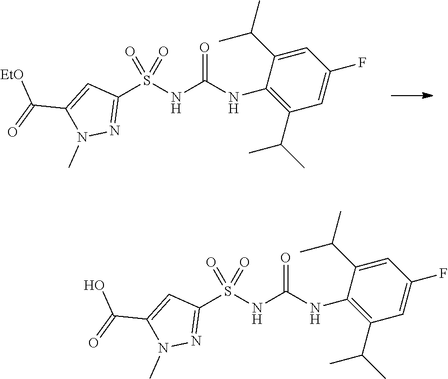 [SMILES: CCOC(=O)c1cc(S(=O)(=O)NC(=O)Nc2c(C(C)C)cc(F)cc2C(C)C)nn1C ; CC(C)c1cc(F)cc(C(C)C)c1NC(=O)NS(=O)(=O)c1cc(C(=O)O)n(C)n1]

Prepared according to the general procedure for 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) from ethyl 3-(N-((4-fluoro-2,6-diisopropylphenyl) carbamoyl)sulfamoyl)-1-methyl-1H-pyrazole-5-carboxylate, sodium salt to afford the title compound (1.65 g, 98%) as a colourless solid.

¹H NMR (DMSO-d₆) δ 7.41 (s, 1H), 6.77 (d, J=10.1 Hz, 2H), 6.45 (s, 1H), 4.01 (s, 3H), 3.15-3.02 (m, 2H), 1.10-0.93 (m, 12H).

LCMS; m/z 427.4 (M+H)⁺ (ES⁺).

### Intermediate P11: 3-(N-((4-Chloro-2,6-diisopropylphenyl)carbamoyl) sulfamoyl)-1-methyl-1H-pyrazole-5-carboxylic Acid, Disodium Salt

### Step A: Ethyl 3-(N-((4-chloro-2,6-diisopropylphenyl)carbamoyl) sulfamoyl)-1-methyl-1H-pyrazole-5-carboxylate, Sodium Salt

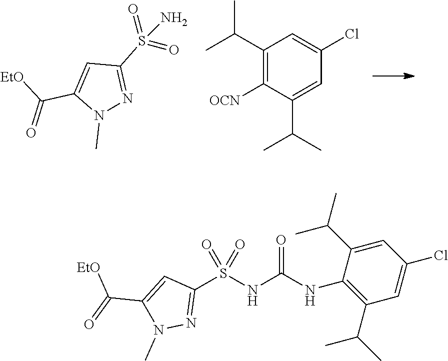

Prepared according to the general procedure for ethyl 3-(N-((1,2,3,5,6,7-hexahydro-s-indacen-4-yl)carbamoyl)sulfamoyl)-1-methyl-1H-pyrazole-5-carboxylate, Sodium Salt (Intermediate P8, Step A) from ethyl 1-methyl-3-sulfamoyl-H-pyrazole-5-carboxylate and 5-chloro-2-isocyanato-1,3-diisopropylbenzene (Intermediate A12) to afford the title compound (1.32 g, 92%) as a colourless solid.

¹H NMR (DMSO-d₆); δ 7.41 (s, 1H), 7.01 (s, 2H), 6.92 (s, 1H), 4.29 (q, J=7.1 Hz, 2H), 4.05 (s, 3H), 3.13 (br s, 2H), 1.29 (t, J=7.1 Hz, 3H), 1.04 (d, J=6.8 Hz, 12H).

LCMS; m/z 471.4 (M+H)⁺ (ES⁺).

### Step B: 3-(N-((4-Chloro-2,6-diisopropylphenyl)carbamoyl)sulfamoyl)-1-methyl-1H-pyrazole-5-carboxylic Acid, Disodium Salt

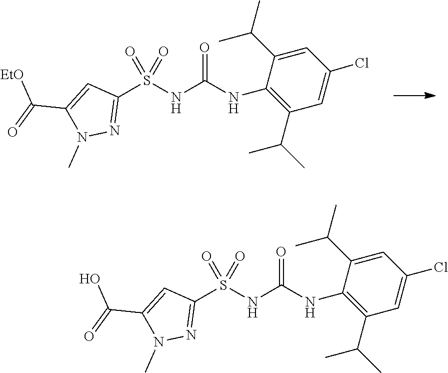 [SMILES: CCOC(=O)c1cc(S(=O)(=O)NC(=O)Nc2c(C(C)C)cc(Cl)cc2C(C)C)nn1C ; CC(C)c1cc(Cl)cc(C(C)C)c1NC(=O)NS(=O)(=O)c1cc(C(=O)O)n(C)n1]

Prepared according to the general procedure for 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) from ethyl 3-(N-((4-chloro-2,6-diisopropylphenyl) carbamoyl)sulfamoyl)-1-methyl-1H-pyrazole-5-carboxylate, sodium salt to afford the title compound (1.0 g, 77%) as a colourless solid.

¹H NMR (DMSO-d₆) δ 7.49 (s, 1H), 7.00 (s, 2H), 6.42 (s, 1H), 4.01 (s, 3H), 3.09 (br s, 2H), 1.02 (d, J=6.8 Hz, 12H).

LCMS; m/z 443.4 (M+H)⁺ (ES⁺).

### Intermediate P12: 5-(Azetidine-1-carbonyl)-1-isopropyl-1H-pyrazole-3-sulfonamide

### Step A: Ethyl 3-(N,N-bis(4-methoxybenzyl)sulfamoyl)-1H-pyrazole-5-carboxylate

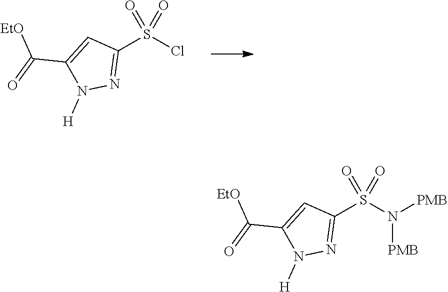

Ethyl 3-(chlorosulfonyl)-1H-pyrazole-5-carboxylate (4.9 g, 15-19 mmol) in DCM (52 mL) was added dropwise to bis(4-methoxybenzyl)amine (4.34 g, 15-19 mmol), DCM (100 mL) and triethylamine (2.54 mL, 18.23 mmol). The reaction was left to stir at room temperature for 18 hours and then poured onto water (50 mL). The organic layer was separated. The aqueous layer was extracted with DCM (2×50 mL). The combined organic layers were dried (MgSO₄), filtered and concentrated to dryness to give a pale yellow oil which was purified by chromatography on silica gel (120 g column, 0-70% EtOAc in isohexane) to afford the title compound (5-7 g, 81%) as a white solid.

¹H NMR (DMSO-d₆) δ 14.87 (s, 1H), 7.28-6.98 (m, 5H), 6.98-6.47 (m, 4H), 4.35 (q, J=7.1 Hz, 2H), 4.24 (br s, 4H), 3.71 (s, 6H), 1.33 (t, J=7.1 Hz, 3H).

LCMS; m/z 482.1 (M+Na)⁺ (ES⁺).

### Step B: Ethyl 3-(N,N-bis(4-methoxybenzyl)sulfamoyl)-1-isopropyl-1H-pyrazole-5-carboxylate

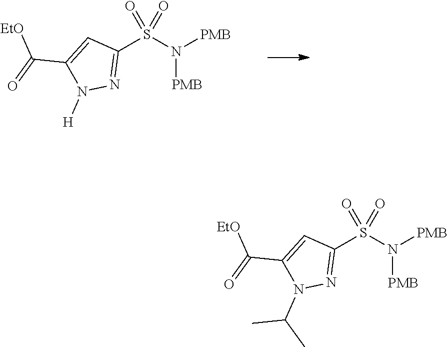

Ethyl 3-(N,N-bis(4-methoxybenzyl)sulfamoyl)-1H-pyrazole-5-carboxylate (1 g, 2.176 mmol), K₂CO₃ (0.391 g, 2.83 mmol) and 2-iodopropane (0.26 mL, 2.61 mmol) were stirred in DMF (10 mL) under a nitrogen atmosphere for 18 hours. The reaction was poured onto brine (100 mL) and EtOAc (50 mL). The aqueous layer was discarded and the organic layer washed with brine (2×100 mL), dried (MgSO₄), filtered and concentrated to dryness to give a yellow oil which was purified by chromatography on silica gel (80 g column, 0-40% EtOAc/isohexane) to afford the title compound (1.0 g, 85%) as a clear colourless oil which solidified on standing.

¹H NMR (DMSO-d₆) δ 7.22-6.93 (m, 5H), 6.93-6.68 (m, 4H), 5.45 (sept, J=6.6 Hz, 1H), 4.32 (q, J=7.1 Hz, 2H), 4.25 (s, 4H), 3.71 (s, 6H), 1.42 (d, J=6.6 Hz, 6H), 1.32 (t, J=7.1 Hz, 3H).

LCMS; m/z 524.2 (M+Na)⁺ (ES⁺).

### Step C: 3-(N,N-Bis(4-methoxybenzyl)sulfamoyl)-1-isopropyl-1H-pyrazole-5-carboxylic Acid, Sodium Salt

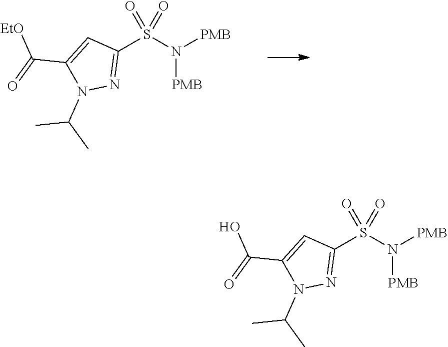

Ethyl 3-(N,N-bis(4-methoxybenzyl)sulfamoyl)-1-isopropyl-1H-pyrazole-5-carboxylate (1 g, 1.994 mmol) was suspended in EtOH (10 mL) and 2 M aqueous sodium hydroxide (1.994 ml, 3.99 mmol). The reaction was left to stir at room temperature for 17 hours, then evaporated to dryness under reduced pressure to afford the title compound as a colourless foam which was used without further purification.

LCMS; m/z 496.1 (M+Na)⁺ (ES⁺).

### Step D: 5-(Azetidine-1-carbonyl)-1-isopropyl-N,N-bis(4-methoxybenzyl)-1H-pyrazole-3-sulfonamide

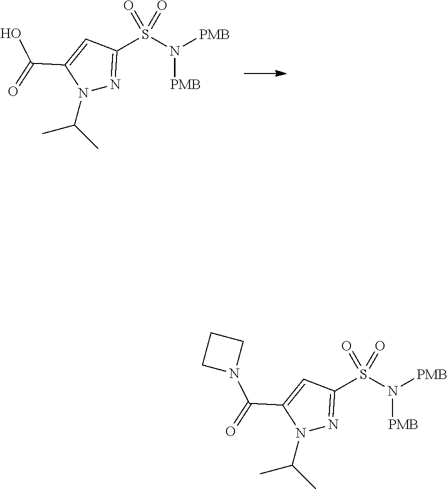

T3P (50 wt % in EtOAc) (2.28 mL, 3.83 mmol) was added to a mixture of sodium 3-(N,N-bis(4-methoxybenzyl)sulfamoyl)-1-isopropyl-1H-pyrazole-5-carboxylate (0.99 g, 1.913 mmol) and azetidine hydrochloride (0.215 g, 2.296 mmol) in THF (10 mL). DIPEA (0.67 mL, 3.84 mmol) was added and the reaction stirred at room temperature for 5 hours. Additional T3P (50 wt % in EtOAc) (2.28 mL, 3.83 mmol), DIPEA (0.67 mL, 3.84 mmol) and azetidine hydrochloride (0.215 g, 2.296 mmol) were added and the reaction was stirred at room temperature for a further 2 days. The reaction mixture was diluted with EtOAc (20 mL) and washed with 2 M aqueous NaOH (2×20 mL) followed by 1 M aqueous HCl (20 mL). The organic layer was dried (MgSO₄), filtered and concentrated to dryness to give crude product still containing starting acid. The mixture was subjected to the reaction procedure above, stirred for 2 days, then diluted with EtOAc (20 mL) and washed with water (2×30 mL) followed by 1 M aqueous HCl (20 mL). The organic layer was dried (MgSO₄), filtered and concentrated to dryness to afford the title compound (786 mg, 75%) as an orange oil which was used without further purification in the next step.

¹H NMR (CDCl₃) δ 7.17-6.99 (m, 4H), 6.85-6.73 (m, 4H), 6.70 (s, 1H), 5.46 (sept, J=6.6 Hz, 1H), 4.37-4.24 (m, 6H), 4.20 (t, J=7.8 Hz, 2H), 3.78 (s, 6H), 2.50-2.29 (m, 2H), 1.47 (d, J=6.6 Hz, 6H).

LCMS; m/z 513.2 (M+H)⁺ (ES⁺).

### Step E: 5-(Azetidine-1-carbonyl)-1-isopropyl-1H-pyrazole-3-sulfonamide

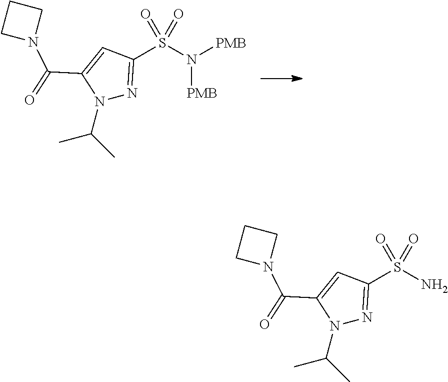

5-(Azetidine-1-carbonyl)-1-isopropyl-N,N-bis(4-methoxybenzyl)-1H-pyrazole-3-sulfonamide (0.79 g, 1.54 mmol) was dissolved in DCM:TFA (5:1.12 mL) and stirred for 18 hours at room temperature. The solvent was removed under reduced pressure and the purple residue obtained was purified by chromatography on silica gel (24 g column, 0-5% MeOH/DCM) to afford the title compound (201 mg, 47%) as a colourless foam. ¹H NMR (DMSO-d₆) δ 7.51 (s, 2H), 6.85 (s, 1H), 5.28 (sept, J=6.6 Hz, 1H), 4.30 (t, J=7.7 Hz, 2H), 4.04 (t, J=7.8 Hz, 2H), 2.27 (p, J=7.8 Hz, 2H), 1.40 (d, J=6.6 Hz, 6H). LCMS; m/z 273.1 (M+H)⁺ (ES⁺).

### Intermediate Pin: (4-(Dimethylamino)pyridin-1-ium-1-carbonyl)((5-(dimethylcarbamoyl)-1-methyl-1H-pyrazol-3-yl)sulfonyl)amide

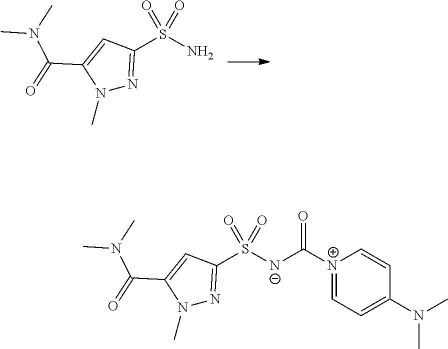

A solution of N,N,1-trimethyl-3-sulfamoyl-1H-pyrazole-5-carboxamide (Intermediate P5) (459 mg, 1.976 mmol) in MeCN (2.3 mL) was treated with N,N-dimethylpyridin-4-amine (483 mg, 3.95 mmol) and the reaction mixture was stirred at room temperature until the sulfonamide had dissolved. Diphenyl carbonate (466 mg, 2.174 mmol) was added and the reaction mixture was left for 16 hours at room temperature. The resulting precipitate was separated by filtration, washed with MeCN and dried to afford the title compound (578 mg, 77%) which was used in the next step without further purification.

¹H NMR (DMSO-d₆) δ 8.77-8.73 (m, 2H), 7.02-6.98 (m, 2H), 6.83 (s, 1H), 3.85 (s, 3H), 3.26 (s, 6H), 3.05 (s, 3H), 3.00 (s, 3H).

### Intermediate P14: 1-(1-Acetylpiperidin-4-yl)-1H-pyrazole-3-sulfonamide

### Step A: 1-(4-(3-Nitro-1H-pyrazol-1-yl)piperidin-1-yl)ethanone

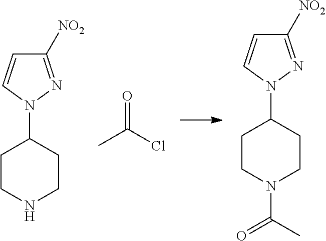

Triethylamine (3.59 ml, 25.8 mmol) was added to 4-(3-nitro-1H-pyrazol-1-yl) piperidine, HCl (2.00 g, 8.60 mmol) in DCM (30 mL), and the reaction was stirred at room temperature for 5 minutes. Then acetyl chloride (0.744 ml, 10.32 mmol) was added dropwise at 0° C. The reaction was stirred for 20 minutes at 0° C. and then allowed to warm to room temperature and stirred for 16 hours. The mixture was then diluted with water (20 mL). The phases were separated and the aqueous layer was extracted with DCM (2×20 mL). The combined organic layers were washed with brine, dried (MgSO₄) and concentrated in vacuo to afford the title compound as a yellow solid (1.85 g, 89%).

¹H NMR (DMSO-d₆) δ 8.13 (d, J=2.6 Hz, 1H), 7.07 (d, J=2.6 Hz, 1H), 4.60 (tt, J=11.5, 4.1 Hz, 1H), 4.52-4.43 (m, 1H), 3.98-3.89 (m, 1H), 3.23-3.15 (m, 1H), 2.76-2.64 (m, 1H), 2.14-2.04 (m, 2H), 2.04 (s, 3H), 1.97-1.85 (m, 1H), 1.75 (qd, J=12.3, 4.6 Hz, 1H). LCMS; m/z 239.2 (M+H)⁺ (ES⁺).

### Step B: 1-(4-(3-Amino-1H-pyrazol-1-yl)piperidin-1-yl)ethanone

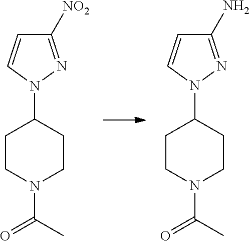 [SMILES: CC(=O)N1CCC(n2ccc([N+](=O)[O-])n2)CC1 ; CC(=O)N1CCC(n2ccc(N)n2)CC1]

A solution of 1-(4-(3-nitro-1H-pyrazol-1-yl)piperidin-1-yl)ethanone (1.75 g, 7.35 mmol) in ethanol (36.7 mL) was treated with saturated aq NH₄Cl (24.48 mL, 7.35 mmol), followed by iron powder (2.05 g, 36.7 mmol) at room temperature. The mixture was then stirred at 75° C. for 1 hour. After cooling, the reaction was filtered through a pad of Celite®, washing with EtOAc (2×20 mL). The organic solvents were evaporated in vacuo. The aqueous solution obtained was then extracted with EtOAc (2×40 mL) and the combined organic layers dried (MgSO₄), filtered and concentrated in vacuo to give the title product as a green solid (250 mg). The aqueous layer was concentrated to dryness, suspended in EtOAc:MeOH (9:1, 50 mL) and the insoluble material was filtered off. The filtrate was concentrated in vacuo to afford further product which was combined with the extracted portion previously obtained to afford the title compound as a green-brown solid (731 mg, 45%).

¹H NMR (DMSO-d₆) δ 7.34 (d, J=2.3 Hz, 1H), 5.38 (d, J=2.2 Hz, 1H), 4.64 (br s, 2H), 4.49-4.28 (m, 1H), 4.15-4.02 (m, 1H), 3.93-3.80 (m, 1H), 3.21-3.08 (m, 1H), 2.64 (td, J=12.8, 2.8 Hz, 1H), 2.01 (s, 3H), 1.98-1.85 (m, 2H), 1.75 (qd, J=12.3, 4.3 Hz, 1H), 1.62 (qd, J=12.3, 4.4 Hz, 1H).

LCMS; m/z 209.1 (M+H)⁺ (ES⁺).

### Step C: 1-(1-Acetylpiperidin-4-yl)-1H-pyrazole-3-sulfonamide

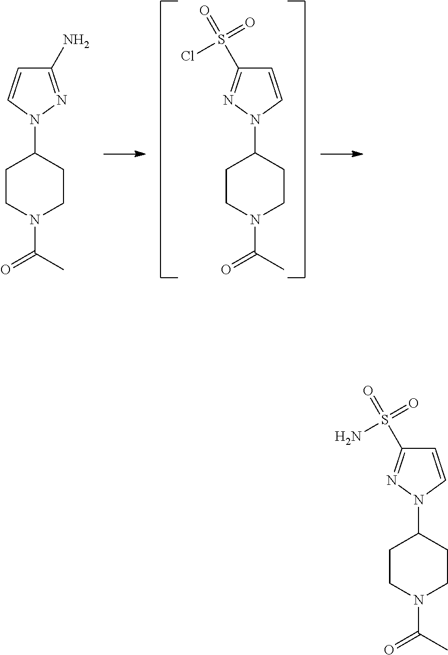

A solution of NaNO₂ (273 mg, 3.95 mmol) in water (0.5 mL) was added dropwise to a solution of HCl (aq 37%,1.2 mL) in water (0.8 mL) and MeCN (4.1 mL) at −10° C., maintaining the internal temperature below 0° C. The solution was stirred for 10 minutes and then a solution of 1-(4-(3-amino-H-pyrazol-1-yl)piperidin-1-yl)ethanone (730 mg, 3.29 mmol) in MeCN (4.1 mL) was added dropwise at 0° C. over 15 minutes. The resulting reaction mixture was stirred at 0° C. for 45 minutes. AcOH (1.7 mL), CuCl₂ (222 mg, 1.65 mmol) and CuCl (16.3 mg, 0.165 mmol) were sequentially added and the reaction mixture was purged with SO₂ gas for 20 minutes at 0° C. The reaction mixture was stirred for a further 50 minutes and then diluted with water (30 mL) and extracted with EtOAc (3×30 mL). The combined organic extracts were dried (MgSO₄), filtered and concentrated in vacuo. The crude product was purified by chromatography on silica gel (24 g column, 0-10% 0.7 N NH₃ in MeOH/DCM) to afford the intermediate 1-(1-acetylpiperidin-4-yl)-1H-pyrazole-3-sulfonyl chloride as a clear yellow oil (217 mg), which was dissolved in THF (1 mL) and treated with ammonia (0.5M in dioxane, 3.37 mL, 1.684 mmol). The reaction mixture was stirred at room temperature for 22 hours and then concentrated in vacuo, quenched with water (20 mL) and extracted with EtOAc (2×20 mL). The combined organic layers were dried (MgSO₄) and concentrated in vacuo. The residue was dissolved in EtOAc (5 mL) and precipitated by addition of isohexane (10 mL). The solid was collected by filtration, washed with isohexane (2×3 mL) and dried under vacuum to afford the title compound as a white solid (81 mg, 37%).

¹H NMR (DMSO-d₆) δ 7.95 (d, J=2.4 Hz, 1H), 7.40 (s, 2H), 6.59 (d, J=2.4 Hz, 1H), 4.62-4.35 (m, 2H), 3.97-3.83 (m, 1H), 3.24-3.18 (m, 1H), 2.77-2.62 (m, 1H), 2.09-1.99 (m, 4H), 1.88 (qd, J=12.2, 4.4 Hz, 1H), 1.73 (qd, J=12.3, 4.5 Hz, 1H), 0.89-0.79 (m, 1H).

LCMS; m/z 273.0 (M+H)⁺ (ES⁺).

### Intermediate P15: N-((1-Isopropyl-3-sulfamoyl-1H-pyrazol-5-yl)methyl)-N-methylacetamide

### Step A: 1-Isopropyl-N,N-bis(4-methoxybenzyl)-1H-pyrazole-3-sulfonamide

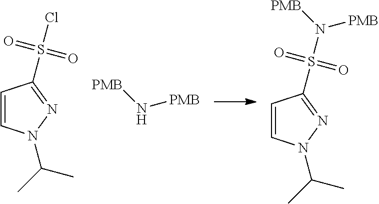

1-Isopropyl-1H-pyrazole-3-sulfonyl chloride (10.0 g, 47.9 mmol) was added portionwise to a solution of bis(4-methoxybenzyl)amine (12.5 g, 48.6 mmol) and Et₃N (13 mL, 93 mmol) in DCM (250 mL) cooled in an ice bath. The mixture was stirred for 30 minutes, warmed to room temperature and stirred for 2 hours. The mixture was washed with water (200 mL), aqueous 1M HCl (200 mL) and water (200 mL), dried (MgSO₄), filtered and evaporated to give a yellow oil. The yellow oil was purified by chromatography on silica gel (330 g column, 0-70% EtOAc/isohexane) to afford the title compound (16.6 g, 80%) as a white solid.

¹H NMR (DMSO-d₆) δ 8.00 (d, J=2.4 Hz, 1H), 7.07-6.96 (m, 4H), 6.85-6.76 (m, 4H), 6.70 (d, J=2.4 Hz, 1H), 4.61 (sept, J=6.7 Hz, 1H), 4.20 (s, 4H), 3.71 (s, 6H), 1.44 (d, J=6.7 Hz, 6H).

LCMS; m/z 452.2 (M+Na)⁺ (ES⁺).

### Step B: 5-Formyl-1-isopropyl-N,N-bis(4-methoxybenzyl)-1H-pyrazole-3-sulfonamide

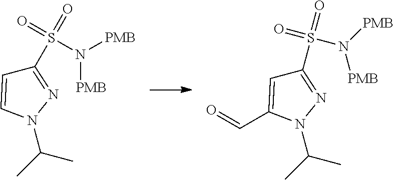

A solution of n-BuLi (2.5 M in hexanes, 2 mL, 5.00 mmol) was added dropwise to a stirred solution of 1-isopropyl-N,N-bis(4-methoxybenzyl)-1H-pyrazole-3-sulfonamide (2 g, 4.66 mmol) in THF (26 mL) at −78° C. The reaction was stirred for 1 hour. Then a solution of morpholine-4-carbaldehyde (1.6 g, 13.90 mmol) in THF (4 mL) was added dropwise, whilst maintaining the temperature below −65′C. The reaction mixture was left at −78° C. for 1 hour and then quenched with saturated NH₄Cl solution (25 mL) and extracted with EtOAc (2×30 mL). The combined organic extracts were washed with saturated brine (50 mL), dried over MgSO₄, filtered and concentrated in vacuo to afford a yellow oil. The crude product was purified by chromatography on SiO₂ (120 g column, 0-100% EtOAc/isohexane) to afford the title compound (1.43 g, 66%) as a white solid.

¹H NMR (DMSO-d₆) δ 9.91 (s, 1H), 7.43 (s, 1H), 7.10-7.03 (m, 4H), 6.86-6.79 (m, 4H), 5.34 (sept, J=6.6 Hz, 1H), 4.26 (s, 4H), 3.72 (s, 6H), 1.43 (d, J=6.5 Hz, 6H). LCMS m/z 480.3 (M+Na)⁺ (ES⁺).

### Step C: 1-Isopropyl-N,N-bis(4-methoxybenzyl)-5-((methylamino)methyl)-1H-pyrazole-3-sulfonamide, Acetic Acid Salt

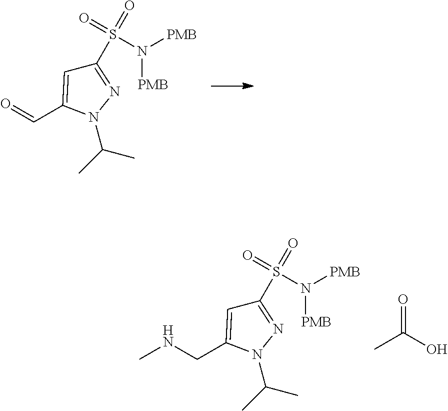

Acetic acid (10 μL, 0.175 mmol) was added to a stirred suspension of 5-formyl-1-isopropyl-N,N-bis(4-methoxybenzyl)-1H-pyrazole-3-sulfonamide (400 mg, 0.874 mmol), methylamine (2 M in THF) (874 μL, 1.748 mmol) and sodium triacetoxy-borohydride (278 mg, 1.311 mmol) in THF (14 mL). The reaction mixture was left to stir at room temperature for 16 hours. Water (1 mL) was added and volatiles were evaporated. The crude product was purified by chromatography on SiO₂ (24 g column, 0-10% MeOH/DCM) to afford the title compound (130 mg, 24%) as a colourless oil. ¹H NMR (DMSO-d₆) δ 7.05-6.98 (m, 4H), 6.85-6.79 (m, 4H), 6.57 (s, 1H), 4.77 (sept, J=6.5 Hz, 1H), 4.19 (s, 4H), 3.74 (s, 2H), 3.72 (s, 6H), 3.37 (bs, 1H), 2.27 (s, 3H), 1.90 (s, 3H), 1.39 (d, J=6.5 Hz, 6H). OH not observed.

LCMS; m/z 473.5 (M+H)⁺ (ES⁺).

### Step D: N-((3-(N,N-Bis(4-methoxybenzyl)sulfamoyl)-1-isopropyl-1H-pyrazol-5-yl)methyl)-N-methylacetamide

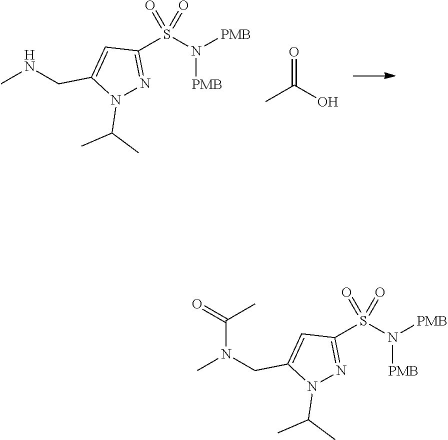

To a solution of 1-isopropyl-N,N-bis(4-methoxybenzyl)-5-((methylamino)methyl)-1H-pyrazole-3-sulfonamide, acetic acid salt (130 mg, 0.248 mmol) in DCM (1 mL) was added pyridine (45 μL, 0-556 mmol) and the mixture was cooled to 0° C. Trifluoroacetic anhydride (53 μL, 0.375 mmol) was added dropwise and the resultant mixture was stirred at 0° C. for 15 minutes, before warming to room temperature for 16 hours. Additional pyridine (45 μL, 0.556 mmol) and trifluoroacetic anhydride (53 μL, 0.375 mmol) were added and the mixture was stir for another 16 hours. The mixture was quenched with saturated sodium bicarbonate (5 mL) and the layers were separated. The aqueous layer was extracted with DCM (2×10 mL) and EtOAc (10 mL) and the combined organic phases were dried with magnesium sulfate. The solvent was removed under reduced pressure. The crude product was purified by chromatography on SiO₂ (12 g column, 0-10% MeOH/DCM) to afford the title compound (88 mg, 57%) as a brown oil.

¹H NMR (DMSO-d₆); rotamers: δ 7.05-6.99 (m, 4H), 6.85-6.79 (m, 4H), 6.63 (s, 1H), 4.75-4.66 (m, 1H), 4.63 (s, 2H), 4.21 (s, 4H), 3.73 (s, 6H), 3.32 (s, 3H), 2.07 (s, 3H), 1.35 (d, J=6.5 Hz, 6H).

LCMS; m/z 537.1 (M+Na)⁺ (ES⁺).

### Step E: N-((1-Isopropyl-3-sulfamoyl-1H-pyrazol-5-yl)methyl)-N-methylacetamide

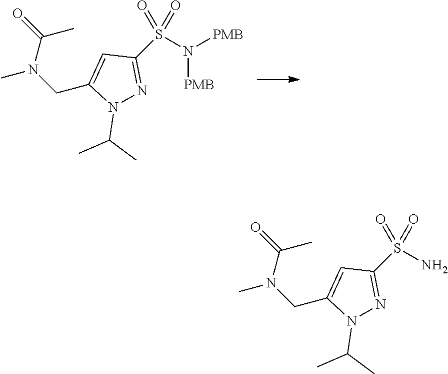

N-((3-(N,N-Bis(4-methoxybenzyl)sulfamoyl)-1-isopropyl-1H-pyrazol-5-yl)methyl)-N-methylacetamide (88 mg, 0.140 mmol) was dissolved in DCM (0.5 mL) and TFA (1.5 mL) was added. The solution was stirred for 16 hours. The reaction mixture was concentrated in vacuo, suspended in toluene (5 mL) and concentrated again. The crude product was purified by chromatography on RP Flash C18 (basic) to afford the title compound (30 mg, 78%) as a white solid.

¹H NMR (DMSO-d₆) δ 7.37 (bs, 2H), 6.51 (s, 1H), 4.73-4.66 (m, 1H), 4.61 (s, 2H), 2.95 (s, 3H), 2.06 (s, 3H), 1.35 (d, J=6.5 Hz, 6H).

LCMS m/z 275.2 (M+H)⁺ (ES⁺).

### Intermediate P16: N,N-Bis(2-methoxyethyl)-1-methyl-3-sulfamoyl-1H-pyrazole-5-carboxamide

### Step A: 1-Methyl-1H-pyrazole-3-sulfonyl Chloride

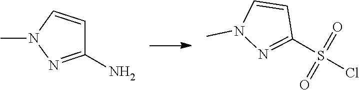

A solution of 1-methyl-1H-pyrazol-3-amine (25 g, 257.42 mmol, 1 eq) in MeCN (600 mL) at 0° C. was treated with concentrated HCl (60 mL) and H₂O (60 mL). Then an aqueous solution of NaNO₂ (21-31 g, 308.90 mmol, 1.2 eq) in H₂O (60 mL) was added slowly. The resulting mixture was stirred at 0° C. for 40 minutes. AcOH (60 mL), CuCl₂ (17-31 g, 128.71 mmol, 0.5 eq) and CuCl (1.27 g, 12.87 mmol, 307.78 μL, 0.05 eq) were added, then SO₂ gas (15 psi) was bubbled into the mixture for 15 minutes at 0° C. The reaction mixture was concentrated in vacuo to remove most of the MeCN. Then the reaction mixture was treated with H₂O (2.5 L) and extracted with EtOAc (2×1.2 L). The combined organic layers were washed with brine (3×2 L), dried over anhydrous Na₂SO₄, filtered and concentrated under reduced pressure. The residue was purified by silica gel column chromatography (petroleum ether:ethyl acetate=15:1 to 5:1) to give the title compound (19 g, 41%) as a yellow oil.

¹H NMR (CDCl₃): δ 7.52 (d, 1H), 6.89 (d, 1H) and 4.07 (s, 3H).

### Step B: N,N-Bis(4-methoxybenzyl)-1-methyl-1H-pyrazole-3-sulfonamide

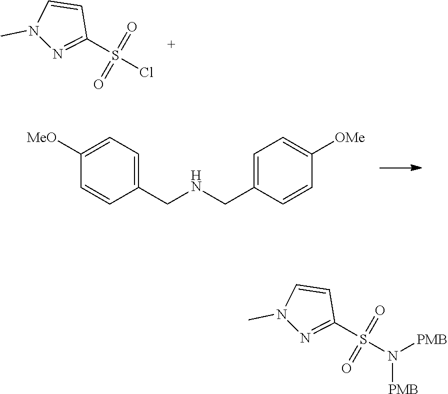

To a solution of bis(4-methoxybenzyl)amine (99.83 g, 387.96 mmol, 0.91 eq) in THF (1 L) was added TEA (86.28 g, 852.65 mmol, 118.68 mL, 2 eq), followed by 1-methyl-1H-pyrazole-3-sulfonyl chloride (77 g, 42633 mmol, 1 eq). Then the reaction mixture was stirred at 25° C. for 12 hours. The reaction mixture was concentrated in vacuo to remove most of the THF. The reaction mixture was quenched by addition of aqueous HCl (1 M, 500 mL) and then extracted with EtOAc (2×500 mL). The combined organic layers were washed with brine (2×600 mL), dried over anhydrous Na₂SO₄, filtered and concentrated under reduced pressure. The residue was triturated with a mixture of petroleum ether and ethyl acetate (70 mL, v:v=5:1) to give the title compound (138 g, 81%) as a white solid.

¹H NMR (CDCl₃): δ 7.40 (d, 1H), 7.08 (d, 4H), 6.78 (d, 4H), 6.65-6.63 (m, 1H), 4.32 (s, 4H), 3.98 (s, 3H) and 3.79 (s, 6H).

LCMS: m/z 402.2 (M+H)⁺ (ES⁺).

### Step C: 3-(N,N-Bis(4-methoxybenzyl)sulfamoyl)-1-methyl-1H-pyrazole-5-carboxylic Acid

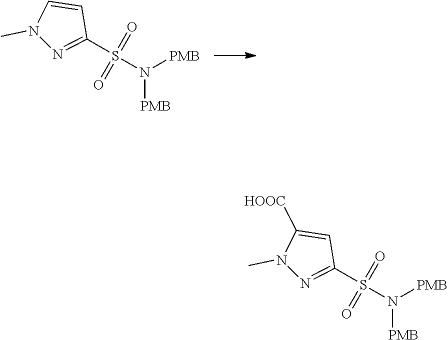

A solution of N,N-bis(4-methoxybenzyl)-1-methyl-1H-pyrazole-3-sulfonamide (100 g, 249.08 mmol, 1 eq) in THF (1.35 L) was cooled to −70° C. Then n-BuLi (2.5 M, 104.61 mL, 1.05 eq) was added dropwise. The reaction mixture was stirred at −70° C. for 1 hour, then CO₂ (15 psi) was bubbled into the mixture for 15 minutes. The reaction mixture was stirred at −70° C. for another 1 hour. The reaction mixture was quenched with H₂O (1.2 L) and adjusted with aqueous HCl (1 M) to pH=3. Then the mixture was extracted with EtOAc (2×1). The combined organic layers were washed with brine (2×1 L), dried over Na₂SO₄, filtered and concentrated in vacuo. The residue was triturated with a mixture of petroleum ether and ethyl acetate (300 mL, v:v=1:1) to give the title compound (94 g, 84% yield, 99% purity on LCMS) as a white solid.

¹H NMR (DMSO-d₆): δ 6.98-7.16 (m, 5H), 6.82 (d, 4H), 4.25 (s, 4H), 4.15 (s, 3H) and 3.72 (s, 6H).

LCMS: m/z 468.2 (M+Na)⁺ (ES⁺).

### Step D: 3-(N,N-Bis(4-methoxybenzyl)sulfamoyl)-N,N-bis(2-methoxyethyl)-1-methyl-1H-pyrazole-5-carboxamide

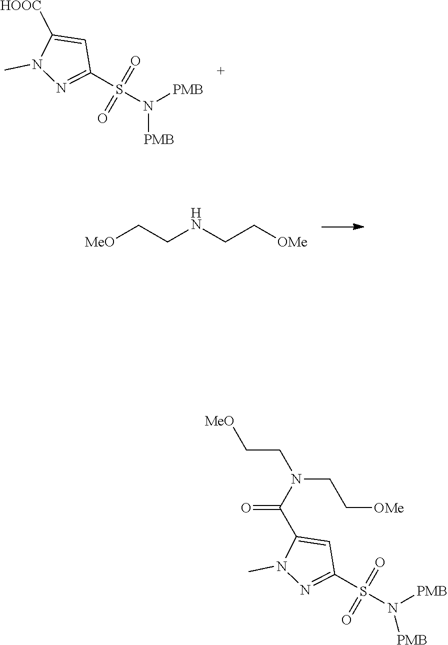

To a solution of 3-(N,N-bis(4-methoxybenzyl)sulfamoyl)-1-methyl-1H-pyrazole-5-carboxylic acid (8 g, 17.96 mmol, 1 eq) in DMF (100 mL) was added with HATU (10.24 g, 26.94 mmol, 1.5 eq), DIPEA (6.96 g, 53.87 mmol, 3 eq) and bis(2-methoxyethyl)amine (2.87 g, 21.55 mmol, 1.2 eq). The reaction mixture was stirred at 25° C. for 1 hour. Then the reaction mixture was diluted with EtOAc (50 mL), washed with saturated aqueous NH₄C solution (3×50 mL) and brine (3×50 mL). The organic layer was dried over anhydrous Na₂SO₄ filtered and concentrated in vacuo. The residue was purified by reversed phase flash chromatography (0.05% NH₃.H₂O-MeCN) to give the title compound (8 g, 79%) as a red oil.

¹H NMR (CD₃OD): δ 7.05 (d, 4H), 6.81-6.77 (m, 5H), 4.29 (s, 4H), 3.90 (s, 3H), 3.79-3.72 (m, 8H), 3.68-3.57 (m, 4H), 3.48-3.46 (m, 2H), 3.38 (s, 3H) and 3.27 (s, 3H). LCMS: m/z 561.3 (M+H)⁺ (ES⁺).

### Step E: N,N-Bis(2-methoxyethyl)-1-methyl-3-sulfamoyl-1H-pyrazole-5-carboxamide

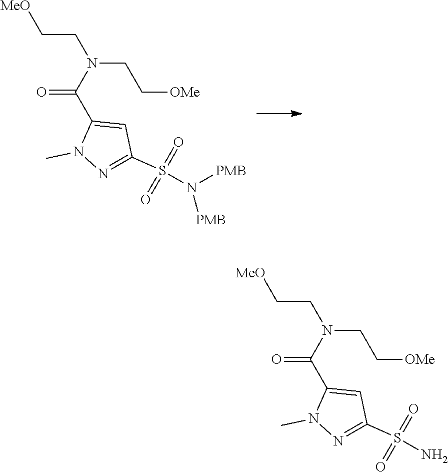

To a solution of 3-(N,N-bis(4-methoxybenzyl)sulfamoyl)-N,N-bis(2-methoxyethyl)-1-methyl-1H-pyrazole-5-carboxamide (8 g, 14.27 mmol, 1 eq) in DCM (50 mL) was added TFA (56 g, 491.13 mmol, 34.42 eq). The reaction mixture was stirred at 25° C. for 12 hours and then concentrated in vacuo. The residue was triturated with a mixture of EtOAc and PE (50 mL, v:v=3:2) to give the title compound (4.0 g, 88%) as a white solid.

¹H NMR (DMSO-d₆): δ 7.50 (s, 2H), 6.74 (s, 1H), 3.84 (s, 3H), 3.63 (t, 4H), 3.43-3.40 (m, 4H), 3.28 (s, 3H) and 3.18 (s, 3H).

### Intermediate P17: 1-(1-(Dimethylamino)-2-methylpropan-2-yl)-1H-pyrazole-3-sulfonamide

### Step A: Lithium 1-(tetrahydro-2H-pyran-2-yl)-1H-pyrazole-5-sulfinate

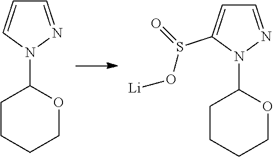

A solution of n-BuLi (100 mL, 250 mmol, 2.5M in hexanes) was added slowly to a solution of 1-(tetrahydro-2H-pyran-2-yl)-1H-pyrazole (36.2 g, 238 mmol) in THF (500 mL) keeping the temperature below −65° C. The mixture was stirred for 1.5 hours, then sulfur dioxide was bubbled through for 10 minutes. The mixture was allowed to warm to room temperature, the solvent evaporated and the residue triturated with TBME (300 mL) and filtered. The solid was washed with TBME and isohexane and dried to afford the crude title compound (54.89 g, 99%).

¹H NMR (DMSO-d₆) δ 7.26 (d, J=1.6 Hz, 1H), 6.10 (d, J=1.7 Hz, 1H), 5.99 (dd, J=10.0, 2.5 Hz, 1H), 3.92-3.87 (m, 1H), 3.56-3.49 (m, 1H), 2.25-2.15 (m, 1H), 2.00-1.91 (m, 1H), 1.75-1.69 (m, 1H), 1.66-1.46 (m, 3H).

LCMS; m/z 215 (M−H)⁻ (ES⁻).

### Step B: N,N-Bis(4-methoxybenzyl)-1-(tetrahydro-2H-pyran-2-yl)-1H-pyrazole-5-sulfonamide

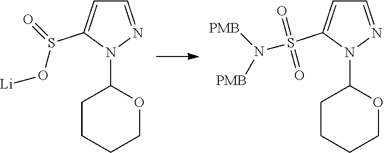

NCS (12.0 g, 90 mmol) was added to a suspension of lithium 1-(tetrahydro-2H-pyran-2-yl)-1H-pyrazole-5-sulfinate (20 g, 90 mmol) in DCM (250 mL) cooled in an ice bath. The mixture was stirred for 4 hours, quenched with water (100 mL), and then partitioned between DCM (300 mL) and water (200 mL). The organic phase was washed with water (200 mL), dried (MgSO₄), filtered and evaporated to ˜50 mL. The solution was added to a mixture of bis(4-methoxybenzyl)amine (24 g, 93 mmol) and triethylamine (40 mL, 287 mmol) in DCM (300 mL) cooled in an ice bath. After stirring for 1 hour, the mixture was warmed to room temperature, and then partitioned between DCM (300 mL) and water (250 mL). The organic layer was washed with water (250 mL), aq 1M HCl (2×250 mL), water (250 mL), dried (MgSO₄), filtered, and evaporated to afford the crude title compound (41.02 g, 97%) as a brown oil.

LCMS; m/z 494.2 (M+Na)⁺ (ES⁺).

### Step C: N,N-Bis(4-methoxybenzyl)-1H-pyrazole-3-sulfonamide

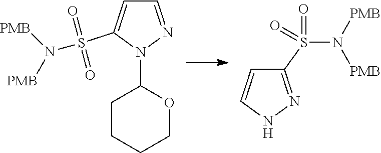

A mixture of N,N-bis(4-methoxybenzyl)-1-(tetrahydro-2H-pyran-2-yl)-1H-pyrazole-5-sulfonamide (41 g, 87 mmol) and aq 1M HCl (30 mL) in THF (300 mL) and MeOH (50 mL) was stirred at room temperature for 18 hours. The solvent was evaporated and the residue partitioned between EtOAc (400 mL) and aq 1M HCl (200 mL). The organic layer was washed with 10% brine (200 mL dried (MgSO₄), filtered and evaporated. The residue was triturated with TBME, filtered and dried to afford the title compound (24.87 g, 69%) as an off-white solid.

¹H NMR (CDCl₃) δ 7.88 (d, J=2.4 Hz, 1H), 7.06-7.02 (m, 4H), 6.79-6.75 (m, 4H), 6.63 (d, J=2.4 Hz, 1H), 4.31 (s, 4H), 3.78 (s, 6H). Exchangeable proton not visible.

LCMS; m/z 388 (M+H)⁺ (ES⁺); 386 (M−H)⁻ (ES⁻).

### Step D: Methyl 2-(3-(N,N-bis(4-methoxybenzyl)sulfamoyl)-H-pyrazol-1-yl)-2-methylpropanoate

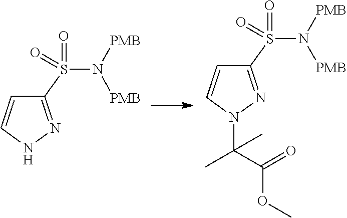

N,N-Bis(4-methoxybenzyl)-1H-pyrazole-3-sulfonamide (2.00 g, 5.16 mmol) and potassium carbonate (2.140 g, 15-49 mmol) were suspended in dry DMF (30 mL). Methyl 2-bromo-2-methylpropanoate (1.002 mL, 7.74 mmol) was added and the mixture was heated to 80° C. overnight. The reaction mixture was cooled to room temperature, diluted with water (20 mL), poured into brine (200 mL) and extracted with TBME (2×50 mL). The combined organic layers were dried (MgSO₄), filtered and evaporated to dryness to give a yellow oil. The crude product was purified by chromatography on silica gel (80 g column, 0-70% EtOAc/isohexane) to afford the title compound (2.45 g, 94%) as a clear colourless oil.

¹H NMR (DMSO-d₆) δ 8.18 (d, J=2.5 Hz, 1H), 7.05-6.95 (m, 4H), 6.85-6.78 (m, 4H), 6.78 (d, J=2.5 Hz, 1H), 4.18 (s, 4H), 3.72 (s, 6H), 3.65 (s, 3H), 1.81 (s, 6H).

LCMS; m/z 511 (M+Na)⁺ (ES⁺).

### Step E: 2-(3-(N,N-Bis(4-methoxybenzyl)sulfamoyl)-H-pyrazol-1-yl)-2-methylpropanoic Acid

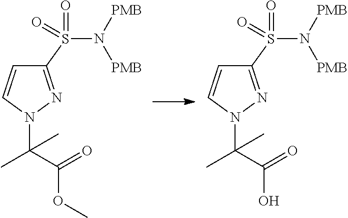

A mixture of methyl 2-(3-(N,N-bis(4-methoxybenzyl)sulfamoyl)-1H-pyrazol-1-yl)-2-methylpropanoate (2.4 g, 4.92 mmol) and aq 2M NaOH (5 mL, 10.00 mmol) in THF (5 mL) and MeOH (3 mL) was stirred at room temperature for 20 hours. The mixture was partitioned between EtOAc (100 mL) and aq. 1M HCl (100 mL), the organic layer washed with brine (50 mL), dried (MgSO₄), filtered and evaporated to afford the title compound (2.38 g, 95%) as a gum that solidified on standing.

¹H NMR (CDCl₃) δ 7.64 (d, J=2.5 Hz, 1H), 7.09-7.05 (m, 4H), 6.80-6.77 (m, 4H), 6.73 (d, J=2.5 Hz, 1H), 4.32 (s, 4H), 3.80 (s, 6H), 1.91 (s, 6H). Exchangeable proton not visible.

LCMS; m/z 472 (M−H)⁻ (ES⁻).

### Step F: 2-(3-(N,N-Bis(4-methoxybenzyl)sulfamoyl)-H-pyrazol-1-yl)-N,N,2-trimethylpropanamide

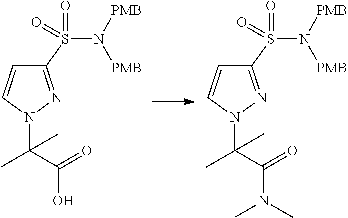

A mixture of 2-(3-(N,N-bis(4-methoxybenzyl)sulfamoyl)-1H-pyrazol-1-yl)-2-methylpropanoic acid (2.1 g, 4.43 mmol), N,N-diisopropylethylamine (3.1 mL, 17.75 mmol) and HATU (1.9 g, 5.00 mmol) in DMF (30 mL) was stirred at 0-5° C. for 10 minutes, and then dimethylamine hydrochloride (0.723 g, 8.87 mmol) was added. The mixture was warmed to room temperature, stirred for 20 hours, and then partitioned between TBME (200 mL) and aq 1M HCl (200 mL). The organic layer was washed with water (100 mL), dried (MgSO₄), filtered, evaporated to dryness, and then purified by chromatography on silica gel (40 g cartridge, 0-100% EtOAc/heptane) to afford the title compound (2.2 g, 98%) as a clear gum.

¹H NMR (CDCl₃, rotamers) δ 7.48 (d, J=2.4 Hz, 1H), 7.14-7.10 (m, 4H), 6.82-6.78 (m, 5H), 4.33 (s, 4H), 3.81 (s, 6H), 2.97 (br s, 3H), 2.37 (br s, 3H), 1.82 (s, 6H).

LCMS; m/z 501 (M+H)⁺ (ES⁺).

### Step G: N,N,2-Trimethyl-2-(3-sulfamoyl-1H-pyrazol-1-yl)propanamide

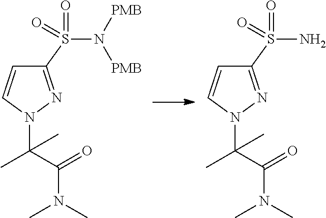

A mixture of 2-(3-(N,N-bis(4-methoxybenzyl)sulfamoyl)-H-pyrazol-1-yl)-N,N,2-trimethylpropanamide (0.8 g, 1.598 mmol) and TFA (6 mL) was stirred for 4 hours. The reaction mixture was concentrated and the crude product was purified by chromatography on silica gel (12 g column, 0-10% MeOH/DCM) to afford the title compound (360 mg, 86%) as a colourless solid.

¹H NMR (DMSO-d₆, rotamers) δ 8.02 (d, J=2.5 Hz, 1H), 7.47 (s, 2H), 6.68 (d, J=2.4 Hz, 1H), 2.82 (br s, 3H), 2.30 (br s, 3H), 1.71 (s, 6H).

### Intermediate A1: 4-Isocyanato-1,2,3,5,6,7-hexahydro-s-indacene

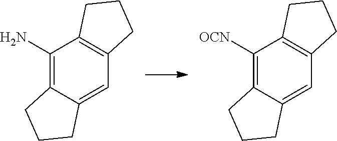

To a solution of phosgene (4.45 mL, 20% weight in toluene, 8.4 mmol) in EtOAc (90 mL) was added dropwise a solution of 1,2,3,5,6,7-hexahydro-s-indacen-4-amine (589 mg, 3.4 mmol) in EtOAc (45 mL) at ambient temperature. The resulting reaction mixture was then heated to reflux for 3 hours and upon cooling was filtered and concentrated in vacuo to afford the title compound as a brown oil (756 mg, 100%). The crude product was used directly in the next step without further purification.

¹H NMR (CDCl₃) δ 6.8 (s, 1H), 2.89 (m, 8H) and 2.09 (m, 4H).

### Intermediate A2: 5-Fluoro-2-isocyanato-,3-diisopropylbenzene

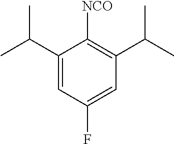

4-Fluoro-2,6-diisopropylaniline (1 g, 5.12 mmol) and triethylamine (0.785 mL, 5.63 mmol) were dissolved in THF (10 mL) and cooled to 0° C. Triphosgene (0.760 g, 2.56 mmol) was added to the mixture portionwise and the reaction mixture was stirred for 16 hours at room temperature. The mixture was concentrated in vacuo. Isohexane (so mL) was added and the suspension filtered through silica (3 g). The filtrate was dried under reduced pressure to afford the title compound (900 mg, 75%) as a colourless oil. ¹H NMR (DMSO-d₆) δ 6.8 (d, J=9.4 Hz, 2H), 3.27-3.12 (m, 2H), 1.23 (d, J=6.8 Hz, 12H).

### Intermediate A3: 4-Fluoro-2-isopropyl-6-(pyridin-3-yl)aniline

### Step A: 2-Bromo-4-fluoro-6-isopropylaniline

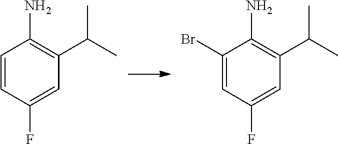

N-Bromosuccinimide (5.64 g, 31.7 mmol) was added portionwise to 4-fluoro-2-isopropylaniline (4.62 g, 30.2 mmol) in DCM (72 mL) at 0° C. The resulting mixture was stirred at 0° C. for 1 hour and then left to warm to room temperature over 21 hours. The reaction mixture was washed with a solution of aqueous sodium hydroxide (2 M, 2×50 mL), dried (MgSO₄), filtered and concentrated in vacuo to give a brown residue. The crude product was then filtered through a plug of silica (50 g) and washed through with 50% DCM in iso-hexane (500 mL). The red filtrate was concentrated to dryness and the crude product was purified by chromatography on silica gel (120 g column, 0-% DCM/iso-hexane) to afford the title compound (4.99 g, 70%) as a red oil.

¹H NMR (CDCl₃) δ 7.07 (dd, 1H), 6.86 (dd, 1H), 4.14 (s, 2H), 2.93 (sept, 1H) and 1.25 (d, 6H). NH₂ not observed.

LCMS; m/z 232.2/234.3 (M+H)⁺ (ES⁺).

### Step B: 4-Fluoro-2-isopropyl-6-(pyridin-3-yl)aniline

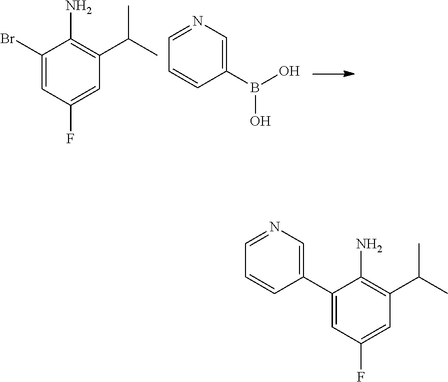

To a stirred, nitrogen-degassed mixture of 2-bromo-4-fluoro-6-isopropylaniline (1.00 g, 4.27 mmol) was added pyridin-3-ylboronic acid (0.577 g, 4.69 mmol), Pd(dppf)Cl₂ (0.156 g, 0.213 mmol) and potassium carbonate (1.769 g, 12.80 mmol) in a 10:1 mixture of 1,4-dioxane:water (33 mL). The reaction mixture was then heated to 80° C. under a nitrogen atmosphere for 2 days, left to cool to room temperature, filtered through a pad of Celite® (10 g) and the filter cake washed with EtOAc (2×30 mL). The filtrate was poured onto water (50 mL) and the organic layer collected. The aqueous layer was extracted with EtOAc (2×20 mL). The combined organic layers were dried (magnesium sulfate), filtered and evaporated to dryness. The crude product was purified by chromatography on silica gel (80 g column, 0-60% EtOAc/iso-hexane) to afford the title compound (273 mg, 27%) as a brown gum.

¹H NMR (CDCl₃) δ 8.70 (dd, 1H), 8.63 (dd, 1H), 7.82 (ddd, 1H), 7.48-7.34 (m, 1H), 6.94 (dd, 1H), 6.70 (dd, 1H), 2.93 (sept, 1H), 3.98-2.44 (br s, 2H) and 1.29 (d, 6H). LCMS; m/z 231.1 (M+H)⁺ (ES⁺).

The following intermediates were synthesised following the general procedure for Intermediate A3:

| Intermediate | Structure | Analytical data |
|---|---|---|
| A4 | 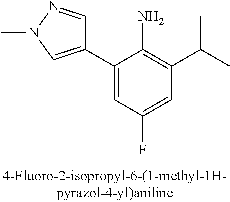 [SMILES: CC(C)c1cc(F)cc(-c2cnn(C)c2)c1N] | ¹H NMR (CDCl₃) δ 7.68 (d, 1H), 7.58 (d, 1H), 6.86 (dd, 1H), 6.78 (dd, 1H), 3.99 (s, 3H), 3.74 (br s, 2H), 2.94 (sept, 1H) and 1.29 (d, 6H). (85 mg, 22%) |
| A5 | 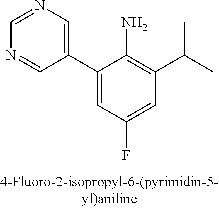 [SMILES: CC(C)c1cc(F)cc(-c2cncnc2)c1N] | ¹H NMR (CDCl₃) δ 9.23 (s, 1H), 8.86 (s, 2H), 6.98 (dd, 1H), 6.69 (dd, 1H), 3.55 (br s, 2H), 2.92 (sept, 1H) and 1.29 (d, 6H). (126 mg, 60%) |

### Intermediate A6: 4-Fluoro-2-isopropyl-6-(tetrahydro-2H-pyran-4-yl) aniline

### Step A: 2-Bromo-4-fluoro-6-(prop-1-en-2-yl)aniline

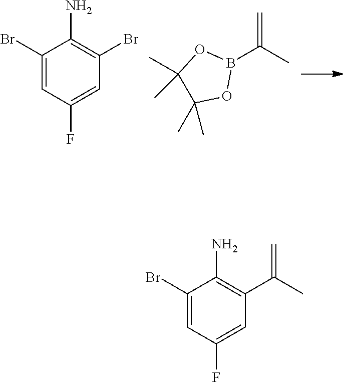

Nitrogen gas was bubbled through a mixture of 2,6-dibromo-4-fluoroaniline (5 g, 18-59 mmol), 4,4,5,5-tetramethyl-2-(prop-1-en-2-yl)-1,3,2-dioxaborolane (4.2 mL, 22.34 mmol) and potassium triphosphate (7.9 g, 37.2 mmol) in dioxane (50 mL) and water (8 mL) for 15 minutes. Then (2-dicyclohexylphosphino-2′,4′,6′-triisopropyl-1,1′-biphenyl)[2-(2′-amino-1,1′-biphenyl)]palladium(II) methanesulfonate [XPhos G3 Pd cat (500 mg, 0.591 mmol)] was added. The mixture was heated at 90° C. for 8 hours and then partitioned between hexane (200 mL) and water (100 mL). The organic layer was dried (magnesium sulfate), filtered, evaporated in vacuo and the residue purified by chromatography on silica gel (120 g column, 0-2% EtOAc/iso-hexane) to afford the title compound (1.95 g, 43%) as an oil.

¹H NMR (CDCl₃) δ 7.13 (dd, 1H), 6.77 (dd, 1H), 5.37-5.35 (m, 1H), 5.12-5.10 (m, 1H), 3.52 (br s, 2H) and 2.08-2.06 (m, 3H).

LCMS; m/z 230.2 (M+H)⁺ (ES⁺).

### Step B: 2-(3,6-Dihydro-2H-pyran-4-yl)-4-fluoro-6-(prop-1-en-2-yl)aniline

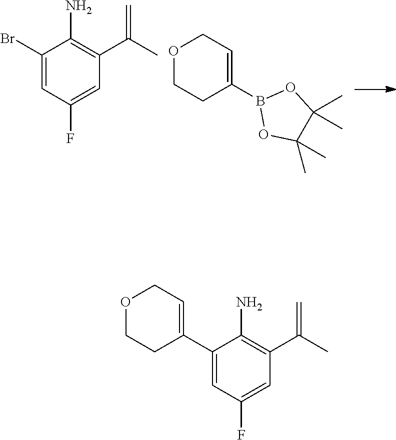

2-(3,6-Dihydro-2H-pyran-4-yl)-4,4,5,5-tetramethyl-1,3,2-dioxaborolane (457 mg, 2.176 mmol), tetrakis(triphenylphosphine)palladium(o) (251 mg, 0.218 mmol), sodium carbonate (923 mg, 8.70 mmol) and water (4 mL) were added to a sealed vial containing a solution of 2-bromo-4-fluoro-6-(prop-1-en-2-yl)aniline (500 mg, 2.173 mmol) in DMF (22 mL). The reaction mixture was heated under nitrogen at 100° C. overnight and then allowed to cool. The reaction mixture was diluted with EtOAc (50 mL), washed with brine (50 mL), dried (sodium sulfate) and concentrated in vacuo. The crude product was purified by chromatography on silica (40 g column, 0-20% EtOAc/iso-hexanes) to afford the title compound (355 mg, 65%) as a brownish oil. ¹H NMR (CDCl₃) δ 6.71 (dd, 1H), 6.67 (dd, 1H), 5.88 (m, 1H), 5.35-5.31 (m, 1H), 5.09 (m, 1H), 4.32 (m, 2H), 3.95 (t, J=5.4 Hz, 2H), 3.82 (br s, 2H), 2.42 (m, 2H) and 2.09-2.07 (m, 3H).

### Step C: 4-Fluoro-2-isopropyl-6-(tetrahydro-2H-pyran-4-yl)aniline

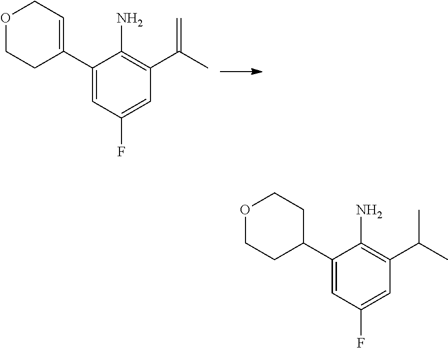 [SMILES: C=C(C)c1cc(F)cc(C2=CCOCC2)c1N ; CC(C)c1cc(F)cc(C2CCOCC2)c1N]

A mixture of 2-(3,6-dihydro-2H-pyran-4-yl)-4-fluoro-6-(prop-1-en-2-yl)aniline (355 mg, 1.522 mmol) and 5% palladium on carbon (156 mg, 0.03 mmol; type 87L (58.5% moisture)) in EtOAc (3.8 mL) was hydrogenated at 5 bar for 1 hour. The mixture was filtered through Celite® and evaporated to afford the title compound (340 mg, 91%). ¹H NMR (CDCl₃) δ 6.80 (dd, 1H), 6.75 (dd, 1H), 4.16-4.14 (m, 1H), 4.13-4.10 (m, 1H), 3.65-3.51 (m, 4H), 3.01-2.89 (m, 1H), 2.85-2.74 (m, 1H), 1.86-1.78 (m, 4H) and 1.28 (d, 6H).

LCMS; m/z 238.1 (M+H)⁺ (ES⁺).

### Intermediate A7: 2-Isopropyl-5-(1-methyl-1H-pyrazol-4-yl)aniline

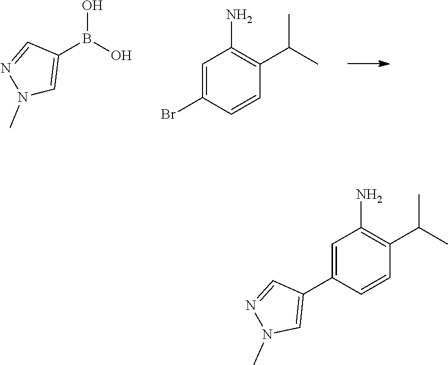

To a sealed vial was added 5-bromo-2-isopropylaniline (250 mg, 1.168 mmol) in DMF (11 mL), followed by the addition of (1-methyl-H-pyrazol-4-yl)boronic acid (147 mg, 1.168 mmol), Pd(PPh₃)₄ (135 mg, 0.117 mmol) and aqueous 2 M Na₂CO₃ (2.335 mL, 4.67 mmol). The reaction mixture is heated under Argon at 100° C. overnight. The reaction mixture was diluted with EtOAc (20 mL), washed with brine (20 mL), dried over anhydrous Na₂SO₄, filtered, concentrated, and then purified by chromatography on silica gel (40 g column, 0-60% EtOAc/iso-hexane) to afford the title compound (90 mg, 36%) as a white solid.

¹H NMR (CDCl₃) δ 7.70 (d, J=0.9 Hz, 1H), 7.54 (s, 1H), 7.13 (d, J=7.9 Hz, 1H), 6.90 (dd, J=7.9, 1.8 Hz, 1H), 6.80 (d, J=1.8 Hz, 1H), 3.93 (s, 3H), 3.69 (bs, 2H), 2.90 (sept, J=6.8 Hz, 1H), 1.27 (d, J=6.8 Hz, 6H).

### Intermediate A8: 2-Isopropyl-5-(pyrimidin-5-yl)aniline

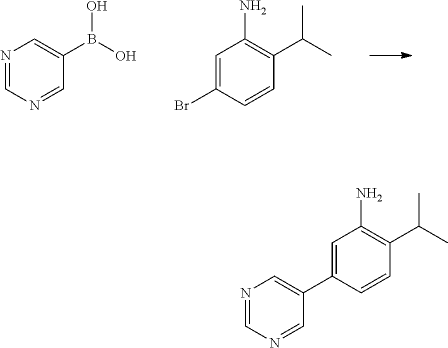 [SMILES: OB(O)c1cncnc1 ; CC(C)c1ccc(Br)cc1N ; CC(C)c1ccc(-c2cncnc2)cc1N]

Prepared according to the general procedure for 2-isopropyl-5-(1-methyl-H-pyrazol-4-yl)aniline (Intermediate A7) from 5-bromo-2-isopropylaniline and pyrimidin-5-ylboronic acid to afford the title compound (130 mg, 51%) as a white solid.

¹H NMR (CDCl₃) δ 9.16 (s, 1H), 8.91 (s, 2H), 7.28 (d, J=7.9 Hz, 1H), 6.98 (dd, J=8.0, 1.9 Hz, 1H), 6.87 (d, J=1.9 Hz, 1H), 3.84 (bs, 2H), 2.95 (sept, J=6.8 Hz, 1H), 1.31 (d, J=6.8 Hz, 6H).

### Intermediate A9: 2-Isocyanato-1,3-diisopropylbenzene

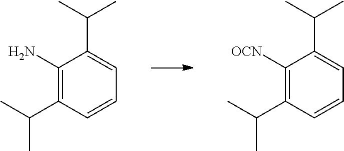

2,6-Diisopropylaniline (3.07 g, 17-14 mmol) was dissolved in dry THF (40 mL) and Et₃N (3 mL, 21.52 mmol) was added. A solution of triphosgene (4.26 g, 14.35 mmol) in dry THF (12 mL) was added over 5 minutes, resulting in the formation of a thick colourless precipitate. The reaction mixture was stirred at room temperature overnight. The THF was removed in vacuo and toluene (50 mL) was added. The mixture was filtered through a short silica plug eluting with toluene (150 mL). The filtrate was concentrated in vacuo to afford the title compound (2.76 g, 92%) as a colourless oil. ¹H NMR (CDCl₃) δ 7.20-7.10 (m, 3H), 3.22 (hept, J=6.9 Hz, 2H), 1.26 (d, J=6.8 Hz, 12H).

### Intermediate A: 4-(5-Fluoro-2-isocyanato-3-isopropylphenyl)-2-isopropoxypyridine

### Step A: 4-Fluoro-2-(prop-1-en-2-yl)aniline

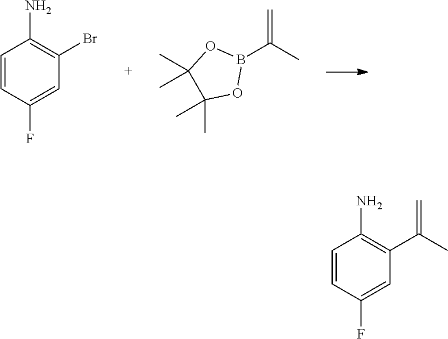

To a mixture of 2-bromo-4-fluoroaniline (39 g, 205.25 mmol, 1 eq), 4,4,5,5-tetramethyl-2-(prop-1-en-2-yl)-1,3,2-dioxaborolane (36.21 g, 215.51 mmol, 1.05 eq) and K₂CO₃ (70.92 g, 513.12 mmol, 2.5 eq) in dioxane (200 mL) and H₂O (40 mL) was added Pd(dppf)Cl₂ (7-51 g, 10.26 mmol, 0.05 eq) under N₂ atmosphere. Then the reaction mixture was stirred at 80° C. for 5 hours. The reaction mixture was quenched by addition of H₂O (600 mL) and extracted with EtOAc (2×500 mL). The combined organic layers were washed with brine (2×600 mL), dried over anhydrous Na₂SO₄, filtered and concentrated under reduced pressure. The residue was purified by silica gel column chromatography (petroleum ether:ethyl acetate=1:0 to 100:1) to give the title compound (27 g, 77% yield, 89% purity on LCMS) as a yellow oil.

¹H NMR (CDCl₃): δ 6.81-6.76 (m, 2H), 6.66-6.62 (m, 1H), 5.38 (s, 1H), 5.08 (s, 1H), 3.69 (br s, 2H) and 1.25 (s, 3H).

LCMS: m/z 152.2 (M+H)⁺ (ES⁺).

### Step B: 4-Fluoro-2-isopropylaniline

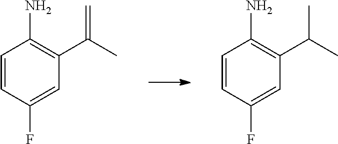

To a solution of 4-fluoro-2-(prop-1-en-2-yl)aniline (21 g, 138.91 mmol, 1 eq) in MeOH (300 mL) was added Pd/C (2.1 g, 178.59 mmol, 10 wt % loading on activated carbon) under N₂ atmosphere. The reaction mixture was degassed in vacuo and purged with H₂ several times. The reaction mixture was stirred at 25° C. for 12 hours under H₂ (50 psi). The reaction mixture was filtered and the filtrate was concentrated in vacuo to give the title compound (20 g, crude) as a yellow oil.

¹H NMR (CDCl₃): δ 6.86 (dd, 1H), 6.75-6.72 (m, 1H), 6.63-6.61 (m, 1H), 3.50 (br s, 2H), 2.95-2.84 (m, 1H) and 1.25 (d, 6H).

LCMS: m/z 154.2 (M+H)⁺ (ES⁺).

### Step C: 2-Bromo-4-fluoro-6-isopropylaniline

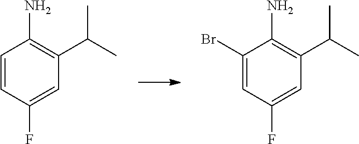

To a solution of 4-fluoro-2-isopropylaniline (20 g, 130.55 mmol, 1 eq) in toluene (250 mL) was added NBS (23.24 g, 130.55 mmol, 1 eq) at 25° C. The reaction mixture was stirred at 25° C. for 10 minutes. Then the reaction mixture was poured into H₂O (300 mL) and extracted with EtOAc (2×250 mL). The organic phases were washed with brine (2×400 mL), dried over anhydrous Na₂SO₄, filtered and concentrated in vacuo. The residue was purified by silica gel column chromatography (eluting only by using petroleum ether) to give the title compound (30 g, 99%) as a black brown oil.

¹H NMR (CDCl₃): δ 6.99 (dd, 1H), 6.78 (dd, 1H), 3.91 (br s, 2H), 2.88-2.71 (m, 1H) and 1.17 (d, 6H).

LCMS: m/z 232.1 (M+H)⁺ (ES⁺).

### Step D: 4-Bromo-2-isopropoxypyridine

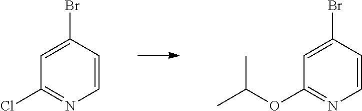

To a solution of 4-bromo-2-chloropyridine (20 g, 103.93 mmol, 1 eq) in THF (400 mL) was added NaH (6.24 g, 155.89 mmol, 60% purity, 1.5 eq) at 0° C. Then the mixture was stirred for 0.5 hour. Propan-2-ol (6.87 g, 114.32 mmol, 8.75 mL, 1.1 eq) was added and the resulting mixture was warmed to 50° C. and stirred for 12 hours. The reaction mixture was quenched with H₂O (1 L) at 25° C. and extracted with EtOAc (2×200 mL). The combined organic layers were washed with brine (200 mL), dried over Na₂SO₄, filtered and concentrated under reduced pressure. The residue was purified by column chromatography (SiO₂, petroleum ether:ethyl acetate=50:1 to 40:1) to give the title compound (22 g, 98%) as a light yellow oil.

¹H NMR (CDCl₃): δ 7.96 (d, 1H), 6.98 (dd, 1H), 6.89 (d, 1H), 5.44-5.24 (m, 1H) and 1.34 (d, 6H).

### Step E: 2-Isopropoxy-4-(4,4,5,5-tetramethyl-1,3,2-dioxaborolan-2-yl)pyridine

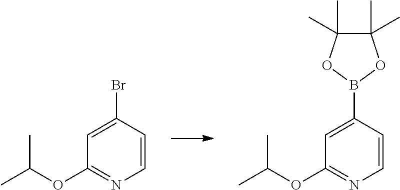 [SMILES: CC(C)Oc1cc(Br)ccn1 ; CC(C)Oc1cc(B2OC(C)(C)C(C)(C)O2)ccn1]

To a solution of 4-bromo-2-isopropoxypyridine (19 g, 87.93 mmol, 1 eq) and 4,4,4′,4′,5,5,5′,5′-octamethyl-2,2′-bi(1,3,2-dioxaborolane) (22.33 g, 87.93 mmol, 1 eq) in 1,4-dioxane (300 mL) was added KOAc (25.89 g, 263.80 mmol, 3 eq) followed by Pd(dppf)Cl₂ (1.93 g, 2.64 mmol, 0.03 eq) under nitrogen. Then the reaction mixture was heated to 80° C. and stirred for 12 hours. The mixture was concentrated in vacuo. The residue was purified by silica gel column chromatography (SiO₂, petroleum ether:ethyl acetate=50:1 to 20:1) to give the title compound (22 g, 95%) as a light yellow oil. ¹H NMR (CDCl₃): δ 8.16 (d, 1H), 7.13 (d, 1H), 7.08 (s, 1H), 5.32-5.24 (m, 1H), 1.34 (s, 12H) and 1.27 (s, 6H).

LCMS: m/z 264.2 (M+H)⁺ (ES⁺).

### Step F: 4-Fluoro-2-(2-isopropoxypyridin-4-yl)-6-isopropylaniline

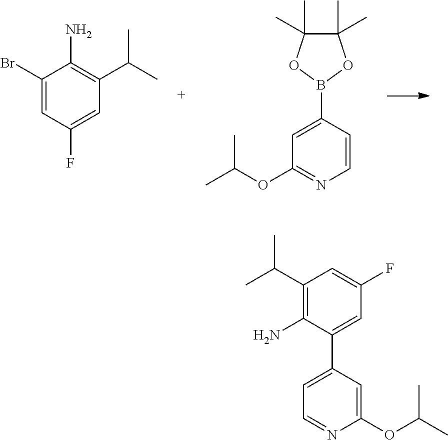 [SMILES: CC(C)c1cc(F)cc(Br)c1N ; CC(C)Oc1cc(B2OC(C)(C)C(C)(C)O2)ccn1 ; CC(C)Oc1cc(-c2cc(F)cc(C(C)C)c2N)ccn1]

To a solution of 2-bromo-4-fluoro-6-isopropylaniline (10.94 g, 47.12 mmol, 1 eq) and 2-isopropoxy-4-(4,4,5,5-tetramethyl-1,3,2-dioxaborolan-2-yl)pyridine (12.4 g, 47.12 mmol, 1 eq) in 1,4-dioxane (200 mL) and H₂O (20 mL) was added Pd(dppf)Cl₂ (1.72 g, 2.36 mmol, 0.05 eq) followed by K₂CO₃ (19.54 g, 141-37 mmol, 3 eq) at 25° C. Then the reaction mixture was heated to 80° C. and stirred for 2 hours. The mixture was filtered and the filtrate was concentrated in vacuo. The residue was purified by silica gel column chromatography (SiO₂, petroleum ether:ethyl acetate=50:1 to 20:1) to give the title compound (10.3 g, 69% yield, 91% purity on LCMS) as a brown oil.

¹H NMR (CDCl₃): δ 8.21 (d, 1H), 6.94-6.91 (m, 2H), 6.76 (s, 1H), 6.72 (dd, 1H), 5.38-5.29 (m, 1H), 3.64 (br s, 2H), 2.98-2.89 (m, 1H), 1.38 (d, 6H) and 1.30-1.27 (m, 6H). LCMS: m/z 289.2 (M+H)⁺ (ES⁺).

### Step G: 4-(5-Fluoro-2-isocyanato-3-isopropylphenyl)-2-isopropoxypyridine

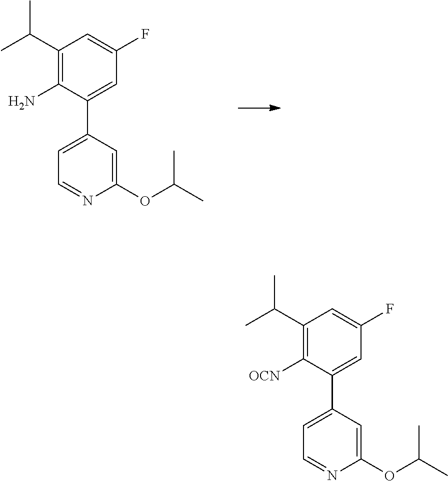

To a solution of 4-fluoro-2-(2-isopropoxypyridin-4-yl)-6-isopropylaniline (4 g, 13.87 mmol, 1 eq) in THF (80 mL) was added TEA (2.81 g, 27.74 mmol, 3.86 mL, 2 eq). The mixture was cooled to 0° C. and then triphosgene (1.65 g, 5.55 mmol, 0.4 eq) was added to the mixture. The resulting mixture was heated to 70° C. and stirred for 1 hour. The mixture was filtered and the filtrate was concentrated in vacuo. The residue was purified by silica gel column chromatography (SiO₂, petroleum ether:ethyl acetate=100:1 to 30:1) to give the title compound (1.9 g, 44% yield) as a yellow oil, which was used directly in the next step.

### Intermediate A11: 4-(4-Isocyanato-2,3-dihydro-1H-inden-5-yl)-2-methoxypyridine

### Step A: 4-Nitro-2,3-dihydro-1H-indene

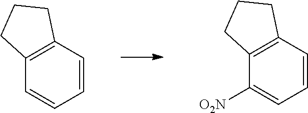 [SMILES: c1ccc2c(c1)CCC2 ; O=[N+]([O-])c1cccc2c1CCC2]

To a mixture of 2,3-dihydro-1H-indene (60 g, 507.72 mmol, 62.50 mL, 1 eq) in concentrated H₂SO₄ (30 mL) was added a mixture of HNO₃ (50 mL, 69 wt % in water) and concentrated H₂SO₄ (50 mL) dropwise at 0° C. over a period of 3.5 hours. The reaction mixture was stirred at 0° C. for 0.5 hour. Then the reaction mixture was poured into ice water (600 mL) and extracted with ethyl acetate (2×400 mL). The combined organic layers were washed with water (500 mL), saturated aqueous NaHCO₃ solution (500 mL) and brine (2×500 mL). The organic layer was dried over anhydrous Na₂SO₄, filtered and concentrated in vacuo. The residue was purified by silica gel column chromatography (SiO₂, petroleum ether:ethyl acetate, 1:0 to 100:1) to give the title compound (55 g, 66%) as a colourless oil.

¹H NMR (CDCl₃): δ 7.98 (d, 1H), 7.51 (d, 1H), 7.30 (t, 1H), 3.41 (t, 2H), 302 (t, 2H) and 2.22-2.20 (m, 2H).

### Step B: 2,3-Dihydro-1H-inden-4-amine

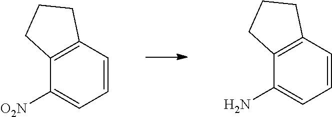 [SMILES: O=[N+]([O-])c1cccc2c1CCC2 ; Nc1cccc2c1CCC2]

To a solution of 4-nitro-2,3-dihydro-1H-indene (55 g, contained another regio-isomer) in MeOH (500 mL) was added Pd/C (5 g, 10 wt % loading on activated carbon) under N₂ atmosphere. The suspension was degassed under vacuum and purged with H₂ several times. The reaction mixture was stirred under H₂ (50 psi) at 20° C. for 12 hours. The reaction mixture was filtered and the filtrate was concentrated in vacuo. The residue was purified by silica gel column chromatography (SiO₂, petroleum ether:ethyl acetate, 1:0 to 100:4) to give the title compound (19.82 g, 43% yield, 96.39% purity on LCMS) as a brown oil.

¹H NMR (CDCl₃): δ 7.01 (t, 1H), 6.71 (d, 1H), 6.51 (d, 1H), 3.57 (br s, 2H), 2.93 (t, 2H), 2.75 (t, 2H) and 2.16-2.08 (m, 2H).

LCMS: m/z 134.2 (M+H)⁺ (ES⁺).

### Step C: N-(2,3-Dihydro-1H-inden-4-yl)acetamide

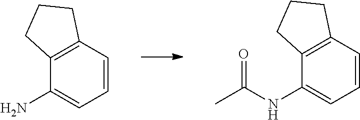

To a solution of 2,3-dihydro-1H-inden-4-amine (19.8 g, 148.66 mmol, 1 eq) and TEA (19.56 g, 193.26 mmol, 1.3 eq) in DCM (300 mL) was added dropwise Ac₂O (17.45 g, 170.96 mmol, 1.15 eq) over 6 minutes at 0° C. Then the reaction mixture was warmed to 16° C. and stirred for 1.4 hours. The mixture was poured into water (500 mL) and extracted with DCM (2×300 mL). The combined organic phases were washed with brine (2×500 mL), dried over anhydrous Na₂SO₄, filtered and concentrated in vacuo to give the title compound (25.74 g, 96% yield, 96.69% purity on LCMS) as a white solid.

¹H NMR (CDCl₃): δ 7.70 (d, 1H), 7.15 (t, 1H), 7.02 (d, 1H), 2.95 (t, 2H), 2.81 (t, 2H), 2.18 (s, 3H) and 2.15-2.08 (m, 2H).

LCMS: m/z 176.2 (M+H)⁺ (ES⁺)

### Step D: N-(5-Bromo-2,3-dihydro-1H-inden-4-yl)acetamide

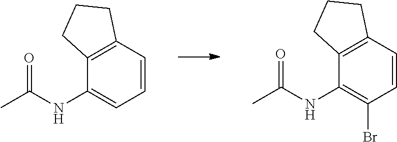

N-(2,3-dihydro-1H-inden-4-yl)acetamide (34.6 g, 197.46 mmol, 1 eq), p-toluenesulfonic acid (18.70 g, 108.60 mmol, 0.55 eq) and Pd(OAc)₂ (2.22 g, 9.87 mmol, 0.05 eq) were suspended in toluene (400 mL) and stirred at 20° C. for 0.5 hour under air atmosphere. NBS (38.66 g, 217.20 mmol, 1.1 eq) was added. Then the reaction mixture was stirred at 20° C. for 2 hours. The reaction mixture was poured into water (500 mL) and extracted with ethyl acetate (2×500 mL). The combined organic phases were washed with brine (2×500 mL), dried over anhydrous NaSO₄, filtered and concentrated in vacuo. The residue was purified by silica gel column chromatography (SiO₂, petroleum ether:ethyl acetate, 10: 1 to 2:1) to give the title compound (13.9 g, 27% yield, 98.1% purity on LCMS) as a white solid.

¹H NMR (CDCl₃): δ 7.33 (d, 1H), 7.16 (s, 1H), 6.98 (d, 1H), 2.92-2.83 (m, 4H), 2.21 (s, 3H) and 2.10-2.02 (m, 2H).

LCMS: m/z 254.1 (M+H)⁺ (ES⁺).

### Step E: 5-Bromo-2,3-dihydro-1H-inden-4-amine

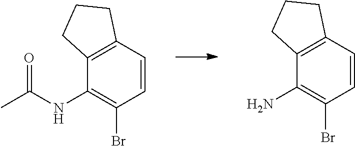

A mixture of N-(5-bromo-2,3-dihydro-1H-inden-4-yl)acetamide (45.68 g, 179.76 mmol, 1 eq) in EtOH (200 mL) and concentrated HCl (300 mL, 36 wt % in water) was stirred at 80° C. for 36 hours. The reaction mixture was cooled to 0° C. in an ice bath and some solid precipitated. The suspension was filtered. The filter cake was washed with ice water (50 mL) and dried in vacuo to give the title compound (34.1 g, 72% yield, 94.08% purity on LCMS, HCl salt) as a grey solid.

¹H NMR (DMSO-d₆): δ 7.67 (br S, 2H), 7.24 (d, 1H), 6.69 (d, 1H), 2.85 (t, 2H), 2.79 (t, 2H) and 2.04-1.96 (m, 2H).

LCMS: m/z 212.0 (M+H)⁺ (ES⁺).

### Step F: 5-(2-Methoxypyridin-4-yl)-2,3-dihydro-1H-inden-4-amine

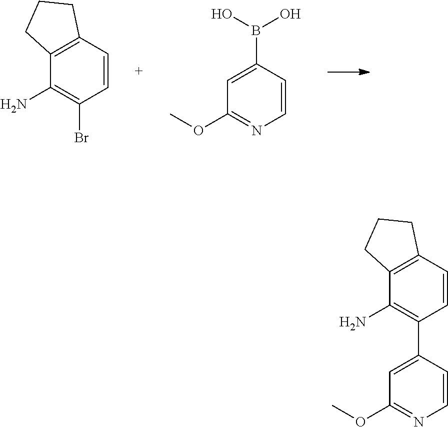 [SMILES: Nc1c(Br)ccc2c1CCC2 ; COc1cc(B(O)O)ccn1 ; COc1cc(-c2ccc3c(c2N)CCC3)ccn1]

A solution of (2-methoxypyridin-4-yl)boronic acid (25.11 g, 164.15 mmol, 1.2 eq), 5-bromo-2,3-dihydro-1H-inden-4-amine (34 g, 136.80 mmol, 1 eq, HCl salt) and K₂CO₃ (60.50 g, 437.74 mmol, 3.2 eq) in dioxane (500 mL) and H₂O (100 mL) was degassed with nitrogen for 15 minutes before Pd(dppf)Cl₂.CH₂Cl₂ (6 g, 7.35 mmol, 0.053 eq) was added. The reaction mixture was heated to 80° C. for 12 hours. The mixture was poured into water (500 mL) and extracted with ethyl acetate (2×500 mL. The combined organic phases were washed with brine (2×700 mL), dried over anhydrous Na₂SO₄, filtered and concentrated in vacuo. The residue was purified by silica gel column chromatography (SiO₂, petroleum ether:ethyl acetate, 1:0 to 10:1) to give the title compound (27.4 g, 79% yield, 95% purity on LCMS) as a white solid.

¹H NMR (CDCl₃): δ 8.22 (d, 1H), 7.03-7.00 (m, 1H), 6.99 (d, 1H), 6.87 (s, 1H), 6.77 (d, 1H), 3.99 (s, 3H), 3.77 (br s, 2H), 2.97 (t, 2H), 2.77 (t, 2H) and 2.21-2.13 (m, 2H).

LCMS: m/z 241.2 (M+H)⁺ (ES⁺).

### Step G: 4-(4-Isocyanato-2,3-dihydro-1H-inden-5-yl)-2-methoxypyridine

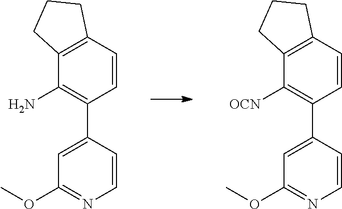

To a solution of 5-(2-methoxypyridin-4-yl)-2,3-dihydro-1H-inden-4-amine (11 g, 45.78 mmol, 1 eq) and TEA (5.10 g, 50-35 mmol, 1.1 eq) in THF (275 mL) was added bis(trichloromethyl) carbonate (4.93 g, 16.61 mmol, 0.36 eq) in portions at 0° C. Then the reaction mixture was stirred at 16° C. for 0.5 hour. The reaction mixture was filtered and the filter cake was washed with THF (2 L). The filtrate was concentrated in vacuo to give the title compound (9.04 g, 74%) as a light yellow solid.

¹H NMR (CDCl₃): δ 8.28 (d, 1H), 7.20-7.16 (m, 3H), 7.02 (s, 1H), 4.16 (s, 3H), 3.04-2.99 (m, 4H) and 2.23-2.15 (m, 2H).

### Intermediate A12: 5-Chloro-2-isocyanato-1,3-diisopropylbenzene

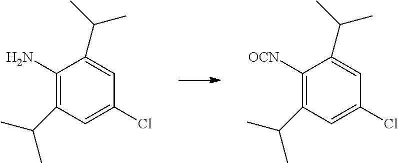

To a solution of 4-chloro-2,6-diisopropylaniline (0.105 g, 0.496 mmol) in toluene (1 mL) was added a phosgene solution (0.65 mL, 20 wt % in toluene, 1.22 mmol) and the reaction mixture was refluxed for 1 hour. Upon cooling, the mixture was concentrated in vacuo to afford the title compound as an orange oil (0.111 g, 94%).

¹H NMR (CDCl₃) δ 7.07 (d, 2H), 3.17 (h, 2H),1.24 (d, 12H).

### PREPARATION OF EXAMPLES

### Example 1: 2-(3-(N-((1,2,3,5,6,7-Hexahydro-s-indacen-4-yl)carbamoyl) sulfamoyl)-1H-pyrazol-1-yl)-N,N-dimethylacetamide, Sodium Salt

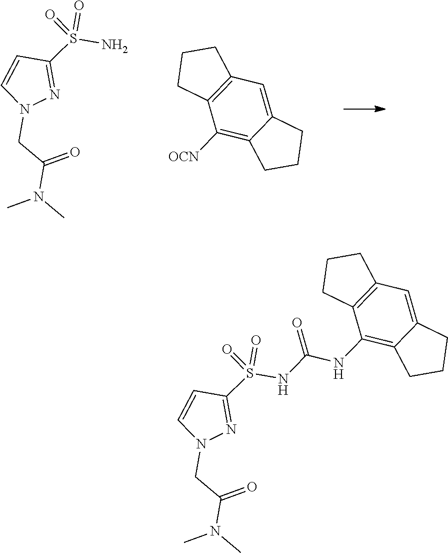

N,N-dimethyl-2-(3-sulfamoyl-H-pyrazol-1-yl)acetamide (Intermediate P1) (67 mg, 0.287 mmol) was dissolved in dry THF (2 mL) and sodium tert-butoxide (2 M in THF) (0.151 mL, 0.301 mmol) was added. After stirring for 1 hour, a solution of 4-isocyanato-1,2,3,5,6,7-hexahydro-s-indacene (Intermediate A1) (60 mg, 0.301 mmol) in THF (1 mL) was added. The reaction mixture was stirred overnight at room temperature. EtOAc (6 mL) was added and the suspension stirred for 1 hour. The resultant colourless precipitate was collected by filtration, washed with EtOAc, and dried in vacuo to afford the title compound (15 mg, 11%) as a white solid.

¹H NMR (DMSO-d₆) δ 7.55-7.54 (m, 2H), 6.77 (s, 1H), 6.42 (d, J=2.2 Hz, 1H), 5.08 (s, 2H), 3.03 (s, 3H), 2.86 (s, 3H), 2.76 (t, J=7.4 Hz, 4H), 2.67 (t, J=7.3 Hz, 4H), 1.95-1.87 (m, 4H).

LCMS; m/z 432 (M+H)⁺ (ES⁺).

### Example 2: 2-(3-(N-((1,2,3,5,6,7-Hexahydro-s-indacen-4-yl)carbamoyl) sulfamoyl)-1H-pyrazol-1-yl)-N-methylacetamide

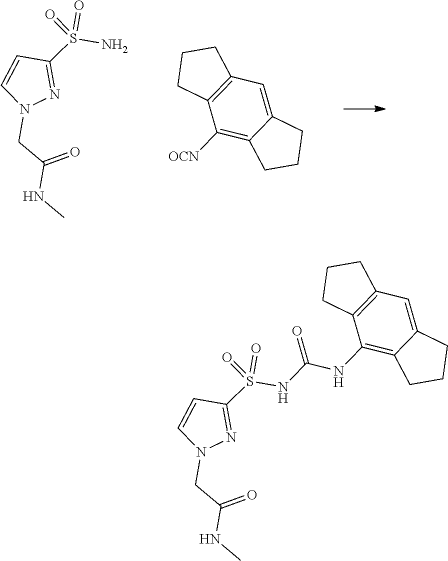

N-Methyl-2-(3-sulfamoyl-1H-pyrazol-1-yl)acetamide (Intermediate P2) (58 mg, 0.251 mmol) was dissolved in dry THF (2 mL) and sodium tert-butoxide (2 M in THF) (0.125 mL, 0.251 mmol) was added. After stirring for 1 hour, a solution of 4-isocyanato-1,2,3,5,6,7-hexahydro-s-indacene (Intermediate A11 (so mg, 0.251 mmol) in THF (1 mL) was added. The reaction mixture was stirred overnight at room temperature. The reaction mixture was concentrated in vacuo, dissolved in DMSO (2 mL) and purified by reversed phase prep-HPLC (General Methods, basic prep) to afford the title compound (29.6 mg, 27%) a colourless powder.

¹H NMR (DMSO-d₆) δ 8.06 (s, 1H), 7.84 (s, 1H), 7.81 (d, J=1.5 Hz, 1H), 6.88 (s, 1H), 6.64 (d, J=1.7 Hz, 1H), 4.84 (s, 2H), 2.78 (t, J=7.4 Hz, 4H), 2.62 (t, J=7.4 Hz, 4H), 2.60 (d, J=4.6 Hz, 3H), 2.05-1.82 (m, 4H). One exchangeable proton not visible. LCMS; m/z 418 (M+H)⁺ (ES⁺), 416 (M−H)⁻ (ES⁻).

### Example 3: 1-(1-Acetylazetidin-3-yl)-N-((1,2,3,5,6,7-hexahydro-s-indacen-4-yl)carbamoyl)-1H-pyrazole-3-sulfonamide

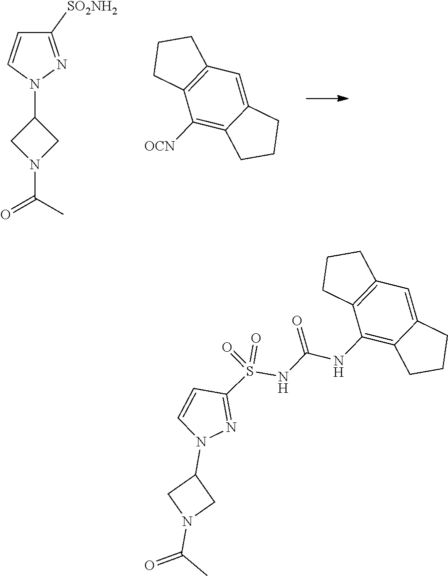

Prepared according to the general procedure of 2-(3-(N-((1,2,3,5,6,7-hexahydro-s-indacen-4-yl)carbamoyl)sulfamoyl)-1H-pyrazol-1-yl)-N-methylacetamide (Example 2) from 1-(1-acetylazetidin-3-yl)-1H-pyrazole-3-sulfonamide (Intermediate P3) and 4-isocyanato-1,2,3,5,6,7-hexahydro-s-indacene (Intermediate A1) to afford the title compound (8 mg, 11%) as a white solid.

¹H NMR (DMSO-d₆) δ 7.95 (s, 1H), 7.73 (s, 1H), 6.85 (s, 1H), 6.61 (s, 1H), 5.35-5.22 (m, 1H), 4.63-4.54 (m, 1H), 4.41-4.22 (m, 2H), 4.09 (dd, J=10.1, 5.5 Hz, 1H), 2.77 (t, J=7.4 Hz, 4H), 2.62 (t, J=7.3 Hz, 4H), 1.99-1.87 (m, 4H), 1.81 (s, 3H). One exchangeable proton not visible.

LCMS; m/z 444 (M+H)⁺ (ES⁺).

### Example 4: N-((1,2,3,5,6,7-Hexahydro-s-indacen-4-yl)carbamoyl)-5-methyl-4-oxo-4,5,6,7-tetrahydropyrazolo[1,5-a]pyrazine-2-sulfonamide, Sodium Salt

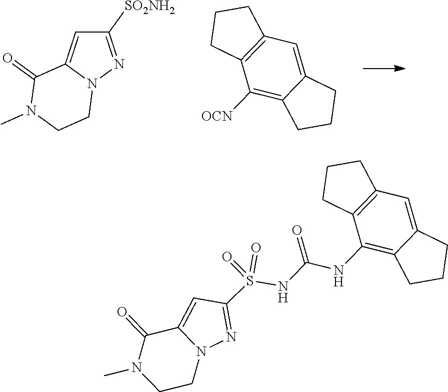

5-Methyl-4-oxo-4,5,6,7-tetrahydropyrazolo[1,5-a]pyrazine-2-sulfonamide (Intermediate P4) (59.8 mg, 0.26 mmol) was dissolved in THF:DMF (1:1) (4 mL) and sodium tert-butoxide (2M in THF) (0.136 ml, 0.273 mmol) was added. After stirring for 1 hour, 4-isocyanato-1,2,3,5,6,7-hexahydro-s-indacene (Intermediate A1) (54.3 mg, 0.273 mmol) was added and the reaction mixture was stirred at room temperature for 6 hours. Ethyl acetate (6 mL) was added and the suspension stirred for hours. The resultant precipitate was collected by filtration, washed with ethyl acetate (2 mL), triturated with ethyl acetate (5 mL) for 1 hour, filtered, and dried under reduced pressure to afford the title compound (73 mg, 60%) as a white solid.

¹H NMR (DMSO-d₆) δ 7.47 (s, 1H), 6.79-6.74 (m, 2H), 4.38-4.32 (m, 2H), 3.80-3.72 (m, 2H), 3.00 (s, 3H), 2.79-2.71 (m, 4H), 2.67 (t, J=7.2 Hz, 4H), 1.95-1.86 (m, 4H).

LCMS; m/z 430 (M+H)⁺ (ES⁺).

### Example 5: 1-(1-Acetylpiperidin-4-yl)-N-((1,2,3,5,6,7-hexahydro-s-indacen-4-yl)carbamoyl)-1H-pyrazole-3-sulfonamide, Sodium Salt

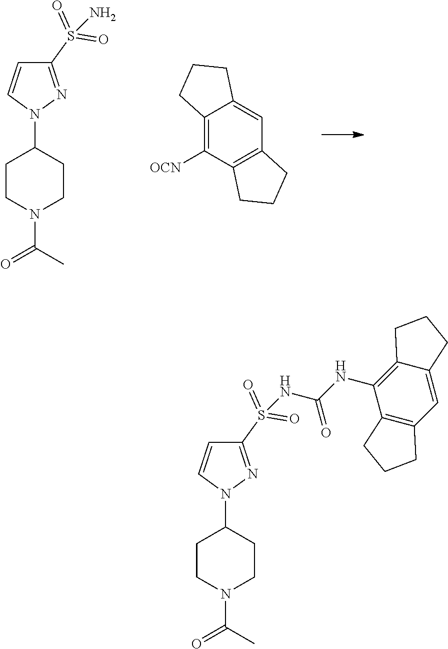

1-(1-Acetylpiperidin-4-yl)-1H-pyrazole-3-sulfonamide (Intermediate P14) (42.8 mg, 0.157 mmol) was dissolved in THF (10 mL) by heating to 60° C. Sodium tert-butoxide (2M in THF, 0.083 ml, 0.165 mmol) was added and the mixture was allowed to cool to room temperature and stirred for 1 hour. Then 4-isocyanato-1,2,3,5,6,7-hexahydro-s-indacene (Intermediate A1) (31.3 mg, 0.157 mmol) was added and the mixture was stirred at room temperature for 15 hours. The suspension was filtered, washing with THF (1 mL). The solid was triturated with EtOAc (5 mL) and filtered. The solid was further triturated with MeCN (5 mL), filtered and dried under vacuum to afford the title compound as a white solid (35 mg, 43%).

¹H NMR (DMSO-d₆) δ 7.72 (d, J=2.3 Hz, 1H), 7.55 (s, 1H), 6.76 (s, 1H), 6.38 (d, J=2.3 Hz, 1H), 4.54-4.25 (m, 2H), 3.99-3.80 (m, 1H), 3.22-3.07 (m, 1H), 2.74 (t, J=7.4 Hz, 4H), 2.71-2.58 (m, 5H), 2.03 (s, 3H), 2.00-1.65 (m, 8H).

LCMS; m/z 472 (M+H)⁺ (ES⁺).

### Example 6: N-(3-(Dimethylamino)propyl)-3-(N-((1,2,3,5,6,7-hexahydro-s-indacen-4-yl)carbamoyl)sulfamoyl)-N,1-dimethyl-1H-pyrazole-5-carboxamide

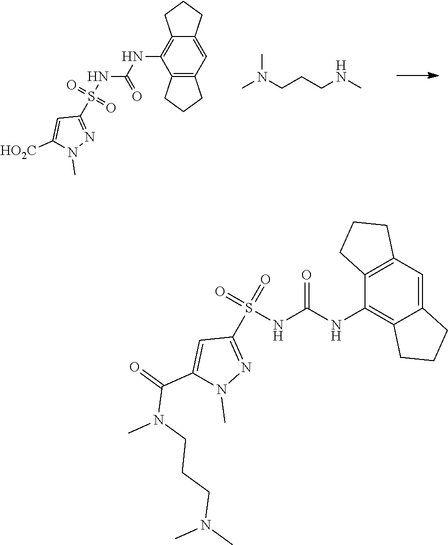

N1,N1,N3-Trimethylpropane-1,3-diamine (28 μl, 0.19 mmol), sodium bicarbonate (16 mg, 0.19 mmol) and HATU (72 mg, 0.19 mmol) were successively added to a suspension of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (70 mg, 0.156 mmol) in DMF (1 mL) and stirred at room temperature for 2 days. The reaction was quenched with water (1 mL) and purified by reversed phase prep-HPLC (General Methods, basic prep) to afford the title compound as a white solid (34 mg, 43%).

¹H NMR (DMSO-d₆) (rotamers) δ 7.77 (s, 1H), 6.83 (s, 1H), 6.80 & 6.72 (2×s, 1H), 3.85 &3.81 (2×s, 3H),3.47 & 3.30 (t, J=6.9 & 7.3 Hz, 2H),3.01 & 2.96 (2×s, 3H),2.84 & 2.76 (t, J=7.7 Hz, 2H), 2.76 (t, J=7.4 Hz, 4H), 2.64 (t, J=6.1 Hz, 4H), 2.60 & 2.32 (2×s, 6H), 1.89 (quin, J=7.2 Hz, 4H), 1.90 & 1.77 (2×m, 2H). NH not observed.

LCMS; m/z 503.5 (M+H)⁺ (ES⁺); 501.3 (M−H)⁻ (ES⁻).

### Example 7: N-Cyclobutyl-3-(N-((1,2,3,5,6,7-hexahydro-s-indacen-4-yl)carbamoyl)sulfamoyl)-N,1-dimethyl-1H-pyrazole-5-carboxamide, Partial Ammonium Salt

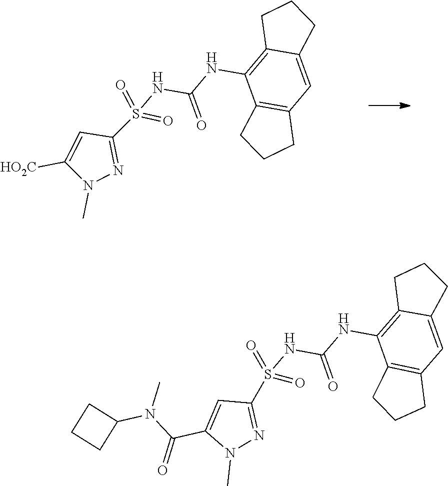 [SMILES: Cn1nc(S(=O)(=O)NC(=O)Nc2c3c(cc4c2CCC4)CCC3)cc1C(=O)O ; CN(C(=O)c1cc(S(=O)(=O)NC(=O)Nc2c3c(cc4c2CCC4)CCC3)nn1C)C1CCC1]

Prepared according to the general procedure of N-(3-(dimethylamino)propyl)-3-(N-((1,2,3,5,6,7-hexahydro-s-indacen-4-yl)carbamoyl)sulfamoyl)-N,1-dimethyl-1H-pyrazole-5-carboxamide (Example 6) from 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) and N-methylcyclobutanamine, HCl salt to afford the title compound (40 mg, 54%) as a white solid.

¹H NMR (DMSO-d₆), rotamers; δ 7.74 (s, 1H), 7.39 (br s, 1H), 6.83 (s, 1H), 6.80-6.32 (m, 1H), 5.00-4.14 (m, 1H), 3.83 (s, 3H), 2.98 (s, 3H), 2.77 (t, J=7.4 Hz, 4H), 2.64 (t, J=7.4 Hz, 4H),2.36-2.12 (m, 2H), 2.13-1.85 (m, 6H), 1.81-1.24 (m, 2H).

LCMS; m/z 472.5 (M+H)⁺ (ES⁺); 470.3 (M−H)⁻ (ES⁻).

### Example 8: N-Cyclobutyl-3-(N-((2,6-diisopropylphenyl)carbamoyl) sulfamoyl)-N,1-dimethyl-1H-pyrazole-5-carboxamide

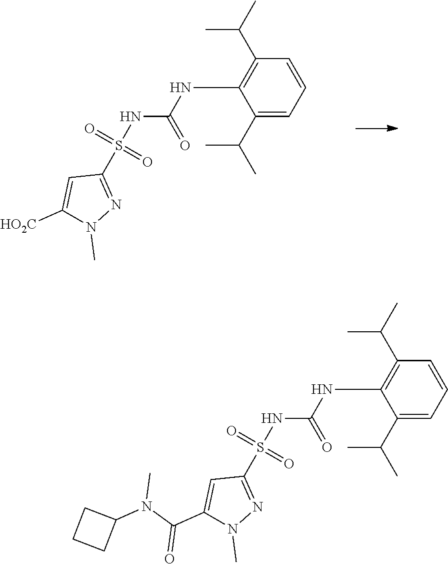 [SMILES: CC(C)c1cccc(C(C)C)c1NC(=O)NS(=O)(=O)c1cc(C(=O)O)n(C)n1 ; CC(C)c1cccc(C(C)C)c1NC(=O)NS(=O)(=O)c1cc(C(=O)N(C)C2CCC2)n(C)n1]

HATU (68.5 mg, 0.180 mmol) was added to a solution of 3-(N-((2,6-diisopropylphenyl) carbamoyl)sulfamoyl)-1-methyl-1H-pyrazole-5-carboxylic acid, disodium salt (Intermediate P9) (67.9 mg, 0.150 mmol) and N-methylcyclobutanamine, HCl (20.07 mg, 0.165 mmol) in DMF (1 mL). Triethylamine (23.01 μL, 0.165 mmol) was added and the mixture stirred for 20 hours. Water (1 mL) was slowly added and the suspension stirred for 1 hour. The suspension was filtered and the collected solid triturated in water (3 mL) for 0.5 hour. The suspension was filtered and the collected solid was purified by chromatography on RP Flash C18 (13 g column, 0-50% MeCN/10 mM ammonium bicarbonate) and triturated with TBME (2 mL) for 1 hour to afford the title compound (30 mg, 40%) as a white solid.

¹H NMR (DMSO-d₆), rotamers; δ 11.09 (s, 1H), 7.90 (s, 1H), 7.28-7.20 (m, 1H), 7.12 (d, J=7.7 Hz, 2H), 7.03-6.86 (2×m, 1H), 4.78-4.24 (2×m, 1H), 3.91 (s, 3H), 3.02-2.91 (m, 5H), 2.27-1.91 (m, 4H), 1.72-1.36 (m, 2H), 1.17-0.94 (m, 12H).

LCMS; m/z 476 (M+H)⁺ (ES⁺).

### Example 9: N-(2-Cyanoethyl)-3-(N-((1,2,3,5,6,7-hexahydro-s-indacen-4-yl)carbamoyl)sulfamoyl)-N,1-dimethyl-1H-pyrazole-5-carboxamide

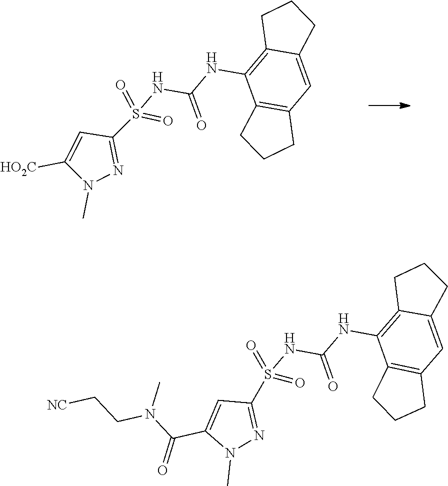 [SMILES: Cn1nc(S(=O)(=O)NC(=O)Nc2c3c(cc4c2CCC4)CCC3)cc1C(=O)O ; CN(CCC#N)C(=O)c1cc(S(=O)(=O)NC(=O)Nc2c3c(cc4c2CCC4)CCC3)nn1C]

HATU (77 mg, 0.201 mmol) was added to a solution of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (75.2 mg, 0.168 mmol) and 3-(methylamino)propanenitrile (18.83 μL, 0.201 mmol) in DMF (1 mL) and the mixture stirred at room temperature for hours. Water (1 mL) was slowly added and the suspension stirred for 1 hour. The suspension was filtered and the collected solid triturated in water (3 mL) for 0.5 hour. The suspension was filtered and the collected solid was washed with water (0.5 mL) and TBME (1 mL). The solid was dried under reduced pressure for 6 hours to afford the title compound (25 mg, 31%) as a white solid.

¹H NMR (DMSO-d₆), rotamers; δ 10.96 (s, 1H), 8.09 (s, 1H), 7.06 (s, 1H), 6.95 (s, 1H), 3.95-3.92 (2×s, 3H), 3.80-3.65 (m, 2H), 3.07-2.87 (2×s, 3H), 2.88 (t, J=6.6 Hz, 2H), 2.79 (t, J=7.4 Hz, 4H), 2.60 (t, J=7.5 Hz, 4H), 1.99-1.92 (m, 4H).

LCMS: m/z 471 (M+H)⁺ (ES⁺).

### Example 10: N-(2-(Dimethylamino)ethyl)-3-(N-((1,2,3,5,6,7-hexahydro-s-indacen-4-yl)carbamoyl)sulfamoyl)-N,1-dimethyl-1H-pyrazole-5-carboxamide

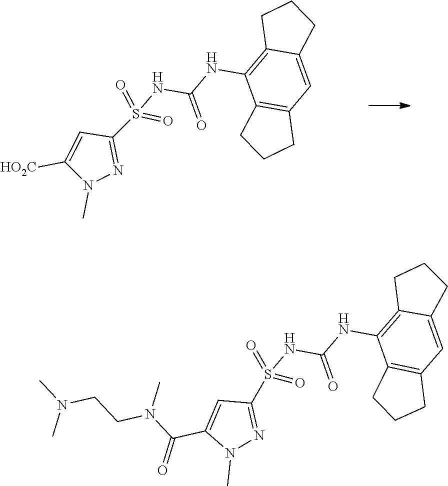 [SMILES: Cn1nc(S(=O)(=O)NC(=O)Nc2c3c(cc4c2CCC4)CCC3)cc1C(=O)O ; CN(C)CCN(C)C(=O)c1cc(S(=O)(=O)NC(=O)Nc2c3c(cc4c2CCC4)CCC3)nn1C]

N1,N1,N2-Trimethylethane-1,2-diamine (29 μL, 0.223 mmol) and HATU (83 mg, 0.219 mmol) were successively added to a suspension of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (70 mg, 0.156 mmol) in DMF (1 mL) and stirred for 20 hours. The reaction was quenched with water (0.1 mL) and purified by reversed phase prep-HPLC (General Methods, basic prep) to afford the title compound (34 mg, 44%) as a white solid.

¹H NMR (DMSO-d₆), rotamers; 610.36 (s, 1H), 7.87 & 7.80 (2×s, 1H), 6.91-6.80 (m, 2H), 3.88 & 3.83 (2×s, 3H), 3.70-3.62 & 3.48-3.40 (2×m, 2H), 3.04 & 2.96 (2×s, 3H), 2.86 & 2.40 (2×m, 2H), 2.77 (t, J=7.4 Hz, 4H), 2.62 (t, J=7.4 Hz, 4H), 2.01-1.85 (m, 7H). CH₃ not visible.

LCMS; m/z 489.4 (M+H)⁺ (ES⁺); 487.3 (M−H)⁻ (ES⁻).

### Example 11: N-Cyclopentyl-3-(N-((1,2,3,5,6,7-hexahydro-s-indacen-4-yl)carbamoyl)sulfamoyl)-N,1-dimethyl-1H-pyrazole-5-carboxamide, Sodium Salt

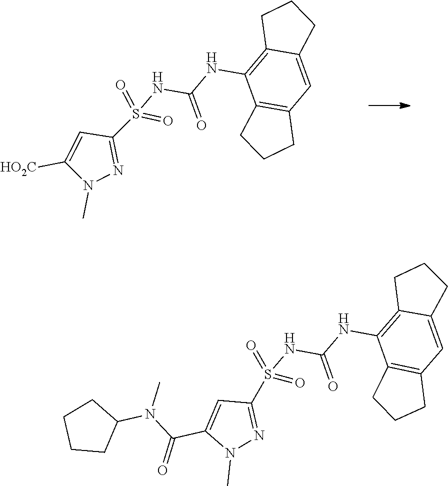 [SMILES: Cn1nc(S(=O)(=O)NC(=O)Nc2c3c(cc4c2CCC4)CCC3)cc1C(=O)O ; CN(C(=O)c1cc(S(=O)(=O)NC(=O)Nc2c3c(cc4c2CCC4)CCC3)nn1C)C1CCCC1]

N-Methylcyclopentanamine (0.012 mL, 0.102 mmol) and HATU (39 mg, 0.103 mmol) were successively added to a suspension of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (60 mg, 0.134 mmol) in DMF (1 mL) and stirred for 20 hours. TBME (5 mL) was added and the resulting solid filtered off. The filtrate was treated with NaHCO₃ (5-7 mg, 0.068 mmol) and the solution purified by reversed phase chromatography (12 g column, 5-50% MeCN/10 mM ammonium bicarbonate) to afford the crude product as a white solid. This material (33 mg) was dissolved in MeOH (3 mL), 0.1 M aqueous NaHCO₃ (0.68 mL, 0.068 mmol) and water (1 mL), sonicated for 10 minutes, evaporated in vacuo and dried to afford the title compound (29 mg, 83%) as a white solid.

¹H NMR (DMSO-d₆), rotamers; δ 7.51 (s, 1H), 6.76 (s, 1H), 6.53 (br s, 1H), 4.31-414 (m, 1H), 3.78 (s, 3H), 2.86 (s, 3H), 2.74 (t, J=7.4 Hz, 4H), 2.64 (t, J=7.5 Hz, 4H), 1.89 (p, J=7.5 Hz, 4H), 1.82-1.47 (m, 8H).

LCMS; m/z 486.5 (M+H)⁺ (ES⁺); 484.3 (M−H)⁻ (ES⁻).

### Example 12: 3-(N-((1,2,3,5,6,7-Hexahydro-s-indacen-4-yl)carbamoyl) sulfamoyl)-N,1-dimethyl-N-(2-(N-methylacetamido)ethyl)-1H-pyrazole-5-carboxamide, Partial Ammonium Salt

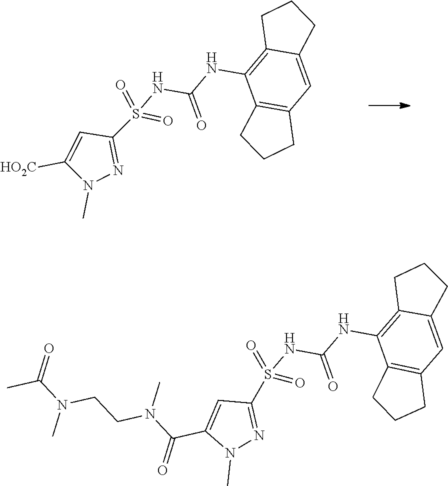 [SMILES: Cn1nc(S(=O)(=O)NC(=O)Nc2c3c(cc4c2CCC4)CCC3)cc1C(=O)O ; CC(=O)N(C)CCN(C)C(=O)c1cc(S(=O)(=O)NC(=O)Nc2c3c(cc4c2CCC4)CCC3)nn1C]

Prepared according to the general procedure of N-(3-(dimethylamino)propyl)-3-(N-((1,2,3,5,6,7-hexahydro-s-indacen-4-yl)carbamoyl)sulfamoyl)-N,1-dimethyl-1H-pyrazole-5-carboxamide (Example 6) from 3-(N-((1,2,3,5,6,7-hexahydro-s-indacen-4-yl)carbamoyl)sulfamoyl)-1-methyl-1H-pyrazole-5-carboxylic Acid, Disodium Salt (Intermediate P8) and N-methyl-N-(2-(methylamino)ethyl)acetamide to afford the title compound (38 mg, 47%) as a white solid.

¹H NMR (DMSO-d₆), rotamers; δ 10.98 (br s, 1H), 7.90 (br s, 1H), 6.94-6.74 (m, 2H), 3.92-3.77 (m, 3H), 3.65-3.37 (m, 4H), 3.08-2.51 (m, 14H), 2.04-1.76 (m, 7H). LCMS; m/z 517.4 (M+H)⁺ (ES⁺); 515.3 (M−H)⁻ (ES⁻).

### Example 13: N-(2-Acetamidoethyl)-3-(N-((1,2,3,5,6,7-hexahydro-s-indacen-4-yl)carbamoyl)sulfamoyl)-N,1-dimethyl-1H-pyrazole-5-carboxamide, Partial Ammonium Salt

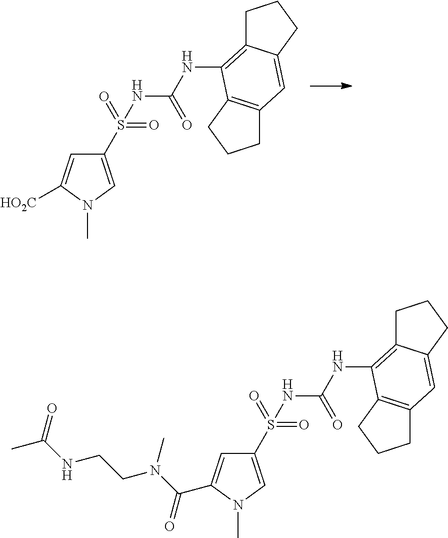 [SMILES: Cn1cc(S(=O)(=O)NC(=O)Nc2c3c(cc4c2CCC4)CCC3)cc1C(=O)O ; CC(=O)NCCN(C)C(=O)c1cc(S(=O)(=O)NC(=O)Nc2c3c(cc4c2CCC4)CCC3)cn1C]

Prepared according to the general procedure of N-(3-(dimethylamino)propyl)-3-(N-((1,2,3,5,6,7-hexahydro-s-indacen-4-yl)carbamoyl)sulfamoyl)-N,1-dimethyl-1H-pyrazole-5-carboxamide (Example 6) from 3-(N-((1,2,3,5,6,7-hexahydro-s-indacen-4-yl)carbamoyl)sulfamoyl)-1-methyl-1H-pyrazole-5-carboxylic Acid, Disodium Salt (Intermediate P8) and N-(2-(methylamino)ethyl)acetamide, HCl to afford the title compound (26 mg, 32%) as a white solid.

¹H NMR (DMSO-d₆), rotamers; δ 10.94 (br s, 1H), 8.32-7.69 (m, 2H), 6.94-6.74 (m, 2H), 3.92-3.78 (m, 3H), 3.54-3.36 (m, 2H), 3.31-3.12 (m, 2H), 3.08-2.93 (m, 3H), 2.77 (t, J=7.4 Hz, 4H), 2.61 (t, J=7.4 Hz, 4H), 1.92 (p, J=7.5 Hz, 4H), 1.81-1.62 (m, 3H).

LCMS; m/z 503.5 (M+H)⁺ (ES⁺); 501.3 (M−H)⁻ (ES⁻).

### Example 14: 3-(N-((1,2,3,5,6,7-Hexahydro-s-indacen-4-yl)carbamoyl) sulfamoyl)-N,1-dimethyl-N-(tetrahydro-2H-pyran-4-yl)-1H-pyrazole-5-carboxamide, Sodium Salt

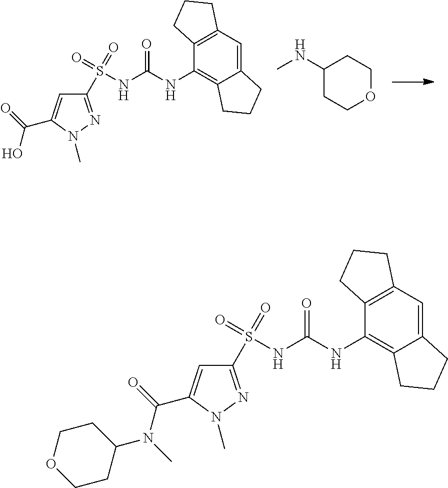

HATU (49.3 mg, 0.130 mmol) was added to a solution of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (67.3 mg, 0.150 mmol) and N-methyltetrahydro-2H-pyran-4-amine (14.93 mg, 0.130 mmol) in DMF (1 mL) and the reaction mixture was stirred at room temperature for 20 hours. Water (1 mL) was slowly added and the reaction mixture was stirred for 1 hour. The suspension was filtered and the collected solid triturated in water (3 mL) for 0.5 hour. The suspension was filtered and the collected solid was washed with water (0.5 mL) and TBME (1 mL). The solid was dried under reduced pressure for 6 hours to afford 3-(N-((1,2,3,5,6,7-hexahydro-s-indacen-4-yl)carbamoyl)sulfamoyl)-N,1-dimethyl-N-(tetrahydro-2H-pyran-4-yl)-1H-pyrazole-5-carboxamide. The solid was dissolved in THF (2 mL) and 2 M sodium tert-butoxide in THF (54.0 μL, 0.108 mmol) was added. The suspension was stirred at room temperature for 2 hours and filtered. The collected solid was washed with EtOAc (2 mL) and dried under reduced pressure for 6 hours to afford the title compound (46 mg, 80%) as a white solid.

¹H NMR (DMSO-d₆), rotamers; δ 7.47 (s, 1H), 6.77 (s, 1H), 6.58 (s, 1H), 4.53-4.49 (m, 0.6H), 3.94-3.90 (m, 2.4H), 3.80 (s, 3H), 3.43-3.41 (m, 1H), 3.16-3.12 (m, 1H), 2.89 (s, 3H), 2.75 (t, J=7.4 Hz, 4H), 2.66 (t, J=7.6 Hz, 4H), 1.93-1.80 (m, 6H), 1.59-1.55 (m, 2H).

LCMS; m/z 502 (M+H)⁺ (ES⁺).

### Example 15: 3-(N-((1,2,3,5,6,7-Hexahydro-s-indacen-4-yl)carbamoyl) sulfamoyl)-N,N-bis(2-hydroxyethyl)-1-methyl-1H-pyrazole-5-carboxamide, Partial Ammonium Salt

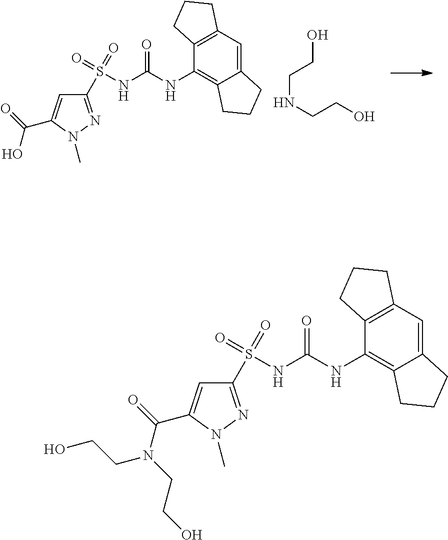

HATU (67.9 mg, 0.178 mmol) was added to a solution of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (66.7 mg, 0.149 mmol) and 2,2′-azanediylbis(ethan-1-ol) (17.11 μL, 0.178 mmol) in DMF (1 mL) and the reaction mixture was stirred at room temperature for 20 hours. Water (1 mL) was slowly added and the reaction mixture was stirred for 1 hour. The suspension was filtered and the collected solid triturated in water (3 mL) for 0.5 hour. The suspension was filtered and the collected solid was washed with water (0.5 mL) and TBME (1 mL). The solid was dried under reduced pressure for 6 hours to afford the title compound (32 mg, 42%) as a white solid.

¹H NMR (DMSO-d₆), rotamers; δ 7.60 (d, J=10.0 Hz, 1H), 7.24 (br s, 1H), 6.80 (s, 1H), 6.69 (s, 1H), 4.95-4.77 (m, 2H), 3.78 (s, 3H), 3.78-3.52 (m, 8H), 2.76 (t, J=7.4 Hz, 4H), 2.65 (t, J=7.6 Hz, 4H), 1.95-1.88 (m, 4H).

LCMS; m/z 492 (M+H)⁺ (ES⁺).

### Example 16: 3-(N-((1,2,3,5,6,7-Hexahydro-s-indacen-4-yl)carbamoyl) sulfamoyl)-N-(2-hydroxyethyl)-N-(2-methoxyethyl)-1-methyl-1H-pyrazole-5-carboxamide, Partial Ammonium Salt

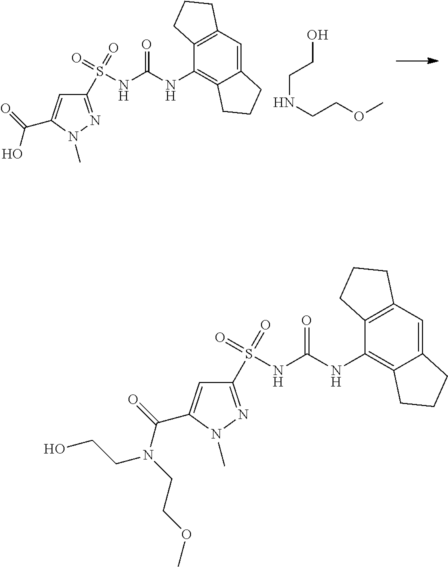

Prepared according to the general procedure of 3-(N-((1,2,3,5,6,7-hexahydro-s-indacen-4-yl)carbamoyl)sulfamoyl)-N,N-bis(2-hydroxyethyl)-1-methyl-1H-pyrazole-5-carboxamide, partial ammonium salt (Example 15) from 3-(N-((1,2,3,5,6,7-hexahydro-s-indacen-4-yl)carbamoyl)sulfamoyl)-1-methyl-1H-pyrazole-5-carboxylic acid disodium salt (Intermediate P8) and 2-((2-methoxyethyl)amino)ethan-1-ol to afford the title compound as a white solid (7 mg, 9%).

¹H NMR (DMSO-d₆), rotamers; δ 7.61 (s, 1H), 7.11 (br s, 1H), 6.81 (s, 1H), 6.60 (s, 1H), 4.89-4.82 (m, 1H), 3.78 (s, 3H), 3.67-3.36 (m, 8H), 3.28-3.15 (2×2s, 3H), 2.76 (t, J=7.4 Hz, 4H), 2.65 (t, J=7.4 Hz, 4H), 1.95-1.88 (m, 4H).

LCMS; m/z 506 (M+H)⁺ (ES⁺).

### Example 17: 3-(N-((1,2,3,5,6,7-Hexahydro-s-indacen-4-yl)carbamoyl) sulfamoyl)-N,1-dimethyl-N-(oxetan-3-yl)-1H-pyrazole-5-carboxamide

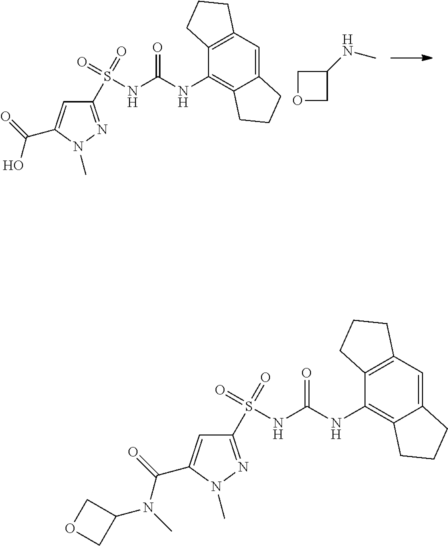

Prepared according to the general procedure of 3-(N-((1,2,3,5,6,7-hexahydro-s-indacen-4-yl)carbamoyl)sulfamoyl)-N,N-bis(2-hydroxyethyl)-1-methyl-1H-pyrazole-5-carboxamide, partial ammonium salt (Example 15) from 3-(N-((1,2,3,5,6,7-hexahydro-s-indacen-4-yl)carbamoyl)sulfamoyl)-1-methyl-1H-pyrazole-5-carboxylic acid disodium salt (Intermediate P8) and N-methyloxetan-3-amine to afford the title compound (27 mg, 36%) as a white solid.

¹H NMR (DMSO-d₆) rotamers: δ 10.95 (br s, 1H), 8.09 (s, 1H), 7.11 (s, 0.5H), 6.95 (s, 1H), 6.89 (s, 0.5H), 5.25-5.22 (m, 0.5H), 5.06-5.03 (m, 0.5H), 4.69-4.60 (m, 4H), 3.92 (s, 3H), 3.19-3.06 (m, 3H), 2.79 (t, J=7.4 Hz, 4H), 2.61 (t, J=7.5 Hz, 4H), 2.00-1.92 (m, 4H).

LCMS; m/z 474 (M+H)⁺ (ES⁺).

### Example 18: 3-(N-((1,2,3,5,6,7-Hexahydro-s-indacen-4-yl)carbamoyl) sulfamoyl)-N-(3-hydroxypropyl)-N,1-dimethyl-1H-pyrazole-5-carboxamide, Partial Ammonium Salt

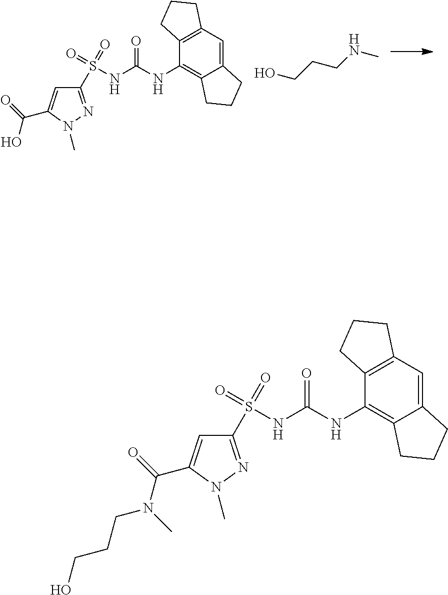

3-(Methylamino)propan-1-ol (19 μL, 0.195 mmol), NaHCO₃ (16 mg, 0.190 mmol) and HATU (72 mg, 0.189 mmol) were successively added to a suspension of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (70 mg, 0.156 mmol) in DMF (1 mL) and stirred for 2 days. The reaction was quenched with water (1 mL) and purified by reversed phase prep-HPLC (General Methods, basic prep) to afford the title compound (37 mg, 48%) as a white solid.

¹H NMR (DMSO-d6), rotamers; δ 7.81 (s, 1H), 7.61-6.99 (br s, 1H), 6.85 (s, 1H), 6.82-6.71 (m, 1H), 4.48 (br s, 1H), 3.94-3.78 (m, 3H), 3.59-3.13 (m, 4H), 3.07-2.90 (m, 3H), 2.76 (t, J=7.4 Hz, 4H), 2.62 (t, J=7.4 Hz, 4H), 1.92 (p, J=7.4 Hz, 4H), 1.79-1.57 (m, 2H).

LCMS; m/z 476.4 (M+H)⁺ (ES⁺); 474.4 (M−H)⁻ (ES⁻).

### Example 19: N-(2,3-Dihydroxypropyl)-3-(N-((1,2,3,5,6,7-hexahydro-s-indacen-4-yl)carbamoyl)sulfamoyl)-N,1-dimethyl-1H-pyrazole-5-carboxamide, Partial Ammonium Salt

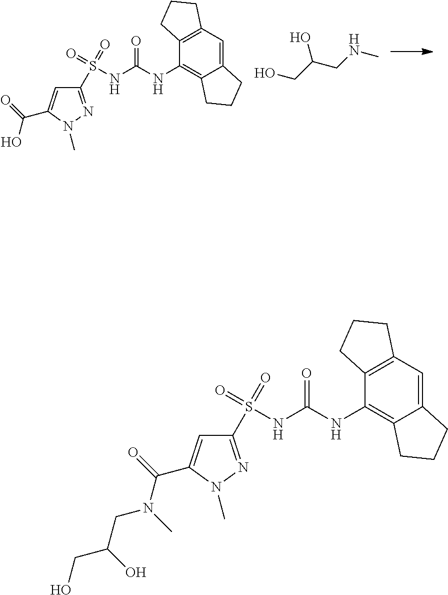

Prepared according to the general procedure of 3-(N-((1,2,3,5,6,7-hexahydro-s-indacen-4-yl)carbamoyl)sulfamoyl)-N-(3-hydroxypropyl)-N,1-dimethyl-1H-pyrazole-5-carboxamide, partial ammonium salt (Example 18) from 3-(N-((1,2,3,5,6,7-hexahydro-s-indacen-4-yl)carbamoyl)sulfamoyl)-1-methyl-1H-pyrazole-5-carboxylic acid disodium salt (Intermediate P8) and 3-(methylamino)propane-1,2-diol to afford the title compound (43 mg, 54%) as a white solid.

¹H NMR (DMSO-d6), rotamers; δ 7.83-7.64 (m, 1H), 7.61-7.01 (m, 1H), 6.87-6.80 (m, 1H), 6.78-6.71 (m, 1H), 5.13-4.99 (m, 1H), 4.96-4.84 (m, 1H), 4.62 (s, 1H), 3.90-3.73 (m, 3H), 3.71-3.09 (m, 5H), 3.07 (s, 1H), 2.98 (s, 1H), 2.76 (t, J=7.4 Hz, 4H), 2.63 (t, J=7.4 Hz, 4H), 2.07-1.79 (m, 4H).

LCMS; m/z 492.4 (M+H)⁺ (ES⁺); 490.2 (M−H)⁻ (ES⁻).

### Example 20: N-Ethyl-3-(N-((1,2,3,5,6,7-hexahydro-s-indacen-4-yl) carbamoyl)sulfamoyl)-N-(2-hydroxyethyl)-1-methyl-1H-pyrazole-5-carboxamide, Partial Ammonium Salt

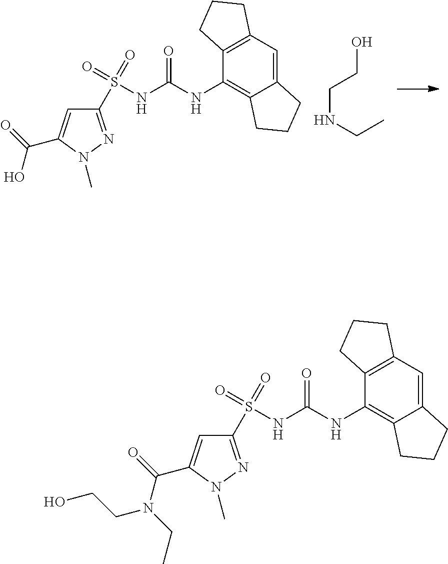

Prepared according to the general procedure of 3-(N-((1,2,3,5,6,7-hexahydro-s-indacen-4-yl)carbamoyl)sulfamoyl)-N-(3-hydroxypropyl)-N,1-dimethyl-1H-pyrazole-5-carboxamide, partial ammonium salt (Example 18) from 3-(N-((1,2,3,5,6,7-hexahydro-s-indacen-4-yl)carbamoyl)sulfamoyl)-1-methyl-1H-pyrazole-5-carboxylic acid disodium salt (Intermediate P8) and 2-(ethylamino)ethan-1-ol to afford the title compound (39 mg, 52%) as a white solid.

¹H NMR (DMSO-d₆), rotamers; δ 8.16-6.99 (br s, 1H), 7.82 & 7.80 (2×s, 1H), 6.86 (s, 1H), 6.79 & 6.74 (2×s, 1H), 4.93 & 4.82 (2×m, 1H), 3.82 (s, 3H), 3.65-3.33 (m, 6H), 2.77 (t, J=7.4 Hz, 4H), 2.63 (t, J=7.3 Hz, 4H), 1.93 (quin, J=7.4 Hz, 4H), 1.15 & 1.08 (2×t, J=7.1 Hz, 3H).

LCMS; m/z 476.5 (M+H)⁺ (ES⁺); 474.3 (M−H)⁻ (ES⁻).

### Example 21: N-((2,6-Diisopropylphenyl)carbamoyl)-1-methyl-5-(morpholine-4-carbonyl)-1H-pyrazole-3-sulfonamide

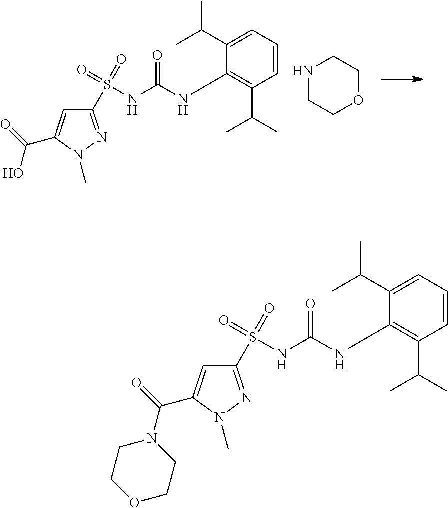

Morpholine (19 μL, 0.220 mmol) and HATU (82 mg, 0.217 mmol) were successively added to a solution of 3-(N-((2,6-diisopropylphenyl)carbamoyl)sulfamoyl)-1-methyl-1H-pyrazole-5-carboxylic acid, disodium salt (Intermediate P9) (70 mg, 0.155 mmol) in DMF (1 mL) and stirred for 20 hours. The reaction was quenched with water (0.1 mL) and purified by reversed phase prep-HPLC (General Methods, basic prep) to afford the title compound (41 mg, 53%) as a white solid.

¹H NMR (DMSO-d₆), rotamers; δ 11.11 (br s, 1H), 7.79 (s, 1H), 7.22 (t, J=7.7 Hz, 1H), 7.10 (d, J=7.7 Hz, 2H), 6.91 (s, 1H), 3.93 (s, 3H), 3.72-3.39 (m, 8H), 2.98 (sept, J=6.9 Hz, 2H), 1.05 (d, J=6.8 Hz, 12H).

LCMS; m/z 478.4 (M+H)⁺ (ES⁺); 476.4 (M−H)⁻ (ES⁻).

### Example 22: 3-(N-((2,6-Diisopropylphenyl)carbamoyl)sulfamoyl)-N-(2-methoxyethyl)-N,1-dimethyl-1H-pyrazole-5-carboxamide

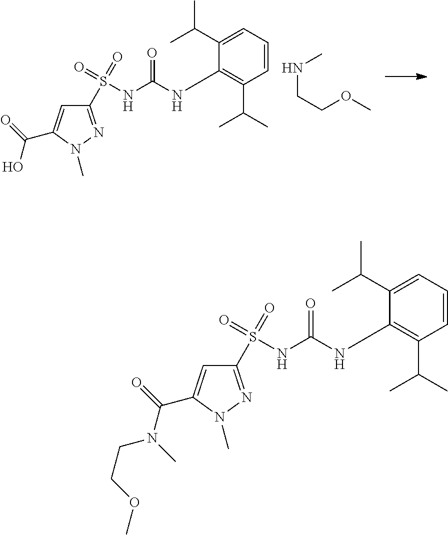

HATU (66.5 mg, 0.175 mmol) was added to a solution of 3-(N-((2,6-diisopropylphenyl) carbamoyl)sulfamoyl)-1-methyl-1H-pyrazole-5-carboxylic acid, disodium salt (Intermediate P9) (65.9 mg, 0.146 mmol) and 2-methoxy-N-methylethanamine (19.00 μL, 0.175 mmol) in DMF (1 mL) and the mixture stirred at room temperature for 20 hours. Water (1 mL) was slowly added and the reaction mixture was stirred at room temperature for 1 hour. The suspension was filtered and the collected solid triturated in water (3 mL) for 0.5 hour. The suspension was filtered and the collected solid was washed with water (0.5 mL) and TBME (1 mL). The solid was dried under reduced pressure for 6 hours to afford the title compound (18 mg, 25%) as a white solid.

¹H NMR (DMSO-d₆), rotamers; δ 11.11 (s, 1H), 8.00-7.72 (m, 1H), 7.28-7.20 (m, 1H), 7.15-7.09 (m, 2H), 7.02-6.86 (m, 1H), 3.93 (s, 1H), 3.86 (s, 2H), 3.66-3.57 (m, 1H), 3.57-3.45 (m, 2H), 3.44-3.37 (m, 1H), 3.27 (s, 1H), 3.16 (s, 2H), 3.03-2.87 (m, 4H), 1.17-0.77 (m, 12H).

LCMS; m/z 480 (M+H)⁺ (ES⁺).

### Example 23:3-(N-((2,6-Diisopropylphenyl)carbamoyl)sulfamoyl)-N,N-bis(2-methoxyethyl)-1-methyl-1H-pyrazole-5-carboxamide

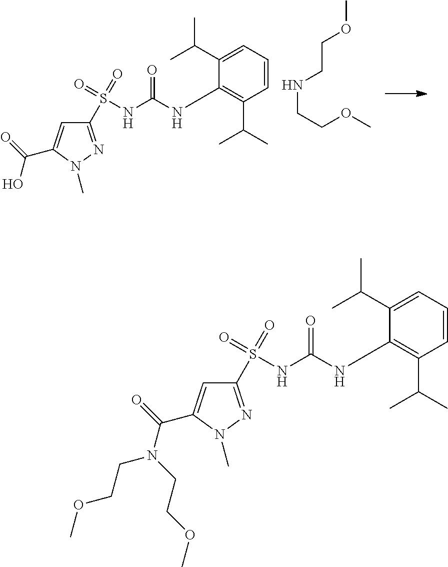

Prepared according to the general procedure of 3-(N-((2,6-diisopropylphenyl) carbamoyl)sulfamoyl)-N-(2-methoxyethyl)-N,1-dimethyl-1H-pyrazole-5-carboxamide (Example 22) from 3-(N-((2,6-diisopropylphenyl)carbamoyl)sulfamoyl)-1-methyl-1H-pyrazole-5-carboxylic acid, disodium salt (Intermediate P9) and bis(2-methoxyethyl)amine to afford the title compound (22 mg, 23%) as a white solid.

¹H NMR (DMSO-d₆), rotamers; δ 11.11 (s, 1H), 7.91 (s, 1H), 7.31-7.18 (m, 1H), 7.13 (s, 1H), 7.11 (s, 1H), 6.92 (s, 1H), 3.86 (s, 3H), 3.66-3.58 (m, 2H), 3.56-3.47 (m, 4H), 3.43-3.35 (m, 2H), 3.27 (s, 3H), 3.14 (s, 3H), 2.94 (sept, J=6.8 Hz, 2H), 1.18-0.87 (m, 12H).

LCMS; m/z 524 (M+H)⁺ (ES⁺).

### Example 24: N-((1,2,3,5,6,7-Hexahydro-s-indacen-4-yl)carbamoyl)-5-(4-hydroxypiperidine-1-carbonyl)-1-methyl-1H-pyrazole-3-sulfonamide, Partial Ammonium Salt

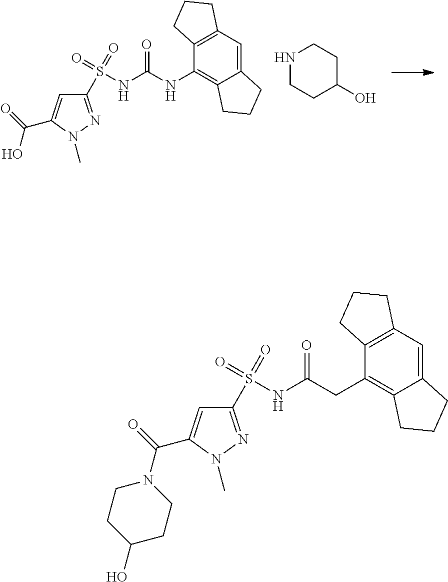

Prepared according to the general procedure of 3-(N-((1,2,3,5,6,7-hexahydro-s-indacen-4-yl)carbamoyl)sulfamoyl)-N-(3-hydroxypropyl)-N,1-dimethyl-1H-pyrazole-5-carboxamide, partial ammonium salt (Example 18) from 3-(N-((1,2,3,5,6,7-hexahydro-s-indacen-4-yl)carbamoyl)sulfamoyl)-1-methyl-1H-pyrazole-5-carboxylic acid disodium salt (Intermediate P8) and piperidin-4-ol to afford the title compound (44 mg, 57%) as a white solid.

¹H NMR (DMSO-d₆), rotamers; δ 7.88 (s, 1H), 7.48-6.92 (m, 1H), 6.88 (s, 1H), 6.78 (s, 1H), 4.83 (d, J=3.9 Hz, 1H), 3.96 (m, 1H), 3.87 (s, 3H), 3.77 (m, 1H), 3.63 (m, 1H), 3.32 (m, 2H), 2.78 (t, J=7.4 Hz, 4H), 2.61 (t, J=7.4 Hz, 4H), 1.94 (quin, J=7.5 Hz, 4H), 1.77 (m, 2H), 1.39 m, 2H).

LCMS; m/z 488.4 (M+H)⁺ (ES⁺); 486.3 (M−H)⁻ (ES⁻).

### Example 25: N-((1,2,3,5,6,7-Hexahydro-s-indacen-4-yl)carbamoyl)-5-(3-methoxypyrrolidine-1-carbonyl)-1-methyl-1H-pyrazole-3-sulfonamide, Partial Ammonium Salt

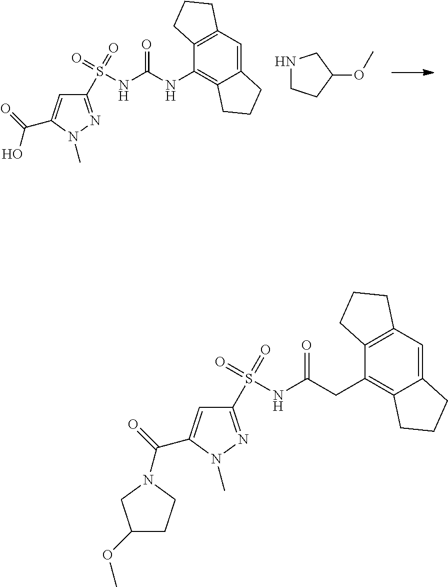

3-Methoxypyrrolidine (19 mg, 0.188 mmol) and HATU (72 mg, 0.189 mmol) were successively added to a suspension of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (70 mg, 0.156 mmol) in DMF (1 mL) and stirred for 20 hours. The solution was purified by chromatography on RP Flash C18 (12 g column, 5-50% MeCN/10 mM ammonium bicarbonate) to afford the title compound (25 mg, 32%) as a white solid.

¹H NMR (DMSO-d₆), rotamers; δ 7.82 (s, 1H), 7.08 (s, 1H), 6.96 (d, J=2.6 Hz, 1H), 6.86 (s, 1H), 4.07-3.90 (m, 4H), 3.75-3.67 (m, 1H), 3.64-3.39 (m, 4H), 3.26 (s, 1H), 3.19 (s, 1H), 2.76 (t, J=7.4 Hz, 4H), 2.61 (t, J=7.4 Hz, 4H), 2.06-1.84 (m, 6H). LCMS; m/z 488.43 (M+H)⁺ (ES⁺); 486.35 (M−H)⁻ (ES⁻).

### Example 26: 3-(N-((1,2,3,5,6,7-Hexahydro-s-indacen-4-yl)carbamoyl) sulfamoyl)-N,N-bis(2-methoxyethyl)-1-methyl-1H-pyrazole-5-carboxamide, Partial Ammonium Salt

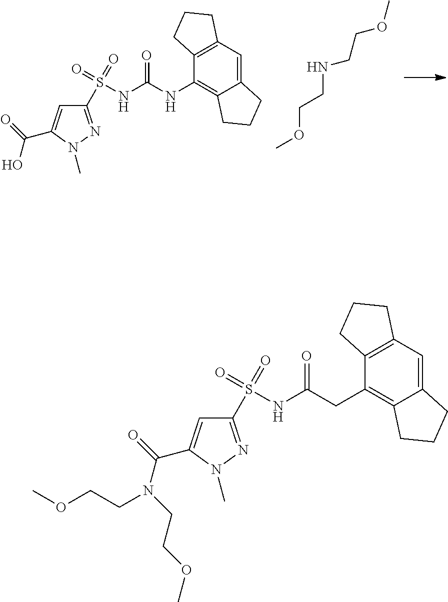

Prepared according to the general procedure of N-((1,2,3,5,6,7-hexahydro-s-indacen-4-yl)carbamoyl)-5-(3-methoxypyrrolidine-1-carbonyl)-1-methyl-1H-pyrazole-3-sulfonamide, partial ammonium salt (Example 25) from 3-(N-((1,2,3,5,6,7-hexahydro-s-indacen-4-yl)carbamoyl)sulfamoyl)-1-methyl-1H-pyrazole-5-carboxylic acid disodium salt (Intermediate P8) and bis(2-methoxyethyl)amine to afford the title compound (36 mg, 44%) as a white solid.

¹H NMR (DMSO-d₆), rotamers; δ 7.79 (s, 1H), 7.10 (br s, 1H), 6.85 (s, 1H), 6.73 (s, 1H), 3.80 (s, 3H), 3.63 (t, J=5.8 Hz, 2H), 3.58-3.49 (m 4H), 3.39 (t, J=5.0 Hz, 2H), 3.28 (s, 3H), 3.14 (s, 3H), 2.77 (t, J=7.4 Hz, 4H), 2.63 (t, J=7.3 Hz, 4H), 1.92 (quin, J=7.5 Hz, 4H).

LCMS; m/z 520.44 (M+H)⁺ (ES⁺); 518.30 (M−H)⁻ (ES⁻).

### Example 27:3-(N-((1,2,3,5,6,7-Hexahydro-s-indacen-4-yl)carbamoyl) sulfamoyl)-N-(2-methoxyethyl)-N,1-dimethyl-1H-pyrazole-5-carboxamide, Sodium Salt

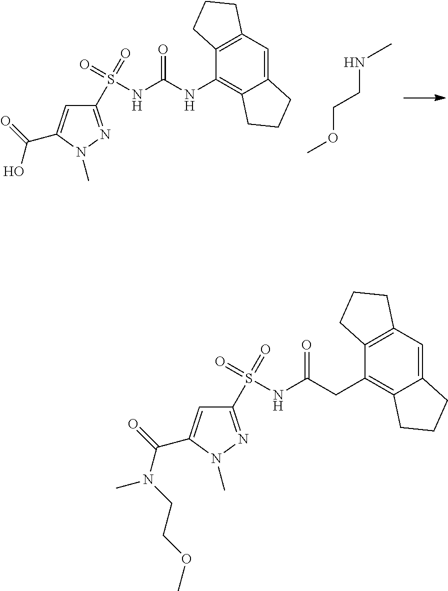

2-Methoxy-N-methylethanamine (19 μL, 0.173 mmol), HATU (71 mg, 0.187 mmol) and Hunig's base (65 μL, 0.372 mmol) were successively added to a solution of 3-(N-((1,2,3,5,6,7-hexahydro-s-indacen-4-yl)carbamoyl)sulfamoyl)-1-methyl-1H-pyrazole-5-carboxylic acid (Intermediate P6) (50 mg, 0.124 mmol) in DMF (1 mL) and stirred for 20 hours. The reaction was quenched with water (1 mL) and purified by chromatography on RP Flash C18 (12 g column, 5-50% MeCN/10 mM ammonium bicarbonate) to afford crude product. 18 mg of the crude product were partitioned between TBME (1 mL) and 0.1 M aqueous NaHCO₃ (340 μL, 0.034 mmol). The layers were separated and the aqueous layer washed with further TBME (2×1 mL). The aqueous layer was evaporated in vacuo and dried to afford the title compound (17 mg, 27%) as a white solid.

¹H NMR (DMSO-d₆), rotamers; δ 7.52 (s, 1H), 6.77 (s, 1H), 6.6 & 6.55 (2×s, 1H), 3.81 & 3.76 (2×s, 3H), 3.61 (m, 1H), 3.56 (m, 2H), 3.4 (m, 1H), 3.28 & 3.16 (2×s, 3H), 3.05 & 2.97 (2×s, 3H), 2.75 (t, J=7.4 Hz, 4H), 2.65 (t, J=7.5 Hz, 4H), 1.89 (quin, J=7.5 Hz, 4H).

LCMS; m/z 476.4 (M+H)⁺ (ES⁺); 474.3 (M−H)⁻ (ES⁻).

### Example 28: N-((1,2,3,5,6,7-Hexahydro-s-indacen-4-yl)carbamoyl)-1-methyl-5-(morpholine-4-carbonyl)-1H-pyrazole-3-sulfonamide, Sodium Salt

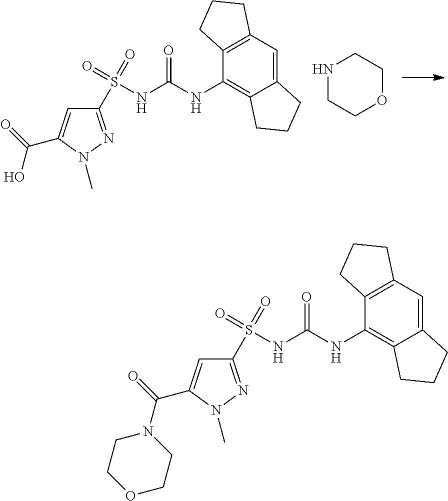

Prepared according to the general procedure of 3-(N-((1,2,3,5,6,7-hexahydro-s-indacen-4-yl)carbamoyl)sulfamoyl)-N-(2-methoxyethyl)-N,1-dimethyl-1H-pyrazole-5-carboxamide, sodium salt (Example 27) from 3-(N-((1,2,3,5,6,7-hexahydro-s-indacen-4-yl)carbamoyl)sulfamoyl)-1-methyl-1H-pyrazole-5-carboxylic acid disodium salt (Intermediate P8) and morpholine to afford the title compound (12 mg, 16%) as a white solid.

¹H NMR (DMSO-d₆), rotamers; δ 7.54 (s, 1H), 6.77 (s, 1H), 6.59 (s, 1H), 3.84 (s, 3H), 3.76-3.43 (m, 8H), 2.75 (t, J=7.4 Hz, 4H), 2.64 (t, J=7.3 Hz, 4H), 1.90 (quin, J=7.5 Hz, 4H).

LCMS; m/z 474.4 (M+H)⁺ (ES⁺); 472.3 (M−H)⁻ (ES⁻).

### Example 29: N-((3-(N-((1,2,3,5,6,7-Hexahydro-s-indacen-4-yl)carbamoyl) sulfamoyl)-1-isopropyl-1H-pyrazol-5-yl)methyl)-N-methylacetamide

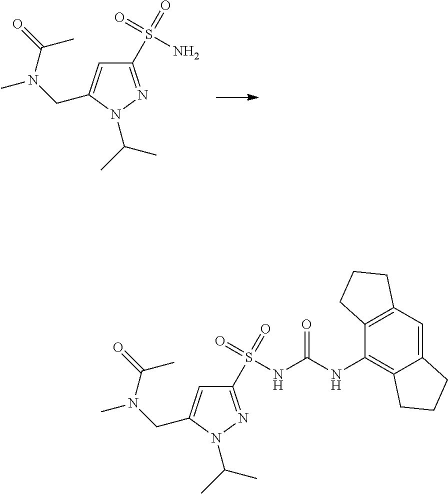 [SMILES: CC(=O)N(C)Cc1cc(S(N)(=O)=O)nn1C(C)C ; CC(=O)N(C)Cc1cc(S(=O)(=O)NC(=O)Nc2c3c(cc4c2CCC4)CCC3)nn1C(C)C]

Sodium tert-butoxide (2 M in THF) (0.057 mL, 0.115 mmol) was added to a solution of N-((1-isopropyl-3-sulfamoyl-1H-pyrazol-5-yl)methyl)-N-methylacetamide (Intermediate P15) (30 mg, 0.109 mmol) in THF (2.2 mL) and stirred at room temperature for 1 hour. Then 4-isocyanato-1,2,3,5,6,7-hexahydro-s-indacene (Intermediate A1) (24 mg, 0.120 mmol) was added and stirred at room temperature overnight. The suspension was filtered and the collected solid washed with EtOAc (2 mL). The solid was dissolved in DMSO (2 mL) and purified by reversed phase prep-HPLC (General Methods, basic prep) to afford the title compound (20 mg, 38%) as a white solid.

¹H NMR (DMSO-d₆) δ 10.77 (br s, 1H), 7.93 (s, 1H), 6.88 (s, 1H), 6.62 (s, 1H), 4.75-4.61 (m, 1H), 4.59 (s, 2H), 2.90 (s, 3H), 2.75 (t, J=7.4 Hz, 4H), 2.56 (t, J=7.3 Hz, 4H), 2.02 (s, 3H), 1.90 (p, J=7.6 Hz, 4H), 1.31 (d, J=6.5 Hz, 6H).

LCMS; m/z 474.5 (M+H)⁺ (ES⁺)

### Example 30: 5-(Azetidine-1-carbonyl)-N-((4-fluoro-2,6-diisopropylphenyl) carbamoyl)-1-isopropyl-1H-pyrazole-3-sulfonamide, Sodium Salt

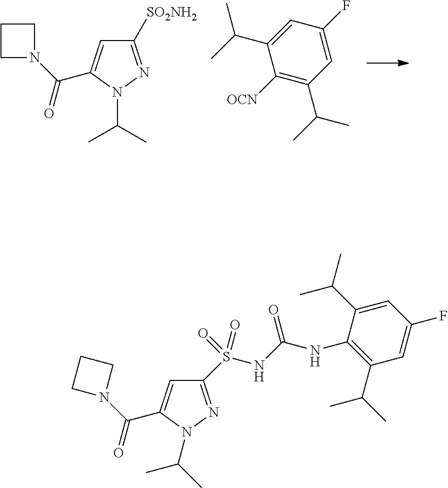

5-(Azetidine-1-carbonyl)-1-isopropyl-H-pyrazole-3-sulfonamide (Intermediate P12) (50 mg, 0.184 mmol) was dissolved in THF (2 mL), and 2M sodium tert-butoxide in THF (0.101 mL, 0.202 mmol) was added. After 1 hour, 5-fluoro-2-isocyanato-1,3-diisopropylbenzene (Intermediate A2) (44.7 mg, 0.202 mmol) followed by THF (2 mL) was added and the mixture stirred at room temperature for 16 hours. Additional isocyanate (20 mg) and 2 M sodium tert-butoxide in THF (0.05 mL) were added and the reaction was left to stir for a further 4 hours. The emulsion obtained was filtered and dried under vacuum at 40° C. for 4 days to afford the title compound (46 mg, 48%) as a white solid.

¹H NMR (DMSO-d₆) δ 7.33 (s, 1H), 6.79 (d, J=10.1 Hz, 2H), 6.65 (s, 1H), 5.26 (sept, J=6.7 Hz, 1H), 4.25 (t, J=7.7 Hz, 2H), 4.02 (t, J=7.8 Hz, 2H), 3.22-2.93 (m, 2H), 2.26 (app. pent, J=7.7 Hz, 2H), 1.37 (d, J=6.6 Hz, 6H), 1.03 (d, J=6.8 Hz, 12H).

LCMS; m/z 494.4 (M+H)⁺ (ES⁺); 492.3 (M−H)⁻ (ES⁻).

### Example 31: 5-(Azetidine-1-carbonyl)-N-((1,2,3,5,6,7-hexahydro-s-indacen-4-yl)carbamoyl)-1-isopropyl-1H-pyrazole-3-sulfonamide, partial Ammonium Salt

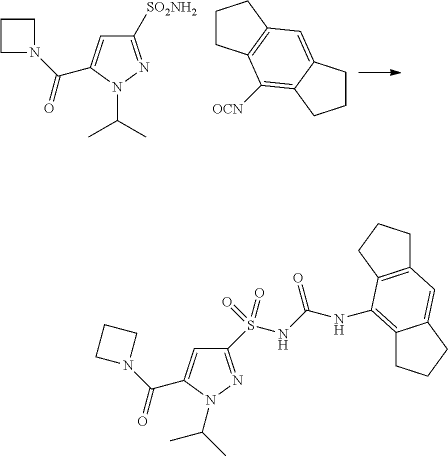

Prepared according to the general procedure for 5-(azetidine-1-carbonyl)-N-((4-fluoro-2,6-diisopropylphenyl) carbamoyl)-1-isopropyl-1H-pyrazole-3-sulfonamide (Example 30) from 5-(azetidine-1-carbonyl)-1-isopropyl-1H-pyrazole-3-sulfonamide (Intermediate P12) and 4-isocyanato-1,2,3,5,6,7-hexahydro-s-indacene (Intermediate A1). The crude product was purified by chromatography on RP Flash C18 (basic) to afford the title compound (47 mg, 52%) as a white solid.

¹H NMR (DMSO-d₆) δ 7.77 (s, 1H), 6.92-6.68 (m, 2H), 5.28 (sept, J=6.5 Hz, 1H), 4.27 (t, J=7.7 Hz, 2H), 4.03 (t, J=7.7 Hz, 2H), 2.76 (t, J=7.4 Hz, 4H), 2.60 (t, J=7.3 Hz, 4H), 2.31-2.19 (m, 2H), 1.90 (p, J=7.4 Hz, 4H), 1.38 (d, J=6.6 Hz, 6H).

LCMS; m/z 472.5 (M+H)⁺ (ES⁺); 470.3 (M−H)⁻ (ES⁻).

### Example 32: 3-(N-((4-Chloro-2,6-diisopropylphenyl)carbamoyl) sulfamoyl)-N,1-dimethyl-1H-pyrazole-5-carboxamide

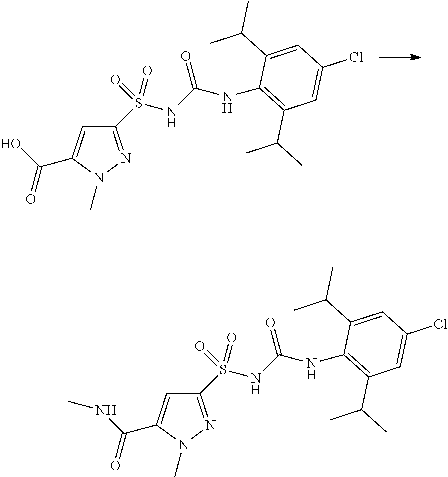 [SMILES: CC(C)c1cc(Cl)cc(C(C)C)c1NC(=O)NS(=O)(=O)c1cc(C(=O)O)n(C)n1 ; CNC(=O)c1cc(S(=O)(=O)NC(=O)Nc2c(C(C)C)cc(Cl)cc2C(C)C)nn1C]

HATU (68.8 mg, 0.181 mmol) was added to a solution of 3-(N-((4-chloro-2,6-diisopropylphenyl)carbamoyl)sulfamoyl)-1-methyl-1H-pyrazole-5-carboxylic acid, disodium salt (Intermediate P11) (73.4 mg, 0.151 mmol) and methylamine (83 μL, 0.166 mmol) in DMF (1 mL). TEA (21 μL, 0.151 mmol) was added and the mixture stirred at room temperature for 20 hours. Water (1 mL) was slowly added and the mixture stirred for 1 hour, filtered, and the collected solid triturated in water (3 mL) for 0.5 hour. The suspension was filtered, the solid washed with water (0.5 mL) and MTBE (1 mL), and then purified by chromatography on RP Flash C18 (13 g column, 0-50% MeCN/10 mM ammonium bicarbonate). The product was triturated with MTBE (2 mL) for 1 hour, filtered and dried under vacuum for 15 hours to afford the title compound (7 mg, 10%) as a white solid.

¹H NMR (DMSO-d₆) δ 11.21 (s, 1H), 8.64 (s, 1H), 7.89 (s, 1H), 7.31 (s, 1H), 7.12 (s, 2H), 4.13 (s, 3H), 3.00-2.90 (m, 2H), 2.74 (d, J=4.5 Hz, 3H), 1.05-1.01 (m, 12H).

LCMS; m/z 456.4 and 458.4 (M+H)⁺ (ES⁺).

### Example 33: 3-(N-((4-Fluoro-2-isopropyl-6-(tetrahydro-2H-pyran-4-yl)phenyl)carbamoyl)sulfamoyl)-N,N,1-trimethyl-1H-pyrazole-5-carboxamide, Sodium Salt

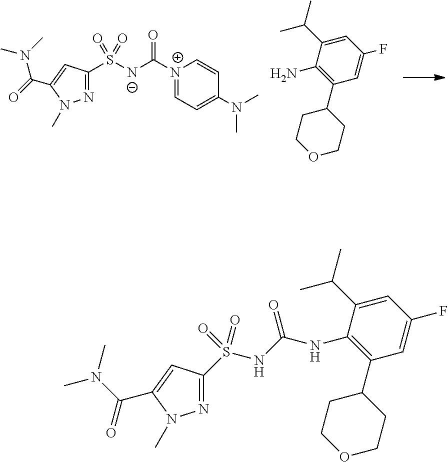

A mixture of (4-(dimethylamino)pyridin-1-ium-1-carbonyl)((5-(dimethylcarbamoyl)-1-methyl-1H-pyrazol-3-yl)sulfonyl)amide (Intermediate P13) (70 mg, 0.184 mmol) and 4-fluoro-2-isopropyl-6-(tetrahydro-2H-pyran-4-yl)aniline (Intermediate A6) (40 mg, 0.167 mmol) in MeCN (1 mL) was stirred at 50° C. for 1 hour. The crude product was purified by reversed phase prep-HPLC (General Methods, basic prep) to afford the desired carboxamide as a white solid (21 mg). To a solution of the carboxamide (21 mg) in THF (0.5 mL), a solution of 2.0 M NaO^{t}Bu in THF (1.0 eq) was added. The mixture was stirred for 1 hour, the solvent evaporated and the solid triturated with THF/MTBE. The precipitate was collected by filtration, washing with ether, and dried in vacuo to afford the title compound (5 mg, 6%) as a white solid.

¹H NMR (DMSO-d₆), rotamers; δ 7.39 (s, 1H), 6.81 (td, J=10.6, 2.9 Hz, 2H), 6.61 (s, 1H), 3.90-3.81 (m, 5H), 3.28-3.11 (m, 3H), 3.04-2.97 (m, 7H), 1.57-1.43 (m, 4H), 1.04 (d, J=6.8 Hz, 6H).

LCMS; m/z 496.5 (M+H)⁺ (ES⁺); 494.3 (M−H)⁻ (ES⁻).

### Example 34: 3-(N-((2-Isopropyl-5-(pyrimidin-5-yl)phenyl)carbamoyl) sulfamoyl)-N,N,1-trimethyl-1H-pyrazole-5-carboxamide, Sodium Salt

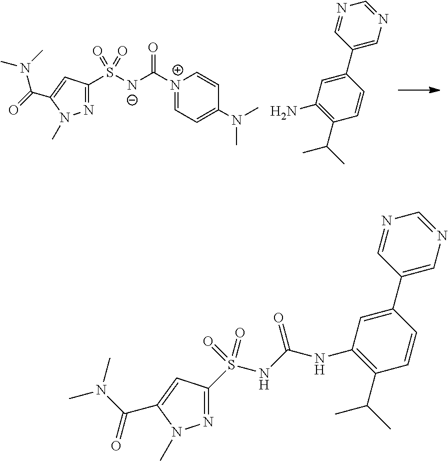

Prepared according to the general procedure for 3-(N-((4-fluoro-2-isopropyl-6-(tetrahydro-2H-pyran-4-yl)phenyl)carbamoyl)sulfamoyl)-N,N,1-trimethyl-1H-pyrazole-5-carboxamide, sodium salt (Example 33) from (4-(dimethylamino)pyridin-1-ium-1-carbonyl)((5-(dimethylcarbamoyl)-1-methyl-1H-pyrazol-3-yl)sulfonyl)amide (Intermediate P13) and 2-isopropyl-5-(pyrimidin-5-yl)aniline (Intermediate A8) to afford the title compound (27 mg, 23%) as a white solid.

¹H NMR (DMSO-d₆), rotamers; δ 9.15 (s, 1H), 9.01 (s, 2H), 8.14 (s, 1H), 7.77 (s, 1H), 7.31 (s, 2H), 6.63 (s, 1H), 3.82 (s, 3H), 3.19 (sept, J=6.8 Hz, 1H), 3.03 (s, 3H), 2.98 (s, 3H), 1.17 (d, J=6.8 Hz, 6H).

LCMS; m/z 472.4 (M+H)⁺ (ES⁺); 470.4 (M−H)⁻ (ES⁻).

### Example 35: 3-(N-((4-Fluoro-2-isopropyl-6-(1-methyl-H-pyrazol-4-yl) phenyl)carbamoyl)sulfamoyl)-N,N,1-trimethyl-1H-pyrazole-5-carboxamide, Sodium Salt

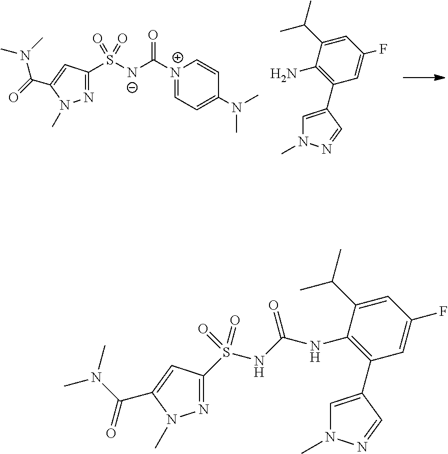

Prepared according to the general procedure for 3-(N-((4-fluoro-2-isopropyl-6-(tetrahydro-2H-pyran-4-yl)phenyl)carbamoyl)sulfamoyl)-N,N,1-trimethyl-1H-pyrazole-5-carboxamide, sodium salt (Example 33) from (4-(dimethylamino)pyridin-1-ium-1-carbonyl)((5-(dimethylcarbamoyl)-1-methyl-1H-pyrazol-3-yl)sulfonyl)amide (Intermediate P13) and 4-fluoro-2-isopropyl-6-(1-methyl-H-pyrazol-4-yl)aniline (Intermediate A4) to afford the title compound (40 mg, 20%) as a white solid.

¹H NMR (DMSO-d₆), rotamers; δ 7.95 (s, 1H), 7.76 (s, 1H), 7.25 (s, 1H), 7.10 (dd, J=9.9, 3.0 Hz, 1H), 6.86 (dd, J=9.8, 2.9 Hz, 1H), 6.58 (s, 1H), 3.82 (s, 3H), 3.80 (s, 3H), 3.20 (m, 1H), 2.99 (s, 6H), 1.06 (d, J=6.8 Hz, 6H).

LCMS; m/z 492.4 (M+H)⁺ (ES⁺); 490.3 (M−H)⁻ (ES⁻).

### Example 16: 3-(N-((2-Isopropyl-5-(1-methyl-1H-pyrazol-4-yl)phenyl) carbamoyl)sulfamoyl)-N,N,1-trimethyl-1H-pyrazole-5-carboxamide, Sodium Salt

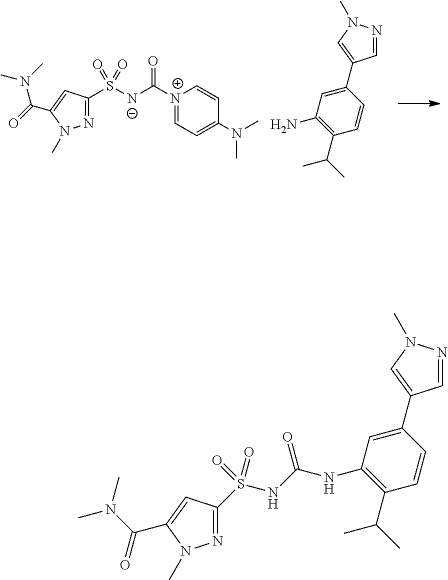

Prepared according to the general procedure for 3-(N-((4-fluoro-2-isopropyl-6-(tetrahydro-2H-pyran-4-yl)phenyl)carbamoyl)sulfamoyl)-N,N,1-trimethyl-1H-pyrazole-5-carboxamide, sodium salt (Example 33) from (4-(dimethylamino)pyridin-1-ium-1-carbonyl)((5-(dimethylcarbamoyl)-1-methyl-1H-pyrazol-3-yl)sulfonyl)amide (Intermediate P13) and 2-isopropyl-5-(1-methyl-1H-pyrazol-4-yl)aniline (Intermediate A7) to afford the title compound (6 mg, 5%) as a white solid.

¹H NMR (DMSO-d₆), rotamers; δ 7.94 (s, 1H), 7.89 (s, 1H), 7.65 (s, 1H), 7.57 (s, 1H), 7.11 (d, J=8.1 Hz, 1H), 7.10-7.05 (m, 1H), 6.61 (s, 1H), 3.85 (s, 3H), 3.82 (s, 3H), 3.09 (sept, J=6.8 Hz, 1H), 3.03 (s, 3H), 2.98 (s, 3H), 1.14 (d, J=6.8 Hz, 6H).

LCMS; m/z 474.5 (M+H)⁺ (ES⁺); 472.3 (M−H)⁻ (ES⁻).

### Example 37: 3-(N-((4-Chloro-2,6-diisopropylphenyl)carbamoyl)sulfamoyl)-N-(cyanomethyl)-N,1-dimethyl-1H-pyrazole-5-carboxamide

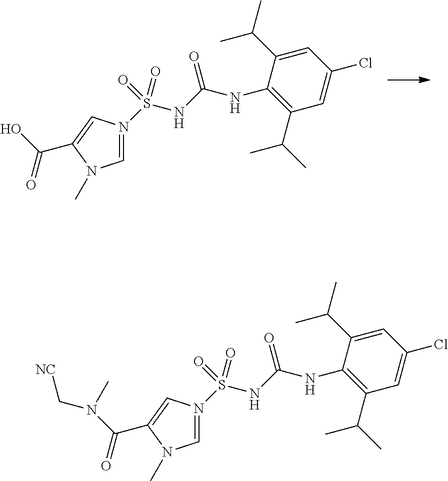

Prepared according to the general procedure for 3-(N-((4-chloro-2,6-diisopropylphenyl) carbamoyl)sulfamoyl)-N,1-dimethyl-1H-pyrazole-5-carboxamide (Example 32) from 3-(N-((4-chloro-2,6-diisopropylphenyl)carbamoyl)sulfamoyl)-1-methyl-1H-pyrazole-5-carboxylic acid, disodium salt (Intermediate P11) and 2-(methylamino)acetonitrile, HCl to afford the title compound (26 mg, 42%).

¹H NMR (DMSO-d₆), rotamers; δ 11.25 (s, 1H), 7.99 (s, 1H), 7.14 (s, 3H), 4.58 (s, 2H), 3.97 (s, 3H), 3.12 (s, 3H), 2.98-2.85 (m, 2H), 1.05 (br s, 12H).

LCMS; m/z 495.5 and 497.5 (M+H)⁺ (ES⁺).

### Example 38:3-(N-((2,6-Diisopropylphenyl)carbamoyl)sulfamoyl)-N-isopropyl-1-methyl-1H-pyrazole-5-carboxamide

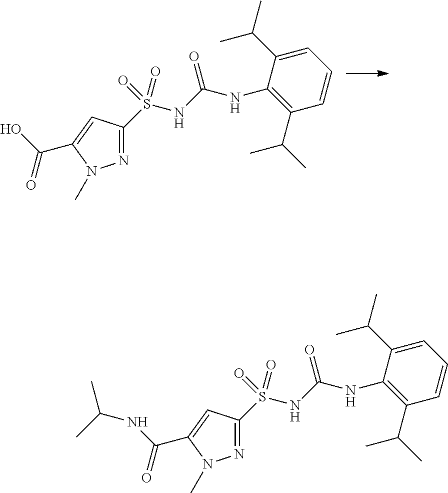 [SMILES: CC(C)c1cccc(C(C)C)c1NC(=O)NS(=O)(=O)c1cc(C(=O)O)n(C)n1 ; CC(C)NC(=O)c1cc(S(=O)(=O)NC(=O)Nc2c(C(C)C)cccc2C(C)C)nn1C]

Prepared according to the general procedure for 3-(N-((4-chloro-2,6-diisopropylphenyl) carbamoyl)sulfamoyl)-N,1-dimethyl-1H-pyrazole-5-carboxamide (Example 32) from 3-(N-((2,6-diisopropylphenyl)carbamoyl)sulfamoyl)-1-methyl-1H-pyrazole-5-carboxylic acid, disodium salt (Intermediate P9) and isopropylamine to afford the title compound (28 mg, 45%) as a white solid.

¹H NMR (DMSO-d₆) δ 11.06 (s, 1H), 8.49 (d, J=7.8 Hz, 1H), 7.85 (s, 1H), 7.47 (s, 1H), 7.27-7.20 (m, 1H), 7.11 (d, J=7.6 Hz, 2H), 4.15 (s, 3H), 4.09-3.99 (m, 1H), 2.90-2.86 (m, 2H), 1.13 (d, J=6.6 Hz, 6H), 1.05-1.03 (m, 12H).

LCMS; m/z 450 (M+H)⁺ (ES⁺).

### Example 39: N-((2,6-Diisopropylphenyl)carbamoyl)-5-(3-fluoroazetidine-1-carbonyl)-1-methyl-1H-pyrazole-3-sulfonamide, Sodium Salt

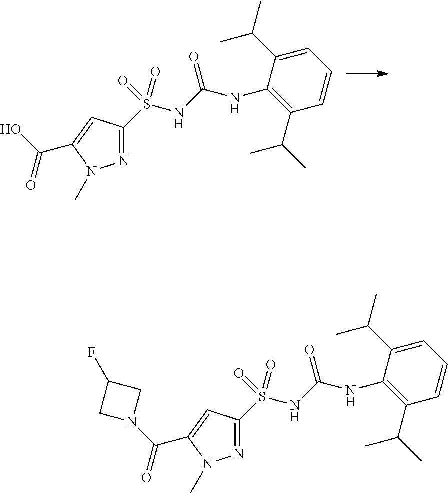 [SMILES: CC(C)c1cccc(C(C)C)c1NC(=O)NS(=O)(=O)c1cc(C(=O)O)n(C)n1 ; CC(C)c1cccc(C(C)C)c1NC(=O)NS(=O)(=O)c1cc(C(=O)N2CC(F)C2)n(C)n1]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((2,6-diisopropylphenyl)carbamoyl)sulfamoyl)-1-methyl-1H-pyrazole-5-carboxylic acid, disodium salt (Intermediate P9) and 3-fluoroazetidine, HCl. The sodium salt was generated by dissolving the free acid (19 mg, 0.041 mmol) in THF (1 mL) and adding 2 M solution of sodium tert-butoxide (20.50 μL, 0.041 mmol) in THF. The suspension was stirred for 2 hours and filtered. The collected solid was washed with EtOAc (2 mL) and dried under reduced pressure for 6 hours to afford the title compound (6 mg, 8%) as a white solid.

¹H NMR (DMSO-d₆), rotamers; δ 7.32 (s, 1H), 7.11-7.10 (m, 1H), 7.01 (d, J=7.3 Hz, 2H), 6.71 (s, 1H), 5.51-5.33 (m, 1H), 4.63-4.59 (m, 1H), 4.40-4.32 (m, 2H), 4.11-4.01 (m, 1H), 3.98 (s, 3H), 3.16-3.12 (m, 2H), 1.04 (d, J=6.8 Hz, 12H).

LCMS; m/z 466 (M+H)⁺ (ES⁺).

### Example 40: N-(Cyanomethyl)-3-(N-((2,6-diisopropylphenyl)carbamoyl) sulfamoyl)-N,1-dimethyl-1H-pyrazole-5-carboxamide

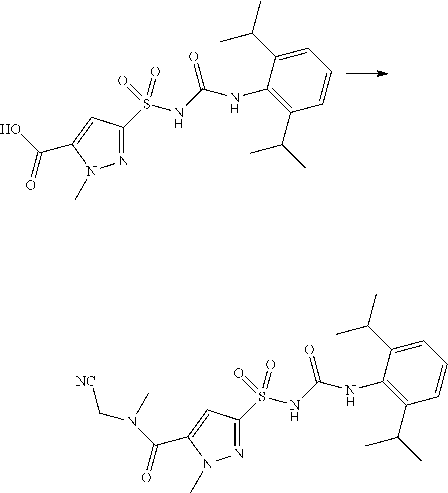 [SMILES: CC(C)c1cccc(C(C)C)c1NC(=O)NS(=O)(=O)c1cc(C(=O)O)n(C)n1 ; CC(C)c1cccc(C(C)C)c1NC(=O)NS(=O)(=O)c1cc(C(=O)N(C)CC#N)n(C)n1]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((2,6-diisopropylphenyl)carbamoyl)sulfamoyl)-1-methyl-1H-pyrazole-5-carboxylic acid, disodium salt (Intermediate P9) and 2-(methylamino)acetonitrile, HCl to afford the title compound (25 mg, 33%) as a white solid.

¹H NMR (DMSO-d₆) δ 7.59 (s, 1H), 7.20-7.12 (m, 1H), 7.06 (d, J=7.6 Hz, 2H), 6.91 (s, 1H), 4.57 (s, 2H), 3.91 (s, 3H), 3.13 (s, 3H), 3.08-3.05 (m, 2H), 1.05 (d, J=6.9 Hz, 12H). Acidic NH not observed

LCMS; m/z 461 (M+H)⁺ (ES⁺).

### Example 41: 3-(N-((4-Fluoro-2-isopropyl-6-(pyridin-3-yl)phenyl) carbamoyl)sulfamoyl)-N,N,1-trimethyl-1H-pyrazole-5-carboxamide, Sodium Salt

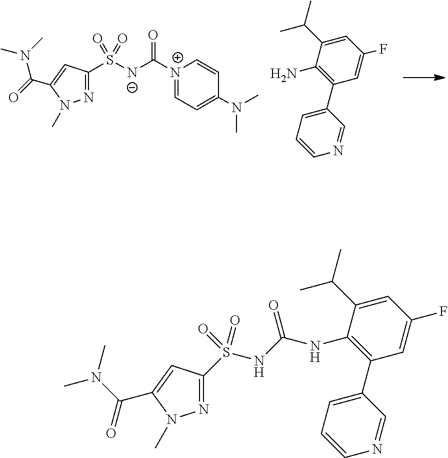

Prepared according to the general procedure for 3-(N-((4-fluoro-2-isopropyl-6-(tetrahydro-2H-pyran-4-yl)phenyl)carbamoyl)sulfamoyl)-N,N,1-trimethyl-1H-pyrazole-5-carboxamide, sodium salt (Example 33) from (4-(dimethylamino)pyridin-1-ium-1-carbonyl)((5-(dimethylcarbamoyl)-1-methyl-1H-pyrazol-3-yl)sulfonyl)amide (Intermediate P13) and 4-fluoro-2-isopropyl-6-(pyridin-3-yl)aniline (Intermediate A3) to afford the title compound (23 mg, 9%) as a white solid.

¹H NMR (DMSO-d₆, 70° C.) δ 8.55 (m, 1H), 8.45 (dd, J=4.8, 1.7 Hz, 1H), 7.77 (dt, J=7.8, 2.0 Hz, 1H), 7.25 (ddd, J=7.9, 4.8, 0.9 Hz, 1H), 7.06 (dd, J=10.2, 3.0 Hz, 1H), 6.91 (dd, J=9.1, 3.0 Hz, 1H), 6.44 (s, 1H), 3.84 (s, 3H), 3.26 (sept, J=6.9 Hz, 1H), 3.04 (s, 6H), 1.13 (d, J=6.9 Hz, 6H). NH not observed.

LCMS; m/z 489.4 (M+H)⁺ (ES⁺).

### Example 42: 3-(N-((4-Fluoro-2-isopropyl-6-(pyrimidin-5-yl)phenyl) carbamoyl)sulfamoyl)-N,N,1-trimethyl-1H-pyrazole-5-carboxamide, Sodium Salt

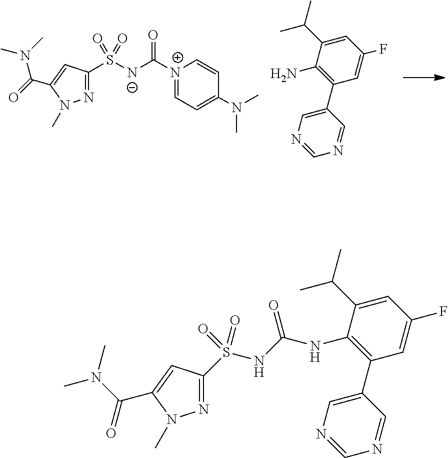

Prepared according to the general procedure for 3-(N-((4-fluoro-2-isopropyl-6-(tetrahydro-2H-pyran-4-yl)phenyl)carbamoyl)sulfamoyl)-N,N,1-trimethyl-1H-pyrazole-5-carboxamide, sodium salt (Example 33) from (4-(dimethylamino)pyridin-1-ium-1-carbonyl)((5-(dimethylcarbamoyl)-1-methyl-1H-pyrazol-3-yl)sulfonyl)amide (Intermediate P13) and 4-fluoro-2-isopropyl-6-(pyrimidin-5-yl)aniline (Intermediate A5) to afford the title compound (44 mg, 16%) as a white solid.

¹H NMR (DMSO-d₆, 70° C.) δ 9.03 (s, 1H), 8.76 (s, 2H), 7.30 (bs, 1H), 7.11 (dd, J=10.2, 3.0 Hz, 1H), 7.03 (dd, J=9.0, 3.0 Hz, 1H), 6.43 (s, 1H), 3.85 (s, 3H), 3.26 (sept, J=6.8 Hz, 1H), 3.04 (s, 6H), 1.14 (d, J=6.8 Hz, 6H).

LCMS; m/z 490.4 (M+H)⁺ (ES⁺).

### Example 43: N-Cyclopropyl-3-(N-((2,6-diisopropylphenyl)carbamoyl) sulfamoyl)-N,1-dimethyl-1H-pyrazole-5-carboxamide

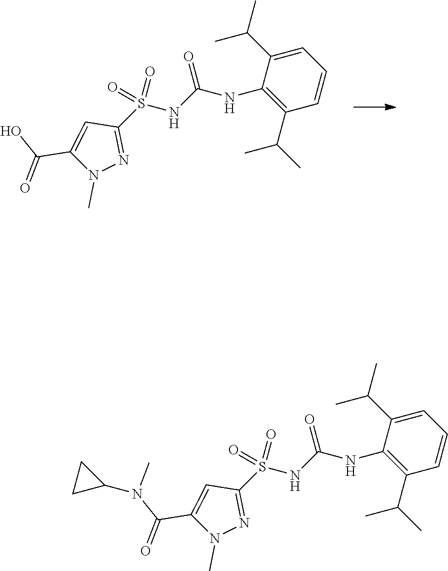 [SMILES: CC(C)c1cccc(C(C)C)c1NC(=O)NS(=O)(=O)c1cc(C(=O)O)n(C)n1 ; CC(C)c1cccc(C(C)C)c1NC(=O)NS(=O)(=O)c1cc(C(=O)N(C)C2CC2)n(C)n1]

Prepared according to the general procedure for 3-(N-((4-chloro-2,6-diisopropylphenyl) carbamoyl)sulfamoyl)-N,1-dimethyl-1H-pyrazole-5-carboxamide (Example 32) from 3-(N-((2,6-diisopropylphenyl)carbamoyl)sulfamoyl)-1-methyl-1H-pyrazole-5-carboxylic acid, disodium salt (Intermediate P9) and N-methylcyclopropanamine, HCl to afford the title compound (45 mg, 55%) as a white solid.

¹H NMR (DMSO-d₆), rotamers; 611.04 (s, 1H), 7.96-7.86 (2×s, 1H), 7.26-7.23 (m, 1H), 7.16-7.11 (m, 3H), 3.95 (s, 3H), 3.09-2.84 (m, 6H), 1.18-1.14 (m, 12H), 0.55-0.52 (m, 4H).

LCMS; m/z 462 (M+H)⁺ (ES⁺).

### Example 44: 3-(N-((2,5-Diisopropylphenyl)carbamoyl)sulfamoyl)-N,N,1-trimethyl-1H-pyrazole-5-carboxamide, Sodium Salt

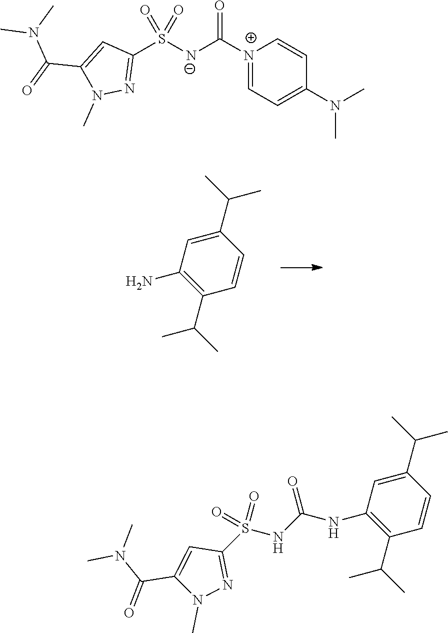

Prepared according to the general procedure for 3-(N-((4-fluoro-2-isopropyl-6-(tetrahydro-2H-pyran-4-yl)phenyl)carbamoyl)sulfamoyl)-N,N,1-trimethyl-1H-pyrazole-5-carboxamide, sodium salt (Example 33) from (4-(dimethylamino)pyridin-1-ium-1-carbonyl)((5-(dimethylcarbamoyl)-1-methyl-1H-pyrazol-3-yl)sulfonyl)amide (Intermediate P13) and 2,5-diisopropylaniline to afford the title compound (8 mg, 6%) as a white solid.

¹H NMR (DMSO-d₆), rotamers; δ 7.51-7.47 (m, 2H), 7.05 (d, J=8.0 Hz, 1H), 6.80 (dd, J=8.0, 2.0 Hz, 1H), 6.62 (s, 1H), 3.83 (s, 3H), 3.04 (m, 4H), 2.99 (s, 3H), 2.75 (sept, J=6.9 Hz, 1H), 1.15 (d, J=6.9 Hz, 6H), 1.10 (d, J=6.8 Hz, 6H).

LCMS; m/z 436.5 (M+H)⁺ (ES⁺).

### Example 45: 5-(Azetidine-1-carbonyl)-N-((4-fluoro-2,6-diisopropylphenyl) carbamoyl)-1-methyl-1H-pyrazole-3-sulfonamide, Sodium Salt

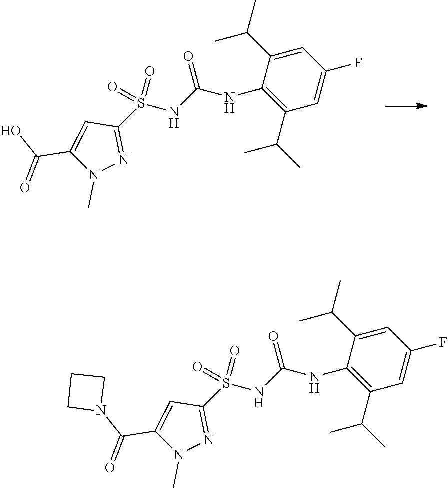 [SMILES: CC(C)c1cc(F)cc(C(C)C)c1NC(=O)NS(=O)(=O)c1cc(C(=O)O)n(C)n1 ; CC(C)c1cc(F)cc(C(C)C)c1NC(=O)NS(=O)(=O)c1cc(C(=O)N2CCC2)n(C)n1]

Azetidine hydrochloride (17 mg, 0.182 mmol), NaHCO₃ (30 mg, 0.357 mmol) and HATU (68 mg, 0.179 mmol) were successively added to a solution of 3-(N-((4-fluoro-2,6-diisopropylphenyl)carbamoyl)sulfamoyl)-1-methyl-1H-pyrazole-5-carboxylic acid, disodium salt (Intermediate P10) (70 mg, 0.149 mmol) in DMF (1 mL) and stirred for 20 hours. The reaction was quenched with water (1 mL) and purified by chromatography on RP Flash C18 (12 g column, 5-50% MeCN/10 mM ammonium bicarbonate) to afford the free acid (59 mg, 84%) as a white solid. The sodium salt was generated by dissolving the free acid (55 mg, 0.12 mmol) in THF (3 mL) and adding a 2 M solution of sodium tert-butoxide (63 μL, 0.126 mmol) in THF. The suspension was stirred for 30 minutes and filtered. The collected solid was washed with EtOAc (2 mL), slurried in MeCN (3 mL), filtered and dried under vacuum to afford the title compound (29 mg, 40%) as a white solid.

¹H NMR (DMSO-d₆) δ 7.33 (s, 1H), 6.79 (d, J=10.1 Hz, 2H), 6.67 (s, 1H), 4.29 (t, J=7.7 Hz, 2H), 4.03 (t, J=7.7 Hz, 2H), 3.98 (s, 3H), 3.11 (m, 2H), 2.27 (p, J=7.7 Hz, 2H), 1.02 (d, J=7.7 Hz, 12H).

LCMS; m/z 466.4 (M+H)⁺ (ES⁺); 464.3 (M−H)⁻ (ES⁻).

### Example 46: 3-(N-((1,2,3,5,6,7-Hexahydro-s-indacen-4-yl)carbamoyl) sulfamoyl)-N-isopropyl-1-methyl-1H-pyrazole-5-carboxamide

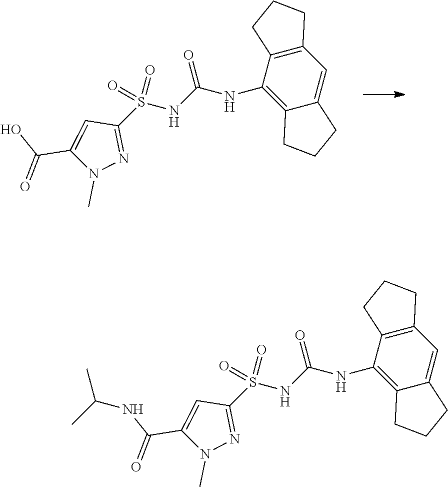 [SMILES: Cn1nc(S(=O)(=O)NC(=O)Nc2c3c(cc4c2CCC4)CCC3)cc1C(=O)O ; CC(C)NC(=O)c1cc(S(=O)(=O)NC(=O)Nc2c3c(cc4c2CCC4)CCC3)nn1C]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((1,2,3,5,6,7-hexahydro-s-indacen-4-yl)carbamoyl)sulfamoyl)-1-methyl-1H-pyrazole-5-carboxylic Acid, Disodium Salt (Intermediate P8) and isopropylamine to afford the title compound (14 mg, 24%) as a white solid.

¹H NMR (DMSO-d₆) δ 10.94 (s, 1H), 8.50 (d, J=7.8 Hz, 1H), 8.02 (s, 1H), 7.46 (s, 1H), 6.94 (s, 1H), 4.13 (s, 3H), 4.09-3.98 (m, 1H), 2.79 (t, J=7.4 Hz, 4H), 2.60 (t, J=7.4 Hz, 4H), 1.98-1.91 (m, 4H), 1.14 (d, J=6.6 Hz, 6H).

LCMS; m/z 446 (M+H)⁺ (ES⁺).

### Example 47: 3-(N-((4-Fluoro-2,6-diisopropylphenyl)carbamoyl)sulfamoyl)-N-isopropyl-N,1-dimethyl-1H-pyrazole-5-carboxamide

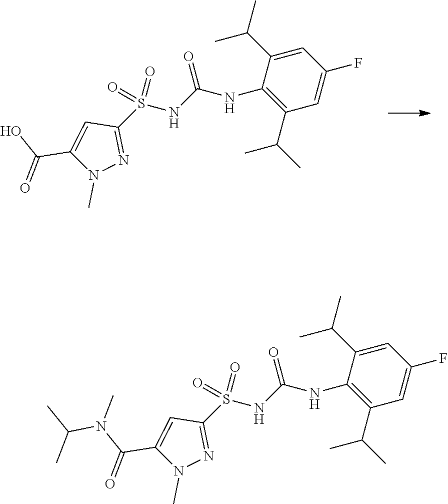 [SMILES: CC(C)c1cc(F)cc(C(C)C)c1NC(=O)NS(=O)(=O)c1cc(C(=O)O)n(C)n1 ; CC(C)c1cc(F)cc(C(C)C)c1NC(=O)NS(=O)(=O)c1cc(C(=O)N(C)C(C)C)n(C)n1]

Prepared according to the general procedure for 5-(azetidine-1-carbonyl)-N-((4-fluoro-2,6-diisopropylphenyl)carbamoyl)-1-methyl-1H-pyrazole-3-sulfonamide, sodium salt (Example 45) from 3-(N-((4-fluoro-2,6-diisopropylphenyl)carbamoyl)sulfamoyl)-1-methyl-1H-pyrazole-5-carboxylic acid, disodium salt (Intermediate P10) and N-methylpropan-2-amine to afford the title compound (33 mg, 45%) as a white solid. ¹H NMR (DMSO-d₆), rotamers; 611.18 (bs, 1H), 7.82 (s, 1H), 6.92 & 6.80 (2×s, 1H), 6.90 (d, J=10.0 Hz, 2H), 4.67 & 3.96 (2×m, 1H), 3.89 & 3.87 (2×s, 3H), 2.99 (m, 2H), 2.85 & 2.82 (2×s, 3H), 1.14 (d, J=6.7 Hz, 6H), 1.04 (bs, 12H).

LCMS; m/z 482.4 (M+H)⁺ (ES⁺); 480.3 (M−H)⁻ (ES⁻).

### Example 48: N,N-Diethyl-3-(N-((4-fluoro-2,6-diisopropylphenyl) carbamoyl)sulfamoyl)-1-methyl-1H-pyrazole-5-carboxamide, Sodium Salt

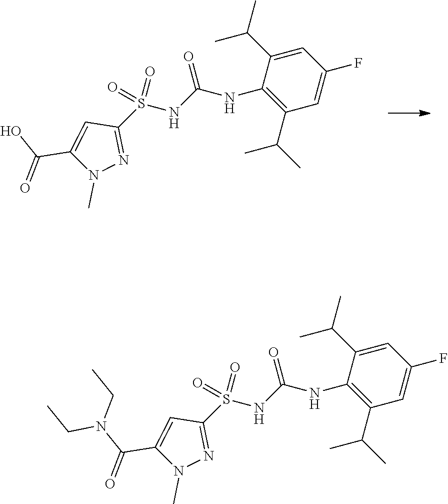 [SMILES: CC(C)c1cc(F)cc(C(C)C)c1NC(=O)NS(=O)(=O)c1cc(C(=O)O)n(C)n1 ; CCN(CC)C(=O)c1cc(S(=O)(=O)NC(=O)Nc2c(C(C)C)cc(F)cc2C(C)C)nn1C]

Prepared according to the general procedure for 5-(azetidine-1-carbonyl)-N-((4-fluoro-2,6-diisopropylphenyl)carbamoyl)-1-methyl-1H-pyrazole-3-sulfonamide, sodium salt (Example 45) from 3-(N-((4-fluoro-2,6-diisopropylphenyl)carbamoyl)sulfamoyl)-1-methyl-1H-pyrazole-5-carboxylic acid, disodium salt (Intermediate P10) and diethylamine to afford the title compound (26 mg, 34%) as a white solid.

¹H NMR (DMSO-d₆), rotamers; δ 7.35 (s, 1H), 6.79 (d, J=10.0 Hz, 2H), 6.51 (s, 1H), 3.77 (s, 3H), 3.50-3.28 (m, 4H), 3.14 (m, 2H), 1.12 (bs, 6H), 1.03 (d, J=6.8 Hz, 12H). LCMS; m/z 482.4 (M+H)⁺ (ES⁺); 480.4 (M−H)⁻ (ES⁻).

### Example 49: N-Ethyl-3-(N-((4-fluoro-2,6-diisopropylphenyl)carbamoyl) sulfamoyl)-N,1-dimethyl-1H-pyrazole-5-carboxamide, Sodium Salt

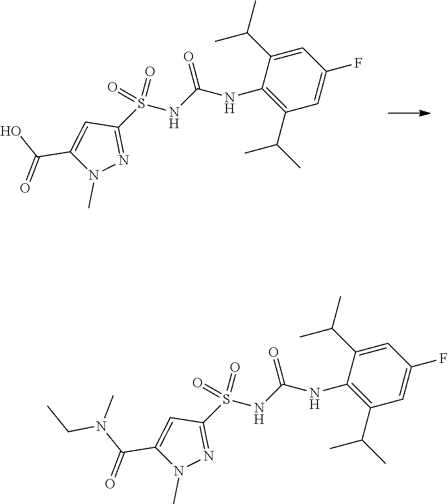 [SMILES: CC(C)c1cc(F)cc(C(C)C)c1NC(=O)NS(=O)(=O)c1cc(C(=O)O)n(C)n1 ; CCN(C)C(=O)c1cc(S(=O)(=O)NC(=O)Nc2c(C(C)C)cc(F)cc2C(C)C)nn1C]

Prepared according to the general procedure for 5-(azetidine-1-carbonyl)-N-((4-fluoro-2,6-diisopropylphenyl)carbamoyl)-1-methyl-1H-pyrazole-3-sulfonamide, sodium salt (Example 45) from 3-(N-((4-fluoro-2,6-diisopropylphenyl)carbamoyl)sulfamoyl)-1-methyl-1H-pyrazole-5-carboxylic acid, disodium salt (Intermediate P10) and N-methylethanamine to afford the title compound (24 mg, 33%) as a white solid.

¹H NMR (DMSO-d₆), rotamers; δ 7.34 (s, 1H), 6.79 (d, J=10.1 Hz, 2H), 6.58 & 6.52 (2×s, 1H), 3.80 (s, 3H), 3.52-3.35 (m, 2H), 3.14 (m, 2H), 3.0 & 2.96 (2×s, 3H), 1.12 (t, J=7.1 Hz, 3H), 1.03 (d, J=6.9 Hz, 12H).

LCMS; m/z 468.4 (M+H)⁺ (ES⁺); 466.3 (M−H)⁻ (ES⁻).

### Example 50: 3-(N-((4-Fluoro-2,6-diisopropylphenyl)carbamoyl) sulfamoyl)-N,1-dimethyl-1H-pyrazole-5-carboxamide

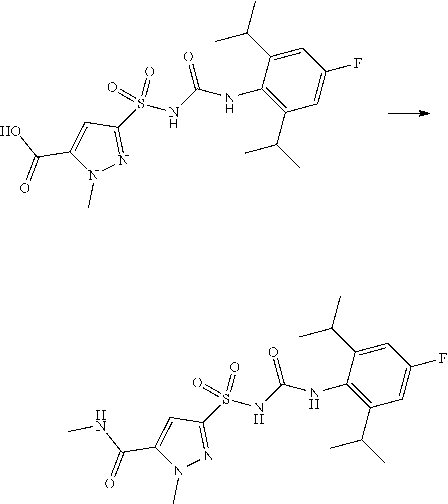 [SMILES: CC(C)c1cc(F)cc(C(C)C)c1NC(=O)NS(=O)(=O)c1cc(C(=O)O)n(C)n1 ; CNC(=O)c1cc(S(=O)(=O)NC(=O)Nc2c(C(C)C)cc(F)cc2C(C)C)nn1C]

Prepared according to the general procedure for 5-(azetidine-1-carbonyl)-N-((4-fluoro-2,6-diisopropylphenyl)carbamoyl)-1-methyl-1H-pyrazole-3-sulfonamide, sodium salt (Example 45) from 3-(N-((4-fluoro-2,6-diisopropylphenyl)carbamoyl)sulfamoyl)-1-methyl-1H-pyrazole-5-carboxylic acid, disodium salt (Intermediate P10) and methylamine to afford the title compound (26 mg, 39%) as a white solid.

¹H NMR (DMSO-d₆) δ 11.16 (bs, 1H), 8.62 (q, J=4.6 Hz, 1H), 7.76 (s, 1H), 7.28 (s, 1H), 6.89 (d, J=10.0 Hz, 2H), 4.12 (s, 3H), 2.95 (sept, J=6.4 Hz, 2H),2.74 (d, J=4.6 Hz, 3H), 1.02 (bs, 12H).

LCMS; m/z 440.4 (M+H)⁺ (ES⁺); 438.4 (M−H)⁻ (ES⁻).

### Example 51:5-(Azetidine-1-carbonyl)-N-((2,6-diisopropylphenyl) carbamoyl)-1-methyl-1H-pyrazole-3-sulfonamide

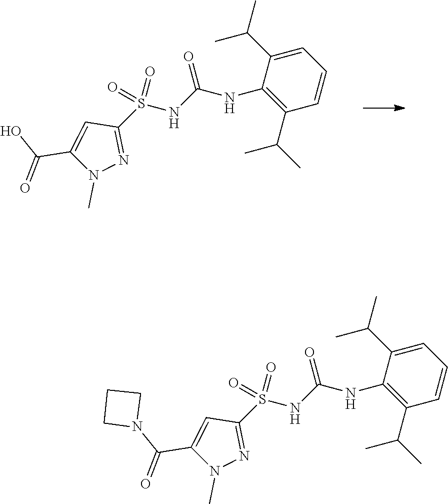 [SMILES: CC(C)c1cccc(C(C)C)c1NC(=O)NS(=O)(=O)c1cc(C(=O)O)n(C)n1 ; CC(C)c1cccc(C(C)C)c1NC(=O)NS(=O)(=O)c1cc(C(=O)N2CCC2)n(C)n1]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((2,6-diisopropylphenyl)carbamoyl)sulfamoyl)-1-methyl-1H-pyrazole-5-carboxylic acid, disodium salt (Intermediate P9) and azetidine, HCl to afford the title compound (6 mg, 8%) as a white solid.

¹H NMR (DMSO-d₆) δ 7.38 (s, 1H), 7.11 (dd, J=8.4, 6.8 Hz, 1H), 7.06-6.99 (m, 2H), 6.71 (s, 1H), 4.29 (t, J=7.7 Hz, 2H), 4.07-3.97 (m, 5H), 3.12-3.08 (m, 2H), 2.31-2.22 (m, 2H), 1.04 (d, J=6.8 Hz, 12H). Acidic NH not observed.

LCMS; m/z 448 (M+H)⁺ (ES⁺).

### Example 52: N-Cyclopropyl-3-(N-((1,2,3,5,6,7-hexahydro-s-indacen-4-yl) carbamoyl)sulfamoyl)-N,1-dimethyl-1H-pyrazole-5-carboxamide

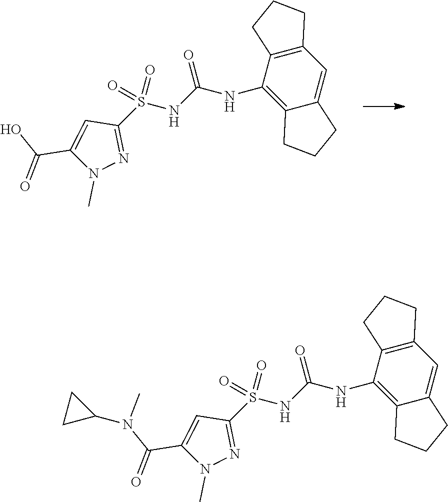 [SMILES: Cn1nc(S(=O)(=O)NC(=O)Nc2c3c(cc4c2CCC4)CCC3)cc1C(=O)O ; CN(C(=O)c1cc(S(=O)(=O)NC(=O)Nc2c3c(cc4c2CCC4)CCC3)nn1C)C1CC1]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((1,2,3,5,6,7-hexahydro-s-indacen-4-yl)carbamoyl) sulfamoyl)-1-methyl-1H-pyrazole-5-carboxylic acid, disodium salt (Intermediate P8) and N-methylcyclopropanamine, HCl to afford the title compound (28 mg, 34%) as a white solid.

¹H NMR (DMSO-d₆) δ 10.90 (br s, 1H), 8.06 (s, 1H), 7.16 (br s, 1H), 6.95 (s, 1H), 3.94 (s, 3H), 2.99 (s, 3H), 2.79 (t, J=7.4 Hz, 4H), 2.59 (t, J=7.4 Hz, 4H), 1.99-1.91 (m, 4H), 0.60-0.50 (m, 4H). One exchangeable proton not observed.

LCMS; m/z 458 (M+H)⁺ (ES⁺).

### Example 53: N-(Cyanomethyl)-3-(N-((1,2,3,5,6,7-hexahydro-s-indacen-4-yl) carbamoyl)sulfamoyl)-N,1-dimethyl-1H-pyrazole-5-carboxamide, Partial Ammonium Salt

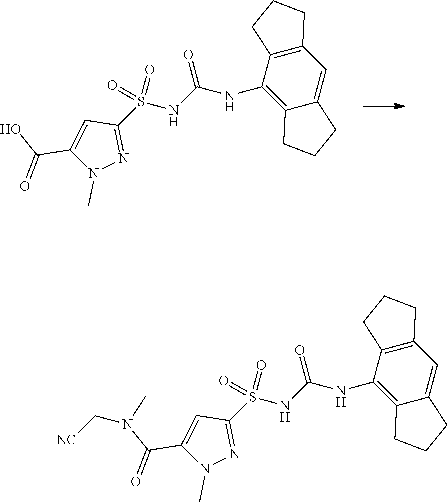 [SMILES: Cn1nc(S(=O)(=O)NC(=O)Nc2c3c(cc4c2CCC4)CCC3)cc1C(=O)O ; CN(CC#N)C(=O)c1cc(S(=O)(=O)NC(=O)Nc2c3c(cc4c2CCC4)CCC3)nn1C]

Prepared according to the general procedure for 5-(azetidine-1-carbonyl)-N-((4-fluoro-2,6-diisopropylphenyl)carbamoyl)-1-methyl-1H-pyrazole-3-sulfonamide, sodium salt (Example 45) from 3-(N-((1,2,3,5,6,7-hexahydro-s-indacen-4-yl)carbamoyl) sulfamoyl)-1-methyl-H-pyrazole-5-carboxylic acid, disodium salt (Intermediate P8) and N 2-(methylamino)acetonitrile, HCl to afford the title compound (33 mg, 46%) as a white solid.

¹H NMR (DMSO-d₆) δ 7.75 (s, 1H), 6.91 (s, 1H), 6.84 (s, 1H), 4.58 (s, 2H), 3.90 (s, 3H), 3.14 (s, 3H), 2.77 (t, J=7.4 Hz, 4H), 2.63 (t, J=7.4 Hz, 4H), 1.92 (quin, J=7.4 Hz, 4H).

LCMS; m/z 457.4 (M+H)⁺ (ES⁺); 455.3 (M−H)⁻ (ES⁻).

### Example 54: 5-(Azetidine-1-carbonyl)-N-((1,2,3,5,6,7-hexahydro-s-indacen-4-yl)carbamoyl)-1-methyl-1H-pyrazole-3-sulfonamide

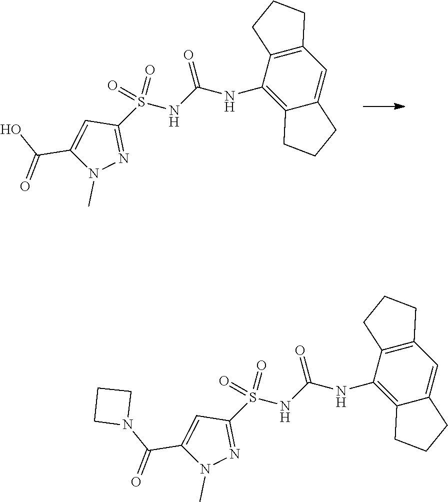 [SMILES: Cn1nc(S(=O)(=O)NC(=O)Nc2c3c(cc4c2CCC4)CCC3)cc1C(=O)O ; Cn1nc(S(=O)(=O)NC(=O)Nc2c3c(cc4c2CCC4)CCC3)cc1C(=O)N1CCC1]

Prepared according to the general procedure for 5-(azetidine-1-carbonyl)-N-((4-fluoro-2,6-diisopropylphenyl)carbamoyl)-1-methyl-1H-pyrazole-3-sulfonamide, sodium salt (Example 45) from 3-(N-((1,2,3,5,6,7-hexahydro-s-indacen-4-yl)carbamoyl) sulfamoyl)-1-methyl-H-pyrazole-5-carboxylic acid, disodium salt (Intermediate P8) and azetidine, HCl to afford the title compound (40 mg, 55%), as a white solid.

¹H NMR (DMSO-d₆) δ 10.93 (bs, 1H), 8.09 (s, 1H), 7.06 (s, 1H), 6.95 (s, 1H), 4.34 (t, J=7.7 Hz, 2H), 4.08 (s, 3H), 4.05 (t, J=7.7 Hz, 2H), 2.79 (t, J=7.4 Hz, 4H), 2.60 (t, J=7.3 Hz, 4H), 2.33-2.20 (m, 2H), 1.95 (quin, J=7.4 Hz, 4H).

LCMS; m/z 444.5 (M+H)⁺ (ES⁺); 442.3 (M−H)⁻ (ES⁻).

### Example 5:3-(N-((2,6-Diisopropylphenyl)carbamoyl)sulfamoyl)-N,1-dimethyl-1H-pyrazole-5-carboxamide

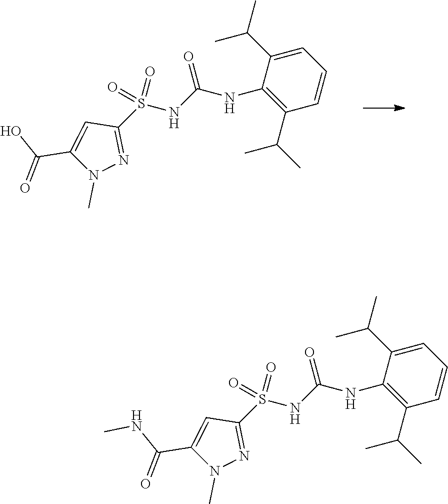 [SMILES: CC(C)c1cccc(C(C)C)c1NC(=O)NS(=O)(=O)c1cc(C(=O)O)n(C)n1 ; CNC(=O)c1cc(S(=O)(=O)NC(=O)Nc2c(C(C)C)cccc2C(C)C)nn1C]

Methylamine in THF (108 μL, 0.217 mmol) and HATU (82 mg, 0.217 mmol) were successively added to a solution of 3-(N-((2,6-diisopropylphenyl)carbamoyl) sulfamoyl)-1-methyl-H-pyrazole-5-carboxylic acid, disodium salt (Intermediate P9) (70 mg, 0.155 mmol) in DMF (1 mL) and stirred for 20 hours. The reaction was quenched with water (0.1 mL) and purified by reversed phase prep-HPLC (General Methods, basic prep) to afford the title compound (26 mg, 40%) as a white solid.

¹H NMR (DMSO-d₆) δ 11.07 (bs, 1H), 8.62 (d, J=5.1 Hz, 1H), 7.74 (s, 1H), 7.28 (s, 1H), 7.21 (t, J=7.7 Hz, 1H), 7.09 (d, J=7.7 Hz, 2H), 4.13 (s, 3H), 2.96 (m, 2H), 2.73 (d, J=4.5 Hz, 3H), 1.06-1.00 (m, 12H).

LCMS; m/z 422.4 (M+H)⁺ (ES⁺); 420.4 (M−H)⁻ (ES⁻).

### Example 56:3-(N-((2,6-Diisopropylphenyl)carbamoyl)sulfamoyl)-N-ethyl-N,1-dimethyl-1H-pyrazole-5-carboxamide

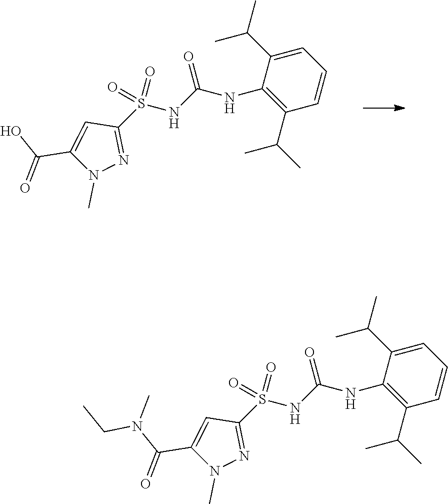 [SMILES: CC(C)c1cccc(C(C)C)c1NC(=O)NS(=O)(=O)c1cc(C(=O)O)n(C)n1 ; CCN(C)C(=O)c1cc(S(=O)(=O)NC(=O)Nc2c(C(C)C)cccc2C(C)C)nn1C]

Prepared according to the general procedure for 3-(N-((2,6-diisopropylphenyl) carbamoyl)sulfamoyl)-N,1-dimethyl-1H-pyrazole-5-carboxamide (Example 55) from 3-(N-((2,6-diisopropylphenyl)carbamoyl)sulfamoyl)-1-methyl-1H-pyrazole-5-carboxylic acid, disodium salt (Intermediate P9) and N-methylethanamine to afford the title (10 mg, 14%) as a white solid.

¹H NMR (DMSO-d₆), rotamers; δ 11.09 (bs, 1H), 7.81 (s, 1H), 7.23 (t, J=7.7 Hz, 1H), 7.11 (d, J=7.6 Hz, 2H), 6.96 & 6.87 (2×s, 1H), 3.92 & 3.90 (2×s, 3H), 3.46 & 3.31 (2×q, J=7.1 Hz, 2H),2.98 (brs, 5H), 1.16-1.0 (m, 15H).

LCMS; m/z 450.5 (M+H)⁺ (ES⁺); 448.4 (M−H)⁻ (ES⁻).

### Example 57: 3-(N-((2,6-Diisopropylphenyl)carbamoyl)sulfamoyl)-N,N-diethyl-1-methyl-1H-pyrazole-5-carboxamide

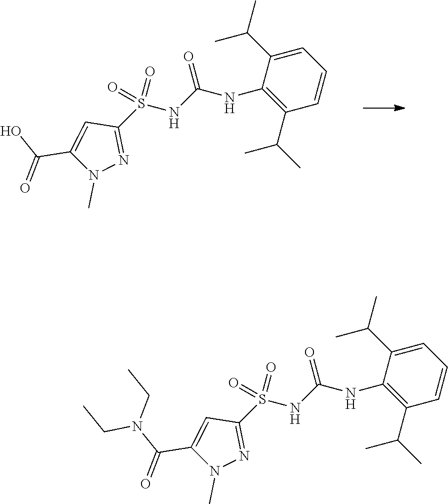 [SMILES: CC(C)c1cccc(C(C)C)c1NC(=O)NS(=O)(=O)c1cc(C(=O)O)n(C)n1 ; CCN(CC)C(=O)c1cc(S(=O)(=O)NC(=O)Nc2c(C(C)C)cccc2C(C)C)nn1C]

Prepared according to the general procedure for 3-(N-((2,6-diisopropylphenyl) carbamoyl)sulfamoyl)-N,1-dimethyl-1H-pyrazole-5-carboxamide (Example 55) from 3-(N-((2,6-diisopropylphenyl)carbamoyl)sulfamoyl)-1-methyl-1H-pyrazole-5-carboxylic acid, disodium salt (Intermediate P9) and diethylamine to afford the title compound (33 mg, 45%) as a white solid.

¹H NMR (DMSO-d₆), rotamers; δ 11.09 (bs, 1H), 7.83 (s, 1H), 7.23 (t, J=7.7 Hz, 1H), 7.11 (d, J=7.7 Hz, 2H), 6.87 (s, 1H), 3.88 (s, 3H), 3.45 (m, 2H), 3.31 (m, 2H), 2.98 (sept, J=6.8 Hz, 2H), 1.20-0.95 (m, 18H).

LCMS; m/z 464.5 (M+H)⁺ (ES⁺); 462.4 (M−H)⁻ (ES⁻).

### Example 58: 3-(N-((2,6-Diisopropylphenyl)carbamoyl)sulfamoyl)-N-isopropyl-N,1-dimethyl-1H-pyrazole-5-carboxamide

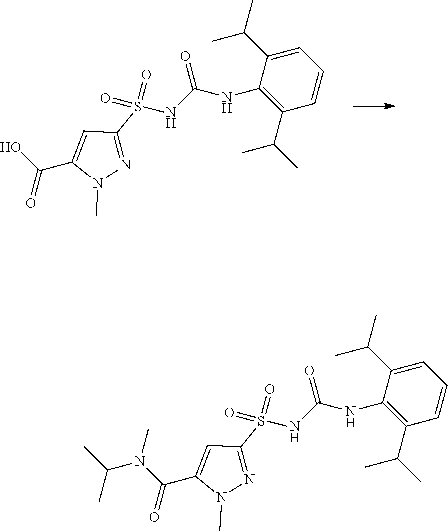 [SMILES: CC(C)c1cccc(C(C)C)c1NC(=O)NS(=O)(=O)c1cc(C(=O)O)n(C)n1 ; CC(C)c1cccc(C(C)C)c1NC(=O)NS(=O)(=O)c1cc(C(=O)N(C)C(C)C)n(C)n1]

Prepared according to the general procedure for 3-(N-((2,6-diisopropylphenyl) carbamoyl)sulfamoyl)-N,1-dimethyl-1H-pyrazole-5-carboxamide (Example 55) from 3-(N-((2,6-diisopropylphenyl)carbamoyl)sulfamoyl)-1-methyl-1H-pyrazole-5-carboxylic acid, disodium salt (Intermediate P9) and N-methylpropan-2-amine to afford the title compound (47 mg, 8%) as a white solid.

¹H NMR (DMSO-d₆), rotamers; δ 11.13 (br s, 1H), 7.91 (s, 1H), 7.30-7.19 (m, 1H), 7.12 (d, J=7.7 Hz, 2H), 7.02 (s, 0.5H), 6.90 (s, 0.5H), 4.74-4.57 (m, 1H), 3.92-3.78 (m, 3H), 2.95-2.93 (m, 2H), 2.85-2.82 (m, 3H), 1.11-1.08 (m, 18H).

LCMS; m/z 464 (M+H)⁺ (ES⁺).

### Example 59: N-((1,2,3,5,6,7-Hexahydro-s-indacen-4-yl)carbamoyl)-1-methyl-5-(pyrrolidine-1-carbonyl)-1H-pyrazole-3-sulfonamide

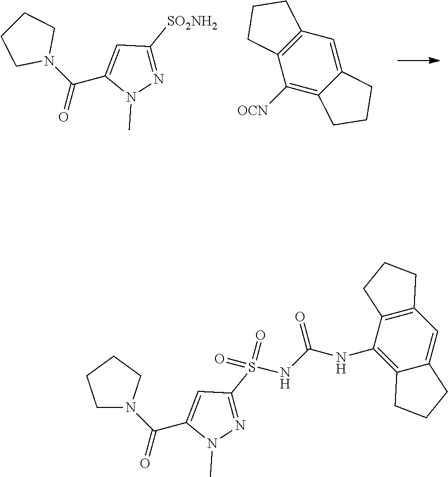

Prepared according to the general procedure for 5-(azetidine-1-carbonyl)-N-((4-fluoro-2,6-diisopropylphenyl) carbamoyl)-1-isopropyl-1H-pyrazole-3-sulfonamide (Example 30) from 1-methyl-5-(pyrrolidine-1-carbonyl)-1H-pyrazole-3-sulfonamide (Intermediate P7) and 4-isocyanato-1,2,3,5,6,7-hexahydro-s-indacene (Intermediate A1). The crude product was purified by chromatography on RP Flash C18 (basic) to afford the title compound (25 mg, 28%) as a white solid.

¹H NMR (DMSO-d₆), rotamers; δ 10.9 (br s, 1H), 7.99 (s, 1H), 7.06 (s, 1H), 6.91 (s, 1H), 3.98 (s, 3H), 3.52 and 3.47 (2×t, J=6.2 Hz, 4H),2.78 (t, J=7.4 Hz, 4H), 2.61 (t, J=7.4 Hz, 4H), 2.02-1.75 (m, 8H).

LCMS; m/z 458 (M+H)⁺ (ES⁺).

### Example 60: 3-(N-((4-Fluoro-2,6-diisopropylphenyl)carbamoyl) sulfamoyl)-N,N,1-trimethyl-1H-pyrazole-5-carboxamide, Sodium Salt

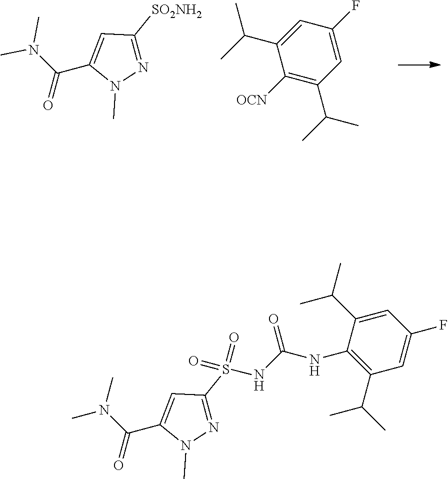

Prepared according to the general procedure for 5-(azetidine-1-carbonyl)-N-((4-fluoro-2,6-diisopropylphenyl) carbamoyl)-1-isopropyl-1H-pyrazole-3-sulfonamide (Example 30) from N,N,1-trimethyl-3-sulfamoyl-1H-pyrazole-5-carboxamide (Intermediate P5) and 5-fluoro-2-isocyanato-1,3-diisopropylbenzene (Intermediate A2) to afford the title compound (mg, 67%) as a white solid.

¹H NMR (DMSO-d₆), rotamers; δ 7.35 (s, 1H), 6.79 (d, J=10.1 Hz, 2H), 6.59 (s, 1H), 3.81 (s, 3H), 3.18-3.07 (m, 2H), 3.03 (s, 3H), 2.98 (s, 3H), 1.02 (d, J=6.9 Hz, 12H).

LCMS; m/z 454 (M+H)⁺ (ES⁺).

### Example 61: 5-(3-Fluoroazetidine-1-carbonyl)-N-((1,2,3,5,6,7-hexahydro-s-indacen-4-yl)carbamoyl)-1-methyl-1H-pyrazole-3-sulfonamide, Partial Ammonium Salt

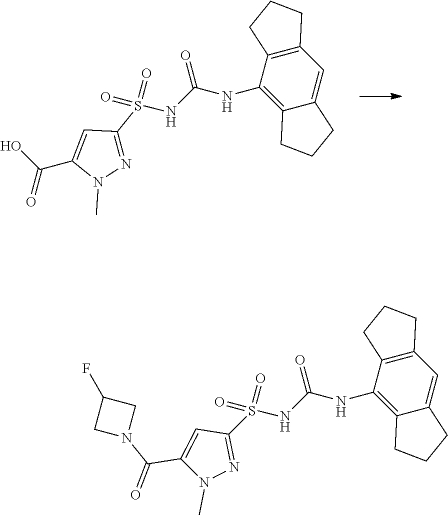 [SMILES: Cn1nc(S(=O)(=O)NC(=O)Nc2c3c(cc4c2CCC4)CCC3)cc1C(=O)O ; Cn1nc(S(=O)(=O)NC(=O)Nc2c3c(cc4c2CCC4)CCC3)cc1C(=O)N1CC(F)C1]

Prepared according to the general procedure for 5-(azetidine-1-carbonyl)-N-((4-fluoro-2,6-diisopropylphenyl) carbamoyl)-1-methyl-1H-pyrazole-3-sulfonamide, sodium salt (Example 45) from 3-(N-((1,2,3,5,6,7-hexahydro-s-indacen-4-yl)carbamoyl) sulfamoyl)-1-methyl-1H-pyrazole-5-carboxylic acid, disodium salt (Intermediate P8) and 3-fluoroazetidine, HCl to afford the title compound (36 mg, 50%) as a white solid. ¹H NMR (DMSO-d₆), rotamers; δ 7.74 (s, 1H), 6.87 (s, 1H), 6.78 (s, 1H), 5.45-5.26 (m, 1H), 4.61-4.47 (m, 1H), 4.44-4.22 (m, 2H), 4.08-3.95 (m, 1H), 3.95 (s, 3H), 2.70 (t, J=7.4 Hz, 4H), 2.55 (t, J=7.4 Hz, 4H), 1.85 (quin, J=7.5 Hz, 4H).

LCMS; m/z 462.4 (M+H)⁺ (ES⁺); 460.4 (M−H)⁻ (ES⁻).

### Example 62: N-Ethyl-3-(N-((1,2,3,5,6,7-hexahydro-s-indacen-4-yl) carbamoyl)sulfamoyl)-N,1-dimethyl-1H-pyrazole-5-carboxamide

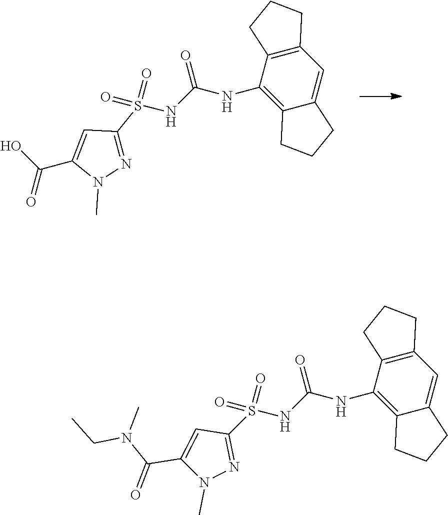 [SMILES: Cn1nc(S(=O)(=O)NC(=O)Nc2c3c(cc4c2CCC4)CCC3)cc1C(=O)O ; CCN(C)C(=O)c1cc(S(=O)(=O)NC(=O)Nc2c3c(cc4c2CCC4)CCC3)nn1C]

Prepared according to the general procedure for 5-(azetidine-1-carbonyl)-N-((4-fluoro-2,6-diisopropylphenyl) carbamoyl)-1-methyl-1H-pyrazole-3-sulfonamide, sodium salt (Example 45) from 3-(N-((1,2,3,5,6,7-hexahydro-s-indacen-4-yl)carbamoyl) sulfamoyl)-1-methyl-1H-pyrazole-5-carboxylic acid, disodium salt (Intermediate P8) and N-methylethanamine to afford the title (31 mg, 44%) as a white solid.

¹H NMR (DMSO-d₆), rotamers; δ 10.98 (s, 1H), 8.00 (s, 1H), 6.96-6.87 (m, 2H), 3.90 & 3.88 (2×s, 3H), 3.47 and 3.33 (2×q, J=7.0 Hz, 2H), 3.00 and 2.97 (2×s, 3H), 2.78 (t, J=7.4 Hz, 4H), 2.60 (t, J=7.4 Hz, 4H), 1.94 (p, J=7.4 Hz, 4H), 1.15-1.08 (m, 3H). LCMS; m/z 446.4 (M+H)⁺ (ES⁺); 444.4 (M−H)⁻ (ES⁻).

### Example 63: N-((2,6-Diisopropylphenyl)carbamoyl)-1-methyl-5-(pyrrolidine-1-carbonyl)-1H-pyrazole-3-sulfonamide

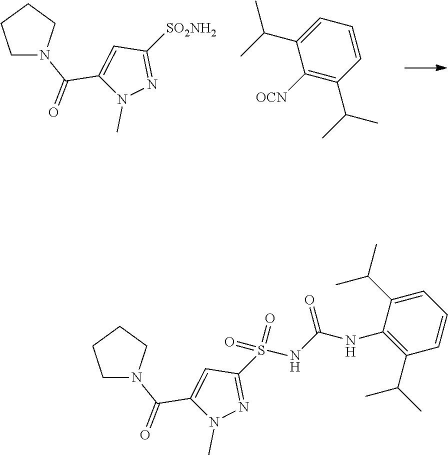

Prepared according to the general procedure for 5-(azetidine-1-carbonyl)-N-((4-fluoro-2,6-diisopropylphenyl) carbamoyl)-1-isopropyl-1H-pyrazole-3-sulfonamide (Example 30) from 1-methyl-5-(pyrrolidine-1-carbonyl)-1H-pyrazole-3-sulfonamide (Intermediate P7) and 2-isocyanato-1,3-diisopropylbenzene (Intermediate A9). The crude product was purified by chromatography on RP Flash C18 (12 g column, 5-50% MeCN/10 mM ammonium bicarbonate) to afford the title compound (40 mg, 43%) as a colourless solid.

¹H NMR (DMSO-d₆), rotamers; δ 11.11 (s, 1H), 7.89 (s, 1H), 7.30-7.18 (m, 1H), 7.16-7.07 (m, 3H), 4.01 (s, 3H), 3.57-3.40 (m, 4H), 3.03-2.85 (m, 2H), 1.91-1.78 (m, 4H), 1.04 (br s, 12H).

LCMS; m/z 462.5 (M+H)⁺ (ES⁺).

### Example 64: 3-(N-((1,2,3,5,6,7-Hexahydro-s-indacen-4-yl)carbamoyl) sulfamoyl)-N-isopropyl-N,1-dimethyl-1H-pyrazole-5-carboxamide

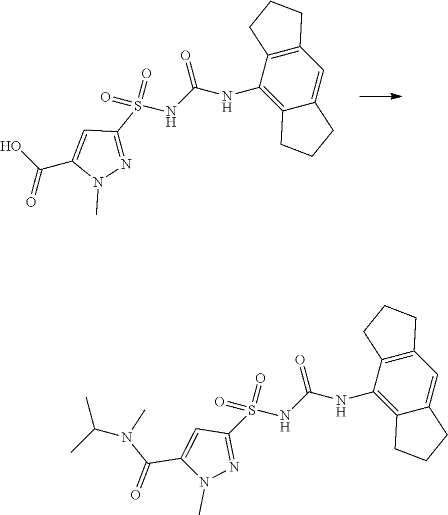 [SMILES: Cn1nc(S(=O)(=O)NC(=O)Nc2c3c(cc4c2CCC4)CCC3)cc1C(=O)O ; CC(C)N(C)C(=O)c1cc(S(=O)(=O)NC(=O)Nc2c3c(cc4c2CCC4)CCC3)nn1C]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((1,2,3,5,6,7-hexahydro-s-indacen-4-yl)carbamoyl)sulfamoyl)-1-methyl-1H-pyrazole-5-carboxylic acid (Intermediate P6) and N-methylpropan-2-amine to afford the title compound (25 mg, 42%) as a white solid.

¹H NMR (DMSO-d₆), rotamers; δ 7.74 (s, 1H), 6.85 (s, 1H), 6.78 & 6.67 (2×s, 1H), 4.68 & 4.03 (s, 1H), 3.83 (s, 3H), 2.85 (s, 3H), 2.77 (t, J=7.4 Hz, 4H), 2.63 (t, J=7.4 Hz, 4H), 1.92 (quin, J=7.4 Hz, 4H), 1.15 (d, J=6.7 Hz, 6H). Acidic proton not observed. LCMS; m/z 460.5 (M+H)⁺ (ES⁺); 458.3 (M−H)⁻ (ES⁻).

### Example 65: N,N-Diethyl-3-(N-((1,2,3,5,6,7-hexahydro-s-indacen-4-yl) carbamoyl)sulfamoyl)-1-methyl-1H-pyrazole-5-carboxamide

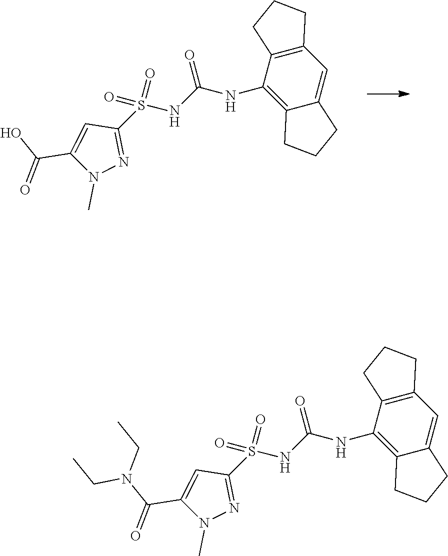 [SMILES: Cn1nc(S(=O)(=O)NC(=O)Nc2c3c(cc4c2CCC4)CCC3)cc1C(=O)O ; CCN(CC)C(=O)c1cc(S(=O)(=O)NC(=O)Nc2c3c(cc4c2CCC4)CCC3)nn1C]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((1,2,3,5,6,7-hexahydro-s-indacen-4-yl)carbamoyl) sulfamoyl)-1-methyl-1H-pyrazole-5-carboxylic acid (Intermediate P6) and diethylamine to afford the title compound (35 mg, 58%) as a white solid.

¹H NMR (DMSO-d₆), rotamers; δ 10.96 (s, 1H), 7.92 (s, 1H), 6.90 (s, 1H), 6.83 (s, 1H), 3.86 (s, 3H), 3.45 & 3.30 (2×m, 4H),2.78 (t, J=7.3 Hz, 4H), 2.61 (t, J=7.4 Hz, 4H), 1.94 (quin, J=7.4 Hz, 4H), 1.18-1.05 (m, 6H).

LCMS; m/z 460.5 (M+H)⁺ (ES⁺); 458.4 (M−H)⁻ (ES⁻).

### Example 66: 3-(N-((1,2,3,5,6,7-Hexahydro-s-indacen-4-yl)carbamoyl) sulfamoyl)-N,1-dimethyl-1H-pyrazole-5-carboxamide

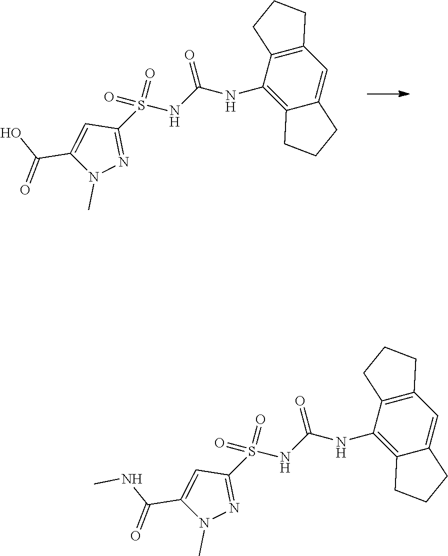 [SMILES: Cn1nc(S(=O)(=O)NC(=O)Nc2c3c(cc4c2CCC4)CCC3)cc1C(=O)O ; CNC(=O)c1cc(S(=O)(=O)NC(=O)Nc2c3c(cc4c2CCC4)CCC3)nn1C]

Prepared according to the general procedure for 3-(N-((4-chloro-2,6-diisopropylphenyl)carbamoyl)sulfamoyl)-N,1-dimethyl-1H-pyrazole-5-carboxamide (Example 32) from 3-(N-((1,2,3,5,6,7-hexahydro-s-indacen-4-yl)carbamoyl) sulfamoyl)-1-methyl-1H-pyrazole-5-carboxylic acid (Intermediate P6) and methylamine to afford the title compound (18 mg, 34%) as a white solid.

¹H NMR (DMSO-d₆) δ 1.88 (s, 1H), 8.61 (q, J=4.6 Hz, 1H), 7.97 (s, 1H), 7.28 (s, 1H), 6.87 (s, 1H), 4.07 (s, 3H), 2.71 (t, J=7.4 Hz, 4H), 2.67 (d, J=4.6 Hz, 3H), 2.52 (t, J=7.4 Hz, 4H), 1.87 (quin, J=7.5 Hz, 4H).

LCMS; m/z 418.4 (M+H)⁺ (ES⁺); 416.3 (M−H)⁻ (ES⁻).

### Example 67: 3-(N-((2,6-Diisopropylphenyl)carbamoyl)sulfamoyl)-N,N,1-trimethyl-1H-pyrazole-5-carboxamide, Sodium Salt

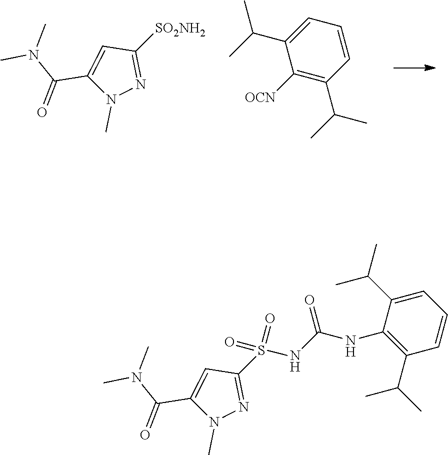

N,N,1-Trimethyl-3-sulfamoyl-1H-pyrazole-5-carboxamide (Intermediate P5) (105 mg, 0.452 mmol) was dissolved in THF (5 mL) and 2 M sodium tert-butoxide in THF (0.237 mL, 0.475 mmol) added. After 1 hour, 2-isocyanato-1,3-diisopropylbenzene (Intermediate A9) (92 mg, 0.452 mmol) was added and the mixture stirred at room temperature for 15 hours. The suspension was filtered and washed with THF (1 mL). The collected solid was triturated with EtOAc (5 mL) for 1 hour, filtered, and dried under vacuum to afford the title compound (137 mg, 64%) as a white solid.

¹H NMR (DMSO-d₆), rotamers; δ 7.37 (br s, 1H), 7.14-7.05 (m, 1H), 7.01 (d, J=7.5 Hz, 2H), 6.61 (s, 1H), 3.81 (s, 3H), 3.15-3.13 (m, 2H), 3.03 (s, 3H), 2.99 (s, 3H), 1.03 (d, J=6.8 Hz, 12H).

LCMS; m/z 436 (M+H)⁺ (ES⁺).

### Example 68:3-(N-((4-Chloro-2,6-diisopropylphenyl)carbamoyl) sulfamoyl)-N,N,1-trimethyl-1H-pyrazole-5-carboxamide

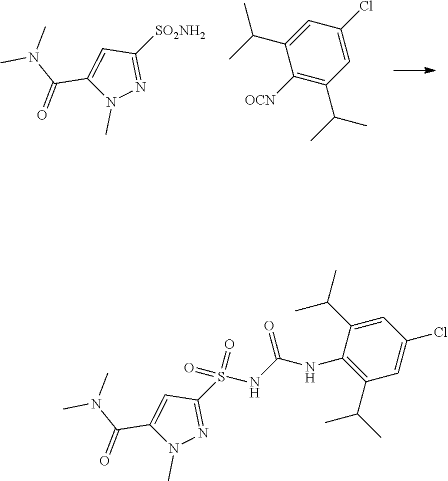

4-Chloro-2,6-diisopropylaniline, HCl (51.9 mg, 0.209 mmol) and triethylamine (0.064 ml, 0.460 mmol) were dissolved in dry THF (5 mL). Triphosgene (49.6 mg, 0.167 mmol) was added to the mixture at room temperature and stirred for 5 hours. The mixture was concentrated in vacuo and dried azeotropically with toluene (1 mL×3). Dry THF (2 mL) was added to the residue and N,N,1-trimethyl-3-sulfamoyl-1H-pyrazole-5-carboxamide (Intermediate P5) (48.6 mg, 0.209 mmol) added to the mixture. After 30 minutes, 60% sodium hydride (20.91 mg, 0.523 mmol) was added and the mixture heated at 60° C. for 15 hours. After cooling to room temperature, saturated aqueous ammonium chloride (10 mL) was added and the mixture extracted with EtOAc (10 mL×3). The combined organic phases were washed with brine (5 mL), dried over MgSO₄, concentrated in vacuo and the residue purified by chromatography on silica gel (25 g column, 5-100% EtOAc/isohexane) to afford the title compound (17 mg, 17%) as a white solid.

¹H NMR (DMSO-d₆), rotamers; δ 11.24 (s, 1H), 7.98 (s, 1H), 7.14 (s, 2H), 7.01 (s, 1H), 3.93 (s, 3H), 3.00 (s, 3H), 2.99 (s, 3H), 2.98-2.91 (m, 2H), 1.17-0.93 (br d, 12H).

LCMS; m/z 469 and 471 (M+H)⁺ (ES⁺).

### Example 6A: 3-(N-((1,2,3,5,6,7-Hexahydro-s-indacen-4-yl)carbamoyl) sulfamoyl)-N,N,1-trimethyl-1H-pyrazole-5-carboxamide, Sodium Salt

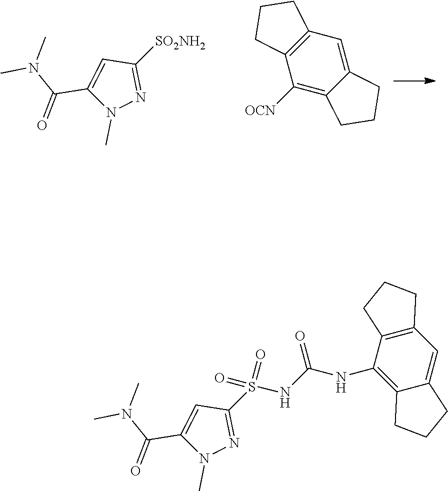

Prepared according to the general procedure for 3-(N-((2,6-diisopropylphenyl) carbamoyl)sulfamoyl)-N,N,1-trimethyl-1H-pyrazole-5-carboxamide, sodium salt (Example 67) from N,N,1-trimethyl-3-sulfamoyl-1H-pyrazole-5-carboxamide (Intermediate P5) and 4-isocyanato-1,2,3,5,6,7-hexahydro-s-indacene (Intermediate A1) to afford the title compound (235 mg, 64%) as a white solid.

¹H NMR (DMSO-d₆), rotamers; δ 7.51 (s, 1H), 6.77 (s, 1H), 6.62 (s, 1H), 3.82 (s, 3H), 3.04 (s, 3H), 2.99 (s, 3H), 2.75 (t, J=7.4 Hz, 4H), 2.65 (t, J=7.4 Hz, 4H), 1.93-1.86 (m, 4H).

LCMS; m/z 432 (M+H)⁺ (ES⁺).

### Example 70: 3-(N-((1,2,3,5,6,7-Hexahydro-s-indacen-4-yl)carbamoyl) sulfamoyl)-N,1-dimethyl-N-(thiazol-2-ylmethyl)-1H-pyrazole-5-carboxamide

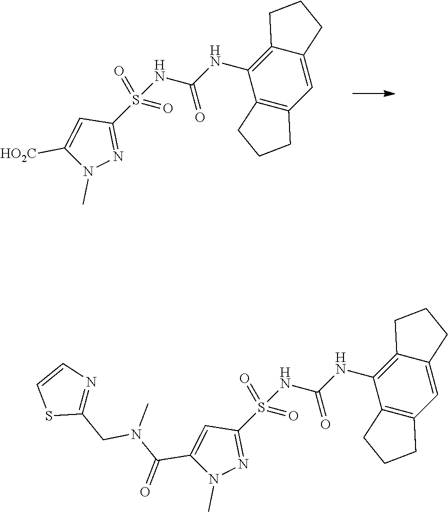 [SMILES: Cn1nc(S(=O)(=O)NC(=O)Nc2c3c(cc4c2CCC4)CCC3)cc1C(=O)O ; CN(Cc1nccs1)C(=O)c1cc(S(=O)(=O)NC(=O)Nc2c3c(cc4c2CCC4)CCC3)nn1C]

Prepared according to the general procedure of N-(2-cyanoethyl)-3-(N-((1,2,3,5,6,7-hexahydro-s-indacen-4-yl)carbamoyl)sulfamoyl)-N,1-dimethyl-1H-pyrazole-5-carboxamide (Example 9) from 3-(N-((1,2,3,5,6,7-hexahydro-s-indacen-4-yl)carbamoyl)sulfamoyl)-1-methyl-1H-pyrazole-5-carboxylic Acid, Disodium Salt (Intermediate P8) and N-methyl-1-(thiazol-2-yl)methanamine to afford the title compound (18 mg, 26%) as a white solid.

¹H NMR (DMSO-d₆), rotamers; δ 10.95 (br s, 1H), 8.09-8.07 (2×s, 1H), 7.89-7.65 (m, 2H), 7.11-7.08 (2×s, 1H), 6.94 (s, 1H), 4.97-4.92 (2×s, 2H), 3.97-3.95 (2×s, 3H), 3.12-3.01 (2×s, 3H),2.78 (t, J=7.6 Hz, 4H), 2.64-2.53 (m, 4H), 1.94-1.92 (m, 4H).

LCMS; m/z 515 (M+H)⁺ (ES⁺).

### Example 71: 3-(N-((1,2,3,5,6,7-Hexahydro-s-indacen-4-yl)carbamoyl) sulfamoyl)-N,1-dimethyl-N-((1-methyl-1H-imidazol-2-yl)methyl)-1H-pyrazole-5-carboxamide

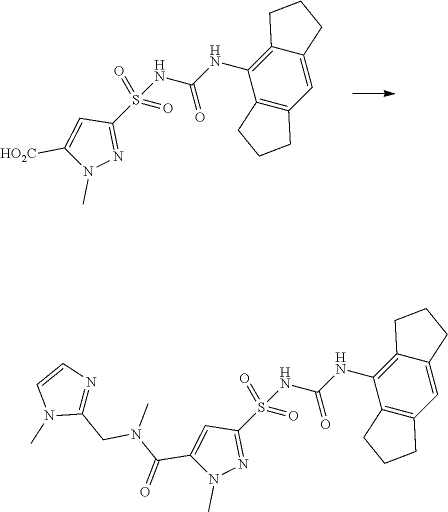 [SMILES: Cn1nc(S(=O)(=O)NC(=O)Nc2c3c(cc4c2CCC4)CCC3)cc1C(=O)O ; CN(Cc1nccn1C)C(=O)c1cc(S(=O)(=O)NC(=O)Nc2c3c(cc4c2CCC4)CCC3)nn1C]

Prepared according to the general procedure of N-(2-cyanoethyl)-3-(N-((1,2,3,5,6,7-hexahydro-s-indacen-4-yl)carbamoyl)sulfamoyl)-N,1-dimethyl-1H-pyrazole-5-carboxamide (Example 9) from 3-(N-((1,2,3,5,6,7-hexahydro-s-indacen-4-yl)carbamoyl)sulfamoyl)-1-methyl-1H-pyrazole-5-carboxylic Acid, Disodium Salt (Intermediate P8) and N-methyl-1-(1-methyl-H-imidazol-2-yl)methanamine to afford the title compound (10 mg, 14%) as a white solid.

¹H NMR (DMSO-d₆), rotamers; δ 7.71 (s, 1H), 7.20 (s, 0.4H), 7.14 (s, 0.6H), 7.05 (s, 0.6H), 6.93 (s, 0.4H), 6.85-6.83 (m, 2H), 4.73-4.17 (m, 2H), 3.89 (s, 3H), 3.64-3.45 (2×s, 3H), 2.98-2.94 (2×s, 3H), 2.76 (t, J=7.4 Hz, 4H), 2.63-2.59 (m, 4H), 1.94-1.87 (m, 4H). NH not observed.

LCMS; m/z 512 (M+H)⁺ (ES⁺).

### Example 72: 3-(N-((1,2,3,5,6,7-Hexahydro-s-indacen-4-yl)carbamoyl) sulfamoyl)-N,1-dimethyl-N-(thiazol-2-yl)-1H-pyrazole-5-carboxamide

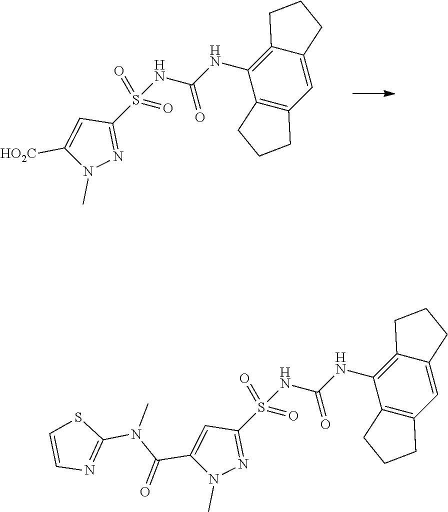 [SMILES: Cn1nc(S(=O)(=O)NC(=O)Nc2c3c(cc4c2CCC4)CCC3)cc1C(=O)O ; CN(C(=O)c1cc(S(=O)(=O)NC(=O)Nc2c3c(cc4c2CCC4)CCC3)nn1C)c1nccs1]

Prepared according to the general procedure of N-(2-cyanoethyl)-3-(N-((1,2,3,5,6,7-hexahydro-s-indacen-4-yl)carbamoyl)sulfamoyl)-N,1-dimethyl-1H-pyrazole-5-carboxamide (Example 9) from 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) and N-methylthiazol-2-amine to afford the title compound (4 mg, 7%) as a white solid.

¹H NMR (DMSO-d₆) δ 11.03 (br s, 1H), 8.11 (br s, 1H), 7.66 (d, J=3.6 Hz, 1H), 7.45 (d, J=3.6 Hz, 1H), 7.38 (s, 1H), 6.95 (s, 1H), 4.05 (s, 3H), 3.72 (s, 3H), 2.79 (t, J=7.4 Hz, 4H), 2.61 (t, J=7.3 Hz, 4H), 1.99-1.92 (m, 4H).

LCMS; m/z 501 (M+H)⁺ (ES⁺).

### Example 73: 3-(N-((1,2,3,5,6,7-Hexahydro-s-indacen-4-yl)carbamoyl) sulfamoyl)-N,1-dimethyl-N-((1-methyl-1H-pyrazol-5-yl)methyl)-1H-pyrazole-5-carboxamide, Partial Ammonium Salt

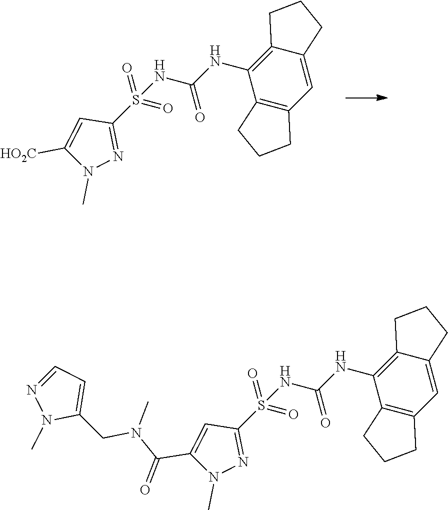 [SMILES: Cn1nc(S(=O)(=O)NC(=O)Nc2c3c(cc4c2CCC4)CCC3)cc1C(=O)O ; CN(Cc1ccnn1C)C(=O)c1cc(S(=O)(=O)NC(=O)Nc2c3c(cc4c2CCC4)CCC3)nn1C]

N-Methyl-1-(1-methyl-1H-pyrazol-5-yl)methanamine (23.45 mg, 0.187 mmol), NaHCO₃ (16 mg, 0.190 mmol) and HATU (72 mg, 0.189 mmol) were successively added to a suspension of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (70 mg, 0.156 mmol) in DMF (1 mL) and stirred for 2 days. The reaction was quenched with water (1 mL) and purified by reversed phase prep-HPLC (General Methods, basic prep) to afford the title compound (14 mg, 17%) as a white solid.

¹H NMR (DMSO-d₆), rotamers; δ 7.65 & 7.58 (2×s, 1H), 7.50-7.00 (br s, 1H), 7.36 (d, J=1.9 Hz, 1H), 6.81 (s, 1H), 6.81 & 6.60 (2×s, 1H), 6.27 & 6.14 (2×s, 1H), 4.76 & 4.70 (2×s, 2H), 3.87 & 3.80 (2×s, 3H), 3.80 & 3.58 (2×s, 3H), 2.99 (s, 3H), 2.75 (t, J=7.4 Hz, 4H), 2.63 (t, J=7.5 Hz, 4H), 1.89 (p, J=7.5 Hz, 4H).

LCMS; m/z 512.4 (M+H)⁺ (ES⁺); 510.3 (M−H)⁻ (ES⁻).

### Example 74:3-(N-((4-Chloro-2,6-diisopropylphenyl)carbamoyl) sulfamoyl)-N-(2-hydroxyethyl)-N,1-dimethyl-1H-pyrazole-5-carboxamide

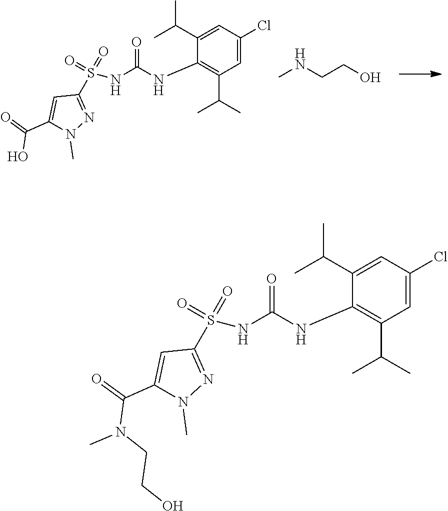

HATU (51.4 mg, 0.135 mmol) was added to a solution of 3-(N-((4-chloro-2,6-diisopropylphenyl)carbamoyl)sulfamoyl)-1-methyl-1H-pyrazole-5-carboxylic acid, disodium salt (Intermediate P11) (54.8 mg, 0.113 mmol) and 2-(methylamino)-ethanol (9.19 μL, 0.124 mmol) in DMF (1 mL) and the mixture was stirred at room temperature for 20 hours. Water (1 mL) was slowly added and the reaction mixture was stirred for 1 hour. The suspension was filtered and the collected solid triturated in water (3 mL) for 0.5 hour. The suspension was filtered and the collected solid was washed with water (0.5 mL) and TBME (1 mL). The solid was dried under reduced pressure for 6 hours to afford the title compound (10 mg, 18%) as a white solid.

¹H NMR (DMSO-d₆) rotamers: δ 11.18 (s, 1H), 7.95 (s, 1H), 7.14 (s, 2H), 6.98 (s, 1H), 4.96-4.79 (m, 1H), 3.93-3.86 (m, 3H), 3.64-3.41 (m, 4H), 3.06-2.88 (m, 5H), 1.08 (br s, 12H).

LCMS m/z 500.4/502.4 (M+H)⁺ (ES⁺).

### Example 75: 3-(N-((2,6-Diisopropylphenyl)carbamoyl)sulfamoyl)-N-(2-hydroxyethyl)-N,1-dimethyl-1H-pyrazole-5-carboxamide

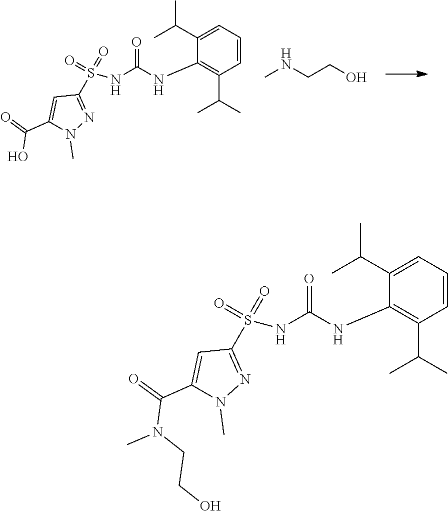

2-(Methylamino)ethanol (18 μL, 0.224 mmol) and HATU (82 mg, 0.217 mmol) were successively added to a solution of 3-(N-((2,6-diisopropylphenyl)carbamoyl) sulfamoyl)-1-methyl-1H-pyrazole-5-carboxylic acid, disodium salt (Intermediate P9) (70 mg, 0.155 mmol) in DMF (1 mL) and stirred for 20 hours. The reaction was quenched with water (0.1 mL) and purified by reversed phase prep-HPLC (General Methods, basic prep) to afford a white solid. The solid was dissolved in DMF (0.5 mL), diluted with water (1 mL) and stirred for 20 hours. The resulting precipitate was filtered off, washed with water and TBME, and dried to afford the title compound (8 mg, 11%), as a white solid.

¹H NMR (DMSO-d₆), rotamers; δ 11.07 (s, 1H), 7.88 (s, 1H), 7.25 (t, J=7.7 Hz, 1H), 7.12 (d, J=7.7 Hz, 2H), 7.02 & 7.0 (2×s, 1H),4.96 & 4.81 (t, J=5.0 & 5-7 Hz, 1H),3.94 & 3.89 (2×s, 3H),3.59 & 3.40 (2×t, J=5.6 & 5.1 Hz, 2H),3.50 (2×t, J=5.6 & 5.2 Hz, 2H), 3.03 & 2.98 (2×s, 3H), 2.95 (m, 2H), 1.06 (br s, 12H).

LCMS; m/z 466.5 (M+H)⁺ (ES⁺); 464.4 (M−H)⁻ (ES⁻).

### Example 76: 3-(N-((1,2,3,5,6,7-Hexahydro-s-indacen-4-yl)carbamoyl) sulfamoyl)-N-(2-hydroxyethyl)-1-methyl-1H-pyrazole-5-carboxamide, Partial Ammonium Salt

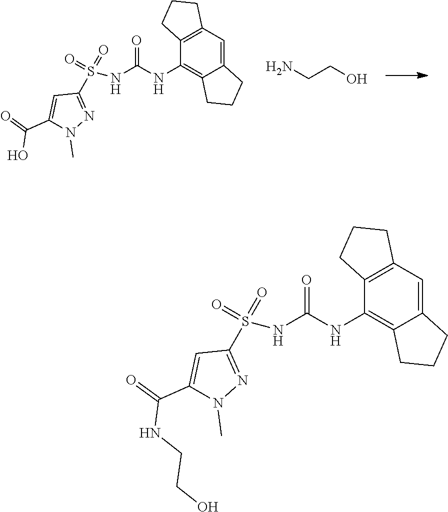

2-Aminoethanol (14 μL, 0.232 mmol) and HATU (83 mg, 0.219 mmol) were successively added to a suspension of 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) (70 mg, 0.156 mmol) in DMF (1 mL) and stirred for 20 hours. The reaction was quenched with water (0.1 mL) and purified by reversed phase prep-HPLC (General Methods, basic prep) to afford the title compound (18 mg, 25%) as a white solid.

¹H NMR (DMSO-d₆) δ 8.61 (t, J=5.7 Hz, 1H), 7.84 (s, 1H), 7.32 (s, 1H), 6.88 (s, 1H), 4.73 (t, J=5.7 Hz, 1H), 4.10 (s, 3H), 3.48 (app q, J=6.0 Hz, 2H), 3.27 (app q, J=6.0 Hz, 2H), 2.78 (t, J=7.4 Hz, 4H), 2.60 (t, J=7.4 Hz, 4H), 1.93 (p, J=7.4 Hz, 4H). One exchangeable proton not seen.

LCMS; m/z 448.4 (M+H)⁺ (ES⁺); 446.4 (M−H)⁻ (ES⁻).

### Example 77: 3-(N-((1,2,3,5,6,7-Hexahydro-s-indacen-4-yl)carbamoyl) sulfamoyl)-N-(2-methoxyethyl)-1-methyl-1H-pyrazole-5-carboxamide, Partial Ammonium Salt

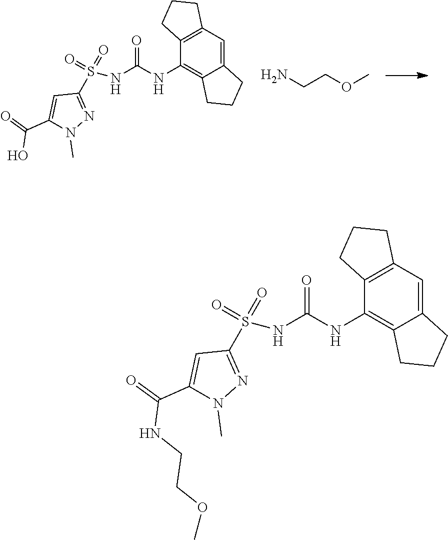

Prepared according to the general procedure of 3-(N-((1,2,3,5,6,7-hexahydro-s-indacen-4-yl)carbamoyl)sulfamoyl)-N-(2-hydroxyethyl)-1-methyl-1H-pyrazole-5-carboxamide, partial ammonium salt (Example 76) from 3-(N-((1,2,3,5,6,7-hexahydro-s-indacen-4-yl)carbamoyl)sulfamoyl)-1-methyl-1H-pyrazole-5-carboxylic acid, disodium salt (Intermediate P8) and 2-methoxyethan-1-amine to afford the title compound as a colourless solid (37 mg, 51%).

¹H NMR (DMSO-d₆) δ 10.93 (br s, 1H), 8.74 (t, J=5.5 Hz, 1H), 7.90 (s, 1H), 7.36 (s, 1H), 6.90 (s, 1H), 4.11 (s, 3H), 3.44 (t, J=5.5 Hz, 2H), 3.39 (t, J=5.5 Hz, 2H), 3.26 (s, 3H), 2.78 (t, J=7.4 Hz, 4H), 2.61 (t, J=7.3 Hz, 4H), 1.94 (p, J=7.5 Hz, 4H).

LCMS; m/z 462.4 (M+H)⁺ (ES⁺); 460.3 (M−H)⁻ (ES⁻).

### Example 78: 3-(N-((1,2,3,5,6,7-Hexahydro-s-indacen-4-yl)carbamoyl) sulfamoyl)-N-(2-hydroxyethyl)-N,1-dimethyl-1H-pyrazole-5-carboxamide, Partial Ammonium Salt

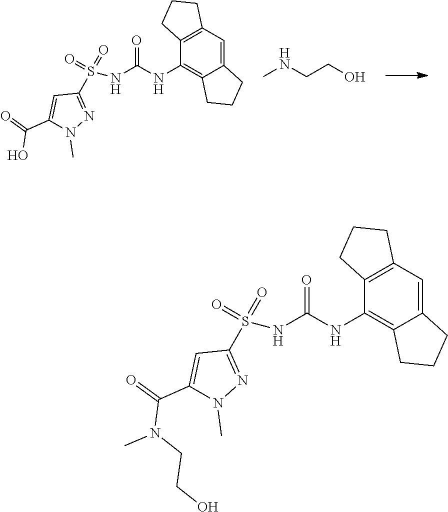

2-(Methylamino)ethanol (19.25 μL, 0.240 mmol) and HATU (72 mg, 0.189 mmol) were successively added to a suspension of 3-(N-((1,2,3,5,6,7-hexahydro-s-indacen-4-yl) carbamoyl)sulfamoyl)-1-methyl-1H-pyrazole-5-carboxylic acid, disodium salt (Intermediate P8) (70 mg, 0.156 mmol) in DMF (1 mL) and stirred for 20 hours. The solution was purified by chromatography on RP Flash C18 (12 g column, 5-50% MeCN/10 mM ammonium bicarbonate) to afford the title compound (39 mg, 54%) as a white solid.

¹H NMR (DMSO-d₆), rotamers; δ 7.69 (s, 1H), 7.10 (s, 1H), 7.10 & 6.74 (2×s, 1H), 6.82 (s, 1H), 4.96 & 4.84 (2×t, J=5.5 Hz, 1H), 3.84 & 3.80 (2×s, 3H), 3.59 (m, 1H), 3.55-3.41 (m, 3H), 3.06 & 2.97 (2×s, 3H), 2.76 (t, J=7.4 Hz, 4H), 2.64 (t, J=7.5 Hz, 4H), 1.92 (p, J=7.5 Hz, 4H).

LCMS; m/z 462.42 (M+H)⁺ (ES⁺); 460.30 (M−H)⁻ (ES⁻).

### Example 70: 3-(N-((4-Fluoro-2-(2-isopropoxypyridin-4-yl)-6-isopropyl-phenyl)carbamoyl)sulfamoyl)-N,N-bis(2-methoxyethyl)-1-methyl-1H-pyrazole-5-carboxamide, Sodium Salt

### Step A: 3-(N-((4-Fluoro-2-(2-isopropoxypyridin-4-yl)-6-isopropylphenyl)carbamoyl) sulfamoyl)-N,N-bis(2-methoxyethyl)-1-methyl-1H-pyrazole-5-carboxamide

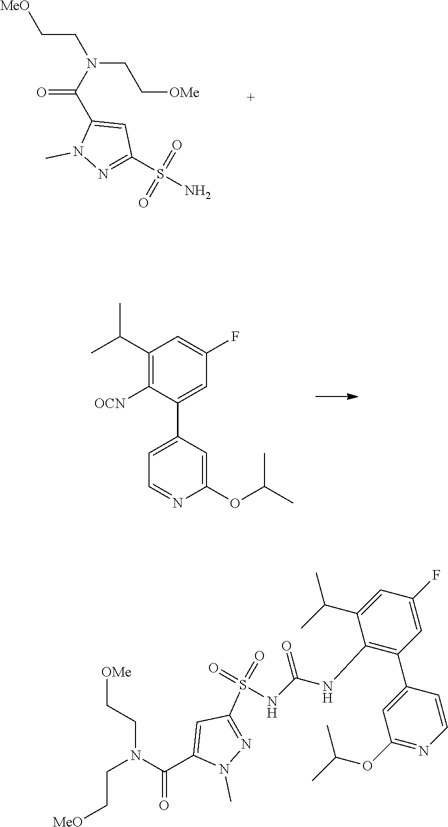

A solution of N,N-bis(2-methoxyethyl)-1-methyl-3-sulfamoyl-1H-pyrazole-5-carboxamide (Intermediate P16) (2.2 g, 6.87 mmol, 1 eq), 4-(5-fluoro-2-isocyanato-3-isopropylphenyl)-2-isopropoxypyridine (Intermediate A10) (2.16 g, 6.87 mmol, 1 eq) and t-BuONa (659 mg, 6.87 mmol, 1 eq) in THF (100 mL) was stirred at 25° C. for 30 minutes. The reaction mixture was concentrated in vacuo. The residue was purified by reversed phase flash chromatography (column: Welch Ultimate XB_C18, 41 mm*235 mm*20/40 μm, mobile phase: [A: water (10 mM NH₄HCO₃); B: MeCN]; B %: 0%-30%, 35 min) to give the title compound (2.5 g, 56% yield, 98% purity on LCMS) as a white solid.

¹H NMR (DMSO-d₆): δ 11.10 (br s, 1H), 8.06 (d, 1H), 7.79 (br s, 1H), 7.18 (d, 1H), 7.02 (d, 1H), 6.83-6.72 (m, 2H), 6.70 (s, 1H), 5.29-5.23 (m, 1H), 3.83 (s, 3H), 3.64-3.61 (m, 2H), 3.55-3.50 (m, 4H), 3.45-3.40 (m, 2H), 3.28 (s, 3H), 3.14 (s, 3H), 3.03-300 (m, 1H), 1.30 (d, 6H) and 1.09-1.05 (m, 6H).

LCMS: m/z 635.4 (M+H)⁺ (ES⁺).

### Step B: 3-(N-((4-Fluoro-2-(2-isopropoxypyridin-4-yl)-6-isopropylphenyl)carbamoyl) sulfamoyl)-N,N-bis(2-methoxyethyl)-1-methyl-1H-pyrazole-5-carboxamide, Sodium Salt

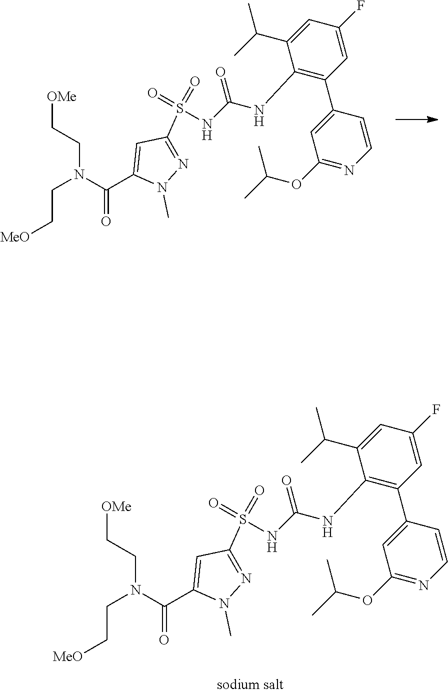 [SMILES: COCCN(CCOC)C(=O)c1cc(S(=O)(=O)NC(=O)Nc2c(-c3ccnc(OC(C)C)c3)cc(F)cc2C(C)C)nn1C ; COCCN(CCOC)C(=O)c1cc(S(=O)(=O)NC(=O)Nc2c(-c3ccnc(OC(C)C)c3)cc(F)cc2C(C)C)nn1C]

To a solution of 3-(N-((4-fluoro-2-(2-isopropoxypyridin-4-yl)-6-isopropylphenyl) carbamoyl)sulfamoyl)-N,N-bis(2-methoxyethyl)-1-methyl-1H-pyrazole-5-carboxamide (2.5 g, 3-94 mmol, 1 eq, free form) in THF (100 mL) was added with t-BuONa (378 mg, 3.94 mmol, 1 eq). The reaction mixture was stirred at 25° C. for 1 hour and then concentrated in vacuo. The residue was triturated with isopropyl ether (20 mL) to give the title compound (2.2 g, 85% yield, 99% purity on LCMS, sodium salt) as a white solid.

¹H NMR (DMSO-d₆): 7.99-7.88 (m, 1H), 7.53-7.40 (m, 1H), 7.15-7.08 (m, 1H), 6.94-6.82 (m, 2H), 6.68 (s, 1H), 6.51-6.44 (m, 1H), 5.28-5.22 (m, 1H), 3.75 (s, 3H), 3.74-3.56 (m, 6H), 3.45-3.38 (m, 2H), 3.29 (s, 3H), 3.17 (s, 3H), 3.12-307 (m, 1H), 1.29 (d, 6H) and 1.20-1.04 (m, 6H).

LCMS: m/z 635.1 (M+H)⁺ (ES⁺).

### Example 80: N,N-Bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide, Sodium Salt

### Step A: N,N-Bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide

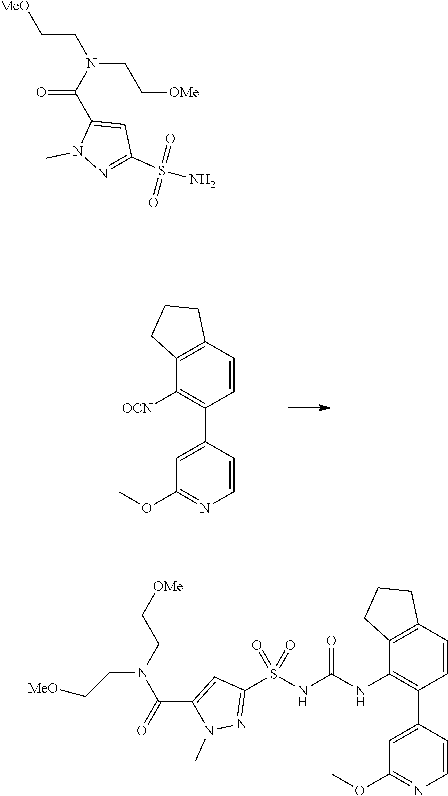

A solution of N,N-bis(2-methoxyethyl)-1-methyl-3-sulfamoyl-1H-pyrazole-5-carboxamide (Intermediate P16) (2.56 g, 7.99 mmol, 1 eq) and t-BuONa (768 mg, 7.99 mmol, 1 eq) in THF (200 mL) was stirred at 25° C. for 30 minutes. Then 4(4-isocyanato-2,3-dihydro-H-inden-5-yl)-2-methoxypyridine (Intermediate A11) (3.34 g, 8.79 mmol, 1.1 eq) was added. The reaction mixture was stirred at 70° C. for 2 hours and then concentrated in vacuo. The residue was purified by reversed phase flash chromatography (column: Welch Ultimate XB_C18, 41 mm*235 mm*20/40 μm, mobile phase: [A: water (0.05% ammonium hydroxide); B: MeCN]; B %: 0%-30%,35 min) to give the title compound (1.35 g, 29% yield, 99% purity on LCMS) as a white solid. ¹H NMR (DMSO-d₆): δ 8.08 (d, 1H), 7.14-7.11 (m, 1H), 7.07-7.05 (m, 1H), 6.91 (d, 1H), 6.74 (s, 1H), 6.60 (s, 1H), 3.86 (s, 3H), 3.78 (s, 3H), 3.64-3.62 (m, 2H), 3.56-3.54 (m, 4H), 3.39-3.37 (m, 2H), 3.28 (s, 3H), 3.14 (s, 3H), 2.89 (t, 2H), 2.71 (t, 2H) and 1.99-1.94 (m, 2H).

LCMS: m/z 587.3 (M+H)⁺ (ES⁺).

### Step B: N,N-Bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide, Sodium Salt

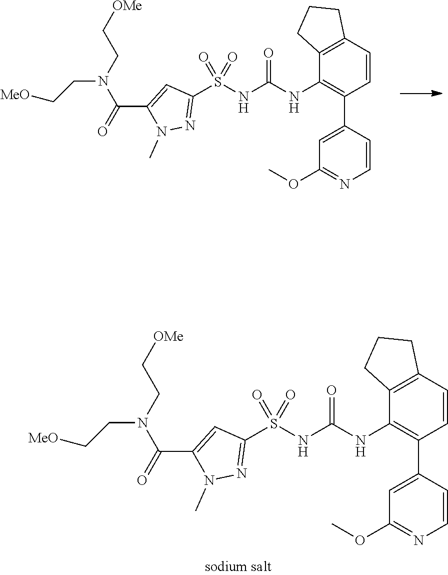 [SMILES: COCCN(CCOC)C(=O)c1cc(S(=O)(=O)NC(=O)Nc2c(-c3ccnc(OC)c3)ccc3c2CCC3)nn1C ; COCCN(CCOC)C(=O)c1cc(S(=O)(=O)NC(=O)Nc2c(-c3ccnc(OC)c3)ccc3c2CCC3)nn1C]

To a solution of N,N-bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide (1.35 g, 2.30 mmol, 1 eq, free form) in THF (20 mL) was added with t-BuONa (221 mg, 2.30 mmol, 1 eq). The reaction mixture was stirred at 25° C. for 1 hour and then concentrated in vacuo. The residue was triturated with isopropyl ether (20 mL) to give the title compound (1.2 g, 85% yield, 99% purity on HPLC) as a white solid.

¹H NMR (DMSO-d₆): δ 8.05 (d, 1H), 7.30 (br s, 1H), 7.04 (dd, 2H), 6.92 (d, 1H), 6.76 (s, 1H), 6.48 (d, 1H), 3.85 (s, 3H), 3.75 (s, 3H), 3.64-3.62 (m, 2H), 3.56-3.53 (m, 4H), 3.39-3.37 (m, 2H), 3.29 (s, 3H), 3.15 (s, 3H), 2.87 (t, 2H), 2.73-2.70 (m, 2H) and 1.98-1.91 (m, 2H).

LCMS: m/z 587.1 (M+H)⁺ (ES⁺).

### Example 81: 3-(N-((4-Fluoro-2-(2-isopropoxypyridin-4-yl)-6-isopropyl-phenyl)carbamoyl)sulfamoyl)-N,N,1-trimethyl-1H-pyrazole-5-carboxamide, Sodium Salt

### Step A: 3-(N-((4-Fluoro-2-(2-isopropoxypyridin-4-yl)-6-isopropylphenyl)carbamoyl)sulfamoyl)-N,N,1-trimethyl-1H-pyrazole-5-carboxamide

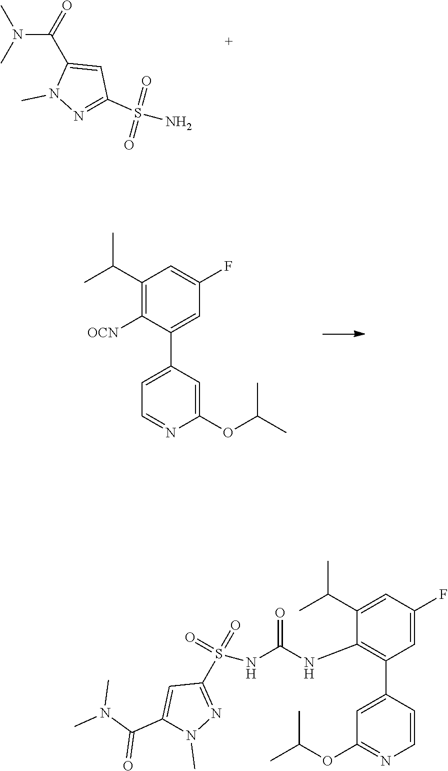

To a solution of N,N,1-trimethyl-3-sulfamoyl-H-pyrazole-5-carboxamide (Intermediate P5) (1.7 g, 7.32 mmol, 1 eq) in THF (20 mL) was added t-BuONa (703 mg, 7.32 mmol, 1 eq) at 25° C. and stirred for 0.5 hour. Then 4-(5-fluoro-2-isocyanato-3-isopropylphenyl)-2-isopropoxypyridine (Intermediate A10) (2.30 g, 7.32 mmol, 1 eq) was added and the resulting mixture was stirred for 0.5 hour. The mixture was concentrated in vacuo. The residue was purified by prep-HPLC (column: Welch Ultimate XB_C18, 41 mm*235 mm*20/40 μm; mobile phase: [water (10 mM NH₄HCO₃)-ACN]; B %: 0%-30%, 35 min) to give the title compound (2.34 g, 59% yield, 98% purity on HPLC) as a white solid.

¹H NMR (DMSO-d₆): δ 8.03 (d, 1H), 7.65 (br s, 1H), 7.16 (d, 1H), 6.98 (d, 1H), 6.85 (d, 1H), 6.74 (s, 1H), 6.70 (s, 1H), 5.30-5.21 (m, 1H), 3.89 (s, 3H), 3.09-3.03 (m, 1H), 3.00 (s, 6H), 1.30 (d, 6H) and 1.07 (d, 6H).

LCMS: m/z 547.4 (M+H)⁺ (ES⁺).

### Step B: 3-(N-((4-Fluoro-2-(2-isopropoxypyridin-4-yl)-6-isopropylphenyl)carbamoyl) sulfamoyl)-N,N,1-trimethyl-1H-pyrazole-5-carboxamide, Sodium Salt

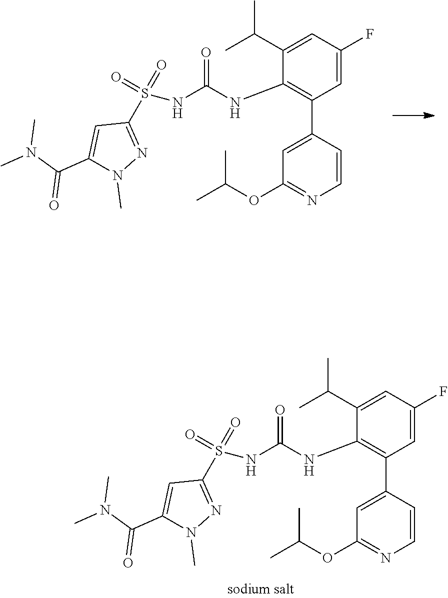 [SMILES: CC(C)Oc1cc(-c2cc(F)cc(C(C)C)c2NC(=O)NS(=O)(=O)c2cc(C(=O)N(C)C)n(C)n2)ccn1 ; CC(C)Oc1cc(-c2cc(F)cc(C(C)C)c2NC(=O)NS(=O)(=O)c2cc(C(=O)N(C)C)n(C)n2)ccn1]

To a solution of 3-(N-((4-fluoro-2-(2-isopropoxypyridin-4-yl)-6-isopropylphenyl) carbamoyl)sulfamoyl)-N,N,1-trimethyl-1H-pyrazole-5-carboxamide (1.71 g, 3.13 mmol, 1 eq, free form) in THF (40 mL) was added t-BuONa (300 mg, 3.13 mmol, 1 eq) at 25° C. Then the mixture was stirred for 1 hour. The mixture was concentrated in vacuo. The residue was triturated with MTBE (100 mL). The solid was dissolved in water (100 mL) and then lyophilized to give the title compound (1.60 g, 90% yield, 99.9% purity on HPLC) as a white solid.

¹H NMR (DMSO-d₆): δ 7.95 (d, 1H), 7.37 (br s, 1H), 7.09 (d, 1H), 6.93-6.90 (m, 2H), 6.69 (s, 1H), 6.53 (s, 1H), 5.29-5.22 (m, 1H), 3.83 (s, 3H), 3.15-3.09 (m, 1H), 3.01 (d, 6H), 1.29 (d, 6H) and 1.05 (d, 6H).

LCMS: m/z 547.3 (M+H)⁺ (ES⁺).

### Example 82: 3-(N-((5-(2-Methoxypyridin-4-yl)-2,3-dihydro-1H-inden-4-yl) carbamoyl)sulfamoyl)-N,N,1-trimethyl-1H-pyrazole-5-carboxamide, Sodium Salt

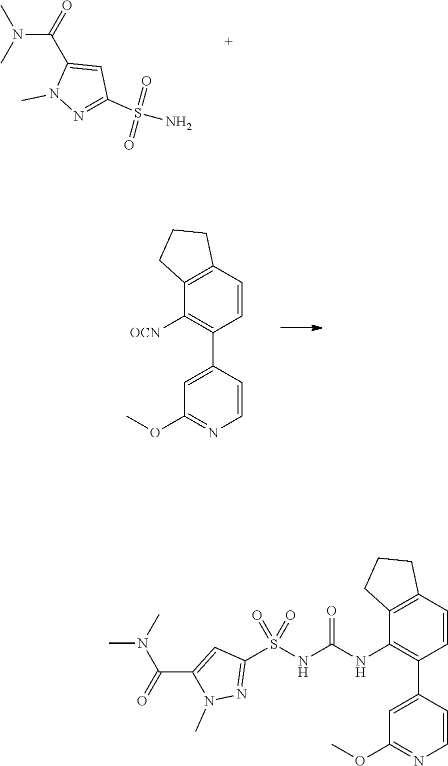

A solution of N,N,1-trimethyl-3-sulfamoyl-1H-pyrazole-5-carboxamide (Intermediate P5) (6.59 g, 28.39 mmol, 0.9 eq) and t-BuONa (3.33 g, 34.70 mmol, 1.1 eq) in THF (200 mL) was stirred at 16° C. for 0.5 hour. Then 4-(4-isocyanato-2,3-dihydro-1H-inden-5-yl)-2-methoxypyridine (Intermediate A11) (8.4 g, 31-54 mmol, 1 eq) was added. The reaction mixture was stirred at 16° C. for 0.5 hour and then filtered. The filter cake was washed with MeCN (125 mL). Then the solid was dissolved in H₂O (100 mL) and filtered. The filtrate was lyophilized to give the title compound (8.02 g, 49% yield, 99.54% purity on LCMS, Na salt) as a white solid.

¹H NMR (DMSO-d₆): δ 8.02 (d, 1H), 7.42 (br s, 1H), 7.10-7.02 (m, 2H), 6.89 (dd, 1H), 6.74 (s, 1H), 6.59 (s, 1H), 3.84 (d, 6H), 3.02 (d, 6H), 2.87 (t, 2H), 2.72 (t, 2H) and 1.97-1.90 (m, 2H).

LCMS: m/z 499.3 (M+H)⁺ (ES⁺).

### Example 83: 2-(3-(N-((1,2,3,5,6,7-Hexahydro-s-indacen-4-yl)carbamoyl) sulfamoyl)-1H-pyrazol-1-yl)-N,N,2-trimethylpropanamide

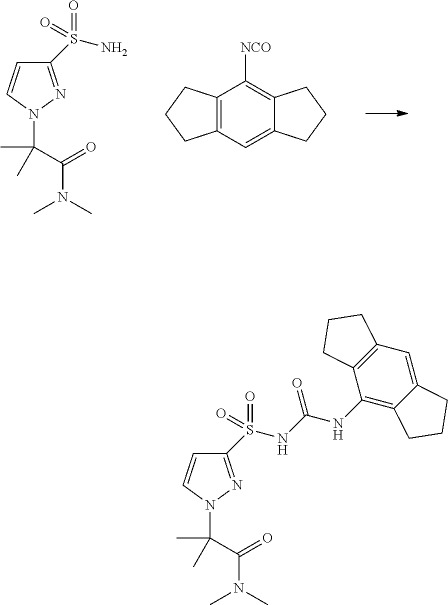

Sodium tert-butoxide (2 M in THF) (0.12 mL, 0.240 mmol) was added to a solution of N,N,2-trimethyl-2-(3-sulfamoyl-1H-pyrazol-1-yl)propanamide (60 mg, 0.230 mmol) (Intermediate P17) in THF (3 mL) and stirred at room temperature for 1 hour. Then 4-isocyanato-1,2,3,5,6,7-hexahydro-s-indacene (Intermediate A1) (50.5 mg, 0.254 mmol) was added and stirred at room temperature overnight. The reaction mixture was concentrated and the crude product was purified by chromatography on RP Flash C18 (13 g cartridge, 5-50% MeCN/10 mM ammonium bicarbonate) to afford the title compound (22 mg, 21%) as a colourless solid.

¹H NMR (DMSO-d₆, rotamers) δ 10.96 (br s, 1H), 8.02 (s, 1H), 7.90 (s, 1H), 6.90 (s, 1H), 6.77 (s, 1H), 2.89-2.68 (m, 7H), 2.60 (t, J=7.5 Hz, 4H), 2.31 (br s, 3H), 2.01-1.86 (m, 4H), 1.70 (s, 6H).

LCMS; m/z 460.4 (M+H)⁺ (ES⁺)

The compound of example 84 was synthesised by methods analogous to those outlined above.

TABLE 1
¹H NMR and MS data

| Ex | Structure and Name | ¹H NMR spectrum | MS spectrum | MW |
|---|---|---|---|---|
| 84 | 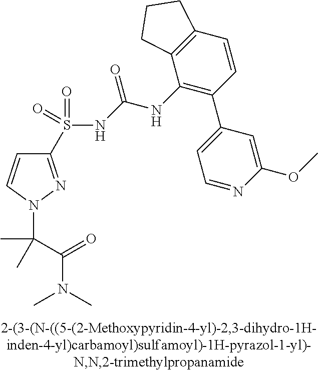 [SMILES: COc1cc(-c2ccc3c(c2NC(=O)NS(=O)(=O)c2ccn(C(C)(C)C(=O)N(C)C)n2)CCC3)ccn1] | ¹H NMR (DMSO-d₆) δ 11.03 (s, 1H), 8.14 (d, J = 5.3 Hz, 1H), 7.99 (s, 1H), 7.86 (s, 1H), 7.19 (d, J = 7.7 Hz, 1H), 7.10 (d, J = 7.6 Hz, 1H), 6.90 (dd, J = 5.3, 1.5 Hz, 1H), 6.73 (s, 1H), 6.68 (s, 1H), 3.89 (s, 3H), 2.91 (t, J = 7.4 Hz, 2H), 2.78 (s, 3H), 2.65 (t, J = 7.3 Hz, 2H), 2.22 (s, 3H), 1.97 (p, J = 7.5 Hz, 2H), 1.70 (s, 6H). | m/z 527.3 (M + H)⁺ (ES⁺); 525.3 (M − H)⁻ (ES⁻). | 526.6 |

### Examples—Biological Studies

NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5p compound (8 points half-log dilution, with 10 μM top dose) or vehicle (DMSO 0.1% FAC) to the appropriate wells
3. Incubate for 3 hrs at 37° C. in 5% CO₂
4. Add 5 μl nigericin (Sigma #N7143) (FAC 5 μM) to all wells
5. Incubate for 1 hr at 37° C. and 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hrs at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

96-Well Plate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Comp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Comp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

High MCC950(10 uM) Compound 8-point half-log dilution
Low Drug free control

The results of the pyroptosis assay performed are summarised in Table 2 below as THP IC₅₀.

Human Whole Blood IL1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hrs at 37° C., 5% CO₂
4. Add 10 μl Nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to anon-linear regression equation (log inhibitor vs response-variable slope 4-parameters)

The results of the human whole blood assay are summarised in Table 2 below as HWB IC₅₀.

TABLE 2
NLRP3 inhibitory activity [THP IC₅₀ (≤0.04 μM = +++++, ≤0.16 μM =
++++, ≤0.64 μM = +++, ≤2.56 μM = ++, ≤10 μM = +, not determined =
ND)] [HWB IC₅₀ (≤0.4 μM = *****, ≤0.8 μM = ****, ≤1.6 μM =
***, ≤3.2 μM = **, ≤10 μM = *, not determined = ND)]

| Example No | THP IC₅₀ | HWB IC₅₀ |
|---|---|---|
| 1 | +++ | * |
| 2 | +++ | ** |
| 3 | ++ | ND |
| 4 | + | ND |
| 5 | + | ND |
| 6 | + | ND |
| 7 | +++ | * |
| 8 | ++ | ND |
| 9 | +++ | ND |
| 10 | ++ | ND |
| 11 | ++ | ND |
| 12 | + | ND |
| 13 | + | ND |
| 14 | + | ND |
| 15 | +++ | ***** |
| 16 | ++++ | **** |
| 17 | ++ | ND |
| 18 | +++ | ** |
| 19 | ++ | ND |
| 20 | +++ | *** |
| 21 | ++ | ND |
| 22 | + | ND |
| 23 | + | ND |
| 24 | + | ND |
| 25 | + | ND |
| 26 | ++++ | **** |
| 27 | ++++ | **** |
| 28 | ++ | ND |
| 29 | ++++ | **** |
| 30 | +++ | ND |
| 31 | ++++ | *** |
| 32 | +++ | ** |
| 33 | ++ | **** |
| 34 | ++ | ND |
| 35 | ++ | ***** |
| 36 | ++ | ND |
| 37 | ++ | ND |
| 38 | + | ND |
| 39 | ++ | *** |
| 40 | + | ND |
| 41 | ++++ | ***** |
| 42 | +++ | ***** |
| 43 | ++++ | *** |
| 44 | +++ | * |
| 45 | ++++ | ***** |
| 46 | + | ND |
| 47 | ++++ | **** |
| 48 | +++ | ND |
| 49 | +++ | **** |
| 50 | +++ | ***** |
| 51 | +++ | ***** |
| 52 | ++++ | **** |
| 53 | +++ | *** |
| 54 | ++++ | ** |
| 55 | +++ | **** |
| 56 | ++ | ** |
| 57 | ++ | ND |
| 58 | +++ | **** |
| 59 | ++ | ND |
| 60 | +++ | ***** |
| 61 | ++ | ND |
| 62 | ++++ | **** |
| 63 | ++ | ND |
| 64 | ++++ | ** |
| 65 | ++++ | *** |
| 66 | ++ | ND |
| 67 | +++ | ***** |
| 68 | ++++ | ** |
| 69 | ++++ | ***** |
| 70 | +++ | ** |
| 71 | ++++ | *** |
| 72 | +++ | ND |
| 73 | ++ | ND |
| 74 | ++++ | *** |
| 75 | ++ | ND |
| 76 | + | ND |
| 77 | + | ND |
| 78 | ++++ | ***** |
| 79 | + | * |
| 80 | +++ | ***** |
| 81 | +++ | ** |
| 82 | +++++ | ***** |
| 83 | ++++ | *** |
| 84 | +++++ | ***** |

PK Protocol

Pharmacokinetic parameters were determined in male Sprague Dawley rats (Charles River, UK, 250-350 g; or Vital River Laboratory Animal Technology Co Ltd, Beijing, China, 7-9 weeks old). Animals were individually housed during the study and maintained under a 12 h light/dark cycle. Animals had free access to food and water except that some orally dosed animals were food deprived overnight prior to the study.

For intravenous administration, compounds were formulated as a solution in water or DMSO:PBS [10:90] in 2 mL/kg dosing volume and administered via tail vein. For oral administration, compounds were formulated as a solution in water or DMSO:water [10:90] in 5 mL/kg dosing volume and administered orally.

Serial blood samples (about 120-300 μL) were taken from each animal at each of 8 time-points post dose (0.083, 0.25, 0.5, 1, 2, 4, 8 and 24 h) or at each of 12 time-points post dose (0.03, 0.1, 0.17, 0.25, 0.5, 1, 2, 4, 6, 8, 12 and 24 h) or pre-dose and at each of 9 time-points post dose (0.25, 0.5, 1, 2, 4, 6, 8, 12 and 24 h). Samples were held on ice for no longer than 30 minutes before centrifugation (10,000 rpm (8,385 g) for 3 minutes; or 5,696 rpm (3,000 g) for 15 minutes) for plasma generation. Plasma was frozen on dry ice prior to bioanalysis. PK parameters were generated from LC-MS/MS data using Dotmatics or Phoenix WinNonlin 6.3 software.

TABLE 3
PK data (intravenous administration)

| Example | Dose | AUC | T_{1/2} | V_{dss} | Cl |
|---|---|---|---|---|---|
| No | (mg/kg) | (ng · hr/mL) | (hr) | (L/kg) | (mL/min/kg) |
| 26 | 2.88 | 329.7 | 0.2 | 1.84 | 149.3 |
| 27 | 1 | 1162.1 | 2.9 | 2.24 | 14.4 |
| 31 | 1 | 302.7 | 1.6 | 1.66 | 55.3 |
| 33 | 1 | 182.5 | 2.3 | 2.72 | 91.3 |
| 35 | 1 | 661.2 | 12.1 | 3.03 | 25.2 |
| 41 | 1 | 274.4 | 6.3 | 3.95 | 60.7 |
| 45 | 1 | 415.0 | 6.4 | 7.96 | 40.3 |
| 60 | 1 | 283.3 | 3.3 | 4.08 | 59.2 |
| 62 | 2.46 | 1723.1 | 0.1 | 0.24 | 25.0 |
| 67 | 0.83 | 333.7 | 0.5 | 0.99 | 48.7 |
| 68 | 3.34 | 353.3 | 1.3 | 10.55 | 160.9 |
| 69 | 5.05 | 3389.9 | 25.3 | 17.78 | 24.8 |
| 78 | 2.41 | 786.6 | 0.3 | 2.37 | 114.1 |

TABLE 4
PK data (oral administration)

| Example | Dose | Cₘₐₓ | AUC | Tₘₐₓ | T_{1/2} | Cl/F | |
|---|---|---|---|---|---|---|---|
| No | (mg/kg) | (ng/mL) | (ng · hr/mL) | (hr) | (hr) | (mL/min/kg) | Bioavailability |
| 27 | 3 | 700.6 | 2415.2 | 0.08 | 4.9 | 21.5 | 67 |
| 60 | 5 | 494.7 | 769.7 | 0.17 | 9.7 | 109.8 | 54.3 |
| 69 | 3 | 599.6 | 1052.0 | 0.25 | 3.4 | 49.5 | 62.0 |

As is evident from the results presented in Table 2, surprisingly in spite of the structural differences versus the prior art compounds, the compounds of the invention show high levels of NLRP3 inhibitory activity in the pyroptosis assay and in the human whole blood assay.

As is evident from the results presented in Tables 3 and 4, the compounds of the invention show advantageous pharmacokinetic properties, for example half-life T_{1/2}, area under the curve AUC, clearance Cl and/or bioavailability, compared to the prior art compounds. In particular, it is evident from the pharmacokinetic data that the compounds of the invention are particularly suited to topical routes of administration.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 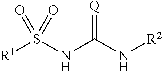
Formula (I)
or a pharmaceutically acceptable salt, solvate or prodrug thereof, wherein:
Q is selected from O or S;
R¹ is a 5-membered heteroaryl group substituted with at least one group R^{X}, wherein R^{X} is any group comprising an amide group, wherein the 5-membered heteroaryl group may optionally be further substituted; and
R² is a cyclic group substituted at the α-position, wherein R² may optionally be further substituted;
provided that the compound is not:

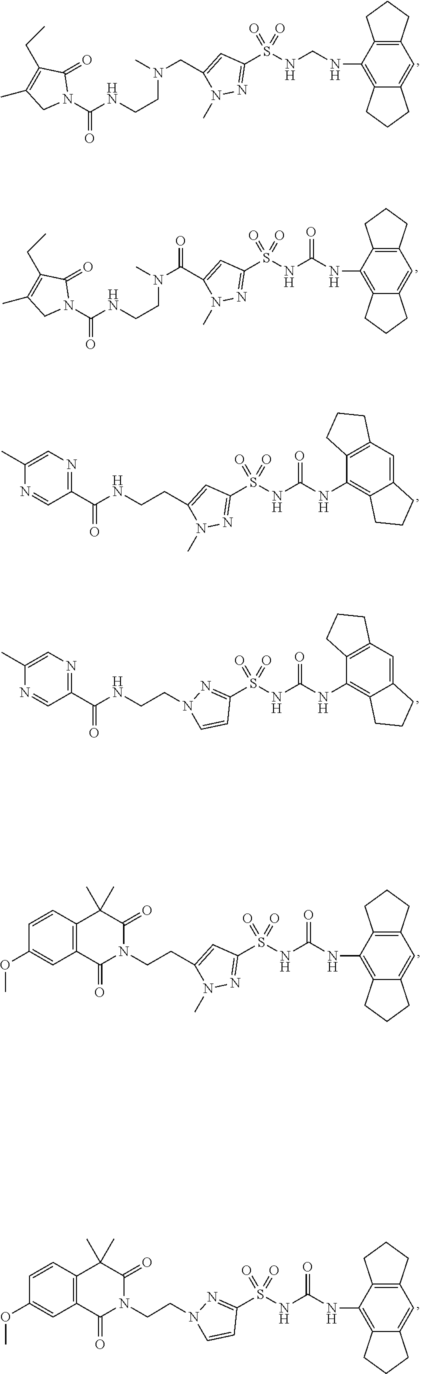 [SMILES: CCC1=C(C)CN(C(=O)NCCN(C)Cc2cc(S(=O)(=O)NCNc3c4c(cc5c3CCC5)CCC4)nn2C)C1=O ; CCC1=C(C)CN(C(=O)NCCN(C)C(=O)c2cc(S(=O)(=O)NC(=O)Nc3c4c(cc5c3CCC5)CCC4)nn2C)C1=O ; Cc1cnc(C(=O)NCCc2cc(S(=O)(=O)NC(=O)Nc3c4c(cc5c3CCC5)CCC4)nn2C)cn1 ; Cc1cnc(C(=O)NCCn2ccc(S(=O)(=O)NC(=O)Nc3c4c(cc5c3CCC5)CCC4)n2)cn1 ; COc1ccc2c(c1)C(=O)N(CCc1cc(S(=O)(=O)NC(=O)Nc3c4c(cc5c3CCC5)CCC4)nn1C)C(=O)C2(C)C ; COc1ccc2c(c1)C(=O)N(CCn1ccc(S(=O)(=O)NC(=O)Nc3c4c(cc5c3CCC5)CCC4)n1)C(=O)C2(C)C]

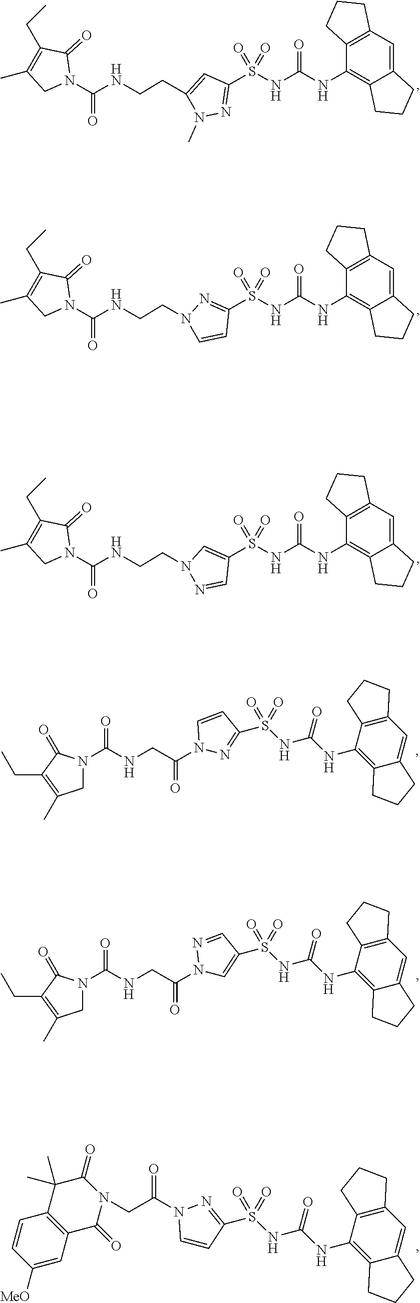 [SMILES: CCC1=C(C)CN(C(=O)NCCc2cc(S(=O)(=O)NC(=O)Nc3c4c(cc5c3CCC5)CCC4)nn2C)C1=O ; CCC1=C(C)CN(C(=O)NCCn2ccc(S(=O)(=O)NC(=O)Nc3c4c(cc5c3CCC5)CCC4)n2)C1=O ; CCC1=C(C)CN(C(=O)NCCn2cc(S(=O)(=O)NC(=O)Nc3c4c(cc5c3CCC5)CCC4)cn2)C1=O ; CCC1=C(C)CN(C(=O)NCC(=O)n2ccc(S(=O)(=O)NC(=O)Nc3c4c(cc5c3CCC5)CCC4)n2)C1=O ; CCC1=C(C)CN(C(=O)NCC(=O)n2cc(S(=O)(=O)NC(=O)Nc3c4c(cc5c3CCC5)CCC4)cn2)C1=O ; COc1ccc2c(c1)C(=O)N(CC(=O)n1ccc(S(=O)(=O)NC(=O)Nc3c4c(cc5c3CCC5)CCC4)n1)C(=O)C2(C)C]

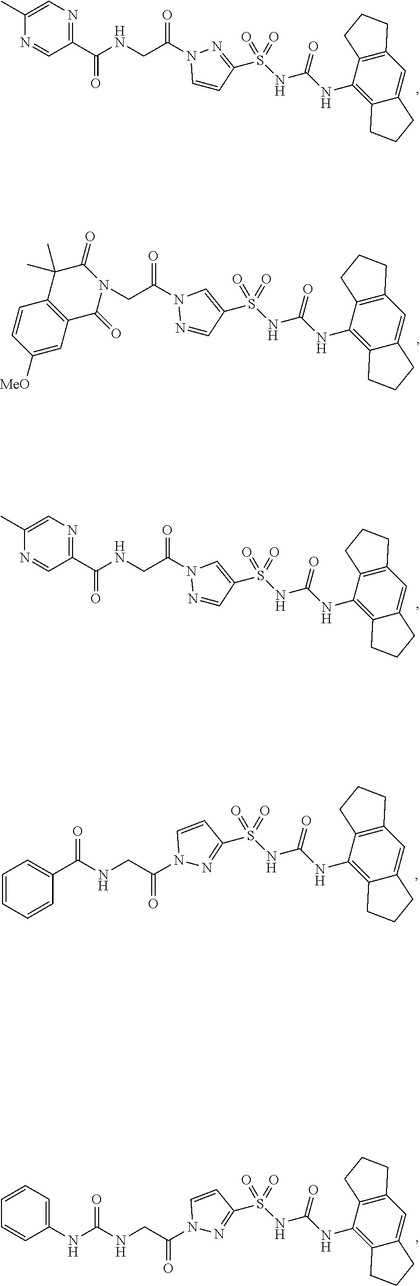 [SMILES: Cc1cnc(C(=O)NCC(=O)n2ccc(S(=O)(=O)NC(=O)Nc3c4c(cc5c3CCC5)CCC4)n2)cn1 ; COc1ccc2c(c1)C(=O)N(CC(=O)n1cc(S(=O)(=O)NC(=O)Nc3c4c(cc5c3CCC5)CCC4)cn1)C(=O)C2(C)C ; Cc1cnc(C(=O)NCC(=O)n2cc(S(=O)(=O)NC(=O)Nc3c4c(cc5c3CCC5)CCC4)cn2)cn1 ; O=C(Nc1c2c(cc3c1CCC3)CCC2)NS(=O)(=O)c1ccn(C(=O)CNC(=O)c2ccccc2)n1 ; O=C(NCC(=O)n1ccc(S(=O)(=O)NC(=O)Nc2c3c(cc4c2CCC4)CCC3)n1)Nc1ccccc1]

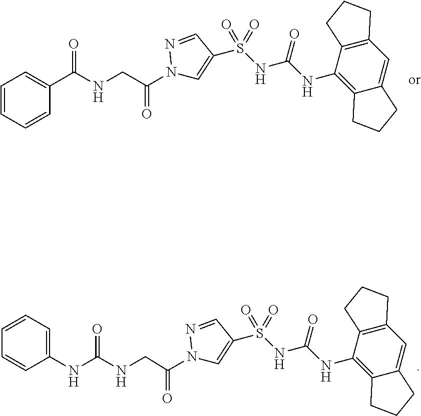 [SMILES: O=C(Nc1c2c(cc3c1CCC3)CCC2)NS(=O)(=O)c1cnn(C(=O)CNC(=O)c2ccccc2)c1 ; O=C(NCC(=O)n1cc(S(=O)(=O)NC(=O)Nc2c3c(cc4c2CCC4)CCC3)cn1)Nc1ccccc1]

2. A compound or a pharmaceutically acceptable salt, solvate or prodrug thereof, as claimed in claim 1, wherein R^{X} is monovalent, and optionally wherein —R^{X} is any saturated hydrocarbyl group, wherein the hydrocarbyl group may be straight-chained or branched, or be or include cyclic groups, wherein the hydrocarbyl group may optionally be substituted with one or more groups selected from halo, —CN, —OH, —NH₂, oxo (═O) and ═NH, wherein the hydrocarbyl group includes at least one amide group in its carbon skeleton, and wherein the hydrocarbyl group may optionally include one, two or three further heteroatoms N and/or O in its carbon skeleton.

3. (canceled)

4. A compound or a pharmaceutically acceptable salt, solvate or prodrug thereof, as claimed in claim 1, wherein the 5-membered heteroaryl group of R¹ is monocyclic.

5. A compound or a pharmaceutically acceptable salt, solvate or prodrug thereof, as claimed in claim 1, wherein R^{X} is divalent, and optionally wherein —R^{X}— is any saturated or unsaturated hydrocarbylene group, wherein the hydrocarbylene group may be straight-chained or branched, or be or include cyclic groups, wherein the hydrocarbylene group may optionally be substituted with one or more groups selected from halo, —CN, —OH, —NH₂, oxo (═O) and ═NH, wherein the hydrocarbylene group includes at least one amide group in its carbon skeleton, and wherein the hydrocarbylene group may optionally include one or more further heteroatoms N, O or S in its carbon skeleton.

6. (canceled)

7. A compound or a pharmaceutically acceptable salt, solvate or prodrug thereof, as claimed in claim 1, wherein R^{X} contains only atoms selected from the group consisting of carbon, hydrogen, nitrogen, oxygen and halogen atoms.

8. A compound or a pharmaceutically acceptable salt, solvate or prodrug thereof, as claimed in claim 1, wherein:
(i) R^{X} contains from 4 to 11 atoms other than hydrogen or halogen; and/or
(ii) R¹ contains from 9 to 16 atoms other than hydrogen or halogen.

9. A compound or a pharmaceutically acceptable salt, solvate or prodrug thereof, as claimed in claim 1, wherein
(i) the 5-membered heteroaryl group of R¹ contains at least one nitrogen or sulfur atom in the 5-membered ring structure; or
(ii) the 5-membered heteroaryl group of R¹ contains at least one nitrogen atom in the 5-membered ring structure; or
(iii) the 5-membered heteroaryl group of R¹ contains only carbon and nitrogen atoms in the 5-membered ring structure.

10-12. (canceled)

13. A compound or a pharmaceutically acceptable salt, solvate or prodrug thereof, as claimed in claim 1, wherein
(i) Q is O; and/or
(ii) the 5-membered heteroaryl group of R¹ is further substituted with one, two or three substituents independently selected from
halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR; —SH; —SR^{β}; —SOR^{β}; —SO₂H; —SO₂R^{β}; —SO₂NH₂; —SO₂NHR^{β}; —SO₂N(R^{β})₂; —R α-SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂ N(R^{β})₂; —NH₂; —NHR^{β}; —N(R^{β})₂; —R^{α}—NH₂; —R^{α}—NHR^{β}; —R^{α}—N(R^{β})₂; —CHO; —CORP; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β};
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH, oxo (═O), or 4- to 6-membered heterocyclic group.

14. A compound or a pharmaceutically acceptable salt, solvate or prodrug thereof, as claimed in claim 1, wherein R² is an aryl or a heteroaryl group, wherein the aryl or the heteroaryl group is substituted at the α-position, and wherein R² may optionally be further substituted, and optionally wherein:
(i) R² is an aryl or a heteroaryl group, wherein the aryl or the heteroaryl group is substituted at the α and α′ positions, and wherein R² may optionally be further substituted; or
(ii) R² is a fused aryl or a fused heteroaryl group, wherein a first cycloalkyl, cycloalkenyl, non-aromatic heterocyclic, aryl or heteroaryl ring is fused to the aryl or heteroaryl group across the α,β positions and a second cycloalkyl, cycloalkenyl, non-aromatic heterocyclic, aryl or heteroaryl ring is fused to the aryl or heteroaryl group across the α′,β′ positions, and wherein R² may optionally be further substituted.

15-16. (canceled)

17. A compound or a pharmaceutically acceptable salt, solvate or prodrug thereof, as claimed in claim 1, wherein R² is a cyclic group substituted at the α-position with a monovalent heterocyclic group or a monovalent aromatic group, wherein a ring atom of the heterocyclic or aromatic group is directly attached to the α-ring atom of the cyclic group, wherein the heterocyclic or aromatic group may optionally be substituted, and wherein the cyclic group may optionally be further substituted.

18. A compound or a pharmaceutically acceptable salt, solvate or prodrug thereof, as claimed in claim 1, wherein R² is a cyclic group substituted at the α and α′ positions, wherein R² may optionally be further substituted, and wherein optionally each substituent at the α and α′ positions comprises a carbon atom.

19-20. (canceled)

21. A compound or a pharmaceutically acceptable salt, solvate or prodrug thereof, as claimed in claim 1, wherein the compound is selected from the group consisting of:

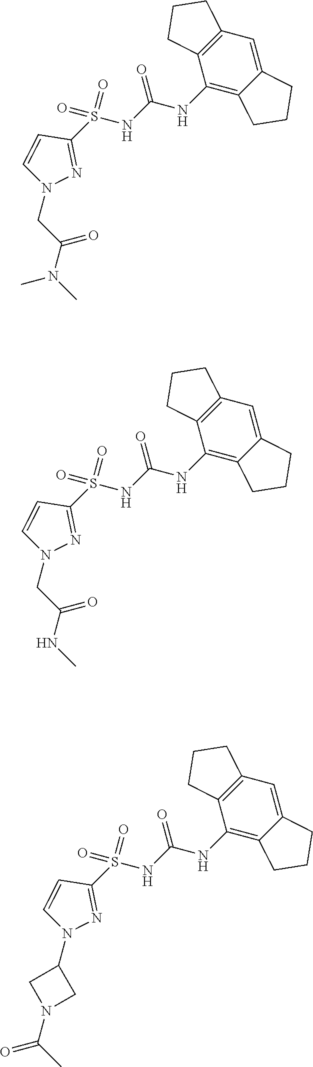 [SMILES: CN(C)C(=O)Cn1ccc(S(=O)(=O)NC(=O)Nc2c3c(cc4c2CCC4)CCC3)n1 ; CNC(=O)Cn1ccc(S(=O)(=O)NC(=O)Nc2c3c(cc4c2CCC4)CCC3)n1 ; CC(=O)N1CC(n2ccc(S(=O)(=O)NC(=O)Nc3c4c(cc5c3CCC5)CCC4)n2)C1]

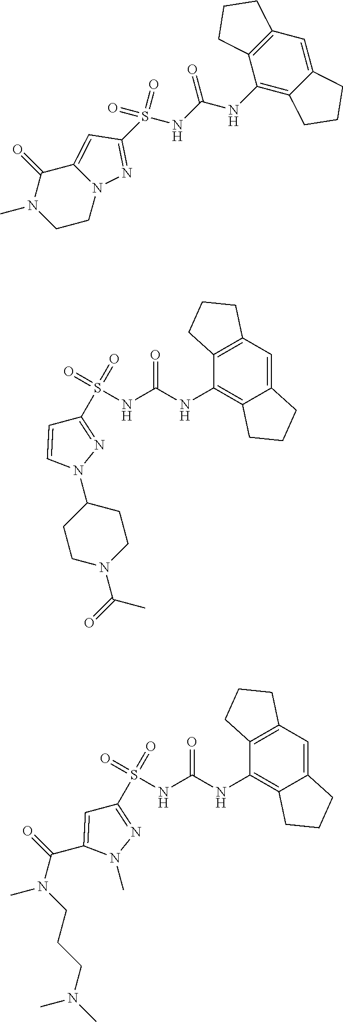 [SMILES: CN1CCn2nc(S(=O)(=O)NC(=O)Nc3c4c(cc5c3CCC5)CCC4)cc2C1=O ; CC(=O)N1CCC(n2ccc(S(=O)(=O)NC(=O)Nc3c4c(cc5c3CCC5)CCC4)n2)CC1 ; CN(C)CCCN(C)C(=O)c1cc(S(=O)(=O)NC(=O)Nc2c3c(cc4c2CCC4)CCC3)nn1C]

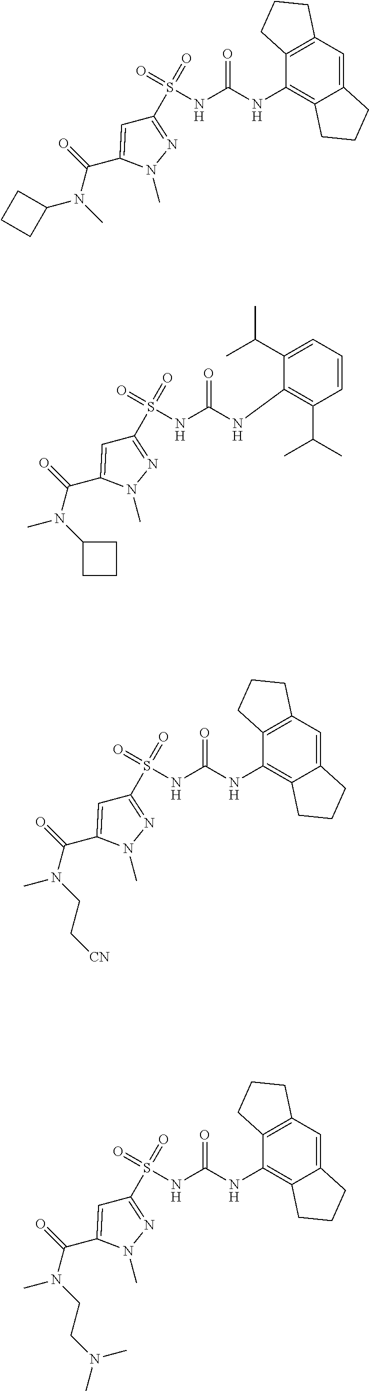

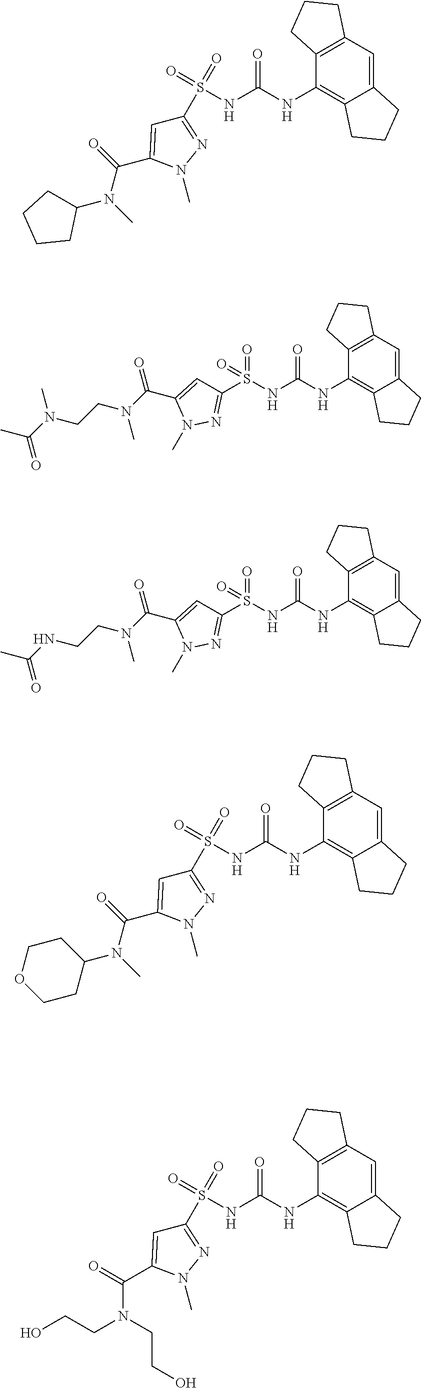 [SMILES: CN(C(=O)c1cc(S(=O)(=O)NC(=O)Nc2c3c(cc4c2CCC4)CCC3)nn1C)C1CCCC1 ; CC(=O)N(C)CCN(C)C(=O)c1cc(S(=O)(=O)NC(=O)Nc2c3c(cc4c2CCC4)CCC3)nn1C ; CC(=O)NCCN(C)C(=O)c1cc(S(=O)(=O)NC(=O)Nc2c3c(cc4c2CCC4)CCC3)nn1C ; CN(C(=O)c1cc(S(=O)(=O)NC(=O)Nc2c3c(cc4c2CCC4)CCC3)nn1C)C1CCOCC1 ; Cn1nc(S(=O)(=O)NC(=O)Nc2c3c(cc4c2CCC4)CCC3)cc1C(=O)N(CCO)CCO]

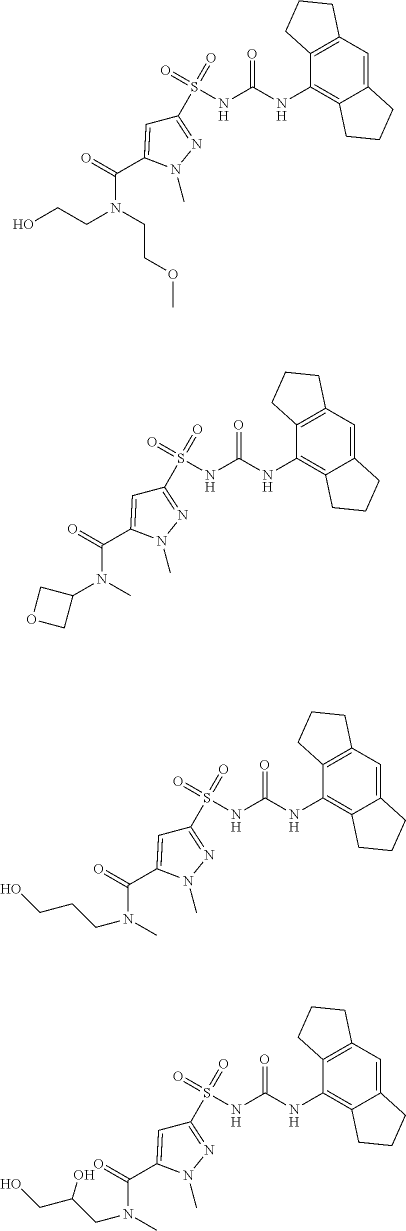 [SMILES: COCCN(CCO)C(=O)c1cc(S(=O)(=O)NC(=O)Nc2c3c(cc4c2CCC4)CCC3)nn1C ; CN(C(=O)c1cc(S(=O)(=O)NC(=O)Nc2c3c(cc4c2CCC4)CCC3)nn1C)C1COC1 ; CN(CCCO)C(=O)c1cc(S(=O)(=O)NC(=O)Nc2c3c(cc4c2CCC4)CCC3)nn1C ; CN(CC(O)CO)C(=O)c1cc(S(=O)(=O)NC(=O)Nc2c3c(cc4c2CCC4)CCC3)nn1C]

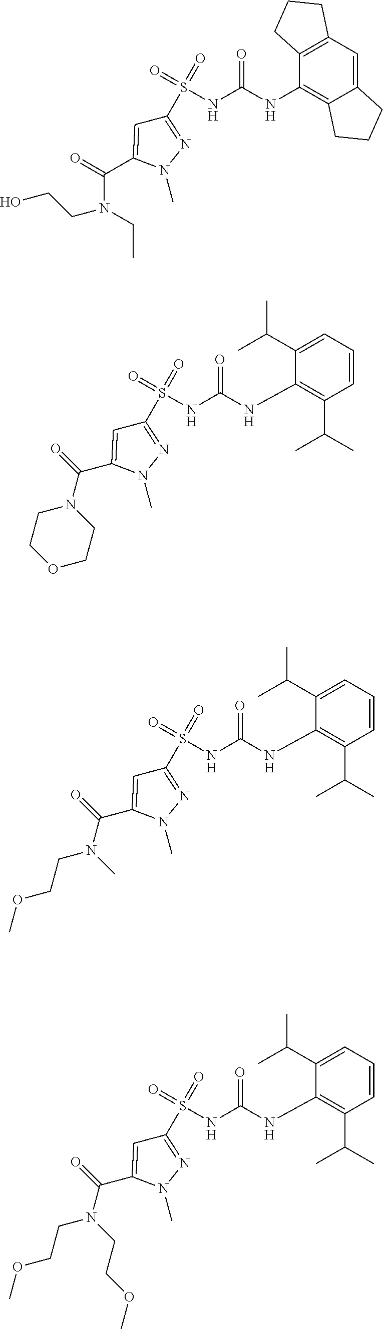

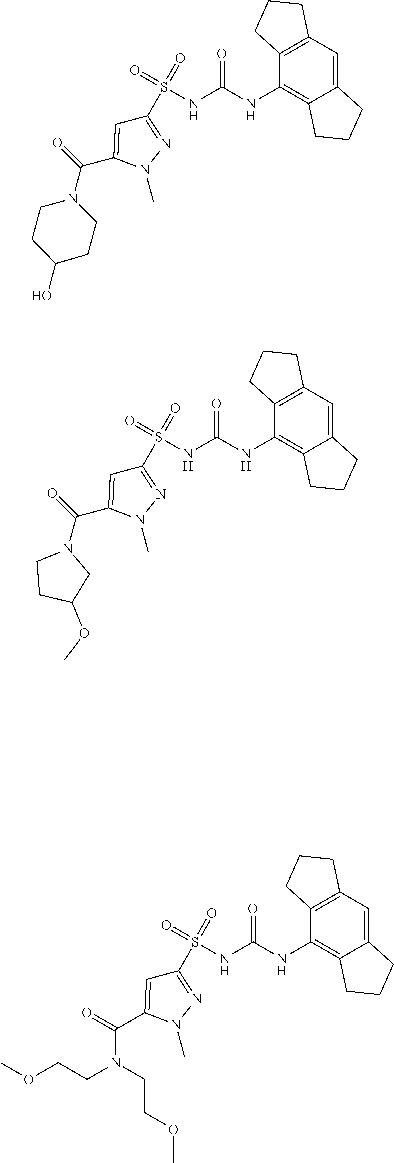 [SMILES: Cn1nc(S(=O)(=O)NC(=O)Nc2c3c(cc4c2CCC4)CCC3)cc1C(=O)N1CCC(O)CC1 ; COC1CCN(C(=O)c2cc(S(=O)(=O)NC(=O)Nc3c4c(cc5c3CCC5)CCC4)nn2C)C1 ; COCCN(CCOC)C(=O)c1cc(S(=O)(=O)NC(=O)Nc2c3c(cc4c2CCC4)CCC3)nn1C]

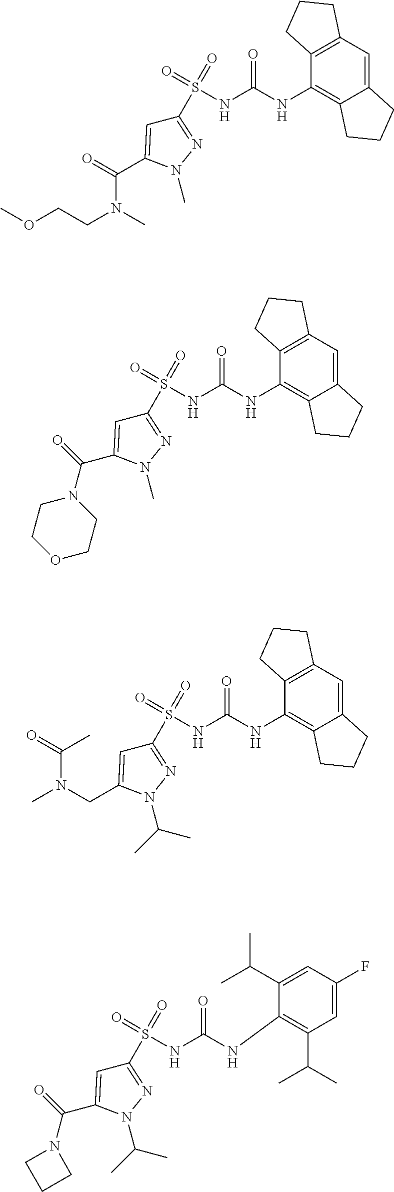 [SMILES: COCCN(C)C(=O)c1cc(S(=O)(=O)NC(=O)Nc2c3c(cc4c2CCC4)CCC3)nn1C ; Cn1nc(S(=O)(=O)NC(=O)Nc2c3c(cc4c2CCC4)CCC3)cc1C(=O)N1CCOCC1 ; CC(=O)N(C)Cc1cc(S(=O)(=O)NC(=O)Nc2c3c(cc4c2CCC4)CCC3)nn1C(C)C ; CC(C)c1cc(F)cc(C(C)C)c1NC(=O)NS(=O)(=O)c1cc(C(=O)N2CCC2)n(C(C)C)n1]

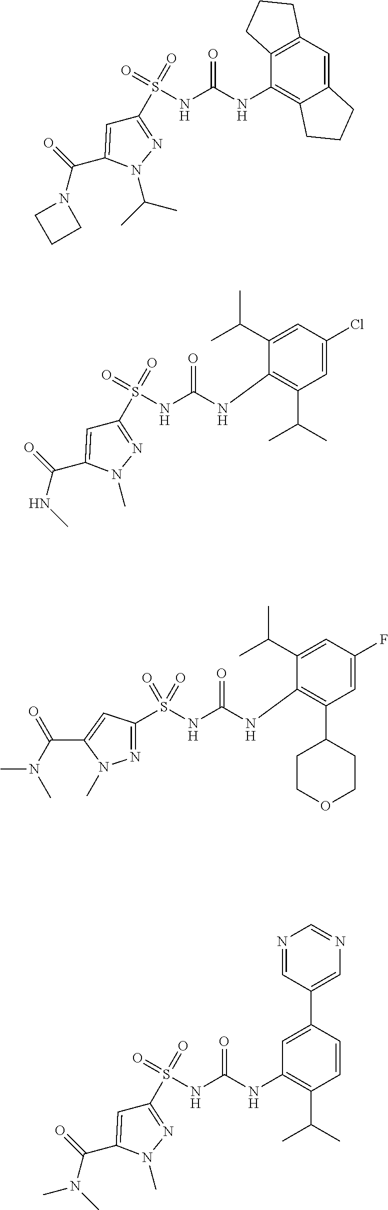 [SMILES: CC(C)n1nc(S(=O)(=O)NC(=O)Nc2c3c(cc4c2CCC4)CCC3)cc1C(=O)N1CCC1 ; CNC(=O)c1cc(S(=O)(=O)NC(=O)Nc2c(C(C)C)cc(Cl)cc2C(C)C)nn1C ; CC(C)c1cc(F)cc(C2CCOCC2)c1NC(=O)NS(=O)(=O)c1cc(C(=O)N(C)C)n(C)n1 ; CC(C)c1ccc(-c2cncnc2)cc1NC(=O)NS(=O)(=O)c1cc(C(=O)N(C)C)n(C)n1]

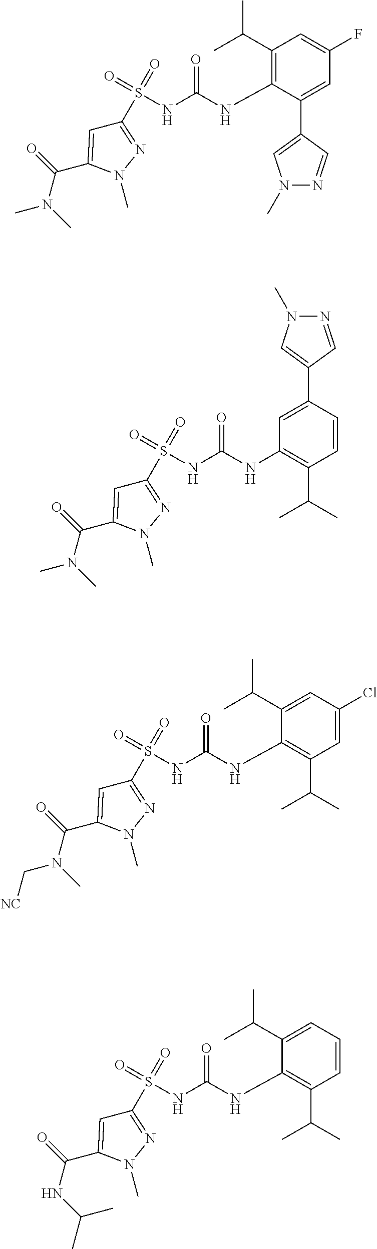 [SMILES: CC(C)c1cc(F)cc(-c2cnn(C)c2)c1NC(=O)NS(=O)(=O)c1cc(C(=O)N(C)C)n(C)n1 ; CC(C)c1ccc(-c2cnn(C)c2)cc1NC(=O)NS(=O)(=O)c1cc(C(=O)N(C)C)n(C)n1 ; CC(C)c1cc(Cl)cc(C(C)C)c1NC(=O)NS(=O)(=O)c1cc(C(=O)N(C)CC#N)n(C)n1 ; CC(C)NC(=O)c1cc(S(=O)(=O)NC(=O)Nc2c(C(C)C)cccc2C(C)C)nn1C]

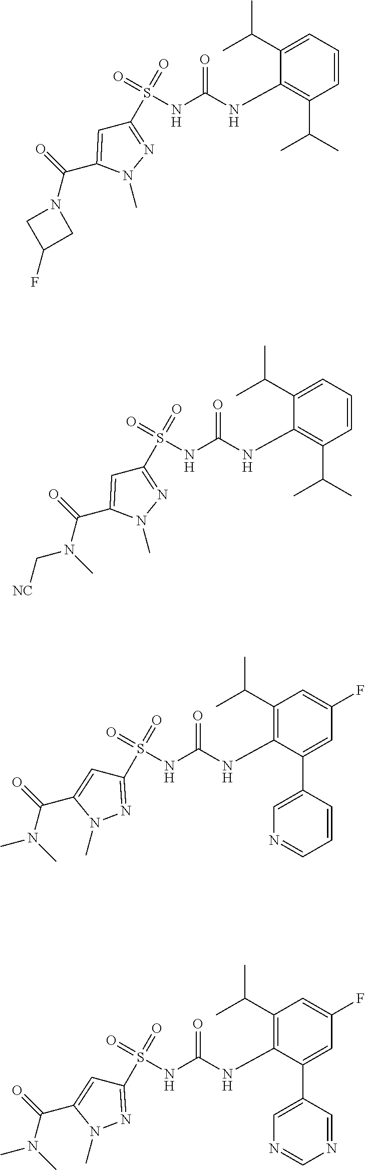

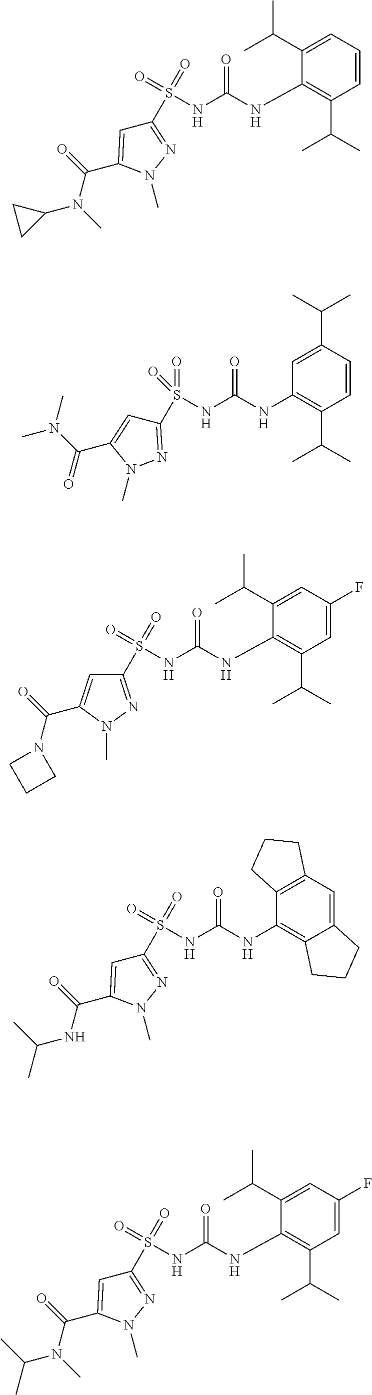

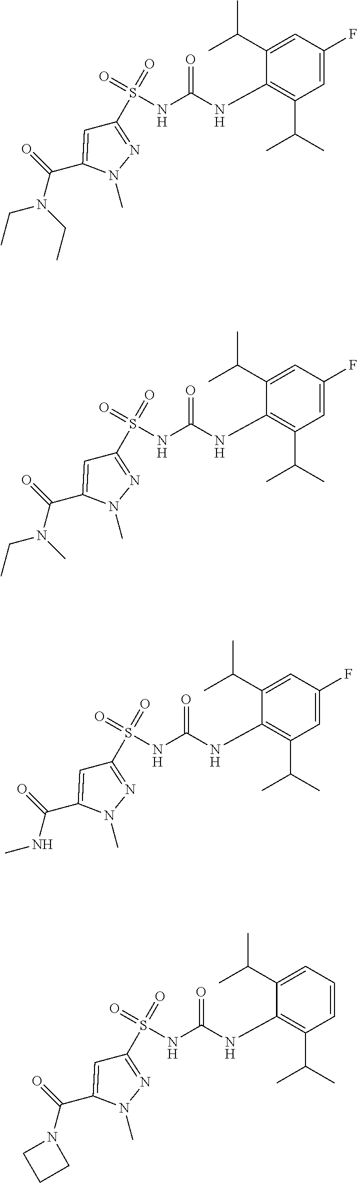 [SMILES: CCN(CC)C(=O)c1cc(S(=O)(=O)NC(=O)Nc2c(C(C)C)cc(F)cc2C(C)C)nn1C ; CCN(C)C(=O)c1cc(S(=O)(=O)NC(=O)Nc2c(C(C)C)cc(F)cc2C(C)C)nn1C ; CNC(=O)c1cc(S(=O)(=O)NC(=O)Nc2c(C(C)C)cc(F)cc2C(C)C)nn1C ; CC(C)c1cccc(C(C)C)c1NC(=O)NS(=O)(=O)c1cc(C(=O)N2CCC2)n(C)n1]

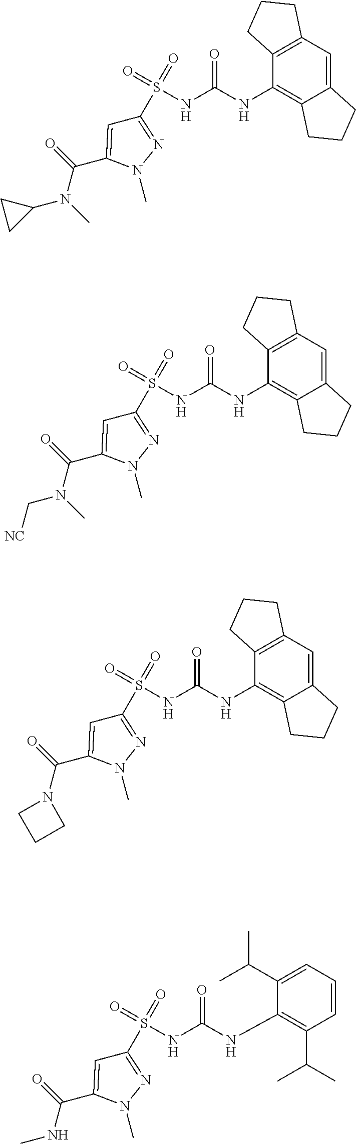 [SMILES: CN(C(=O)c1cc(S(=O)(=O)NC(=O)Nc2c3c(cc4c2CCC4)CCC3)nn1C)C1CC1 ; CN(CC#N)C(=O)c1cc(S(=O)(=O)NC(=O)Nc2c3c(cc4c2CCC4)CCC3)nn1C ; Cn1nc(S(=O)(=O)NC(=O)Nc2c3c(cc4c2CCC4)CCC3)cc1C(=O)N1CCC1 ; CNC(=O)c1cc(S(=O)(=O)NC(=O)Nc2c(C(C)C)cccc2C(C)C)nn1C]

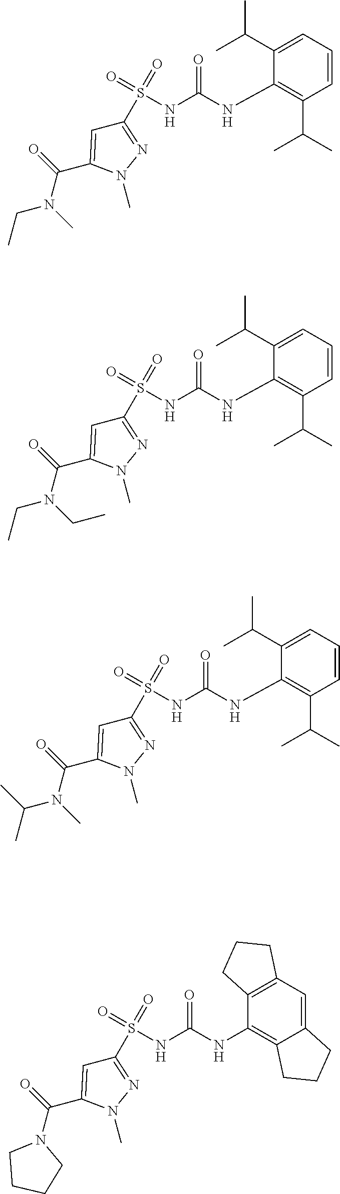

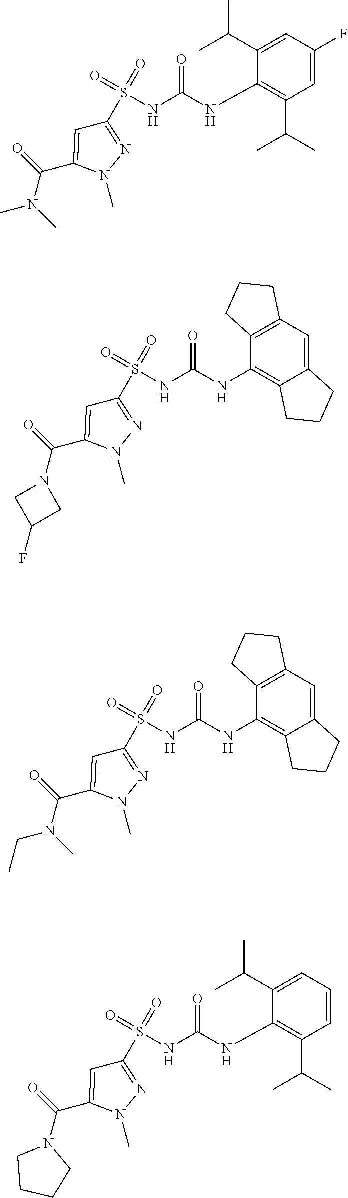 [SMILES: CC(C)c1cc(F)cc(C(C)C)c1NC(=O)NS(=O)(=O)c1cc(C(=O)N(C)C)n(C)n1 ; Cn1nc(S(=O)(=O)NC(=O)Nc2c3c(cc4c2CCC4)CCC3)cc1C(=O)N1CC(F)C1 ; CCN(C)C(=O)c1cc(S(=O)(=O)NC(=O)Nc2c3c(cc4c2CCC4)CCC3)nn1C ; CC(C)c1cccc(C(C)C)c1NC(=O)NS(=O)(=O)c1cc(C(=O)N2CCCC2)n(C)n1]

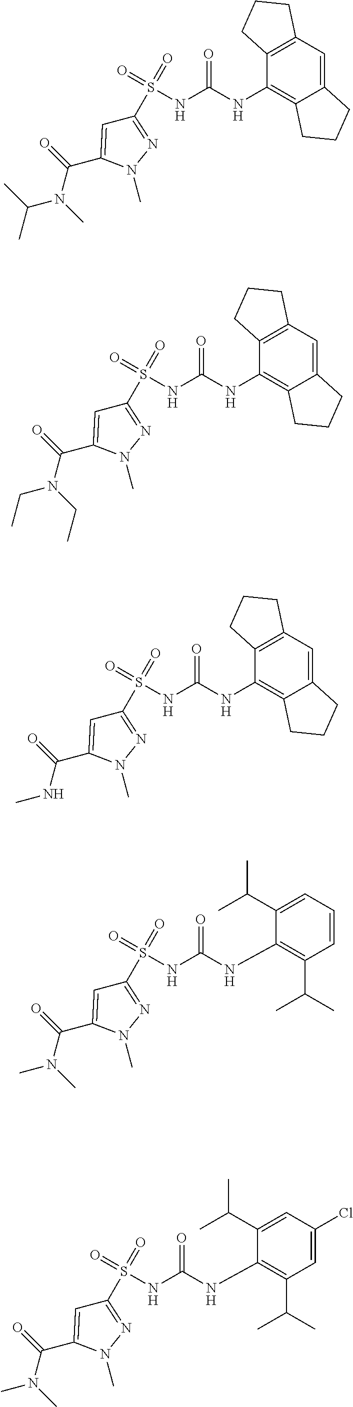

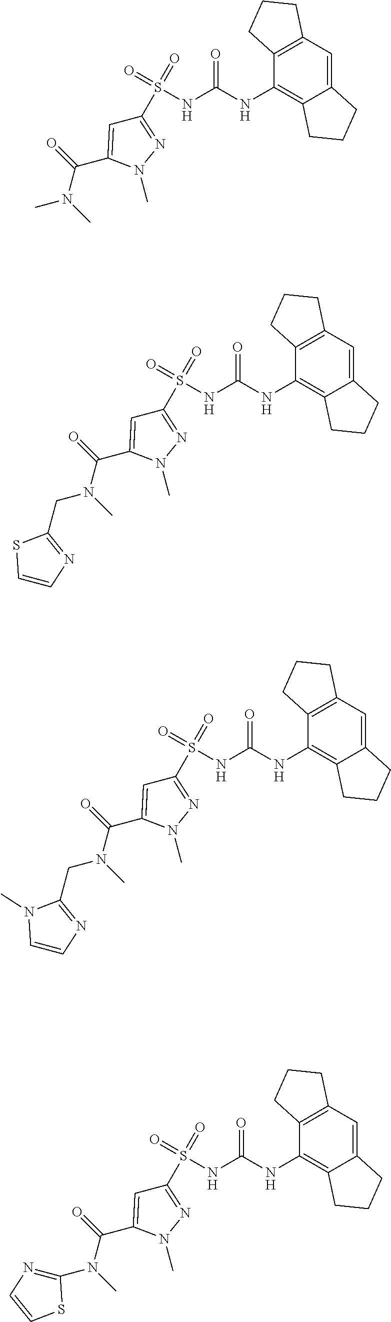 [SMILES: CN(C)C(=O)c1cc(S(=O)(=O)NC(=O)Nc2c3c(cc4c2CCC4)CCC3)nn1C ; CN(Cc1nccs1)C(=O)c1cc(S(=O)(=O)NC(=O)Nc2c3c(cc4c2CCC4)CCC3)nn1C ; CN(Cc1nccn1C)C(=O)c1cc(S(=O)(=O)NC(=O)Nc2c3c(cc4c2CCC4)CCC3)nn1C ; CN(C(=O)c1cc(S(=O)(=O)NC(=O)Nc2c3c(cc4c2CCC4)CCC3)nn1C)c1nccs1]

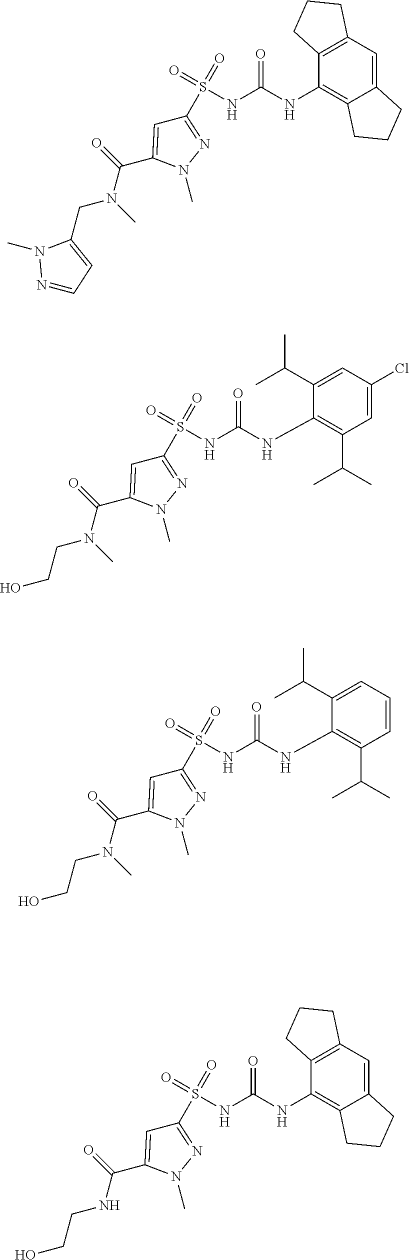 [SMILES: CN(Cc1ccnn1C)C(=O)c1cc(S(=O)(=O)NC(=O)Nc2c3c(cc4c2CCC4)CCC3)nn1C ; CC(C)c1cc(Cl)cc(C(C)C)c1NC(=O)NS(=O)(=O)c1cc(C(=O)N(C)CCO)n(C)n1 ; CC(C)c1cccc(C(C)C)c1NC(=O)NS(=O)(=O)c1cc(C(=O)N(C)CCO)n(C)n1 ; Cn1nc(S(=O)(=O)NC(=O)Nc2c3c(cc4c2CCC4)CCC3)cc1C(=O)NCCO]

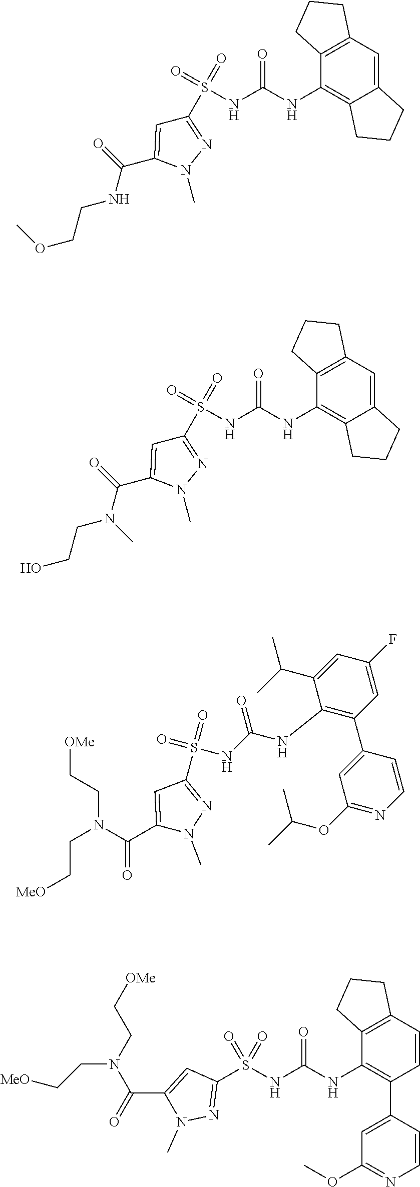 [SMILES: COCCNC(=O)c1cc(S(=O)(=O)NC(=O)Nc2c3c(cc4c2CCC4)CCC3)nn1C ; CN(CCO)C(=O)c1cc(S(=O)(=O)NC(=O)Nc2c3c(cc4c2CCC4)CCC3)nn1C ; COCCN(CCOC)C(=O)c1cc(S(=O)(=O)NC(=O)Nc2c(-c3ccnc(OC(C)C)c3)cc(F)cc2C(C)C)nn1C ; COCCN(CCOC)C(=O)c1cc(S(=O)(=O)NC(=O)Nc2c(-c3ccnc(OC)c3)ccc3c2CCC3)nn1C]

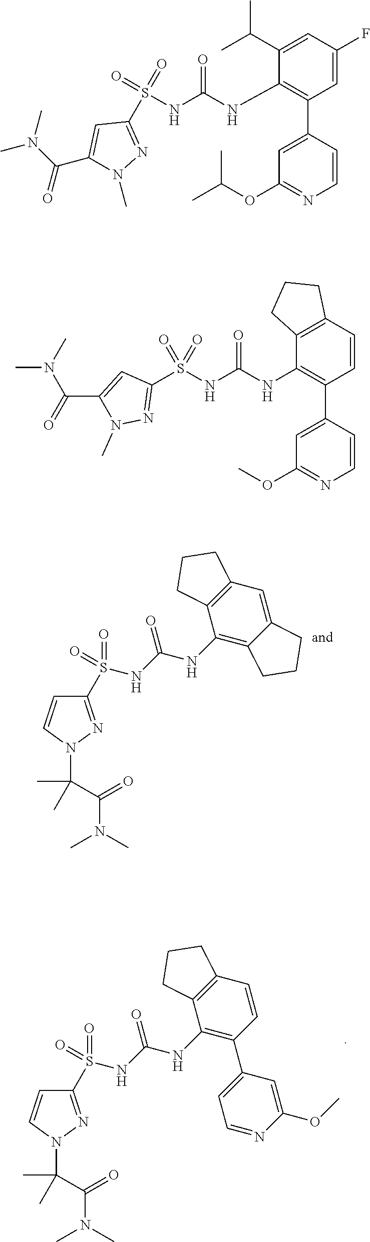 [SMILES: CC(C)Oc1cc(-c2cc(F)cc(C(C)C)c2NC(=O)NS(=O)(=O)c2cc(C(=O)N(C)C)n(C)n2)ccn1 ; COc1cc(-c2ccc3c(c2NC(=O)NS(=O)(=O)c2cc(C(=O)N(C)C)n(C)n2)CCC3)ccn1 ; CN(C)C(=O)C(C)(C)n1ccc(S(=O)(=O)NC(=O)Nc2c3c(cc4c2CCC4)CCC3)n1 ; COc1cc(-c2ccc3c(c2NC(=O)NS(=O)(=O)c2ccn(C(C)(C)C(=O)N(C)C)n2)CCC3)ccn1]

22. (canceled)

23. A pharmaceutical composition comprising a compound or a pharmaceutically acceptable salt, solvate or prodrug thereof, as claimed in claim 1, and a pharmaceutically acceptable excipient, optionally wherein the pharmaceutical composition is a topical pharmaceutical composition.

24. (canceled)

25. A method of treating or preventing a disease, disorder or condition in a subject, the method comprising the step of administering an effective amount of a compound or a pharmaceutically acceptable salt, solvate or prodrug thereof, as claimed in claim 1, to the subject, thereby treating or preventing the disease, disorder or condition, optionally wherein the disease, disorder or condition is responsive to NLRP3 inhibition.

26. (canceled)

27. The method as claimed in claim 25, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia; and
(xvii) any disease where an individual has been determined to carry a germline or somatic non-silent mutation in NLRP3.

28. The method as claimed in claim 27, wherein the disease, disorder or condition is selected from:
(i) a cardiovascular disease;
(ii) a liver disease;
(iii) a renal disease;
(iv) a psychological disorder;
(v) a lymphatic condition; and/or
(vi) any disease, disorder or condition in which an individual has been determined to carry a germline or somatic non-silent mutation in NLRP3.

29. The method as claimed in claim 25, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

30. A method of inhibiting NLRP3 in a subject, the method comprising administering a compound or a pharmaceutically acceptable salt, solvate or prodrug thereof, as claimed in claim 1, to the subject thereby inhibiting NLRP3.

31. A method of analysing inhibition of NLRP3 or an effect of inhibition of NLRP3 by a compound, comprising contacting a cell or non-human animal with a compound or a pharmaceutically acceptable salt, solvate or prodrug thereof, as claimed in claim 1, and analysing inhibition of NLRP3 or an effect of inhibition of NLRP3 in the cell or non-human animal by the compound.

32. The method as claimed in claim 25, wherein the compound is administered as a pharmaceutical composition further comprising a pharmaceutically acceptable excipient.
